(19) United States
(12) Patent Application Publication
(10) Pub. No.: US 2017/0285049 A1
(43) Pub. Date: Oct. 5, 2017

(54) Means and Methods for Diagnosing Heart Failure in a Subject

(71) Applicants: METANOMICS GMBH, Berlin (DE); Ruprecht-Karls-Universität Heidelberg, Heidelberg (DE)

(72) Inventors: Philipp Schatz, Berlin (DE); Henning Witt, Berlin (DE); Martin Dostler, Henningsdorf (DE); Susan Carvalho, Berlin (DE); Erik Peter, Postsdam (DE); Philipp Ternes, Dallgow (DE); Philipp Mappes, Berlin (DE); Hans Dirk, Duengen (BE); Tobias Daniel, Trippel (DE); Elvis Tahirovic, Berlin (DE); Hugo Katus, Heidelberg (DE); Norbert Frey, Kronshagen (DE); Tanja Weis, Wiesenbach (DE)

(21) Appl. No.: 15/507,801

(22) PCT Filed: Sep. 1, 2015
(86) PCT No.: PCT/EP2015/069979

§ 371 Date: Mar. 1, 2017

Related U.S. Application Data
(60) Provisional application No. 62/128,586, filed on Mar. 5, 2015.
(60) Provisional application No. 62/044,367, filed on Sep. 1, 2014.

(30) Foreign Application Priority Data
Sep. 1, 2014 (EP) 14183105.7

(51) Int. Cl. G01N 33/92

(52) U.S. Cl. CPC G01N 33/92; G01N 2800/325; G01N 2405/08; G01N 2405/02; G01N 2405/04; G01N 2405/00

(57) ABSTRACT

The present invention relates to the field of diagnostic methods. Specifically, the present invention contemplates a method for diagnosing heart failure in a subject based on determining the amounts of at least three biomarkers. The invention also relates to tools for carrying out the aforementioned methods, such as diagnostic devices.

The present invention relates to the field of diagnostic methods. Specifically, the present invention contemplates a method for diagnosing heart failure in a subject based on determining the amounts of at least three biomarkers. The invention also relates to tools for carrying out the aforementioned methods, such as diagnostic devices.

Heart failure is a severe problem in modern medicine. The impaired function of the heart can give rise to life-threatening conditions and results in discomfort for the patients suffering from heart failure. Heart failure can affect the right or the left heart ventricle, respectively, and can vary in strength. A classification system was originally developed by the New York Heart Association (NYHA). According to the classification system, the mild cases of heart failure are categorized as class I cases. These patients only show symptoms under extreme exercise. The intermediate cases show more pronounced symptoms already under less exercise (classes II and III) while class IV, shows already symptoms at rest (New York Heart Association. Diseases of the heart and blood vessels. Nomenclature and criteria for diagnosis, 6th ed. Boston: Little, Brown and co, 1964; 114).

The prevalence of heart failure steadily increases in the population of the western developed countries over the last years. One reason for said increase can be seen in an increased average life expectation and improved survival after myocardial infarction due to modern medicine. The mortality rate caused by heart failure, however, could be further reduced by improved diagnostic and therapeutic approaches. The so-called “Framingham” study reported a reduction of the 5 year mortality from 70% to 59% in men and from 57% to 45% in women when comparing a time window of 1950 to 1969 with 1990 to 1999 (Fox C. S. et al., Circulation 110, 522-527, 2004). The “Mayo” study shows a reduction from 65% to 50% for men for a time window of 1996 to 2000 compared to 1979 to 1984 and from 51% to 46% for women (Roger V L, Weston S A, Redfield M M, et al., JAMA-J. Am. Med. Assoc. 292, 344-350, 2004). Notwithstanding this reduction of the mortality rate, the overall mortality due to heart failure is still a major burden to societies. One-year mortality for NYHA class II to III patients under ACE inhibitor therapy is still between 9-12% (SOLVD study; The SOLVD Investigators, NEJM 325, 293-302, 1991; The SOLVD Investigators, New Engl. J. Med. 327, 685-691, 1992) and for NYHA class IV without ACE inhibitor therapy 52% (Consensus study; The Consensus Trial Study Group, New Engl. J. Med. 316, 1429-1435, 1987).

Diagnosis of heart failure today relies predominantly on clinical symptoms, imaging modalities and the biomarkers, brain natriuretic peptide (BNP) and amino-terminal pro-brain natriuretic peptide (NT-proBNP). However, despite a wide acceptance of biomarkers in the medical community, the clinical utility of the gold standards BNP and NT-proBNP is limited by their lack of sensitivity in early stages of heart failure (see Rodeheffer, R. J., J. Am. Coll. Cardiol. 44, 740-749, 2004). In addition, BNP and/or NT-proBNP concentrations are influenced by confounding factors such as gender, age, BMI, or renal function [Balion C. et al. 2013. AHRQ Comparative Effectiveness Reviews. Agency for Healthcare Research and Quality (US), Rockville (Md.)]. This represents a huge medical problem since many patients progress through an asymptomatic phase of left ventricular systolic dysfunction (ALVSD) before the development of overt symptomatic disease. Because pharmacotherapy in asymptomatic patients was shown to positively affect clinical outcome, a reliable diagnosis of treatable early stages and in consequence, guideline-driven treatment is expected to reduce morbidity and mortality and have a high health economic impact. While there is little doubt within the medical community about the need for a cost effective screening for ALSVD (patients at risk to develop symptomatic heart failure), prohibitive costs and/or suboptimal clinical performance of the available diagnostic tests have so far prevented any screening program from being advocated by medical guidelines.

Recently, metabolic biomarkers for diagnosing heart failure and/or for monitoring heart failure progression or regression have been reported (see WO2011/092285 and WO2013/014286, respectively).

It is a goal of modern medicine to reliably identify and treat patients with heart failure, in particular with early stages of heart failure. Accordingly, means and methods for reliably diagnosing heart failure are still highly desired.

Therefore, the present invention relates to a method for diagnosing heart failure in a subject comprising the steps of:
a) determining in a sample of a subject the amounts of, in particular, at least three biomarkers; and
b) comparing the amounts of the said biomarkers as determined in step a) to a reference (or references, in particular a reference for each of the at least three biomarkers), whereby heart failure is to be diagnosed.

The method as referred to in accordance with the present invention includes a method which essentially consists of the aforementioned steps or a method which includes further steps. However, it is to be understood that the method, in a preferred embodiment, is a method carried out ex vivo, i.e. not practised on the human or animal body. The method, preferably, can be assisted by automation.

The term “diagnosing” as used herein refers to assessing whether a subject suffers from heart failure, or not. Accordingly, the presence or the absence of heart failure in the subject can be diagnosed. Preferably, the term refers to ruling in or ruling out heart failure. As will be understood by those skilled in the art, such an assessment, although preferred to be, may usually not be correct for 100% of the investigated subjects. The term, however, requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2 or lower, 0.1 or lower, or 0.05 or lower. Preferably, the diagnosis is based on the biomarkers to be determined in the method of the present invention, i.e. on the at least three biomarkers as specifically referred to in step a) of the method of the present invention and, if determined, on NT-proBNP or BNP.

The term “diagnosing” includes individual diagnosis of heart failure or its symptoms as well as continuous monitoring of a patient. Monitoring, i.e. diagnosing the presence or absence of heart failure or the symptoms accompanying it at various time points, includes monitoring of patients known to suffer from heart failure as well as monitoring of subjects known to be at risk of developing heart failure, in particular symptomatic heart failure. Furthermore, monitoring can also be used to determine whether a patient is treated successfully or whether at least symptoms of heart failure can be ameliorated over time by a certain therapy. Moreover, monitoring may be used for active patient management including deciding on hospitalization, intensive care measures and/or additional qualitative monitoring as well as quantitative monitoring measures, i.e. monitoring frequency.

In an embodiment, the presence of the disease in the test subject could be diagnosed. In another embodiment, the absence of the disease in the test subject could be diagnosed.

The term “heart failure” is well known in the art. As used herein relates to an impaired function of the heart. It is a progressive disorder in which the heart fails to pump oxygenated blood at a rate sufficient to meet the metabolic needs of the tissues. Preferably, the term heart failure as used herein relates to congestive heart failure (CHF).

Heart failure is the final common stage of many cardiovascular diseases and is defined as a clinical syndrome in which patients in the final stage show typical signs and symptoms of effort intolerance and/or fluid retention resulting from an abnormality of cardiac structure or function. As outlined further herein below, the term “heart failure” in the context of the method of the present invention encompasses both symptomatic forms and asymptomatic forms of heart failure. E.g. asymptomatic left ventricular systolic dysfunction or asymptomatic diastolic dysfunction may be diagnosed.

The impaired function of the heart can be a systolic dysfunction resulting in a significantly reduced ejection fraction of blood from the heart and, thus, a reduced blood flow. Specifically, systolic heart failure is characterized by a significantly reduced left ventricular ejection fraction (LVEF), preferably, an ejection fraction of less than 50% (heart failure with reduced ejection fraction, HFrEF). Alternatively, the impairment can be a diastolic dysfunction, i.e. a failure of the ventricle to properly relax. The latter is usually accompanied by a stiffer ventricular wall. The diastolic dysfunction causes inadequate filling of the ventricle and, therefore, results in consequences for the blood flow, in general. Thus, diastolic dysfunction also results in elevated end-diastolic pressures, and the end result is comparable to the case of systolic dysfunction (pulmonary edema in left heart failure, peripheral edema in right heart failure.) Heart failure may, thus, affect the right heart (pulmonary circulation), the left heart (body circulation) or both. Techniques for measuring an impaired heart function and, thus, heart failure, are well known in the art and include echocardiography, electrophysiology, angiography. It will be understood that the impaired function of the heart can occur permanently or only under certain stress or exercise conditions. Dependent on the strength of the symptoms, heart failure can be classified as set forth elsewhere herein. Typical symptoms of heart failure include dyspnea, chest pain, dizziness, confusion, pulmonary and/or peripheral edema. It will be understood that the occurrence of the symptoms as well as their severity may depend on the severity of heart failure and the characteristics and causes of the heart failure, systolic or diastolic or restrictive i.e. right or left heart located heart failure. Further symptoms of heart failure are well known in the art and are described in the standard text books of medicine, such as Stedman or Brunnwald.

Two subsets of heart failure with different pathophysiology are described based on a measurement of left ventricular ejection fraction (LVEF), which is the percentage of the total amount of blood in the left ventricle that is pushed out with each heartbeat: Heart failure with reduced left ventricular ejection fraction (HFrEF) and heart failure with preserved left ventricular ejection fraction (HFpEF). Preferably, the heart failure to be diagnosed in the accordance with the present invention is HFpEF, more preferably the heart failure is HFrEF.

HFrEF, also known as systolic heart failure, is characterized by reduced heart muscle contraction and emptying of the left ventricle. The expression “reduced left ventricular ejection fraction”, preferably, relates to a left ventricular ejection fraction (LVEF) of lower than 50%. Moreover it is envisaged that the LVEF is lower than 40%, in particular lower than 35%. Also the left ventricular ejection fraction may be lower than 50% but larger than 35%.

While there are many causes of HFrEF, the most common is related to ischemic cardiomyopathy resulting from coronary artery disease and prior myocardial infarctions. Ischemic cardiomyopathy (ICMP) occurs when narrowed or blocked coronary arteries restrict blood flow and oxygen supply to the heart tissue, damaging or weakening the heart muscle (loss of functional myocardium). Further, HfrEF may have non-ischemic causes. One of the most common non-ischemic causes of HFrEF is dilatative cardiomyopathy (DCMP, also referred to as dilated cardiomyopathy). DCMP is a condition in which the heart's ability to pump blood is decreased because the heart's main pumping chamber, the left ventricle, is enlarged, dilated and weak. The cause for DCMP can range from heart muscle infection (myocarditis), high blood pressure, heart valve disease, and alcohol abuse to familial (hereditary) forms. Preferably, the terms NICMP (non-ischemic cardiomyopathy) and DCMP are used interchangeably herein.

A subject who suffers from HFrEF may, thus, suffer from dilatative cardiomyopathy (DCMP, frequently also referred to as dilated cardiomyopathy) or ischemic cardiomyopathy (ICMP).

Accordingly, the term HFrEF preferably encompasses ICMP and DCMP. Thus, the HFrEF to be diagnosed may be ICMP or DCMP. Preferred marker combinations for HFrEF, ICMP and DCMP are disclosed elsewhere herein.

A subject who suffers from ICMP, preferably, has a reduced LVEF and more than 50% coronary stenosis.

A subject who suffers from DCMP, preferably, has a reduced LVEF and less than 50% coronary stenosis. In particular, a subject who suffers from DCMP, preferably, has a reduced LVEF, less than 50% coronary stenosis and a left ventricle wall thickness>55 mm. Further, a subject who suffers from DCMP may have a reduced LVEF, less than 50% coronary stenosis and a left ventricular end diastolic diameter of larger than 55 mm.

Preferred combinations of at least three biomarkers for diagnosing HFrEF, or the subforms of HFrEF (DCMP or ICMP) are disclosed elsewhere herein.

HFrEF in accordance with the method of the present invention may be symptomic or asymptomatic. Accordingly, the present invention preferably allows for the diagnosis of symptomic or asymptomatic systolic dysfunction.

As set forth above, the heart failure to be diagnosed may be heart failure with preserved left ventricular ejection fraction (HFpEF) also known as diastolic heart failure. The term “preserved left ventricular ejection fraction” preferably refers to a LVEF of larger than 50%. Also envisaged is a LVEF of larger than 60%. Alternatively, the term “preserved left vetricular ejection fraction” preferably refers to a LVEF of larger than 55%. HFpEF is characterized by a disturbed relaxation and dilatation of the left ventricle. Diastolic left ventricular dysfunction results from LV hypertrophy and cardiac fibrosis resulting in increased myocardial stiffness. The muscle becomes stiff and loses some of its ability to relax, especially during diastole. As a result, the affected chamber has trouble filling with blood during diastole, leading to an elevated left ventricular filling pressure. HFpEF in accordance with the method of the present invention may be symptomic or asymptomatic. Accordingly, the present invention preferably allows for the diagnosis of symptomic or asymptomatic diastolic dysfunction.

A subject suffering from HFpEF preferably has a cardium septum thickness of larger than 12 mm. Alternatively, the subject may have a cardium septum thickness of larger than 11 mm.

Preferred combinations of at least three biomarkers for diagnosing HFpEF are disclosed elsewhere herein.

Moreover, heart failure as used herein relates to symptomatic or asymptomatic heart failure. Accordingly, the method of the present invention allows for diagnosing symptomatic heart failure and, in particular, asymptomatic heart failure. Thus, the present invention, in particular, allows for diagnosing symptomatic or asymptomic CHF, HFrEF, DCMP, ICMP and/or HFpEF.

Symptoms of heart failure are well known in the art and are described above. Asymptomatic heart failure is preferably heart failure according to NYHA class I. A subject with heart failure according to NYHA class I has no limitation of physical activity and ordinary physical activity does not cause undue breathlessness, fatigue, or palpitations. Symptomatic heart failure is preferably heart failure according to NYHA class II, III and/or VI, in particular according to NYHA class II and/or III. A subject with heart failure according to NYHA class II or III has a slight (NYHA class II) or marked (NYHA class III) limitation of physical activity, is comfortable at rest but ordinary (NYHA class II) or less than ordinary (NYHA class III) physical activity results in undue breathlessness, fatigue, or palpitations.

As described herein below in more detail, the subject to be tested preferably shows symptoms of heart failure. In this case, it is in particular diagnosed whether the subject suffers from symptomatic heart failure (or symptomatic HFrEF, DCMP, ICMP or HFpEF). More preferably, the subject does not show symptoms of heart failure. In this case asymptomatic heart failure is diagnosed. In this case, it is in particular diagnosed whether the subject suffers from asymptomatic heart failure (or asymptomatic HFrEF, DCMP, ICMP or HFpEF).

The method of the present invention envisages the determination of the amount of at least three biomarkers, i.e. of a combination of at least three biomarkers. Preferred combinations are described elsewhere herein. The term “biomarker” as used herein refers to a molecular species which serves as an indicator for a disease or effect as referred to in this specification. Said molecular species can be a metabolite itself which is found in a sample of a subject. Moreover, in certain cases the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective medical condition, i.e. for heart failure. In an embodiment of the present invention the triacylglyceride(s) will be determined as such, as disclosed elsewhere herein. In an embodiment of the present invention the phosphatidylcholine(s) will be determined as such, as disclosed elsewhere herein. In an embodiment of the present invention the ceramide(s) will be determined as such, as disclosed elsewhere herein. In an embodiment of the present invention the sphingomyelin(s) will be determined as such, as disclosed elsewhere herein. In an embodiment of the present invention the cholesterylester(s) will be determined as such, as disclosed elsewhere herein. Preferably, the at least three biomarkers to be determined in accordance with the present invention are metabolite biomarkers (with the exception of the further markers BNP and NT-proBNP which are peptides/protein markers, see below). The term “metabolite” is well known in art. Preferably, the metabolite in accordance with the present invention is a small molecule compound.

In the method according to the present invention at least the amounts of at least three biomarkers shall be determined (i.e. of at least three metabolite biomarkers). The term “at least three biomarkers” as used herein, means three or more than three. Accordingly, the amounts of three, four, five, six, seven, eight, nine, ten or even more biomarkers may be determined (and compared to a reference). Preferably, the amounts of three to ten biomarkers, in particular metabolite biomarkers, are determined (and compared to a reference).

Preferably, the at least three biomarkers to be determined are lipid metabolite biomarkers. Thus, the biomarkers are preferably metabolite biomarkers (i.e. lipid biomarkers). More preferably, the at least three biomarkers to be determined are selected from the group consisting of at least one sphingomyelin biomarker, at least one triacylglyceride biomarker, at least one cholesterylester biomarker, at least one phosphatidylcholine biomarker, and at least one ceramide biomarker. Thus, the method of the present invention envisages the determination of one or more sphingomyelin biomarker, one or more triacylglyceride, biomarker, one or more cholesterylesters biomarker, one or more phosphatidylcholine biomarker, and/or one or more ceramide biomarker. Furthermore, the amount of glutamic acid may be determined. In an embodiment, the amounts of i) at least one sphingomyelin biomarker, ii) at least one triacylglyceride biomarker, and iii) at least one cholesterylester biomarker or at least one phosphatidylcholine biomarker are determined.

The at least three biomarkers to be determined preferably belong to at least three of the different compound classes referred to above. For example, one of the at least three biomarkers is a triacylglycerid biomarker, one is a sphingomyelin biomarker, and one is a cholesterylester biomarker. However it is also envisaged that the at least three biomarkers belong to a single compound class, or to two compound classes.

Preferred biomarkers to be determined in accordance with the present invention are disclosed in column 1 of Table 1 of the examples section.

Thus, the at least one triacylglycerid biomarker is preferably selected from the group consisting of OSS2, SOP2, SPP1, SSP2, SSS, PPO1, and PPP, more preferably selected from the group consisting of OSS2, SOP2, SPP1, SSP2, PPO1 and PPP, even more preferably selected from OSS2, SOP2, SPP1, SSP2 and PPO1, in particular OSS2 and SOP2, and most preferably OSS2. Preferably, one, two or three, or more triacylglyceride biomarker are determined.

The at least one sphingomyelin biomarker is preferably selected from the group consisting of SM10, SM18, SM2, SM21, SM23, SM24, SM28, SM29, SM3, SM5, SM8, and SM9 (in particular from SM10, SM18, SM21, SM23, SM24 and SM28). More preferably, the at least one sphingomyelin biomarker is selected from the group consisting of SM23, SM24, and SM18. Even more preferably, the at least one sphingomyelin biomarker is SM18 or SM24, and most preferably SM23. Preferably, one, two, three, four or more sphingomyelin biomarker are determined.

The at least one ceramide biomarker is preferably selected from the group consisting of Cer(d16:1/24:0), Cer(d17:1/24:0), Cer(d18:1/23:0), Cer(d18:1/24:1), and Cer(d18:2/24:0). Preferably, the amounts of one or two, or more ceramide biomarker are determined. More preferably, the at least one ceramide biomarker is selected from the group consisting of Cer(d16:1/24:0), and Cer(d18:1/24:1). Even more preferably, the at least one ceramide biomarker is Cer(d16:1/24:0).

The at least one phosphatidylcholine biomarker is preferably PC4 or PC8. Preferably, the amount of one phosphatidylcholine biomarker is determined, in particular PC4. However, it is also envisaged to determine both biomarker PC4 and PC8.

The at least one cholesterylester biomarker is preferably cholesterylester C18:0 or cholesterylester C18:2. Preferably, the amount of one cholesterylester biomarker is determined, in particular cholesterylester C18:2. However, it also envisaged to determine both cholesterylester C18:0 and cholesterylester C18:2. Preferably, the cholesterylester biomarker is not cholesterylester C18:1.

Accordingly, the at least three biomarkers as referred to in step a) of the method of the present invention are preferably selected from the group of biomarkers consisting of OSS2, SOP2, SPP1, SSP2, SSS, PPO1, PPP, SM10, SM18, SM2, SM21, SM23, SM24, SM28, SM29, SM3, SM5, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0 cholesterylester C18:2, and glutamic acid. The aforementioned biomarkers are metabolite biomarkers.

### Biomarker Definitions

In accordance with the present invention, the amounts of at least three metabolite biomarkers selected from the group consisting of OSS2, SOP2, SPP1, SSP2, SSS, PPO1, PPP, SM10, SM18, SM2, SM21, SM23, SM24, SM28, SM29, SM3, SM5, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0, cholesterylester C18:2 and glutamic acid shall be determined.

A preferred definition of these biomarkers is provided in Table 1 of the Examples section. Accordingly, the biomarkers are preferably defined as follows:

Triacylglyceride (TAG) Biomarkers

The biomarkers OSS2, SOP2, SPP1, SSP2, SSS, PPO1, and PPP are triacylglycerides (TAG: triacylglyceride).
OSS2 is TAG(C18:1, C18:0, C18:0)
PPO1 is TAG(C16:0, C16:0, C18:1)
PPP is TAG(C16:0, C16:0, C16:0)
SOP2 is TAG(C18:0, C18:1, C16:0)
SPP1 is TAG(C18:0, C16:0, C16:0)
SSP2 is TAG(C18:0, C18:0, C16:0)
SSS is TAG(C18:0, C18:0, C18:0)

The triacylglyceride TAG(Cx¹:y¹, Cx²:y², Cx³:y³) is preferably denoted to mean that the triacylglyceride comprises three fatty acid ester residues, wherein one fatty acid ester residue is Cx¹:y¹ which means that this residue comprises x¹ carbon atoms and y¹ double bonds, wherein one fatty acid ester residue is Cx²:y² which means that this residue comprises x² carbon atoms and y² double bonds, and wherein one fatty acid ester residue is Cx³:y³ which means that this residue comprises x³ carbon atoms and y³ double bonds. Preferably, any of these fatty acid ester residues may be attached to any former hydroxyl groups of the glycerol.

For example, SOP2 comprises three fatty acid ester residues, wherein one fatty acid ester residue is C18:0 which means that this residue comprises 18 carbon atoms and 0 double bonds, wherein one fatty acid ester residue is C18:1 which means that this residue comprises 18 carbon atoms and 1 double bond, and wherein one fatty acid ester residue is C16:0 which means that this residue comprises 16 carbon atoms and 0 double bonds. Preferably, any of these fatty acid ester residues may be attached to any former hydroxyl groups of the glycerol.

Ceramide (CER) Biomarkers

The biomarkers Cer(d16:1/24:0), Cer(d17:1/24:0), Cer(d18:1/23:0), Cer(d18:1/24:1), and Cer(d18:2/24:0) are ceramides.

The ceramide CER(dx¹:y¹/x²:y²) is preferably denoted to mean that the ceramide comprises the “sphingosine-backbone” dx¹:y¹, wherein x¹ denotes the number of carbon atoms and y¹ the number of double bonds, and a “fatty acid amid” residue x²:y², wherein x² denotes the number of carbon atoms and y² the number of double bonds thereof. Preferably, “d” indicates that the backbone comprises two hydroxyl groups.
Cer(d16:1/24:0) is Ceramide (d16:1/24:0)
Cer(d17:1/24:0) is Ceramide (d17:1124:0)
Cer(d18:1/23:0) is Ceramide (d18:1/23:0)
Cer(d18:1/24:1) is Ceramide (d18:1/24:1)
Cer(d18:2/24:0) is Ceramide (d18:2/24:0)

For example, the ceramide Cer(d16:1/24:0) is preferably denoted to mean that the ceramide comprises the “sphingosine-backbone” d16:1, comprising 16 carbon atoms and 1 double bond, and a “fatty acid amid” residue 24:0 comprising 24 carbon atoms and 0 double bonds.

Sphingomyelin (SM) Biomarkers

SM is the abbreviation for Sphingomyelin. As set forth herein below, the amounts of the sphingomyelin biomarkers SM10, SM18, SM2, SM21, SM23, SM24, SM5, and SM9 may be determined by determining the amount of a single sphingomyelin species in the sample, or by determining the total amount of two (or even three) sphingomyelin species which have the same or essentially the same molecular weight (see Table 1 in the Examples section). For example, for SM10 the amount of Sphingomyelin(d18:1/18:0) can be determined, or the total amount of Sphingomyelin(d18:1/18:0) and Sphingomyelin(d16:1/20:0). Whether the amount of a single species or of two (or three) species is determined may depend on the assay used for the determination. For example, the amount of a single species is determined, if the assay underlying the determination step (step a.) allows for the determination of a single species. The total amount of two (or three) species may be determined, if the assay underlying the determination step allows for the determination of the total amount of the two (or three) species only (rather than for the determination of the single species), in other words the assay is not capable of differentially determining the amounts of each of the two (or three) species. E.g. if the determination of the amount of a biomarker comprises mass spectrometry, the determination of the amount of the biomarker is preferably based on a single peak in a mass spectrum for the two (or three) species since the peaks of the species overlap. This is taken into account by the skilled person. In the context of the studies underlying the present invention it has been shown that both the determination of a single species and the determination of two (or three) species as described herein below (or in Table 1) allow for a reliable diagnosis. Advantageously, it has been even shown that the determination of two (or three) species may allow for a more reliable diagnosis than the diagnosis based on a single species in certain cases. Moreover, the determination of the total amount of two (or three) species having the same molecular weight is less complex (as compared to the determination of a single species). The same applies to the determination of PC8 (see below).

In Table 1 of the Examples section, the preferred Sphingomyelin species for SM10, SM18, SM2, SM21, SM23, SM24, SM5, and SM9 are listed (the species are referred to as “analyte” in this table).

For SM10, SM2, SM24, SM5, and SM9 two analytes are listed (analyte 1 and analyte 2). The said biomarkers preferably refer to Analyte 2 (for example, for SM2: SM(d16:1/16:0)), more preferably to Analyte 1 (for example, for SM2: SM(d18:1/14:0)) and most preferably to Analyte 1 and 2 (for SM2: Sphingomyelin(d18:1/14:0) and Sphingomyelin(d16:1/16:0)). Accordingly, a biomarker for which two species are listed in table 1 is Analyte 1, Analyte 2 or a combination of Analyte 1 and Analyte 2.

The amount of said biomarker is, thus, preferably determined by determining the amount of Analyte 2, or more preferably of Analyte 1 and most preferably by determining the combined (and thus the total) amount of Analyte 1 and Analyte 2. Accordingly, the amount of said biomarker is the amount of Analyte 1 or 2, or the sum of the amounts of Analyte 1 and 2. The same applies to PC8.

For the biomarkers SM18, SM21 and SM 23 three analytes are listed (Analyte 1, 2 and 3). The said biomarkers preferably refer to Analyte 3, more preferably to Analyte 2, even more preferably to Analyte 1, and most preferably to Analyte 1, 2 and 3. Accordingly, a biomarker for which three species are listed in Table 1 is Analyte 1, Analyte 2, or Analyte 3 or a combination of Analyte 1, Analyte 2 and Analyte 3.

If two analytes are listed for a biomarker (Analyte 1 and Analyte 2), it is envisaged that the biomarker is Analyte 1. Alternatively, the biomarker may be Analyte 2. If three analytes are listed for a biomarker (Analyte 1, Analyte 2 and Analyte 3), it is envisaged that that the biomarker is Analyte 1. Alternatively, the biomarker may be Analyte 2. Also, the biomarker may be Analyte 3. Alternatively the biomarker may be a combination of Analyte 1 and Analyte 2. Alternatively the biomarker may be a combination of Analyte 1 and Analyte 3. Alternatively the biomarker may be a combination of Analyte 2 and Analyte 3. Alternatively the biomarker may be a combination of Analyte 1, Analyte 2 and Analyte 3.

The amount of said biomarker is, thus, preferably determined by determining the amount of Analyte 3, or more preferably of Analyte 2, even more preferably of Analyte 1 and most preferably by determining the combined (and thus the total) amount of Analyte 1, Analyte 2, and Analyte 3. Accordingly, the amount of said biomarker is the amount of Analyte 1, 2, or 3, or the sum of the amounts of Analyte 1, 2 and 3.

The following applies in particular:

The biomarker SM10 preferably refers to Sphingomyelin(d18:1/18:0), or more preferably to Sphingomyelin(d18:1/18:0) and Sphingomyelin(d16:1/20:0). Accordingly, the biomarker SM10 is Sphingomyelin(d18:1/18:0), or a combination of Sphingomyelin(d18:1/18:0) and Sphingomyelin(d16:1/20:0).

The amount of SM10 is, thus, preferably determined by determining the amount of Sphingomyelin(d18:1/18:0), or by determining the combined (and thus the total) amount of Sphingomyelin(d18:1/18:0) and Sphingomyelin(d16:1/20:0). Accordingly, the amount of SM10 is the amount of Sphingomyelin(d18:1/18:0), or the sum of the amounts of Sphingomyelin(d18:1/18:0) and Sphingomyelin(d16:1/20:0).

The biomarker SM18 preferably refers to Sphingomyelin(d18:1/21:0), or more preferably to Sphingomyelin(d18:1/21:0), Sphingomyelin(d16:1/23:0) and Sphingomyelin(d17:1/22:0). Accordingly, the biomarker SM18 is Sphingomyelin(d18:1/21:0), or a combination of Sphingomyelin(d18:1/21:0), Sphingomyelin(d16:1/23:0) and Sphingomyelin(d17:1/22:0).

The amount of SM18 is, thus, preferably determined by determining the amount of Sphingomyelin(d18:1/21:0), or by determining the combined (and thus the total) amount of Sphingomyelin(d18:1/21:0) Sphingomyelin(d16:1/23:0) and Sphingomyelin(d17:1/22:0). Accordingly, the amount of SM18 is the amount of Sphingomyelin(d18:1/21:0), or the sum of the amounts of Sphingomyelin(d18:1/21:0), Sphingomyelin(d16:1/23:0) and Sphingomyelin(d17:1/22:0).

The biomarker SM2 preferably refers to Sphingomyelin(d18:1/14:0) or more preferably to Sphingomyelin(d18:1/14:0) and Sphingomyelin(d16:1/16:0). Accordingly, the biomarker SM2 is Sphingomyelin(d18:1/14:0), or a combination of Sphingomyelin(d18:1/14:0) and Sphingomyelin(d16:1/16:0).

The amount of SM2 is, thus, preferably determined by determining the amount of Sphingomyelin(d18:1/14:0), or by determining the combined (and thus the total) amount of Sphingomyelin(d18:1/14:0) and Sphingomyelin(d16:1/16:0). Accordingly, the amount of SM2 is the amount of Sphingomyelin(d18:1/14:0), or the sum of the amounts of Sphingomyelin(d18:1/14:0) and Sphingomyelin(d16:1/16:0).

The biomarker SM21 preferably refers to Sphingomyelin(d17:1/23:0) or more preferably to Sphingomyelin(d17:1/23:0), Sphingomyelin(d18:1/22:0) and Sphingomyelin(d16:1/24:0). Accordingly, the biomarker SM21 is Sphingomyelin(d17:1/23:0), or a combination of Sphingomyelin(d17:1/23:0), Sphingomyelin(d18:1/22:0), and Sphingomyelin(d16:1/24:0).

The amount of SM21 is, thus, preferably determined by determining the amount of Sphingomyelin(d17:1/23:0), or by determining the combined (and thus the total) amount of Sphingomyelin(d17:1/23:0), Sphingomyelin(d18:1/22:0) and Sphingomyelin(d16:1/24:0). Accordingly, the amount of SM21 is the amount of Sphingomyelin(d17:1/23:0), or the sum of the amounts of Sphingomyelin(d17:1/23:0), Sphingomyelin(d18:1/22:0) and Sphingomyelin(d16:1/24:0).

The biomarker SM23 preferably refers to Sphingomyelin(d18:1/23:1) or more preferably to Sphingomyelin(d18:1/23:1), Sphingomyelin(d18:2/23:0) and Sphingomyelin(d17:1/24:1). Accordingly, the biomarker SM23 is Sphingomyelin(d18:1/23:1), or a combination of Sphingomyelin(d18:1/23:1), Sphingomyelin(d18:2/23:0), and Sphingomyelin(d17:1/24:1). Further, in another embodiment, it is envisaged that SM23 is Sphingomyelin (d17:1/24:1).

The amount of SM23 is, thus, preferably determined by determining the amount of Sphingomyelin(d18:1/23:1), or by determining the combined (and thus the total) amount of Sphingomyelin(d18:1/23:1), Sphingomyelin(d18:2/23:0) and Sphingomyelin(d17:1/24:1). Accordingly, the amount of SM23 is the amount of Sphingomyelin(d18:1/23:1), or the sum of the amounts of Sphingomyelin(d18:1/23:1), Sphingomyelin(d18:2/23:0), and Sphingomyelin(d17:1/24:1). Further, it is envisaged, in another embodiment, that the amount of SM23 is determined by determining the amount of Sphingomyelin (d17:1/24:1).

In an embodiment, the biomarker SM23 preferably refers to Sphingomyelin(d18:2/23:0). In the embodiment, the biomarker SM23 is, thus, preferably determined by determining the amount of Sphingomyelin (d18:2/023:0).

The biomarker SM24 preferably refers to Sphingomyelin(d18:1/23:0) or more preferably to Sphingomyelin(d18:1/23:0) and Sphingomyelin(d17:1/24:0). Accordingly, the biomarker SM24 is Sphingomyelin(d18:1/23:0), or a combination of Sphingomyelin(d18:1/23:0) and Sphingomyelin(d17:1/24:0).

The amount of SM24 is, thus, preferably determined by determining the amount of Sphingomyelin(d18:1/23:0), or by determining the combined (and thus the total) amount of Sphingomyelin(d18:1/23:0) and Sphingomyelin(d17:1/24:0). Accordingly, the amount of SM24 is the amount of Sphingomyelin(d18:1/23:0), or the sum of the amounts of Sphingomyelin(d18:1/23:0) and Sphingomyelin(d17:1/24:0).

The biomarker SM28 is preferably Sphingomyelin(d18:1/24:0).

The biomarker SM29 is preferably Sphingomyelin(d18:2/17:0).

The biomarker SM3 is preferably Sphingomyelin(d17:1/16:0).

The biomarker SM5 preferably refers to Sphingomyelin(d18:1/16:0) or more preferably to Sphingomyelin(d18:1/16:0) and Sphingomyelin(d16:1/18:0). Accordingly, the biomarker SM5 is Sphingomyelin(d18:1/16:0), or a combination of Sphingomyelin(d18:1/16:0) and Sphingomyelin(d16:1/18:0).

The amount of SM5 is, thus, preferably determined by determining the amount of Sphingomyelin(d18:1/16:0), or by determining the combined (and thus the total) amount of Sphingomyelin(d18:1/16:0) and Sphingomyelin(d16:1/18:0). Accordingly, the amount of SM5 is the amount of Sphingomyelin(d18:1/16:0), or the sum of the amounts of Sphingomyelin(d18:1/16:0) and Sphingomyelin(d16:1/18:0).

The biomarker SM8 is preferably Sphingomyelin(d18:2/18:1).

The biomarker SM9 preferably refers to Sphingomyelin(d18:1/18:1) or more preferably to Sphingomyelin(d18:1/18:1) and Sphingomyelin(d18:2/18:0). Accordingly, the biomarker SM9 is Sphingomyelin(d18:1/18:1), or a combination of Sphingomyelin(d18:1/18:1) and Sphingomyelin(d18:2/18:0).

The amount of SM9 is, thus, preferably determined by determining the amount of Sphingomyelin(d18:1/18:1), or by determining the combined (and thus the total) amount of Sphingomyelin(d18:1/18:1) and Sphingomyelin(d18:2/18:0). Accordingly, the amount of SM9 is the amount of Sphingomyelin(d18:1/18:1), or the sum of the amounts of Sphingomyelin(d18:1/18:1) and Sphingomyelin(d18:2/18:0).

As set forth above, the present invention is based on the amount of at least three biomarkers. In this context, it is noted that SM10, SM18, SM2, SM21, SM23, SM24, SM5, and SM9, and PC8 are considered as single biomarkers regardless whether the determination of these biomarkers is based on the determination of a single, two (or three) species. Thus, at least two further biomarkers have to be determined.

The sphingomyelin SM(dx¹:y¹/x²:y²) is preferably denoted to mean that the sphingomyelin comprises the “sphingosine-backbone” dx¹:y¹, wherein x¹ denotes the number of carbon atoms and y¹ the number of double bonds, and a “fatty acid amide” residue x²:y², wherein x² denotes the number of carbon atoms and y² the number of double bonds thereof.

The Sphingomyelin(d18:2/18:0) is preferably denoted to mean that the sphingomyelin comprises the “sphingosine-backbone” d18:2, which comprises 18 carbon atoms and 2 double bonds, and a “fatty acid amide” residue 18:0, which comprises 18 carbon atoms and 0 double bonds.

Cholesterylester (CE) Biomarkers

CE is the abbreviation for Cholesterylester.

In accordance with the present invention, the determination of two different cholesterylesters is contemplated, i.e. of cholesterylester C18:0 and/or cholesterylester C18:2. The biomarkers are well known in the art. The cholesterylester (Cx¹:y¹) is preferably denoted to mean that the cholesterylester comprises a fatty acid ester residue, wherein said fatty acid ester residue is Cx¹:y¹ which means that this residue comprises x¹ carbon atoms and y¹ double bonds.

For example, cholesterylester C18:0 is denoted to mean that the cholesterylester C18:0 comprises a fatty acid ester residue, wherein said fatty acid ester residue is C18:0 which means that this residue comprises 18 carbon atoms and 0 double bonds.

Phosphatidylcholine (PC) Biomarkers

PC is the abbreviation for Phosphatidylcholine. The Phosphatidylcholine PC (Cx¹:y¹ Cx²y²) is preferably denoted to mean that the phosphatidylcholine comprises two fatty acid ester residues, wherein one fatty acid ester residue is Cx¹:y¹ which means that this residue comprises x¹ carbon atoms and y¹ double bonds, wherein one fatty acid ester residue is Cx²:y², which means that this residue comprises x² carbon atoms and y² double bonds.

For example, phosphatidylcholine (C16:0 C18:2) is preferably denoted to mean that the phosphatidylcholine comprises two fatty acid ester residues, wherein one fatty acid ester residue is C16:0 which means that this residue comprises 16 carbon atoms and 0 double bonds, wherein one fatty acid ester residue is C18:2, which means that this residue comprises 18 carbon atoms and 2 double bonds.

The biomarker PC4 is Phosphatidylcholine (C16:0 C18:2). The biomarker PC8 preferably refers to Phosphatidylcholine(C18:0 C18:2) or more preferably to Phosphatidylcholine(C18:0 C18:2) and Phosphatidylcholine(C18:1 C18:1). Accordingly, the biomarker PC8 is Phosphatidylcholine(C18:0 C18:2), or a combination of Phosphatidylcholine(C18:0 C18:2) and Phosphatidylcholine(C18:1 C18:1).

The amount of PC8 is, thus, preferably determined by determining the amount of Phosphatidylcholine (C18:0 C18:2), or by determining the combined (and thus the total) amount of Phosphatidylcholine(C18:0 C18:2) and Phosphatidylcholine(C18:1 C18:1). Accordingly, the amount of PC8 is the amount of Phosphatidylcholine(C18:0 C18:2), or the sum of the amounts of Phosphatidylcholine(C18:0 C18:2) and Phosphatidylcholine(C18:1 C18:1).

Preferred Biomarker Combinations

As set forth above, the method of the present invention for diagnosing heart failure comprises the determination of the amounts of at least three biomarkers in a sample of a subject. Thus, the determination of the amounts of a combination of at least three biomarkers is contemplated. Preferred combinations of at least three biomarkers to be determined in accordance with the present invention are described herein below. Moreover, preferred combinations of at least three markers are described in under “Embodiments/Items of the present invention”. The at least three biomarkers as described under “Embodiments/Items of the present invention” can be used for the diagnosis of heart failure as well.

The at least three biomarkers for the diagnosis of heart failure are preferably:
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin;
iv. at least one phosphatidylcholine, at least one cholesterylester, and at least one sphingomyelin;
v. Cholesterylester C18:2, SSS and Cer(d17:1/24:0);
vi. at least two sphingomyelins selected from the group consisting of SM2, SM3, SM5, SM18, SM23, SM24, and SM28, and at least one triacylglyceride selected from the group consisting of SOP2, SPP1 or PPO1 or selected from the group consisting of SOP2, SPP1 or PPP;
vii. at least two triacylglycerides selected from the group consisting of OSS2, SOP2, SPP1 and SSP2, and at least one sphingomyelin selected from the group consisting of SM23 and SM24;
viii. SM18, SM24 and SM28;
ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 of Table 2, or
x. the biomarkers of panel 200, 201, 202, 203, 204, 205, or 206 of Table 2a.

Preferred cholesterylester, triacylglyceride, phosphatidylcholine and sphingomyelin biomarkers to be determined and further preferred combinations of at least three biomarkers are described below (in particular for items i., ii., iii., and iv.).

Preferably, the at least one triacylglyceride biomarker in i., ii., and iii. is selected from the group consisting of SOP2, OSS2, SPP1, SSP2, PPO1 and PPP, the at least one cholesterylester biomarker in i., iii. and iv. is selected from the group consisting of cholesterylester C18:2 and cholesterylester C18:0, the at least one phosphatidylcholine biomarker in i., ii., and iv. is selected from the group consisting of PC4 and PC8, and the at least one sphingomyelin biomarker in ii., iii. and iv. is selected from the group consisting of SM18, SM24, SM23, SM21, SM28, SM5, SM3, SM29 and SM8.

Alternatively, the least one triacylglyceride biomarker in i., ii., and iii. is selected from the group consisting of SOP2, OSS2, SPP1, SSP2, PPO1 and SPP1.

In a preferred embodiment, at least the amounts of the biomarkers of items i., ii., iii., vi, vii., ix. or x. (in particular of items i., ii., iii., vi, vii. or ix.) as set forth above are determined. In particular, the at least one triacylglyceride biomarker in i., ii. and iii. is selected from the group consisting of SOP2, OSS2, SSP2, PPO1 and PPP, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, SM28, SM5, and SM3. Preferably, at least the amounts of the biomarkers of any panel of Table 2 which comprises these biomarkers are determined. More preferably, at least the amounts of the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 in Table 2 are determined. The combinations of biomarkers as referred to in this paragraph are, preferably, determined if the HF (heart failure) to be diagnosed is heart failure with reduced left ventricular ejection fraction (HFrEF). The subject might show no symptoms of heart failure. Moreover, it is envisaged the HfrEF is selected from DCMP and ICMP.

The term “at least the amounts”, preferably means that, in principle, further biomarkers could be determined. In an embodiment, the term means “the amounts”. Preferably, the amounts of the at least three biomarkers as referred to in step a) of the method disclosed herein, are used for the comparison in step b).

In an even further preferred embodiment, at least the amounts of the biomarkers of i., ii., iii., vi., ix. or x. (in particular of items i., ii., iii., vi. or ix.) are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, and SM3. Preferably, at least the amounts of the biomarkers of any panel of Table 2 which comprises these biomarkers are determined. In particular, at least the amounts of the biomarkers of panel 1, 2, 3, 4, 5, 6, 7, 8, 9, 10, 11, 12, 13, 14, 15, 16, 17, or 18 in Table 2 are determined. The combinations of biomarkers as referred to in this paragraph are, preferably, determined if the HF (heart failure) to be diagnosed is heart failure with reduced left ventricular ejection fraction (HFrEF).

In an even further preferred embodiment, at least the amounts of the biomarkers of i., ii., iii., vi., ix. or x. (in particular of i., ii., iii, vi, or ix.) are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is SM23. Preferably, at least the amounts of the biomarkers of any panel of Table 2 which comprises these biomarkers are determined. In particular, at least the amounts of the biomarkers of panel 1, 2, 3, or 4 in Table 2 are determined. Preferably, at least the amounts of OSS2, PC4 and SM23 are determined (panel 1), in particular in combination with the determination of the amount of NT-proBNP or BNP (see elsewhere herein). Alternatively, the biomarkers of panels 3, 13 and 60 may be determined. Also preferably, at least the amounts of OSS2, cholesterylester C18:2 and SM23 are determined (panel 2). Alternatively, the biomarkers of panels 20, 21, 22, 23, 32 or 60 may be determined. The combinations of biomarkers as referred to in this paragraph are, preferably, determined if the HF (heart failure) to be diagnosed is heart failure with reduced left ventricular ejection fraction (HFrEF).

In an even further preferred embodiment, at least the amounts of the biomarkers of iii. are determined, wherein the at least one triacylglyceride biomarker is SOP2 and/or OSS2, and/or (in particular and) the at least one cholesterylester biomarker is cholesterylester C18:2, and/or (in particular and) the at least one sphingomyelin biomarker is SM23. In particular, at least the amounts of SOP2, OSS2, PC4, Cholesterylester C18:2, SM18, SM28, SM24, SSP2, and SM23 are determined (panel 3). The combinations of biomarkers as referred to in this paragraph are, preferably, determined if the HF (heart failure) to be diagnosed is heart failure with reduced left ventricular ejection fraction (HFrEF).

As set forth in the previous paragraph, it is, in particular, envisaged to determine the amounts (or to determine at least the amounts of) the biomarkers comprised by panel 3, and thus of SOP2, OSS2, PC4, Cholesterylester C18:2, SM18, SM28, SM24, SSP2, and SM23. The determination of the amounts of these biomarkers allows for a reliable diagnosis of heart failure, in particular of HFrEF (or subforms thereof), even in the absence of the determination of NT-proBNP or BNP and in the absence of a correction for confounders. In particular early stages of heart failure can be determined. Thus, the subject may show no symptoms of heart failure. The LVEF may be mildly reduced. Thus, the HFrEF may be heart failure with a left ventricular ejection fraction of lower than 50% but larger than 35%. The same, e.g., applies to panels 1 and 2. Thus, early stages of heart failure can be diagnosed by using the biomarkers of panel 2 and in particular of panel 1. The subject to be tested may show no symptoms of heart failure. HFrEF may be heart failure with a left ventricular ejection fraction of lower than 50% but larger than 35%.

Also preferably, the amounts (or at least the amounts) of at least three biomarkers, of at least four biomarkers, more preferably of at least five or six biomarkers, even more preferably of at least seven biomarkers and most preferably of at least eight biomarkers of the biomarkers of panel 3 are determined in step a) of the method of the present invention.

If the amounts of the biomarkers of panel 3 (or of the biomarkers as set forth in the previous paragraph) are determined, the method preferably does not comprise the further determination of amount of NT-proBNP and/or BNP. In particular, the method may not comprise the further determination of the amount of BNP and/or NT-proBNP and the comparison of the amount of BNP and/or NT-proBNP to a reference. Thus, the diagnosis of heart failure is not based on the determination of NT-proBNP. Accordingly, the method is a non-BNP and non-NT-proBNP based method. The same, e.g., may also apply to panels 1 and 2.

Alternatively, the method may comprise the further determination of amount of NT-proBNP and/or BNP. In particular, the method may comprise the further determination of the amount of BNP or NT-proBNP and the comparison of the amount of BNP or NT-proBNP to a reference.

If the amounts of the biomarkers of panel 3 (of at least three, four, five, six, seven or eight biomarkers of the biomarkers of panel 3) are determined, preferably a correction for confounders is not carried out. Alternatively, a correction for confounders may be carried out. The same may, e.g., apply to panels 1 and 2.

In a preferred embodiment, at least the amounts of the biomarkers shown in i., ii., iii. or vii. are determined, wherein the at least one triacylglyceride biomarker in i., ii. and iii. s selected from the group consisting of SOP2, OSS2, and SSP2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM24, SM23, SM28, and SM3. Preferably, at least the amounts of the biomarkers of any panel of Table 2 which comprises these biomarkers are determined. More preferably, at least the amounts of the biomarkers of panel 19, 20, 21, 22, 23, 24, 25, 26, 27, 28, 29, 30, 31, 32, 33, 34, 35, or 36 in Table 2 are determined. The combinations of biomarkers as referred to in this paragraph are, preferably, determined if the HF is HFrEF, and in particular DCMP (dilated cardiomyopathy). The subject to be tested, preferably, shows no symptoms of HF. Alternatively, the subject shows symptoms of HF.

In an even further preferred embodiment, at least the amounts of the biomarkers shown in ii., or iii. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is SM23 and/or SM24. Preferably, at least the amounts of the biomarkers of any panel of Table 2 which comprises these biomarkers are determined. More preferably, at least the amounts of the biomarkers of panel 31, 32, 33, 34, 35, or 36 in Table 2 are determined. The combinations of biomarkers as referred to in this paragraph are, preferably, determined if the HF is HFrEF, and in particular DCMP (dilated cardiomyopathy).

In a preferred embodiment, at least the amounts of the biomarkers shown ii., iii. or vi. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. is selected from the group consisting of SM18, SM24, and SM23. Preferably, at least the amounts of the biomarkers of any panel of Table 2 which comprises these biomarkers are determined. More preferably, at least the amounts of the biomarkers of panel 37, 38, 39, 40, 41, 42, 43, 44, 45, 46, 47, 48, 49, 50, 51, 52, 53, 54, 55, or 56 in Table 2 are determined. The combinations of biomarkers as referred to in this paragraph are, preferably, determined if the HF is HFrEF, and in particular ICMP (ischemic cardiomyopathy).

In a further preferred embodiment, at least the amounts of the biomarkers shown in iii. are determined, wherein the at least one triacylglyceride biomarker is SOP2, and/or (in particular and) the at least one cholesterylester biomarker is cholesterylester C18:2, and/or (in particular and) the at least one sphingomyelin biomarker is SM18 and/or SM23. Preferably, at least the amounts of the biomarkers of any panel of Table 2 which comprises these biomarkers are determined. More preferably, at least the amounts of the biomarkers of panel 51, 52, 53, 54, 55, or 56 in Table 2 are determined. The combinations of biomarkers as referred to in this paragraph are, preferably, determined if the HF is HFrEF, and in particular ICMP (ischemic cardiomyopathy).

In a preferred embodiment, at least the amounts of the biomarkers of ii., iii., vi., vii., ix. or x. (in particular of ii., iii., vi., vii. or ix.) are determined, and wherein the at least one triacylglyceride biomarker in ii. and iii. is selected from the group consisting of SOP2, OSS2, and PPO1, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, 12, 19, 20, 21, 22, 23, 24, 37, 38, 39, 40, 41, or 42 in Table 2 are determined. The combinations of biomarkers as referred to in this paragraph are, preferably, determined if the HF is HFrEF, in particular asymptomatic HFrEF. Thus the subject to be tested preferably does not show symptoms of heart failure. Accordingly, the combinations of biomarkers are, preferably, determined for the diagnosis of HFrEF in a subject who does not show symptoms of heart failure.

In a further preferred embodiment, wherein at least the amounts of the biomarkers of iii. or vi are determined, wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, or 12 in Table 2 are determined. The combinations of biomarkers as referred to in this paragraph are, preferably, determined if the HF is HFrEF, in particular asymptomatic HFrEF. Thus the subject to be tested preferably does not show symptoms of heart failure. Accordingly, the combinations of biomarkers are, preferably, determined for the diagnosis of HFrEF in a subject who does not show symptoms of heart failure.

In a preferred embodiment, at least the amounts of the biomarkers of iii. or vii. are determined, wherein the at least one triacylglyceride biomarker in iii. is selected from the group consisting of SOP2 and OSS2, and/or (in particular and) the at least one cholesterylester biomarker in iii. is cholesterylester C18:2, and/or (in particular and) the at least one sphingomyelin biomarker in iii. is SM23. Preferably, at least the amounts of the biomarkers of any panel of Table 2 which comprises these biomarkers are determined. More preferably, at least the amounts of the biomarkers of panel 19, 20, 21, 22, 23, or 24 in Table 2 are determined. The combinations of biomarkers as referred to in this paragraph are, preferably, determined if the HF or HFrEF is DCMP, in particular asymptomatic DCMP. Thus the subject to be tested preferably does not show symptoms of heart failure. Accordingly, the combinations of biomarkers are, preferably, determined for the diagnosis of HFrEF, in particular of DCMP, in a subject who does not show symptoms of heart failure.

In a preferred embodiment, at least the amounts of the biomarkers of iii. or vi. are determined, and wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 37, 38, 39, 40, 41, or 42 in Table 2 are determined. The combinations of biomarkers as referred to in this paragraph are, preferably, determined if the HF or HFrEF is ICMP, in particular asymptomatic ICMP. Thus the subject to be tested preferably does not show symptoms of heart failure. Accordingly, the combinations of biomarkers are, preferably, determined for the diagnosis of HFrEF, in particular of ICMP, in a subject who does not show symptoms of heart failure.

In an embodiment, the heart failure to be diagnosed is HF with preserved ejection fraction (HFpEF). For the diagnosis of HFpEF, preferably, at least the amounts of the biomarkers of ii., vi. or vii. as set forth above are determined, and wherein the at least one triacylglyceride biomarker in ii. is selected from the group consisting of SOP2, SSP2, SPP1 and PPO1, and/or (in particular and) the at least one phosphatidylcholine biomarker in ii. is selected from the group consisting of PC4 and PC8, and/or (in particular and) the at least one sphingomyelin biomarker in ii. is selected from the group consisting of SM18, SM24, SM23, SM28, SM5, and SM3. Preferably, at least the amounts of the biomarkers of any panel of Table 2 which comprises these biomarkers are determined. More preferably at least the amounts of the biomarkers of panel 79, 80, 81, 82, 83, 84, 85, 86, 87, 88, 89, 90, 91, 92, 93, 94, 95, 96, 97, 98, 99, 100, 101, or 102 in Table 2 are determined. Even more preferably, at least the amounts of the biomarkers of ii. are determined, and wherein the at least one triacylglyceride biomarker is selected from the group consisting of SSP2, SPP1 and PPO1, and/or (in particular and) the at least one phosphatidylcholine biomarker is PC4, and/or (in particular and) the at least one sphingomyelin biomarker is selected from the group consisting of SM24, SM5, and SM3. In particular, wherein at least the amounts of the biomarkers of panel 79, 81, 83, 85, 87, 89, 91, 93, 95, 97, 99, or 101 in Table 2 are determined for the diagnosis of HFpEF. Most preferably, at least the amounts of the biomarkers of ii. of are determined, and wherein the at least one triacylglyceride biomarker is SSP2, and/or (in particular and) the at least one phosphatidylcholine biomarker is PC4, and/or (in particular and) the at least one sphingomyelin biomarker is selected from the group consisting of SM24 and SM5. Preferably, at least the amounts of the biomarkers of panel 95, 97, 99, or 101 in Table 2 are determined.

In particular, the HFpEF to be diagnosed is asymptomatic. Accordingly, the HFpEF is diagnosed in a subject who does not show symptoms of heart failure. Preferably, at least the amounts of the biomarkers of ii. or vii. (in particular of ii.) are determined in this case, wherein the at least one triacylglyceride biomarker in ii. is selected from the group consisting of SPP1 and SSP2, and/or (in particular and) the at least one phosphatidylcholine biomarker in ii. is PC4, and/or (in particular and) the at least one sphingomyelin biomarker in ii. is selected from the group consisting of SM5 and SM3. Preferably, at least the amounts of the biomarkers of any panel of Table 2 which comprises these biomarkers are determined. In particular, at least the amounts of the biomarkers of panel 79, 80, 81, 82, 83, 84, 85, or 86 in Table 2 are determined. More preferably, at least the amounts of the biomarkers of ii. are determined, wherein the at least one triacylglyceride biomarker is SPP1, and/or (in particular and) the at least one phosphatidylcholine biomarker is PC4, and/or (in particular and) the at least one sphingomyelin biomarker is SM5. In particular, at least the amounts of the biomarkers of panel 79, 81, 83, or 85 in Table 2 are determined, preferably for the diagnosis of HFpEF in a subject who does not show symptoms of heart failure.

In a preferred embodiment, at least the amounts of the biomarkers of viii. (Panel 199) are determined, where CHF is to be diagnosed and NT-proBNP is not included, in particular, at least the amounts of the biomarkers of panel 3, 5, 9, 11, 17, 35, 39, 47, 53, 55, 62, 70, 77, 99, or 199 in Table 2 are determined.

In one embodiment, the amount of NT-proBNP or BNP is determined in addition to the amount of the resulting combinations of the at least three biomarkers set forth above. In another embodiment, the amount of NT-proBNP or BNP is not determined in addition to the amount of the resulting combinations of the at least three biomarkers set forth above (see elsewhere herein).

As set forth above, it is envisaged to determine the amounts of the biomarkers of any one of the panels 1 to 206. The biomarkers of panels 1 to 199 are shown in table 2 (second column). The biomarkers of panels 200 to 206 are shown in table 2a (second column). Preferred panels are panel 1, panel 2, panel 3 and panel 4.

In a preferred embodiment, the amounts of the biomarkers of panel 1, 2, 3 or 4 are determined in step a) of the method of the present invention. In another preferred embodiment the biomarkers of panel 200 are determined.

For example, if the amounts of the biomarkers of panel 1 are determined, the amounts of SM23, OSS2 and PC4 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (d17:1/24:1). In another embodiment, SM23 is Sphingomyelin(d18:1/23:1).

If the biomarkers of panel 1 are determined, it is in particular envisaged to determine:
the amount of OSS2, the amount of Sphingomyelin(d18:1/23:1) and the amount of PC4,
the amount of OSS2, the amount of Sphingomyelin (d17:1/24:1) and the amount of PC4, or
the amount of OSS2, the combined amount of Sphingomyelin(d18:1/23:1), Sphingomyelin(d18:2/23:0), and Sphingomyelin(d17:1/24:1), and the amount of PC4

For example, if the amounts of the biomarkers of panel 2 or 4 are determined, the amounts of SM23, OSS2 and Cholesterylester C18:2 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In particular, SM23 is Sphingomyelin (d17:1/24:1) or Sphingomyelin(d18:1/23:1).

If the biomarkers of panel 2 or 4 are determined, it is in particular envisaged to determine:
the amount of OSS2, the amount of Sphingomyelin(d18:1/23:1) and the amount of Cholesterylester C18:2,
the amount of OSS2, the amount of Sphingomyelin (d17:1124:1) and the amount of Cholesterylester C18:2, or
the amount of OSS2, the combined amount of Sphingomyelin(d18:1/23:1), Sphingomyelin(d18:2/23:0), Sphingomyelin(d17:1/24:1) and the amount of Cholesterylester C18:2

For example, if the amounts of the biomarkers of panel 200 are determined, the amounts of Cholesterylester C18:2, SM23, OSS2 and PC4 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (d17:1/24:1). In another embodiment, SM23 is Sphingomyelin(d18:1/23:1).

If the biomarkers of panel 200 are determined, it is in particular envisaged to determine:
the amount of Cholesterylester C18:2, the amount of OSS2, the amount of Sphingomyelin(d18:1/23:1) and the amount of PC4,
the amount of Cholesterylester C18:2, the amount of OSS2, the amount of Sphingomyelin (d17:1/24:1) and the amount of PC4, or
the amount of Cholesterylester C18:2, the amount of OSS2, the combined amount of Sphingomyelin(d18:1/23:1), Sphingomyelin(d18:2/23:0), and Sphingomyelin(d17:1/24:1), and the amount of PC4

In addition to panel 1, panel 200 further comprises Cholesterylester C18:2.

In addition to panels 2 and 4, panel 200 further comprises PC4.

As set forth elsewhere herein, it is further envisaged to determine the amount of NT-proBNP or BNP in addition to the amounts of the biomarkers of the aforementioned panels (1 to 206). For example, NT-proBNP or BNP is determined in addition to the amounts of the at least three biomarkers of panel 1. Moreover, in an embodiment, it is envisaged that no correction for confounders is carried out (e.g. for panel 1).

With respect to panel 1, preferred embodiments are given in the section “Items for panel 1” (see items 1 to 43). In this section, e.g. preferred subforms of heart failure to be diagnosed are listed. Moreover, the subject is further defined (such as that the subject preferably does not show symptoms of heart failure). The definitions given herein, preferably, apply to the items disclosed in this section. The section is exemplary for the biomarkers of panel 1. However, it is envisaged to replace the biomarkers of panel 1 in this section by the at least three biomarkers as referred to herein in connection with the method of the present invention (e. g. by the biomarkers of any one of panels 1 to 206). For example, instead of the biomarkers of panel 1 in the section “Items for panel 1”, the amounts of the biomarkers of panel 200 can be determined.

In an embodiment the biomarkers of panels 1, 2 or 200, in particular the biomarkers of panel 1, are used in combination with NT-proBNP. Preferably, the determination is carried out for the diagnosis of HFrEF, in particular for the diagnosis of HFrEF with a left ventricular ejection fraction of lower than 50% but larger than 35%. Thus, the subject to be tested preferably shall have a left ventricular ejection fraction of lower than 50% but larger than 35%. Preferably, the subject is asymptomatic and, thus, does not show symptoms of heart failure. Accordingly, asymptomatic heart failure is diagnosed.

Further Preferred Combinations of at Least Three Biomarkers for the Diagnosis of Heart Failure and/or Subforms Thereof are Disclosed in the Following:

Moreover, the at least three biomarkers, preferably are as follows. In particular it is envisaged to determine in step a. the amounts of
i. at least one sphingomyelin (SM) biomarker selected from the group consisting of SM18, SM21, SM23, SM24, SM28, SM3, SM5, SM2, SM9 and SM10, in particular at least one sphingomyelin (SM) biomarker selected from the group consisting of SM18, SM21, SM23, SM24, SM28, SM3 and SM5,
ii. at least one triacylglyceride biomarker selected from the group consisting of OSS2, PPO1, SOP2, SSP2 and SPP1, in particular at least one triacylglyceride biomarker selected from the group consisting of OSS2, PPO1, SOP2, and SSP2, and
iii. at least one further biomarker selected from the group consisting of Cer(d16:1/24:0), Cer(d18:1/24:1), Cholesterylester C18:2, PC4, PC8, Cer(d18:2/24:0), Cer(d17:1/24:0) and glutamic acid 1, in particular at least one further biomarker selected from the group consisting of Cer(d16:1/24:0), Cer(d18:1/24:1), Cholesterylester C18:2, and PC4.

Thus, at least one biomarker of i., at least one biomarker of ii., and at least one biomarker of iii. are determined. The same applies to the combinations below.

The aforementioned combinations (i.e. the determination of the aforementioned at least three biomarkers) are preferably used for the diagnosis of heart failure. In an embodiment, the heart failure is asymptomatic heart failure. Thus, the subject to be tested, preferably, does not show symptoms of heart failure. However, it is also envisaged that the subject may show symptoms of heart failure.

In a preferred embodiment, at least the amounts of
i. at least one sphingomyelin biomarker selected from the group consisting of SM28, SM23, SM21, and SM5,
ii. SOP2 and/or OSS2, and
iii. Cholesterylester C18:2 and/or PC4

are determined.

The aforementioned combinations are preferably used for the diagnosis of heart failure. In an embodiment, the heart failure is asymptomatic heart failure. However, it is also envisaged that the subject may show symptoms of heart failure.

In a further preferred embodiment, at least the amounts of
i. at least one sphingomyelin biomarker selected from the group consisting of SM28, SM21, and SM5, in particular SM28, or at least one sphingomyelin biomarker selected from the group consisting of SM18, SM21, and SM5, in particular SM18, and
ii. SOP2, and
iii. PC4

are determined.

The aforementioned combinations are preferably used for the diagnosis of heart failure. In an embodiment, the heart failure is asymptomatic heart failure.

In a further preferred embodiment, wherein at least the amounts of
i. at least one sphingomyelin biomarker selected from the group consisting of SM18, SM2, SM23, SM24, SM28, SM3, and SM5,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cholesterylester C18:2, PC4 and glutamic acid 1

are determined.

The aforementioned combinations are preferably used for the diagnosis of heart failure. In an embodiment, the heart failure is asymptomatic heart failure. Accordingly, the subject preferably does not show symptoms of heart failure.

In a further preferred embodiment, at least the amounts of
i. at least one sphingomyelin biomarker selected from the group consisting of SM23, SM2, and SM24,
ii. at least one triacylglyceride biomarker selected from the group consisting of SOP2, OSS2, and PPO1, and
iii. Cholesterylester C18:2 and/or PC4, in particular Cholesterylester C18:2,

are determined.

The aforementioned combinations are preferably used for the diagnosis of heart failure. In an embodiment, the heart failure is asymptomatic heart failure. Accordingly, the subject preferably does not show symptoms of heart failure.

In particular, at least the amounts of SM23 and/or SM2, in particular of SM23, of SOP2, and of PC4 are determined. The aforementioned combinations are preferably used for the diagnosis of heart failure. In an embodiment, the heart failure is asymptomatic heart failure. Accordingly, the subject to be tested preferably does not show symptoms of heart failure.

In a preferred embodiment of the present invention, at least the amounts of
i. at least one sphingomyelin biomarker selected from the group consisting of SM10, SM18, SM21, SM23, SM24, SM28, SM3 and SM9,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cer(d18:1/24:1), Cer(d17:1/24:0), Cer (d18:2/24:0), Cholesterylester C18:2, PC4 and PC8

are determined.

The aforementioned combinations are preferably used for the diagnosis of heart failure. In an embodiment, the heart failure is symptomatic heart failure. Accordingly, the subject to be tested preferably shows symptoms of heart failure.

In further preferred embodiment
i. at least one sphingomyelin biomarker is selected from the group consisting of SM18, SM3, SM24, and SM23, in particular SM18,
ii. SOP2 and/or OSS2, in particular SOP2, and
iii. Cholesterylester C18:2 and/or PC4, in particular PC4,

are determined.

In particular, least the amounts of SM18 and/or SM24, SOP2, and PC4 are determined.

The aforementioned combinations are preferably used for the diagnosis of heart failure. In an embodiment, the heart failure is symptomatic heart failure. Accordingly, the subject to be tested preferably shows symptoms of heart failure.

In a preferred embodiment, wherein at least the amounts of
i. at least one sphingomyelin biomarker selected from the group consisting of SM23, SM18, SM24, SM28, SM2 and SM3, in particular at least one sphingomyelin biomarker selected from the group consisting of SM23, SM18, SM24, and SM28,
ii. OSS2 and/or SOP2, in particular OSS2,
iii. Cholesterylester C18:2 and/or PC4

are determined.

In particular, at least the amounts of SM23, OSS2, and Cholesterylester C18:2 are determined.

The aforementioned combinations are preferably used for the diagnosis of heart failure, in particular of HFrEF. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM23 SM18, SM2, SM24 and SM28,
ii. SOP2, and
iii. Cholesterylester C18:2

are determined.

In particular, at least the amounts of biomarkers SM23, SOP2, and Cholesterylester C18:2 are determined.

The aforementioned combinations are preferably used for the diagnosis of heart failure, in particular of HFrEF. In an embodiment, the heart failure is asymptomatic heart failure, in particular asymptomatic HFrEF. Accordingly, the subject to be tested preferably does not show symptoms of heart failure.

In a preferred embodiment, wherein at least the amounts of
i. at least one sphingomyelin biomarker selected from the group consisting of SM18, SM23, SM3 or at least one sphingomyelin biomarker selected from the group consisting of SM28, SM23 and SM3,
ii. OSS2, and
iii. Cholesterylester C18:2 and/or PC4

are determined.

In particular, at least the amounts of SM18 and/or SM28, of OSS2, and of Cholesterylester C18:2 are determined.

The aforementioned combinations are preferably used for the diagnosis of heart failure, in particular of HFrEF. In an embodiment, the heart failure is symptomatic heart failure, in particular symptomatic HFrEF. Accordingly, the subject to be tested preferably shows symptoms of heart failure.

In a preferred embodiment of the present invention, at least the amounts of
i. at least one sphingomyelin biomarker selected from the group consisting of SM23, SM24, SM28, and SM3, in particular at least one sphingomyelin biomarker selected from the group consisting of SM23, SM24, and SM28,
ii. SOP2 and/or OSS2, and
iii. Cholesterylester C18:2 and/or PC4

are determined.

In particular, at least the amounts of SM23, of SOP2, and of Cholesterylester C18:2 are determined.

The aforementioned combinations are preferably used for the diagnosis of heart failure, more preferably of HFrEF, and most preferably of DCMP. Preferably, the subject does not show symptoms of heart failure. Also preferably, the subject shows symptoms of heart failure.

In a further preferred embodiment of the present invention, at least the amounts of
i. SM23,
ii. OSS2 and/or SOP2, and
iii. Cholesterylester C18:2

are determined.

In particular, at least the amounts of SM23, OSS2, and Cholesterylester C18:2 are determined.

The aforementioned combinations are preferably used for the diagnosis of heart failure, more preferably of HFrEF, and most preferably of DCMP, in particular of asymptomatic HFrEF and/or DCMP. Thus, the subject to be tested preferably does not show symptoms of HF.

In a preferred embodiment of the present invention at least the amounts of
i. SM28 and/or SM3,
ii. SOP2 and/or OSS2, and
iii. PC4

are determined.

In particular, at least the amounts of SM28, SOP2, and of PC4 are determined.

The aforementioned combinations are preferably used for the diagnosis of heart failure, more preferably of HFrEF, and most preferably of DCMP, in particular of symptomatic HFrEF and/or DCMP. Thus, the subject to be tested preferably shows symptoms of HF.

In another preferred embodiment, wherein at least the amounts of
i. at least one sphingomyelin biomarker selected from the group consisting of SM23, SM24, SM28 and SM18, in particular at least one sphingomyelin biomarker selected from the group consisting of SM23, SM24, and SM28,
ii. SOP2, and
iii. Cholesterylester C18:2

are determined.

In particular, at least the amounts of SM23, SOP2, and Cholesterylester C18:2 are determined.

The aforementioned combinations are preferably used for the diagnosis of heart failure, more preferably of HFrEF, and most preferably of ICMP. In one (preferred) embodiment, the subject does not show symptoms of heart failure. In another embodiment, the subject shows symptoms of heart failure.

In a further preferred embodiment, at least the amounts of SM18, SOP2, and Cholesterylester C18:2 are determined. The aforementioned combination is preferably used for the diagnosis of heart failure, more preferably of HFrEF, and most preferably of ICMP, in particular of symptomatic HFrEF and/or ICMP. Thus, the subject to be tested preferably shows symptoms of HF.

For the diagnosis of asymptomatic heart failure it is further envisaged that the at least three are biomarkers are:
i. at least one sphingomyelin biomarker selected from the group consisting of SM23, SM2, and SM24,
ii. at least one triacylglyceride biomarker selected from the group consisting of SOP2, OSS2, and PPO1, and
iii. at least one further biomarker selected from Cholesterylester C18:2, PC4, and SM5.

Thus, the subject preferably does not show symptoms of heart failure.

For the diagnosis of asymptomatic ICMP it is further envisaged that the at least three biomarkers are markers are selected from the group consisting of SM24, SM5, SM23, SOP2 and PPO1. Preferably, the amounts of SM24, SM5 and SOP2, or of SM23, SM5 and PPO1 are determined.

In one embodiment, the amount of NT-proBNP or BNP is determined in addition to the amount of the resulting combinations of the at least three biomarkers set forth above. In another embodiment, the amount of NT-proBNP or BNP is not determined in addition to the amount of the resulting combinations of the at least three biomarkers set forth above (see elsewhere herein).

Additional Preferred Combinations of at Least Three Biomarkers for the Diagnosis of Heart Failure and/or Subforms Thereof

In a preferred embodiment, the least three biomarkers are:

i. at least one sphingomyelin (SM) biomarker selected from the group consisting of SM18, SM23, SM24, SM3, SM5, SM2, and SM28, and in particular at least one sphingomyelin (SM) biomarker selected from the group consisting of SM18, SM23, SM24, SM3, and SM5,
ii. at least one triacylglyceride biomarker selected from the group consisting of OSS2, SOP2, SSP2 and PPO1, in particular at least one triacylglyceride biomarker selected from the group consisting of OSS2 and SOP2, and
iii. at least one further biomarker selected from the group consisting of Cholesterylester C18:2 and PC4.

Thus, at least one biomarker of i., at least one biomarker of ii., and at least one biomarker of iii. are determined. The same applies to the combinations below.

In a preferred embodiment, at least the amounts of
i. at least one sphingomyelin biomarker selected from the group consisting of SM18, SM23, SM24, SM3, and SM5,
ii. at least one triacylglyceride biomarker selected from the group consisting of OSS2 and SOP2, and
iii. at least one further biomarker selected from the group consisting of Cholesterylester C18:2 and PC4

are determined.

In particular, at the least the amounts of
i. at least one sphingomyelin biomarker selected from the group consisting of SM18, SM3, and SM5, in particular SM18, and
ii. at least one triacylglyceride biomarker selected from the group consisting of OSS2 and SOP2, in particular SOP2, and
iii. at least one further biomarker selected from the group consisting of Cholesterylester C18:2 and PC4, in particular PC4

are determined.

The aforementioned combinations (i.e. the determination of the aforementioned at least three biomarkers) are preferably used for the diagnosis of heart failure. In an embodiment, the heart failure is asymptomatic heart failure. Thus, the subject to be tested, preferably, does not show symptoms of heart failure. However, it is also envisaged that the subject may show symptoms of heart failure.

In a preferred embodiment, at least the amounts of
i. at least one sphingomyelin biomarker selected from the group consisting of SM18, SM2, SM23, SM24, SM28, and SM5,
ii. at least one triacylglyceride biomarker selected from the group consisting of OSS2, PPO1, SOP2, and SSP2, and
iii. at least one further biomarker selected from the group consisting of Cholesterylester C18:2 and PC4

are determined.

In a more preferred embodiment, at least the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2 and PC4, in particular Cholesterylester C18:2,

are determined.

In an even more preferred embodiment, at least the amounts of
i. at least one sphingomyelin biomarker selected from the group consisting of SM23, SM18, and SM2, in particular SM23,
ii. SOP2, and
iii. Cholesterylester C18:2 and/or PC4, in particular Cholesterylester C18:2,

are determined.

The aforementioned combinations are preferably used for the diagnosis of heart failure. In an embodiment, the heart failure is asymptomatic heart failure. Accordingly, the subject preferably does not show symptoms of heart failure.

In a preferred embodiment,
i. at least one sphingomyelin biomarker selected from the group consisting of SM18, SM23, SM24, SM28, and SM3,
ii. at least one triacylglyceride biomarker selected from the group consisting of OSS2 and SOP2, and
iii. at least one further biomarker selected from the group consisting of Cholesterylester C18:2 and PC4

are determined.

Preferably, at least the amounts of
i. SM18,
ii. SOP2, and
iii. Cholesterylester C18:2,

are determined.

In particular, at least the amounts of SM18 and/or SM24, in particular SM18, of SOP2, and of PC4 are determined.

The aforementioned combinations are preferably used for the diagnosis of heart failure. In an embodiment, the heart failure is symptomatic heart failure. Accordingly, the subject to be tested preferably shows symptoms of heart failure.

In a further preferred embodiment, at least the amounts of
i. at least one sphingomyelin biomarker selected from the group consisting of SM23, SM18, SM3, SM24, SM 28 in particular at least one sphingomyelin biomarker selected from the group consisting of SM23 and SM18,
ii. at least one triacylglyceride biomarker selected from the group consisting of OSS2, SOP2 and PPO1, in particular OSS2, and
iii. at least one further biomarker selected from the group consisting of Cholesterylester C18:2 and PC4

are determined.

In particular, at least the amounts of SM23, OSS2, and Cholesterylester C18:2 are determined.

The aforementioned combinations are preferably used for the diagnosis of heart failure, in particular of HFrEF. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM23 and SM24, in particular SM23,
ii. at least one triacylglyceride biomarker selected from the group consisting of SOP2, and OSS2, in particular SOP2, and
iii. at least one further biomarker selected from the group consisting of Cholesterylester C18:2 and PC4, in particular Cholesterylester C18:2

are determined.

In particular, at least the amounts of SM23, SOP2, and Cholesterylester C18:2 are determined.

The aforementioned combinations are preferably used for the diagnosis of heart failure, in particular of HFrEF. In an embodiment, the heart failure is asymptomatic heart failure, in particular asymptomatic HFrEF. Accordingly, the subject to be tested preferably does not show symptoms of heart failure.

In a further preferred embodiment, at least the amounts of
i. at least one sphingomyelin biomarker selected from the group consisting of SM18, SM23, SM3, SM28 and SM24, in particular at least one sphingomyelin biomarker selected from the group consisting of SM18, SM23 and SM3,
ii. at least one triacylglyceride biomarker selected from the group consisting of SOP2 and OSS2, and
iii. at least one further biomarker selected from the group consisting of Cholesterylester C18:2 and PC4

are determined.

In particular, at least the amounts of SM18, of OSS2, and of Cholesterylester C18:2 are determined.

The aforementioned combinations are preferably used for the diagnosis of heart failure, in particular of HFrEF. In an embodiment, the heart failure is symptomatic heart failure, in particular symptomatic HFrEF. Accordingly, the subject to be tested preferably shows symptoms of heart failure.

In a further preferred embodiment, at least the amounts of
i. at least one sphingomyelin biomarker selected from the group consisting of SM23, SM24, SM3 and SM28, in particular at least one sphingomyelin biomarker selected from the group consisting of SM23 and SM24,
ii. at least one triacylglyceride biomarker selected from the group consisting of SOP2, and OSS2, and
iii. at least one further biomarker selected from the group consisting of Cholesterylester C18:2 and PC4

are determined.

In particular, at least the amounts of SM23, of SOP2, and of Cholesterylester C18:2 are determined.

The aforementioned combinations are preferably used for the diagnosis of heart failure, more preferably of HFrEF, and most preferably of DCMP. Preferably, the subject does not show symptoms of heart failure. Also preferably, the subject shows symptoms of heart failure.

In a further preferred embodiment, wherein at least the amounts of
i. at least one sphingomyelin biomarker selected from the group consisting of SM23 and SM24,
ii. at least one triacylglyceride biomarker selected from the group consisting of SOP2 and OSS2, and
iii. Cholesterylester C18:2

are determined.

In particular, at least the amounts of SM23, OSS2, and Cholesterylester C18:2 are determined.

The aforementioned combinations are preferably used for the diagnosis of heart failure, more preferably of HFrEF, and most preferably of DCMP, in particular of asymptomatic HFrEF and/or DCMP. Thus, the subject to be tested preferably does not show symptoms of HF.

In a further preferred embodiment, at least the amounts of
i. at least one sphingomyelin biomarker selected from the group consisting of SM3, SM24 and SM28,
ii. at least one triacylglyceride biomarker selected from the group consisting of SOP2 and OSS2, and
iii. at least one further biomarker selected from the group consisting of Cholesterylester C18:2 and PC4

are determined.

In particular, at least the amounts of SM3, OSS2, and of PC4 are determined.

The aforementioned combinations are preferably used for the diagnosis of heart failure, more preferably of HFrEF, and most preferably of DCMP, in particular of symptomatic HFrEF and/or DCMP. Thus, the subject to be tested preferably shows symptoms of HF.

In a further preferred embodiment, at least the amounts of
i. at least one sphingomyelin biomarker selected from the group consisting of SM18, SM23, and SM24, in particular at least one sphingomyelin biomarker selected from the group consisting of SM18 and SM23,
ii. at least one triacylglyceride biomarker selected from the group consisting of SOP2 and PPO1, in particular SOP2, and
iii. at least one further biomarker selected from the group consisting of Cholesterylester C18:2 and PC4, in particular Cholesterylester C18:2

are determined.

In particular, at least the amounts of SM18, SOP2, and Cholesterylester C18:2 are determined.

The aforementioned combinations are preferably used for the diagnosis of heart failure, more preferably of HFrEF, and most preferably of ICMP. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM24 and SM23,
ii. at least one triacylglyceride biomarker selected from the group consisting of SOP2 and PPO1, and
iii. at least one further biomarker selected from the group consisting of Cholesterylester C18:2 and PC4, in particular Cholesterylester C18:2

are determined.

In particular, at least the amounts of SM24, SOP2, and Cholesterylester C18:2 are determined.

The aforementioned combinations are preferably used for the diagnosis of heart failure, more preferably of HFrEF, and most preferably of ICMP, in particular of asymptomatic HFrEF and/or ICMP. Thus, the subject to be tested preferably does not show symptoms of HF.

In an even further preferred embodiment, at least the amounts of SM18, SOP2, and Cholesterylester C18:2 are determined. The aforementioned combination is preferably used for the diagnosis of heart failure, more preferably of HFrEF, and most preferably of ICMP, in particular of symptomatic HFrEF and/or ICMP. Thus, the subject to be tested preferably shows symptoms of HF.

Moreover, for the diagnosis of asymptomatic heart failure it is envisaged to determine the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2, PC4, SM5 and SSP2, in particular Cholesterylester C18:2.

Moreover, for the diagnosis of asymptomatic heart failure it is envisaged to determine the amounts of
i. at least one sphingomyelin biomarker selected from the group consisting of SM23, SM18, and SM2, in particular SM23,
ii. SOP2, and
iii. at least one further biomarker selected from the group consisting of SM5, Cholesterylester C18:2 PC4, in particular SM5.

Moreover, for the diagnosis of asymptomatic HFrEF it is envisaged to determine the amounts of
i. at least one sphingomyelin biomarker selected from the group consisting of SM23 and SM24,
ii. at least one triacylglyceride biomarker selected from the group consisting of SOP2, and OSS2, in particular SOP2, and
iii. at least one further biomarker selected from the group consisting of Cholesterylester C18:2 and SM28, in particular Cholesterylester C18:2.

Moreover, for the diagnosis of asymptomatic DCMP it is envisaged to determine the amounts of
i. at least one sphingomyelin biomarker selected from the group consisting of SM23 and SM24, in particular SM23,
ii. at least one triacylglyceride biomarker selected from the group consisting of SOP2 and OSS2, in particular OSS2 and
iii. Cholesterylester C18:2 and SSP2, in particular Cholesterylester C18:2.

Moreover, for the diagnosis of asymptomatic ICMP it is envisaged to determine the amounts of
i. at least one sphingomyelin biomarker selected from the group consisting of SM24 and SM23, in particular SM24,
ii. at least one triacylglyceride biomarker selected from the group consisting of SOP2 and PPO1, in particular SOP2, and
iii. at least one further biomarker selected from the group consisting of SM5, Cholesterylester C18:2 and PC4, in particular Cholesterylester C18:2, in particular SM5, or in particular PC4.

In one embodiment, the amount of NT-proBNP or BNP is determined in addition to the amount of the resulting combinations of the at least three biomarkers set forth above. In another embodiment, the amount of NT-proBNP or BNP is not determined in addition to the amount of the resulting combinations of the at least three biomarkers set forth above (see elsewhere herein).

Preferably, the method of the present invention does not comprise the determination of the amount of cysteine. In particular, the method shall not comprise the determination of amount of cysteine and the comparison of the amount of cysteine to a reference. Accordingly, it is preferably envisaged that the biomarker is not cysteine.

Also preferably, the method of the present invention does not comprise the determination of the amount of cholesterylester C18:1, or the comparison of the amount of cholesterylester C18:1 to a reference. In particular, the method shall not comprise the determination of amount of cholesterylester C18:1 and the comparison of the amount of cholesterylester C18:1 to a reference. Accordingly, it is preferably envisaged that the biomarker is not cholesterylester C18:1.

Also preferably, the method of the present invention does not comprise the determination of the amounts of cysteine and cholesterylester C18:1, or the comparison of the amount of cysteine and cholesterylester C18:1 to reference. In particular, the method shall not comprise the determination of amounts of cysteine and cholesterylester C18:1 and the comparison of the amount of cysteine and cholesterylester C18:1 to reference. Accordingly, it is preferably envisaged that the biomarkers are not cholesterylester C18:1 and cysteine.

Also preferably, the method of the present invention does not comprise the determination of the amount of cholesterylester C15:0, or the comparison of the amount of cholesterylester C15:0 to reference. In particular, the method shall not comprise the determination of the amount of cholesterylester C15:0 and the comparison of the amount of cholesterylester C15:0 to reference. Accordingly, it is preferably envisaged that the biomarker is not cholesterylester C15:0.

Also preferably, the method of the present invention does not comprise the determination of the amount of SM(d17:1, C23:0), or the comparison of the amount of SM(d17:1, C23:0) to reference. In particular, the method shall not comprise the determination of the amount of SM(d17:1, C23:0) and the comparison of the amount of the SM(d17:1, C23:0) to reference. Accordingly, it is preferably envisaged that the biomarker is not SM(d17:1, C23:0).

Also preferably, the method of the present invention does not comprise the determination of the amount of noradrenaline, or the comparison of the amount of noradrenaline to reference. In particular, the method shall not comprise the determination of amount of noradrenaline and the comparison of the amount of noradrenaline to reference. Accordingly, it is preferably envisaged that the biomarker is not noradrenaline.

If a biomarker is a triacylglyceride it is preferably envisaged that the determination of the amount of this biomarker does not encompass the derivatization of this marker. Accordingly, it is envisaged that the determination of this biomarker is not based on the determination of one or more of the fatty acid residues derived from said triacylglyceride. Accordingly, the amount of the entire triacylglyceride is measured.

Further, it is envisaged that method does not comprise the determination of tricosanoic acid and/or threo sphingosine, or the comparison of the amount of tricosanoic acid and/or threo sphingosine to reference.

The term “subject” as used herein relates to animals and, preferably, to mammals. More preferably, the subject is a primate and, most preferably, a human. The subject may be a female or, in particular, a male subject. The subject to be tested, preferably, is suspected to suffer from heart failure. The subject to be tested may have a history of myocardial infarction and/or may suffer from diabetes type II. Alternatively or additionally, the subject may suffer from another cardiac disease such as ischemic heart disease, cardioembolic stroke, hypertensive heart disease, rheumatic heart disease (RHD), aortic aneurysms, cardiomyopathy, atrial fibrillation, congenital heart disease, endocarditis, and peripheral artery disease (PAD), and atherosclerosis. Moreover, the subject may suffer from hypertension. Preferably, the subject is an adult. More preferably, the subject is older than 40 years of age, and most preferably older than 50 years of age. Further, it is envisaged that the subject is older than 54 years or age, but younger than 61 years of age.

More preferably, the subject may already show symptoms of the heart failure. Most preferably, the subject does not show symptoms of heart failure. Also encompassed as subjects are those, which belong into risk groups or subjects that are included in disease screening projects or measures.

In aged and overweight subjects, NT-proBNP and BNP are less reliable markers for heart failure. However, the determination of the at least three biomarkers as referred herein may improve the diagnostic accuracy in aged or overweight subjects. Therefore, the subject may be aged or overweight:

In one embodiment, the subject is older than 54 years of age, in particular older than 60 years of age, or even older than 70 years of age. As compared to the determination of NT-proBNP or BNP alone, the additional determination of the amount of the at least three biomarkers as set forth herein in connection with the method of the present invention allows for a more reliable diagnosis. In particular, the specificity of the diagnosis in said subject is increased.

In another embodiment, the subject has a body mass index (BMI) of more than 26.8 kg/m², in particular of more than 28.0 kg/m², or even more than 30.0 kg/m². As compared to the determination of NT-proBNP or BNP alone, the additional determination of the amount of the at least three biomarkers allows more reliable diagnosis. In particular, the sensitivity of the diagnosis in said subject is increased.

A subject who shows symptoms of heart failure in particular shows symptoms according to NYHA class II and/or III. Accordingly, the subject who shows symptoms of heart failure, preferably, has a slight or marked limitation of physical activity, is comfortable at rest, but ordinary or less than ordinary physical activity results in undue breathlessness, fatigue, or palpitations (in this subject). A subject who does not show symptoms of heart failure preferably has no limitation of physical activity, and ordinary physical activity results in undue breathlessness, fatigue, or palpitations (in this subject).

In a preferred embodiment, the present invention envisages the diagnosis of early stages of heart failure. Thus, the term “heart failure” means, in an embodiment, “early stage of heart failure”. For example, the presence or, in particular, the absence of an early stage of heart failure can be diagnosed.

An early stage of heart failure may be, in an embodiment, heart failure according to NYHA class I. In another embodiment, the early stage of heart failure may be heart failure according to NYHA class II. Thus, heart failure according to NYHA class I or NYHA class II can be diagnosed by using the at least three biomarkers as referred to herein (e.g. the biomarkers of panel 1).

With respect to HFrEF, an early stage of heart failure further may mean that the left ventricular ejection fraction is lower than 50% but larger than 35%. Thus, heart failure with a left ventricular ejection fraction that is lower than 50% but larger than 35% can be diagnosed by using the at least three biomarkers as referred to herein (e.g. the biomarkers of panel 1). In an embodiment, the subject preferably shows no symptoms of heart failure.

In the context of the studies underlying the present invention, it has been shown that the prediction probability is not significantly affected if the patient is treated with at least one 3-hydroxy-3-methyl-glutaryl-CoA reductase (HMGCR) inhibitor and/or with at least one diuretic. Thus, the subject to be tested in accordance with the method of the present invention is preferably treated with a 3-hydroxy-3-methyl-glutaryl-CoA reductase (HMGCR) inhibitor. Also preferably, the subject is treated with a diuretic. Further, it is envisaged that the subject may be treated with a 3-hydroxy-3-methyl-glutaryl-CoA reductase (HMGCR) inhibitors and a diuretic.

Preferred 3-hydroxy-3-methyl-glutaryl-CoA reductase (HMGCR) inhibitors are statins. Preferably, the statin is selected from the group consisting of atorvastatin, cerivastatin, fluvastatin, lovastatin, pitavastatin, pravastatin, mevastatin, rosuvastatin and simvastatin.

Preferably, the diuretic is selected from the group consisting of furosemide, bumethanide, ethacrynic acid, torasemide, chlortalidone, hydrochlorothiazide and metazolone.

Moreover, the subject may not be treated with a statins and/or a diuretic.

In an embodiment of the method of the present invention, the subject is preferably female, older than 54 years of age (in particular older than 60), and has a body mass index (BMI) of less than 25.0 kg/m², in particular of less than 24.0 kg/m². For these subjects it has been shown that the determination of the at least three biomarkers and of NT-proBNP allows to more reliably classify a healthy subject (i.e. a subject who does not suffer from heart failure) as not to suffer from heart failure. In contrast, the determination of NT-proBNP alone might result in a large percentage of false positive results (see Example 8, results for panel 1 combined with NT-proBNP). For subjects who are female, older than 54 years of age, and who have a body mass index of less than 25.0 kg/m², the specificity of the diagnosis of heart failure is increased (as compared to NT-proBNP or BNP alone).

Preferred combinations of at least three biomarkers that are used for diagnosing heart failure, if the subject shows symptoms of heart failure, are described in connection with the diagnosis of symptomatic heart failure. Further preferred combinations of at least three biomarkers for diagnosing heart failure in subjects showing symptoms of heart failure are those biomarker panels in Table 2 for symptomatic subgroups (see column “subgroup”).

Preferred combinations of at least three biomarkers that are used for diagnosing heart failure, if the subject does not show symptoms of heart failure, are described in connection with the diagnosis of asymptomatic heart failure. Further preferred combinations of at least three biomarkers for diagnosing heart failure in subjects not showing symptoms of heart failure are those biomarker panels in Table 2 for asymptomatic subgroups (see column “subgroup”).

Preferably, the subject, however, is besides the aforementioned diseases and disorders apparently healthy. Also preferably, the subject shall not suffer from apoplex (stroke), myocardial infarction within the last 4 month before the sample has been taken or from acute or chronic inflammatory diseases and malignant tumors. Furthermore, the subject is preferably in stable medications within the last 4 weeks before the sample was taken.

In a preferred embodiment, the subject to be tested does not suffer from impaired renal function. However, it is also contemplated that the subject suffers from impaired renal function. Renal function can be assessed e.g. by determining the glomerular filtration rate (GFR). Preferably, a subject suffers from impaired renal function if the GFR is below 60 mL/min/1.73 m², or in particular below 50 mL/min/1.73 m². Preferably, a subject does not suffer from impaired renal function if the GFR is above 60 mL/min/1.73 m², or in particular above 70 mL/min/1.73 m².

More preferably, the subject who does not suffer impaired renal disease does not suffer from chronic kidney disease stages 3 to 5 (for the classification, see e.g. National Kidney Foundation, 2002. K/DOQI clinical practice guidelines for chronic kidney disease: evaluation, classification, and stratification. Am. J. Kidney Dis. 39, S1-266 which herewith is incorporated by reference in its entirety.)

GFR may be accurately calculated by comparative measurements of substances in the blood and urine, or estimated by formulas using just a blood test result (eGFR). Usually these estimates are used in clinical practice in particular in elderly and sick patients where reliable urine collections are difficult. These tests are important in assessing the excretory function of the kidneys, for example in grading of chronic renal insufficiency.

eGFR is associated with GFR via a large clinical study (National Kidney Foundation (February 2002). “K/DOQI clinical practice guidelines for chronic kidney disease: evaluation, classification, and stratification”. American Journal of Kidney Diseases 39 (2 Suppl 1): 51-266. doi:10.1016/50272-6386(02)70081-4. PMID 11904577). For clinical assessment scales of eGFR and GFR can be used interchangeably.

The term “sample” as used herein refers to samples from body fluids, preferably, blood, plasma, serum, saliva or urine, or samples derived, e.g., by biopsy, from cells, tissues or organs, in particular from the heart. More preferably, the sample is a blood, plasma or serum sample, most preferably, a plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

In a preferred embodiment of the present invention, the sample is fasting sample, in particular a fasting blood, serum or plasma sample. Accordingly, the sample shall have been obtained from a fasting subject. A fasting subject, in particular, is a subject who refrained from food and beverages, except for water, prior to obtaining the sample to be tested. Preferably, a fasting subject refrained from food and beverages, except for water, for at least eight hours prior to obtaining the sample to be tested. More preferably, the sample has been obtained from the subject after an overnight fast.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

As set forth herein below in more detail, the determination of the amount of a biomarker as referred to herein, preferably includes a separation step, i.e. a step in which compounds comprised by the sample are separated. Preferably, the separation of the compounds is carried by chromatography, in particular by liquid chromatography (LC) or high performance liquid chromatography (HPLC). Preferably, the pre-treatment of the sample should allow for a subsequent separation of compounds, in particular the metabolite biomarkers as referred to above, comprised by the sample. Molecules of interest, in particular the biomarkers as referred to above may be extracted in an extraction step which comprises mixing of the sample with a suitable extraction solvent. The extraction solvent shall be capable of precipitating the proteins in a sample, thereby facilitating the, preferably, centrifugation-based, removal of protein contaminants which otherwise would interfere with the subsequent analysis of the biomarkers as referred above. Preferably, the at least three biomarkers as referred to herein, in particular all lipid biomarkers as referred above, are soluble in the extraction solvent. More preferably, the extraction solvent is a one phase solvent. Even more preferably, the extraction solvent is a mixture comprising a first solvent selected from the group consisting of dichloromethane (DCM), chloroform, tertiary butyl methyl ether (tBME or MTBE, also known as 2-methoxy-2-methylpropane), ethyl ethanoate, and isooctane, and a second solvent selected from the group consisting of methanol, ethanol, isopropanol and dimethyl sulfoxide (DMSO). In an embodiment the extraction solvent comprises methanol and DCM, in particular a ratio of about 2:1 to about 3:2, preferably a ratio of about 2:1 or about 3:2 (preferably v/v). The term “about” as used herein refers to either the precise value indicated afterwards or to a value differing +/−20%, +/−10%, +/−5%, +/−2% or +/−1% from the said precise value.

Preferably, the pretreatment of the sample comprises an extraction step with a suitable extraction solvent. This extraction step additionally results in the precipitation of proteins comprised by the sample. Subsequently, the proteins comprised by the sample are removed by centrifugation.

As described herein below, the method of the present invention may further comprise the determination of the amount of a protein marker useful in the diagnosis of heart failure, preferably the protein marker NT-proBNP or BNP. These markers are preferably determined by using antibodies which specifically bind to BNP or NT-proBNP (or alternatively by other methods known in the art). The pretreatment as described in the two paragraphs above, thus, does not apply to the determination of protein markers, in particular BNP and NT-proBNP. Preferably, the pre-treatment applies to the determination of the amount of the metabolite biomarkers only, i.e. of sphingomyelins, triacylglycerides, cholesterylesters, phosphatidylcholines, ceramides and/or glutamic acid.

The term “determining the amount”, in particular of the metabolite biomarkers, as used herein refers to determining at least one characteristic feature of a biomarker to be determined by the method of the present invention in the sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present, is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Thus, the determination of the amount of a biomarker as referred to herein is preferably done by a compound separation step and a subsequent mass spectrometry step. Thus, determining as used in the method of the present invention, preferably, includes using a compound separation step prior to the analysis step. Preferably, said compound separation step yields a time resolved separation of the metabolites, in particular of the at least three biomarkers, comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or HPLC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). More preferably, LC-MS, in particular LC-MS/MS, and most preferably HPLC-MS, in particular HPLC-MS/MS, are used as described in detail below. Accordingly, the determination of the amounts to the at least three biomarkers is preferably carried by HPLC-MS, in particular HPLC-MS/MS (high-performance liquid chromatography tandem mass spectrometry). The techniques described above are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference.

As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado.

The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

As described above, said determining of the at least three biomarkers can, preferably, comprise mass spectrometry (MS). Thus, a mass spectrometry step is carried out after the separation step (e.g. by LC or HPLC). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or HPLC-MS, i.e. to mass spectrometry being operatively linked to a prior liquid chromatography separation step. Preferably, the mass spectrometry is tandem mass spectrometry (also known as MS/MS). Tandem mass spectrometry, also known as MS/MS involves two or more mass spectrometry step, with a fragmentation occurring in between the stages. In tandem mass spectrometry two mass spectrometers in a series connected by a collision cell. The mass spectrometers are coupled to the chromatographic device. The sample that has been separated by a chromatography is sorted and weighed in the first mass spectrometer, then fragmented by an inert gas in the collision cell, and a piece or pieces sorted and weighed in the second mass spectrometer. The fragments are sorted and weighed in the second mass spectrometer. Identification by MS/MS is more accurate.

In an embodiment, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once.

More preferably, said mass spectrometry is liquid chromatography (LC) MS such high performance liquid chromatography (HPLC) MS, in particular HPLC-MS/MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. For examples, HPLC can be carried out with commercially available reversed phase separation columns with e.g. C8, C18 or C30 stationary phases. The person skilled in the art is capable to select suitable solvents for the HPLC or any other chromatography method as described herein. The eluate that emerges from the chromatography device shall comprise the biomarkers as referred to above.

A suitable solvent for elution for lipid chromatography can be determined by the skilled person. In an embodiment, the solvents for gradient elution in the HPLC separation consist of a polar solvent and a lipid solvent. Preferably, the polar solvent is a mixture of water and a water miscible solvent with an acid modifier. Examples of suitable organic solvents which are completely miscible with water include the C1-C3-alkanols, tetrahydrofurane, dioxane, C3-C4-ketones such as acetone and acetonitril and mixtures thereof, with methanol being particularly preferred. Additionally the lipid solvent is a mixture of the above mentioned solvents together with hydrophobic solvents from the groups consisting of dichloromethane (DCM), chloroform, tertiary butyl methyl ether (tBME or MTBE), ethyl ethanoate, and isooctane. Examples of acidic modifiers are formic acid or acidic acid. Preferred solvents for gradient elution are disclosed in the Examples section.

Gas chromatography which may be also applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatized prior to gas chromatography. Suitable techniques for derivatization are well known in the art. Preferably, derivatization in accordance with the present invention relates to methoxymation and trimethylsilylation of, preferably, polar compounds and transmethylation, methoxymation and trimethylsilylation of, preferably, nonpolar (i.e. lipophilic) compounds.

For mass spectrometry, the analytes in the sample are ionized in order to generate charged molecules or molecule fragments. Afterwards, the mass-to-charge of the ionized analyte, in particular of the ionized biomarkers, or fragments thereof is measured.

Thus, the mass spectrometry step preferably comprises an ionization step in which the biomarkers to be determined are ionized. Of course, other compounds present in the sample/elulate are ionized as well. Ionization of the biomarkers can be carried out by any method deemed appropriate, in particular by electron impact ionization, fast atom bombardment, electrospray ionization (ESI), atmospheric pressure chemical ionization (APCI), matrix assisted laser desorption ionization (MALDI).

Preferably, the ionization step is carried out by atmospheric pressure chemical ionization (APCI). More preferably, the ionization step (for mass spectrometry) is carried out by electrospray ionization (ESI). Accordingly, the mass spectrometry is preferably ESI-MS (or if tandem MS is carried out: ESI-MS/MS). Electrospray is a soft ionization method which results in the formation of ions without breaking any chemical bonds.

Electrospray ionization (ESI) is a technique used in mass spectrometry to produce ions using an electrospray in which a high voltage is applied to the sample to create an aerosol. It is especially useful in producing ions from macromolecules because it overcomes the propensity of these molecules to fragment when ionized.

Preferably, the electrospray ionization is positive ion mode electrospray ionization. Thus, the ionization is preferably a protonation (or an adduct formation with positive charged ions such as NH₄⁺, Na⁺, or K⁺, in particular NH₄⁺). According a suitable cation, preferably, a proton (Hi) is added to the biomarkers to be determined (and of course to any compound in the sample, i.e. in the eluate from the chromatography column). Therefore, the determination of the amounts of the at least three biomarkers might be the determination of the amount of protonated biomarkers.

The person skilled in the art knows that the ionization step is carried out at the beginning of the mass spectrometry step. If tandem MS is carried out, the ionization, in particular the electrospray ionization, is carried out in the first mass spectrometry step.

The ionization of the biomarkers can be preferably carried out by feeding the liquid eluting from the chromatography column (in particular from the LC or HPLC column) directly to an electrospray. Alternatively the fractions can be collected and are later analyzed in a classical nanoelectrospray-mass spectrometry setup.

As set forth above, the mass spectrometry step is carried out after the separation step, in particular the chromatography step. In an embodiment, the eluate that emerges from the chromatography column (e.g. the LC or HPLC column) may be pre-treated prior to subjecting it to the mass spectrometry step. Preferably, ammonium buffer (most preferably ammonium formate or ammonium acetate) is added to the eluate in order to enhance the ionization efficiency in the electrospray process for some lipids such as ceramides and TAGs. Preferably, the ammonium formate buffer is dissolved in a solvent miscible with the gradient HPLC solvents (most preferably methanol).

In a preferred embodiment of the present invention, the at least three biomarkers are determined together in a single measurement. In particular, it is envisaged to determine the amounts together in a single LC-MS (or LC-MS/MS), HPLC-MS (HPLC-MS/MS) measurement (i.e. run).

In an embodiment, the determination of the amounts of the at least three biomarkers comprises steps as described in the Examples section. For example, the at least three biomarkers can be determined as described in Example 5 or in Examples 11 to 15. In particular, the biomarkers of panels 1, 2 or 200 can be determined as described in the Examples section (preferably, Examples 11 to 15).

For example, a blood, serum, or plasma sample (in particular a plasma sample) can be analyzed. The sample may be a fresh sample or a frozen sample. If frozen, the sample may be thawed at suitable temperature for a suitable time. An aliquot of the sample (e.g. said aliquot of the sample my have a volume of 10 μl) is then transferred to microcentrifuge tube and mixed with a suitable extraction solvent (e.g. about 1:150 v/v in methanol/dichloromethane (2:1 v/v)). An internal standard may be added. E.g. In case the amounts of the at least three biomarkers of panel 1 will be determined, phosphatidylcholine (C19:0 C19:0) dissolved in methanol/dichloromethane (2:1 v/v) may be used as internal standard; for example, 10 μl of respective 0.05 mmolar solution in methanol/dichloromethane (2:1 v/v). Afterwards an extraction is done (e.g. for 5 minutes using a vortexer). After extraction, the sample may be centrifuged (e.g. at about 20.000 g). An aliquot of the supernatant (e.g. 200 μl) could be used for the quantification of the markers. The supernatant may be stored at −20° C. (preferably, up to three months).

In addition, at least one internal standard compound can be added to the sample. As used herein, the term “internal standard compound” refers to a compound which is added to the sample and which is determined (i.e. the amount of the internal standard compound is determined). The at least one internal standard compound can be added before, during, or after extraction of the sample to be tested. In an embodiment, the at least one internal standard compound is added after mixing an aliquot of the sample with the extraction solvent. Preferably, the internal standard compound is thus dissolved in the extraction solvent. In an embodiment, the extraction solvent is a an extraction solvent as described elsewhere herein (such as methanol/dichloromethane (2:1 v/v)). Preferably, the at least one internal standard compound is a lipid metabolite biomarker. More preferably, the internal standard compound is a compound, in particular a lipid, which is essentially not present or which is not present in the sample to be tested. Thus, the compound is preferably not naturally present in the sample to be tested. Preferably, the internal standard is very similar to a respective lipid biomarker according to the present invention. In an embodiment, the at least one standard compound is selected from the group consisting of lysophosphatidylcholine C17:0, ceramide d18:1117:0, phosphatidylcholine C19:0 C19:0, cholesteryl heptadecanoate (CE C17:0), and glyceryl triheptadecanoate (MMM). In particular, the internal standard compound is phosphatidylcholine C19:0 C19:0.

The internal standard compound(s) can be dissolved in a suitable solvent (the solution comprising the internal standard compound and the suitable solvent is herein also refereed to as “internal standard solution”). Preferably, the solvent comprises or is an unpolar liquid solvent, like methylchlorid, dichlormethan, chloroform, 1,2-dichlorethan. Preferably, an extraction solvent as described elsewhere herein is used. Even more preferably the solvent is a mixture comprising dichloromethane (DCM) and methanol. In an embodiment the extraction solvent comprises methanol and DCM, in particular in a ratio of about 2:1 (preferably volume to volume).

Preferably, the internal standard solution is added to the sample to be tested after extracting the samples with an extraction solvent as described elsewhere herein and removing the proteins from the sample by centrifugation. Thus, the internal standard solution shall be added to a pre-treated sample.

If phosphatidylcholine C19:0 C19:0 is used as internal standard compound, the concentration of the internal standard compound in the internal standard solution to be added to the (pretreated) sample is preferably within a range of 1 to 200 μg/ml, more preferably 35 to 50 μg/ml, most preferably 40 to 45 μg/ml. With regard to the sample after preparation (i.e., as used for the actual measurement), the concentration of the internal standard compound is preferably within a range of 0.01-1.3 μg/ml, more preferably 0.23 to 0.33 μg/ml, most preferably 0.26 to 0.30 μg/ml after sample preparation.

In an embodiment, the volume of the internal standard solution to be used is the same volume as the sample before pretreatment. E.g. 10 μl of plasma and internal standard solution are used.

The determination of the amount of the internal standard shall preferably allow for a normalization of the amounts of the at least three biomarkers as referred to herein. Preferably, the determined peak areas for the at least three biomarkers are divided by the peak area of the at least one internal standard compound.

In particular, it is possible to calculate a correction factor for the test samples (samples from subjects to be tested, but also calibration samples). This correction factor can be used in order to correct the peak area of the at least three biomarkers for variations of the devices, inherent system errors, or the like. For each biomarker determined as peak area, the area ratio of the biomarker peak area to the internal standard peak area can be determined.

Preferably, the determination of the at least one standard compound does not interfere with the determination of the amounts of the at least three biomarkers.

The term “amount” as used herein preferably encompasses the absolute amount of a biomarker as referred to herein, the relative amount or concentration of the said biomarker as well as any value or parameter which correlates thereto or can be derived therefrom. Such values or parameters comprise intensity signal values from specific physical or chemical properties obtained from the said biomarker. In particular, encompassed shall be values or parameters which are obtained by indirect measurements specified elsewhere in this description. It is to be understood that values correlating to the aforementioned amounts or parameters can also be obtained by standard mathematical operations.

In an embodiment, the term “amount” refers to the absolute amount. How to determine the absolute amount is e.g. described for OSS2; SM23; CE C18:2, and PC4 in Examples 11 to 15.

In addition, it is contemplated to determine only the amounts of the at least three biomarkers as referred to herein, i.e. no further biomarkers (except for a natriuretic peptide such as NT-proBNP) are determined. For example, if the biomarkers of panel 1 are determined, only the amounts OSS2, PC4 and SM23 are determined (and taken into account for the diagnosis, and optionally a natriuretic peptide). Thus, the group of lipid biomarkers to be determined consists of OSS2, PC4 and SM23. If the biomarkers of panel 2 are determined, the group of lipid biomarkers to be determined consists of OSS2; SM23; CE C18:2. If the biomarkers of panel 200 are determined, the group of lipid biomarkers to be determined consists of OSS2; SM23; CE C18:2; PC4.

In a preferred embodiment of the present invention, the method according to the present invention further comprises the determination of the amount of BNP (brain natriuretic peptide, also known as B-type natriuretic peptide) or, in particular, NT-proBNP (N-terminus of the prohormone brain natriuretic peptide) in a sample from the subject and the thus determined amount of BNP or NT-proBNP is then compared to a reference. In a preferred embodiment the method according to the invention further comprises, in particular in step a) the determination of the amount of BNP (Brain natriuretic peptide, also known as B-type natriuretic peptide) or, in particular, NT-proBNP (N-terminus of the prohormone brain natriuretic peptide) in a sample from the subject. The thus determined amount of BNP or NT-proBNP is then compared to a reference (in step b) for BNP or NT-proBNP. Thus, the at least three biomarkers and BNP or NTproBNP might be determined at the same time (but preferably by varying assays). In another preferred embodiment of the present invention, the amount of NT-proBNP or BNP is determined in a sample in a further step c), and compared to a reference for NT-proBNP or BNP in a further step d). Based on steps b) and d) heart failure is diagnosed. Thus, the may be a time gap between the determination of the amounts of the at least three biomarkers and the determination of the amount of BNP or NT-proBNP (such as one day or one week).

Alternatively, the amount of NT-proBNP or BNP can be derived from the medical record of the subject to be tested. Thus, the method of the present invention may comprise the step of providing and/or retrieving information on the amount of NT-proBNP or BNP (i.e. the value of this marker).

NT-proBNP and BNP are protein markers. The markers NT-proBNP and BNP are well known in the art. BNP is a 32-amino acid polypeptide secreted by the ventricles of the heart in response to excessive stretching of heart muscle cells (cardiomyocytes). NT-proBNP is a 76 amino acid N-terminal inactive protein that is cleaved from proBNP to release brain natriuretic peptide. BNP is the active hormone and has a shorter half-life than the respective inactive counterpart NT-proBNP. The structure of the human BNP and NT-proBNP has been described already in detail, see e.g., WO 02/089657, WO 02/083913.

It is to be understood that if the protein marker BNP or NTproBNP is determined, the protein marker is determined in addition to the at least three (metabolite) biomarkers as referred to herein in accordance with the method of the present invention.

The determination of the amount of BNP/NT-proBNP may differ from the determination of the amount of the at least three biomarkers as referred to in the context of the method of the present invention (since the amounts of the at least three biomarkers are preferably determined by the methods involving chromatography and mass spectrometry, see elsewhere herein). Preferably, the amount of NT-proBNP or BNP is determined in a blood, serum or plasma sample. Preferably, the amount is determined by using at least one antibody which specifically binds to NT-proBNP or BNP. The at least one antibody forms a complex with the marker to determined (NT-proBNP or BNP). Afterwards the amount of the formed complex is measured. The complex comprises the marker and the antibody (which might be labelled in order to allow for a detection of the complex).

It is to be understood that the sample in which NT-proBNP or BNP is determined requires or may require a pretreatment which differs from the pretreatment of the sample in which the other biomarkers as referred to herein are determined. For example, the proteins comprised by the sample in which this marker is determined are not precipitated. This is taken into account by the skilled person. Preferably, however, the amounts of the at least three biomarkers and of BNP or NT-proBNP are measured in aliquots derived from the same sample. Alternatively, the amounts of the at least three biomarkers and of BNP or NT-proBNP may be measured in aliquots derived from separate samples from the subject.

The further determination of the protein marker NT-proBNP or BNP allows for a more reliable diagnosis. In particular, it has been shown in the studies of the present invention that the combination of a limited number of metabolites, i.e. of the at least three biomarkers as referred to above, with NT-proBNP allows for overcoming the limitations of single feature markers in a complex disease such as heart failure.

In an embodiment of the present invention, the amount of the protein marker NT-proBNP or BNP is determined for the marker panels in Table 2 for which NT-proBNP has been determined in the studies underlying the present invention (indicated with “yes” in column “with NT-proBNP”).

In addition, several marker combinations that were tested in the studies underlying the present invention gave very accurate results even in the absence of the determination of NT-proBNP (see results in the Examples section, e.g. Table 2). For example, the determination of the biomarkers of the saturated panels of Table 2 in the Examples section (indicated with “yes” in the column “saturated panel”) resulted in relatively high AUC values, even if the gold-standard NT-proBNP was not taken into account (see e.g. Table 6 and/or Tables 4A, 4B, 4C, and 4D). So, in another embodiment of the present invention the method according to the present invention does not comprise the further determination of the amount of BNP or NT-proBNP. In particular, the method may not comprise the further determination of the amount of BNP or NT-proBNP and the comparison of the amount of BNP or NT-proBNP to a reference. Thus, the diagnosis of heart failure is not based on the determination of BNP and/or NT-proBNP. Accordingly, the method is a non-BNP and/or non-NT-proBNP based method.

Instead of NT-proBNP or BNP, or in addition to NT-proBNP or BNP, the present invention further envisages the determination of the amount of ANP (atrial natriuretic peptide) or NT-proANP (N-terminus of the prohormone brain natriuretic peptide). Alternatively, the amount of C-type natriuretic peptide or natriuretic peptide precursor C can be determined.

In an embodiment, the method of the present invention may further comprise carrying out a correction for confounders. Preferably, the values or ratios determined in a sample of a subject according to the present invention are adjusted for age, BMI, gender or other existing diseases, e.g., the presence or absence of diabetes before comparing to a reference. Alternatively, the references can be derived from values or ratios which have likewise been adjusted for age, BMI, gender (see Examples). Such an adjustment can be made by deriving the references and the underlying values or ratios from a group of subjects the individual subjects of which are essentially identical with respect to these parameters to the subject to be investigated. Alternatively, the adjustment may be done by statistical calculations. Thus, a correction for confounders may be carried out. In an embodiment, a correction for confounders is carried out for those marker panels in Table 2 (and Table 2a) for which an ANOVA correction has been carried out in the studies underlying the present invention (indicated with “yes” in column “with ANOVA”). Preferred confounders are age, BMI (body mass index) and gender.

In another embodiment, a correction for confounders is not carried out. In an embodiment, a correction for confounders is not carried out for those marker panels in Table 2 (and Table 2a) for which an ANOVA correction was not carried out in the studies underlying the present invention (indicated with “no” in column “with ANOVA”). In a preferred embodiment, no correction for the confounders age, BMI and gender is carried out. As it can be derived from the Examples section, a reliable diagnosis is possible even without correction for confounders (see also respective Tables in the Examples section).

The determination of the amounts of the at least three biomarkers allows for a reliable rule-in (as is the diagnosis of the presence of heart failure), in particular of HFrEF (or subforms thereof), even in patients with increased body mass index (BMI>26.8 kg/m²), especially at younger age (such as <61 years). This applies especially in the absence of a correction for confounders (in particular age, body mass index and gender). This applies in particular for the diagnosis of early stages of heart failure.

Subjects not suffering from heart failure can be more accurately identified, even in patients at elevated age (such as >54 years), especially with decreased body mass index (BMI<25.0 kg/m²). In these subjects, a more reliable rule-out (as is the diagnosis of the absence of heart failure) is possible. This applies especially in the absence of a correction for confounders (in particular age, body mass index and gender).

As set forth above, a correction for confounders may not be carried out, i.e. may be not required for a reliable diagnosis. This is advantageous since the diagnosis can be done even without the knowledge of certain patient's characteristics such as age, body mass index and gender. Thus, in an embodiment, the age, body mass index and/or gender, in particular the age and/or body mass index are not known (and thus are not taken into account for the diagnosis).

The term “reference” in connection with diagnostic methods is well known in the art. The reference in accordance with the present invention shall allow for the diagnosis of heart failure. A suitable reference may be established by the skilled person without further ado. The reference to be applied may be an individual reference for each of the at least three biomarkers to be determined in the method of the present invention (and for BNP or NT-proBNP if determined). Accordingly, the amount of each of the at least three biomarkers (and of BNP or NT-proBNP if determined) as referred to in step a) of the method of the present invention is compared to a reference for each of the at least three biomarkers (and for BNP or NT-proBNP if determined). For example, if three biomarkers are determined in step a), three references (a reference for the first, a reference for the second, and a reference for the third marker) are applied in step b). A further reference for BNP or NT-proBNP might be used, if the amount of BNP or NT-proBNP is determined in step a). Based on the comparison of the amounts of the at least three biomarkers (and if BNP or NT-proBNP are determined, the amount of BNP or NT-proBNP), with the references, a diagnosis of heart failure, i.e. whether the subject as referred to herein suffers from heart failure, or not, can be established.

The term “reference” refers to values of characteristic features which can be correlated to a medical condition, i.e. the presence or absence of the disease, diseases status or an effect referred to herein. Preferably, a reference is a threshold value for a biomarker of the at least three biomarkers as referred to in connection with the present invention whereby values found in a sample to be investigated which are higher than (or depending on the marker lower than) the threshold are indicative for the presence of heart failure while those being lower (or depending on the marker higher than) are indicative for the absence of heart failure.

The diagnostic algorithm may depend on the reference. If the reference amount is e.g. derived from a subject or group of subjects known to suffer from heart failure, the presence of heart failure is preferably indicated by amounts in the test sample which are essentially identical to the reference(s). If the reference amount is e.g. derived from an apparently healthy subject or group thereof, the presence of heart failure is preferably indicated by amounts of the at least three biomarkers in the test sample which are different from (e.g. increased (“up”) or decreased (“down”) as compared to) the reference(s).

In accordance with the aforementioned method of the present invention, a reference (or references) is, preferably, a reference (or references) obtained from a sample from a subject or group of subjects known to suffer from heart failure. In such a case, a value for each of the at least three biomarkers found in the test sample being essentially identical is indicative for the presence of the disease, i.e. of heart failure (in particular for the subform thereof such as HFrEF, DCMP, ICMP and/or HFpEF). Moreover, the reference, also preferably, could be from a subject or group of subjects known not to suffer from heart failure, preferably, an apparently healthy subject or group of subjects. In such a case, a value for each of the at least three biomarkers found in the test sample being altered with respect to the reference is indicative for the presence of the disease. Alternatively, a value for each of the at least three biomarkers found in the test sample being essentially identical with respect to the reference is indicative for the absence of the disease. The same applies mutatis mutandis for a calculated reference, most preferably the average or median, for the relative or absolute value of the biomarkers of a population of individuals comprising the subject to be investigated. The absolute or relative values of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

The value for a biomarker of the test sample and the reference values are essentially identical, if the values for the characteristic features and, in the case of quantitative determination, the intensity values are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts or values are essentially identical are well known in the art and are also described elsewhere herein.

An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. Preferred changes and ratios of the medians are described in the Examples (see in particular Table 1A and 1B).

In a preferred embodiment the value for the characteristic feature can also be a calculated output such as score of a classification algorithm like “elastic net” as set forth elsewhere herein.

Preferably, the reference, i.e. a value or values for at least one characteristic feature (e.g. the amount) of the biomarkers or ratios thereof, will be stored in a suitable data storage medium such as a database and are, thus, also available for future assessments.

The term “comparing”, preferably, refers to determining whether the determined value of the biomarkers, or score (see below) is essentially identical to a reference or differs therefrom. Preferably, a value for a biomarker (or score) is deemed to differ from a reference if the observed difference is statistically significant which can be determined by statistical techniques referred to elsewhere in this description. If the difference is not statistically significant, the biomarker value and the reference are essentially identical. Based on the comparison referred to above, a subject can be assessed to suffer from the heart failure (in particular, from a subform thereof), or not.

For the specific biomarkers referred to in this specification, preferred values for the changes in the relative amounts or ratios (i.e. the changes expressed as the ratios of the means) or the kind of direction of change (i.e. “up”- or “down” or increase or decrease resulting in a higher or lower relative and/or absolute amount or ratio) are indicated in the Table 1A and 1B in the Examples section. The ratio of means indicates the degree of increase or decrease, e.g., a value of 2 means that the amount is twice the amount of the biomarker compared to the reference. Moreover, it is apparent whether there is an “up-regulation” or a “down-regulation”. In the case of an “up-regulation” the ratio of the mean shall exceed 1.0 while it will be below 1.0 in case of a “down”-regulation. Accordingly, the direction of regulation can be derived from the Table as well. It will be understood that instead of the means, medians could be used as well.

E.g. with respect to the biomarkers of panel 1, the presence of heart failure is preferably diagnosed in the subject when the amount of OSS2 in the sample is greater than the reference and the amount of SM23 in the sample is less than the reference, and the amount of PC4 in the sample is less than the reference. The absence of heart failure is preferably diagnosed in the subject when the amount of OSS2 in the sample is lower than the reference and their amount of SM23 in the sample is greater than the reference, and the amount of PC4 in the sample is lower than the reference. This e.g. applies if a reference is used for each biomarker. For the reference score, see elsewhere herein.

In the examples section, the reference was derived from healthy control subjects, i.e. subjects known not to suffer from heart failure. However, other references, in principle, could be established as well.

As can be derived from Table 1A and 1B, an increased amount of a triacylglyceride biomarker as compared to the reference shall be indicative for the presence of heart failure (and thus for the diagnosis of heart failure), whereas a decreased or an essentially identical amount as compared to the reference shall be indicative for the absence of heart failure. Preferably, said reference is a reference derived from healthy control subjects (i.e. subjects known not to suffer from heart failure), or from a healthy control subject.

With respect to the other further biomarkers as disclosed herein (i.e. of sphingomyelin biomarker, cholesterylester biomarker, phosphatidylcholine biomarker, ceramide biomarker and glutamic acid) a decreased amount of the biomarker as compared to the reference shall be indicative for the presence of heart failure (and thus for the diagnosis that the patient does not suffer from heart failure), whereas an increased or an essentially identical amount as compared to the reference shall be indicative for the absence of heart failure. Preferably, said reference is a reference derived from healthy control subjects (i.e. subjects known not to suffer from heart failure), or from a healthy control subject.

If the amount of NT-proBNP or BNP is determined, an increased amount of this marker shall be indicative for the presence of heart failure (and thus for the diagnosis of heart failure), whereas a decreased or an essentially identical amount shall be indicative for the absence of heart failure. In an embodiment, the reference amount for NT-proBNP is about 125 pg/mL, preferably in a serum or plasma sample, in particular for a human subject. However, further references may be used (e.g. since the level can depend on the age).

It is to be understood that the diagnostic algorithm might depend on the reference or references to be applied. However, this is taken into account by the skilled person who can establish suitable reference values and/or diagnostic algorithms based on the diagnosis provided herein. For example, the reference might be derived from a subject or group of subjects known to suffer from heart failure. In this case the following applies: With respect to triacylglyceride biomarker, an amount (or amounts) of the triacylglyceride(s) in the sample from the subject which is (are) essential identical or which is (are) increased as compared to the reference is indicative for the presence of heart failure. With respect to the remaining biomarkers that can determined, an amount (or amounts) of the remaining biomarker(s) in the sample from the subject which is (are) decreased or essentially identical as compared to the reference is (are) indicative for the presence of heart failure.

The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In the context of step b) of the present invention, the amounts of a group of biomarkers as referred to in step a) of the methods of the present invention shall be compared to a reference or references. Thereby, the presence or absence of a disease as referred to herein is diagnosed. In an embodiment references for the individual determined biomarkers, i.e. references for each of biomarkers as referred to in step a) are applied. However, it is also envisaged to calculate a score (in particular a single score) based on the amounts of the at least three biomarkers as referred to in step a) of the method of the present invention, i.e. a single score, and to compare this score to a reference score. Preferably, the score is based on the amounts of the at least three biomarkers in the sample from the test subject, and, if NT-proBNP or BNP is determined, on the amounts of the at least three biomarkers and the amount of NT-proBNP or BNP in the sample from the test subject. For example, if the amounts of the biomarkers of panel 1 are determined, the calculated score is based on the amounts of SM23, OSS2 and PC4 in the sample from the test subject. If additionally NT-proBNP is determined, the score is based on the amount of SM23, OSS2, PC4 and NT-proBNP of the test subject.

The calculated score combines information on the amounts of the at least three biomarkers. Moreover, in the score, the biomarkers are, preferably, weighted in accordance with their contribution to the establishment of the diagnosis. Based on the combination of biomarkers applied in the method of the invention, the weight of an individual biomarker may be different.

The score can be regarded as a classifier parameter for diagnosing heart failure. In particular, it enables the person who provides the diagnosis based on a single score based on the comparison with a reference score. The reference score is preferably a value, in particular a cut-off value which allows for differentiating between the presence of heart failure and the absence of heart failure in the subject to be tested. Preferably, the reference is a single value. Thus, the person does not have to interpret the entire information on the amounts of the individual biomarkers.

Using a scoring system as described herein, advantageously, values of different dimensions or units for the biomarkers may be used since the values will be mathematically transformed into the score. Accordingly, as described in Example 5, below, e.g., values for absolute concentrations may be combined in a score with peak area ratios.

Thus, in a preferred embodiment of the present invention, the comparison of the amounts of the biomarkers to a reference as set forth in step b) of the method of the present invention encompasses step b1) of calculating a score based on the determined amounts of the biomarkers as referred to in step a), and step b2) of comparing the, thus, calculated score to a reference score. More preferably, a logistic regression method is used for calculating the score and, most preferably, said logistic regression method comprises elastic net regularization.

Alternatively, the amount of each of the at least three biomarkers is compared to a reference, wherein the result of this comparison is used for the calculation of a score (in particular a single score), and wherein said score is compared to a reference score.

Thus, the present invention, in particular, a method for diagnosing heart failure in a subject comprising the steps of:
a) determining in a sample of a subject as referred to herein the amounts of at least three biomarkers as referred to above; and
b1) calculating a score based on the determined amounts of the at least three biomarkers as referred to in step a), and
b2) comparing the, thus, calculated score to a reference score, whereby heart failure is to be diagnosed.

As set forth elsewhere herein, the aforementioned method may further comprise in step a) the determination of the amount of BNP or NT-proBNP. The amount of BNP or NT-proBNP may contribute to the score calculated in step b). Accordingly, the method comprises the following steps:
a) determining in a sample of a subject as referred to herein the amounts of at least three biomarkers as referred to above and the amount of BNP or NT-proBNP; and
b1) calculating a score based on the determined amounts of the at least three biomarkers and on the amount of BNP or NT-proBNP as referred to in step a), and
b2) comparing the, thus, calculated score to a reference score, whereby heart failure is to be diagnosed.

As set forth elsewhere herein, the amount of NT-proBNP or BNP can be also derived from the medical record of the subject to be tested. In this case, it is not required to the determine the amount of this marker in step a).

Alternatively, the amount of BNP or NT-proBNP may not contribute to the score calculated in step b1). Accordingly, the method comprises the following steps:
a) determining in a sample of a subject as referred to herein the amounts of at least three biomarkers as referred to above and the amount of BNP or NT-proBNP; and
b1) calculating a score based on the determined amounts of the at least three biomarkers, and
b2) comparing the, thus, calculated score to a reference score, and comparing the amount of BNP or NT-proBNP to a reference, whereby heart failure is to be diagnosed.

As set forth elsewhere herein, the amount of NT-proBNP or BNP can be also derived from the medical record of the subject to be tested. In this case, it is not required to the determine the amount of this marker in step a).

Preferably, the reference score shall allow for differentiating whether a subject suffers from heart failure as referred to herein, or not. Preferably, the diagnosis is made by assessing whether the score of the test subject is above or below the reference score. Thus, in an embodiment it is not necessary to provide an exact reference score. A relevant reference score can be obtained by correlating the sensitivity and specificity and the sensitivity/specificity for any score, preferably, using ROC-curve-analysis. A reference score resulting in a high sensitivity results in a lower specificity and vice versa. Thus, the reference score may depend on the desired sensitivity and/or specificity. Preferably, the reference score is based on the same markers (e.g. the at least three biomarkers and NT-proBNP) as the score.

The reference score may be a “Cut-Off” value which allows for differentiating between the presence and the absence of heart failure in the subject.

In accordance with the present invention, a reference score is, preferably, a reference score obtained from a sample from a subject or group of subjects known to suffer from heart failure. In such a case, a score in the test sample being essentially identical is indicative for the presence of the disease, i.e. of heart failure (in particular for the subform thereof such as HFrEF, DCMP, ICMP and/or HFpEF). Moreover, the reference score, also preferably, could be from a subject or group of subjects known not to suffer from heart failure, preferably, an apparently healthy subject or a group of apparently healthy subjects. In such a case, a score in the test sample being altered, in particular increased, with respect to the reference score is indicative for the presence of the disease. Alternatively, a score in the test sample being essentially identical to said reference score is indicative for the absence of the disease.

Preferably, the score is calculated based on a suitable scoring algorithm. Said scoring algorithm, preferably, shall allow for differentiating whether a subject suffers from a disease as referred to herein, or not, based on the amounts of the biomarkers to be determined.

Preferably, said scoring algorithm has been previously determined by comparing the information regarding the amounts of the individual biomarkers as referred to in step a) in samples from patients suffering from heart failure as referred to herein and from patients not suffering from heart failure. Accordingly, step b) may also comprise step b0) of determining or implementing a scoring algorithm. Preferably, this step is carried out prior steps b1) and b2).

In an embodiment the reference score is calculated such that an increased amount of the score of the test subject as compared to the reference score is indicative for the presence of heart failure, and/or a decreased amount of the score of the test subject as compared to the reference score is indicative for the absence of heart failure. In particular, the score may be a cut-off value.

In a preferred embodiment of the present invention (e.g. of the methods, devices, uses etc.), the reference score is a single cut-off value. Preferably, said value allows for allocating the test subject either into a group of subjects suffering from heart failure or a into a group of subjects not suffering from heart failure. Preferably, a score for a subject lower than the reference score is indicative for the absence of heart failure in said subject (and thus can be used for ruling out heart failure), whereas a score for a subject larger than the reference score is indicative for the presence of heart failure in said subject (and thus can be used for ruling in heart failure).

In another preferred embodiment of the present invention (e.g. of the methods, devices, uses etc.), the reference score is a reference score range. In this context, a reference score range indicative for the presence of heart failure, a reference score range indicative for the absence of heart failure, or two reference score ranges (i.e. a reference score range indicative for the presence of heart failure and a reference score range indicative for the absence of heart failure) can be applied.

Preferably, the score of a subject is compared to the reference score range (or ranges). Preferably, the absence of heart failure is diagnosed, if the score is within the reference score range indicative for the absence of heart failure. A score which is not within the reference score range indicative for the absence of heart failure is, preferably, indicative for the presence of heart failure. In this case, the presence of heart failure is diagnosed.

Alternatively, the presence of heart failure is diagnosed, if the score is within the reference score range indicative for the presence of heart failure. A score which is not within the reference score range indicative for the presence of heart failure is, preferably, indicative for the absence of heart failure. In this case, the absence of heart failure is diagnosed.

A suitable scoring algorithm can be determined with the at least three biomarkers referred to in step a) by the skilled person without further ado (and optionally of BNP or NT-proBNP). E.g., the scoring algorithm may be a mathematical function that uses information regarding the amounts of the at least three biomarkers (and optionally of BNP or NT-proBNP) in a cohort of subjects suffering from heart failure and not suffering from heart failure. Methods for determining a scoring algorithm are well known in the art and including Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, lbk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Preferably, the scoring algorithm is determined with or without correction for confounders as set forth elsewhere herein.

In an embodiment, the scoring algorithm is determined with an elastic net with at least three biomarkers and optionally with BNP or NT-proBNP (see also Examples section).

Typically, a classification algorithm such as those implementing the elastic net method may be used for scoring (Zou 2005, Journal of the Royal Statistical Society, Series B: 301-320, Friedman 2010, J. Stat. Sotw. 33). Thus, the score for a subject can be, preferably, calculated with a logistic regression model fitted, e.g., by using the elastic net algorithm such as implemented in the R package glmnet. More specifically, the score may be calculated by the following formula

$$p = \frac{1}{1 + {e}^{- ( {w}_{0} + {\sum }_{i = 1}^{n} {w}_{i} {\hat{x}}_{i} )}}$$

or a mathematically equivalent formula,

with the feature \hat{x}ᵢ being

$${\hat{x}}_{i} = \frac{{x}_{i} - {m}_{i}}{{s}_{i}}$$

wherein xᵢ are the log-transformed measurement values, e.g., peak area ratios and/or concentration values, and mᵢ, sᵢ are feature specific scaling factors and wᵢ are the coefficients of the model [w₀, intercept; w₁, coefficient for the first feature (e.g. NT-proBNP, or any one of the lipid biomarkers as referred to herein); w₂ wₙ, coefficients for the further features; n, number of features in the panel].

A score larger than the reference score is indicative for a subject who suffers from heart failure, whereas a score lower than (or equal to) the reference score is indicative for a subject who does not suffer from heart failure.

The reference scores e.g. can be determined to maximize the Youden index for the detection of heart failure.

A further preferred method to calculate the score and the reference score is described in Example 5 of the Examples section. Moreover, Example 5 discloses preferred reference scores for panels 1, 3 and 4. For example, the reference score for panel 1 in combination with NT-proBNP may be 0.738.

Advantageously, it has been found in the study underlying the present invention that the amounts of the at least three biomarkers referred to above are indicators for heart failure. Accordingly, the at least three biomarkers as specified above in a sample can, in principle, be used for assessing whether a subject suffers from heart failure. This is particularly helpful for an efficient diagnosis of the disease as well as for improving of the pre-clinical and clinical management of heart failure as well as an efficient monitoring of patients. Moreover, the findings underlying the present invention will also facilitate the development of efficient drug-based therapies or other interventions including nutritional diets against heart failure as set forth in detail below.

The definitions and explanations of the terms made above apply mutatis mutandis for the following embodiments of the present invention (e.g. to the kits, devices, uses, further methods etc.) except specified otherwise herein below.

By carrying out the method of the present invention, a subject can be identified who is in need for a therapy of heart failure. Accordingly, the present invention relates to a method for identifying whether a subject is in need for a therapy of heart failure or a change of therapy comprising the steps of the methods of the present invention and the further step of identifying a subject in need if heart failure is diagnosed.

The phrase “in need for a therapy of heart failure” as used herein means that the disease in the subject is in a status where therapeutic intervention is necessary or beneficial in order to ameliorate or treat heart failure or the symptoms associated therewith. Accordingly, the findings of the studies underlying the present invention do not only allow diagnosing heart failure in a subject but also allow for identifying subjects which should be treated by a heart failure therapy or whose heart failure therapy needs adjustment. Once the subject has been identified, the method may further include a step of making recommendations for a therapy of heart failure.

A therapy of heart failure as used in accordance with the present invention, preferably, relates to a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, Vitamin-K-Antagonists, polypeptides of the protein S100 family (as disclosed by DE000003922873A1, DE000019815128A1 or DE000019915485A1 hereby incorporated by reference), natriuretic peptides such as BNP (Nesiritide (human recombinant Brain Natriuretic Peptide—BNP)) or ANP.

As a rule, patients are preferably treated with medication as recommended by the guidelines of the European Society of Cardiology (Ref: European Heart Journal (2012), 33:1787-1847).

In another embodiment the patients are treated as recommended by the 2013 ACCF/AHA guidelines (see Circulation. 2013; 128: e240-e327).

Preferably, the therapy comprises the administration of Mineralocorticoid/Aldosteron Antagonists and/or ACE Inhibitors, if the subject is diagnosed to suffer from HFrEF. Further envisaged is the treatment with a beta blocker.

Moreover, the subject may be treated with angiotensin receptor blockers (ARBs), ivabradine, digoxin and other digitalis glycosides, hydralazine and isosorbide dintrate (vasodilators) and omega-3 polyunsaturated fatty acids.

Preferably, the therapy comprises the administration of Diuretics, Aldosteron Antagonists and/or ACE Inhibitors, if the subject is diagnosed to suffer from DCMP.

Preferably, the therapy comprises the administration of Diuretics, Aldosteron Antagonists and/or ACE Inhibitors, if the subject is diagnosed to suffer from ICMP. Also preferred are Vitamin-K-antagonists and antiplatelet agents.

If the subject suffers from HFpEF, the therapy preferably comprises the administration of diuretics,

ACE inhibitor, receptor blockers (ARBs) and/or beta blockers.

The present invention further relates to a method for determining whether a therapy against heart failure is successful in a subject comprising the steps of the methods of the present invention and the further step of determining whether a therapy is successful if no heart failure is diagnosed.

It is to be understood that a heart failure therapy will be successful if heart failure or at least some symptoms thereof can be treated or ameliorated compared to an untreated subject. Moreover, a therapy is also successful as meant herein if the disease progression can be prevented or at least slowed down compared to an untreated subject.

In a preferred embodiment of the method of the invention, the determination of the at least one biomarker is achieved by mass spectroscopy techniques (preferably GC-MS and/or LC-MS), NMR or others referred to herein above. In such cases, preferably, the sample to be analyzed is pretreated. Said pretreatment, preferably, includes obtaining of the at least one preferably the at least three biomarker from sample material, e.g., plasma or serum may be obtained from whole blood or the at least one, preferably the at least three biomarkers may even be specifically extracted from sample material. Moreover, for GC-MS, further sample pretreatment such as derivatization of the at least one biomarker is, preferably, required. Furthermore, pretreatment also, preferably, includes diluting sample material and adjusting or normalizing the concentration of the components comprised therein. To this end, preferably, normalization standards may be added to the sample in predefined amounts which allow for making a comparison of the amount of the at least one biomarker and the reference and/or between different samples to be analyzed. Preferably, the normalization standard is an internal standard compound as defined elsewhere herein. For example, one standard for each class of biomarkers may be added in order to allow for a normalization, e.g one standard for triacylglyerides, one standard for sphingomyelins etc. In another preferred embodiment the quantification of SM biomarker is achieved by adding commercially available sphingomyeline standards with a different chain length than the target metabolites based on the observation that the detector response is the same. In a further preferred embodiment the calibration solutions are prepared in delipidized plasma (commercially available) to simulate a matrix as close as possible to real plasma.

Accordingly, the present invention further pertains to a composition or kit comprising the at least three biomarkers as set forth in connection with the method of the present invention.

In addition, the present invention relates to a calibration solution comprising said composition comprising said at least three biomarkers. The calibration solution shall allow for the calibration of the device used for the determination of the amounts of the at least three biomarkers. Preferably, the calibration solution serves as stock solution for the preparation of a calibration sample/calibration samples which are defined herein below. Preferably, the at least three biomarkers are dissolved in a suitable solvent to form together the calibration solution. Thus, the calibration solution preferably comprises a suitable solvent and said composition comprising said at least three biomarkers. Preferably, the solvent comprises or is an unpolar liquid solvent, like methylchlorid, dichlormethan, chloroform, 1,2-dichlorethan. Preferably, an extraction solvent as described elsewhere herein is used. Even more preferably the solvent is a mixture comprising dichloromethane (DCM) and methanol. In an embodiment the extraction solvent comprises methanol and DCM, in particular in a ratio of about 2:1 (preferably volume to volume).

Furthermore, the present invention relates to a calibration sample comprising the composition of the present invention (i.e. comprising at least three biomarkers as set forth in connection with the method of the present invention), and delipidized serum or plasma. Preferably, the calibration sample further comprises a suitable solvent, in particular an extraction solvent as defined elsewhere herein.

Said composition, calibration solution or said calibration sample can be used as control(s) when carrying out the present invention, in particular, for controlling and/or calibrating the device(s) for the determination of the amount of the at least three biomarkers.

In a preferred embodiment, the delipidized serum or plasma is defibrinated.

In an embodiment said delipidized sample is delipidized serum. In another embodiment said delipidized sample is delipidized plasma.

Preferably, said composition, calibration solution, or calibration sample comprises the at least three biomarkers in predefined amounts or ratios.

The term “delipidized” (frequently also referred to as “delipidated”) is well known in the art. Preferably, the term means that the lipids (such as triglycerides, cholesterols, phospholipids, and unesterified fatty acids) that are naturally present in said sample (e.g. serum, plasma) have been removed from said sample (see e.g. Cham et al. J Lipid Res. 1976 March; 17(2):176-81. A solvent system for delipidation of plasma or serum without protein precipitation). After delipidizing said sample, the at least three biomarkers are referred herein are added, in particular artificially added, to said delipidized serum or plasma. Thus, the at least three biomarkers are preferably spiked into said delipidized sample. Preferably, the at least three biomarkers are added to the delipidized serum or plasma in predefined amounts. Preferably, said predefined amounts shall allow for a calibration and/or control. Alternatively, said predefined amounts shall represent a reference.

In order to add the at least three biomarkers to the delipidized serum or plasma, the at least three biomarkers are dissolved in a suitable solvent (which is in particular the extraction solvent as defined elsewhere herein). The resulting solution is then added, i.e. combined with delipidized serum or plasma. Preferably, the ratio of the delipidized serum or plasma to the solvent, is about 1:10 to 1:1000 more preferably, about 1:100: to 1:500, or even more preferably about 1:100 to 1:200 and most preferably about 1:150.

For the calibration, an aliquot of the calibration solution can be mixed with delipidized serum or plasma, thereby producing the calibration sample or a series of calibrations samples. Preferably, the further calibration samples may thus prepared be from a stock solution being a calibration solution with the highest concentration of the biomarkers. In case an internal standard is added, preferably the same amount of internal standard is given to each member of the series of calibration samples (see below).

In a preferred embodiment of the present invention, the composition, the calibration solution, the calibration sample, or kit comprises the lipid biomarkers that are used for the diagnosis (i.e. the same combination of lipid biomarkers for example the biomarkers of panels 1, 2 or 200).

However, it is envisaged that the lipid biomarkers are replaced with other biomarkers, said other biomarker preferably belonging to the same compound class. Thus, a triacylglyceride biomarker may be replaced with a different triacylglyceride, a cholesterylester biomarker may be replaced with a different cholesterylester, a phosphatidylcholine biomarker may be replaced with a different phosphatidylcholine r, a sphingomyelin biomarker may be replaced with a different sphingomyelin, a ceramide biomarker may be replaced with a different ceramide. For example, if the method of the present invention comprises the determination of SM23, the delipidized sample may comprise SM27 instead. SM27 preferably refers to Sphingomyelin (d18:1/24:1) or Sphingomyelin(d18:2/24:0), or a combination thereof. In a preferred embodiment, SM27 is Sphingomyelin (dl 8:1/24:1). Preferred triacylglyceride, cholesterylester, phosphatidylcholine, ceramide, and sphingomyelin are described elsewhere herein.

The composition, calibration solution, calibration sample, or kit thus preferably comprises
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin; or
iv. at least one phosphatidylcholine, at least one cholesterylester, and at least one sphingomyelin;

In another preferred embodiment, the composition, calibration solution, calibration sample, or kit may comprise at least one triacylglyceride, at least one cholesterylester, at least one sphingomyelin and at least one phosphatidylcholine.

In a particular preferred embodiment, the composition, calibration solution, calibration sample, or kit comprises OSS2, PC4, and SM23. In another particular preferred embodiment, the composition, calibration solution, or calibration sample comprises OSS2, PC4, and SM27.

In a preferred embodiment, in particular for Panel 1, the ratios of PC4: SM27 (based on molar concentrations) is in the range of 50:1: to 2.5:1, in a more preferred embodiment the ratio is in the range of 25:1 to 5:1. In another preferred embodiment, in particular for Panel 1, the ratios of SM27:OSS2 (based on molar concentrations) is in the range of 30:1: to 1.5:1, in a more preferred embodiment the ratio is in the range of 15:1 to 2:1. In particular the ratios of PC4:SM27:OSS2 (based on molar concentrations) are about 100:7.9:1.1. In case SM23 is part of the composition, calibration solution, calibration sample, or kit instead of SM27 the same ratios as mentioned before apply

In another particular preferred embodiment, the composition, calibration solution, calibration sample, or kit comprises OSS2, CE 18:2, and SM23. In another particular preferred embodiment, the composition, calibration solution, or calibration sample comprises OSS2, CE 18:2, and SM27. In a preferred embodiment, in particular for Panel 2, the ratios of CE18:2:SM27 (based on molar concentrations) is in the range of 500:1: to 2:1, in a more preferred embodiment the ratio is in the range of 100:1 to 10:1. In another preferred embodiment, in particular for for Panel 2, the ratios of SM27:OSS2 (based on molar concentrations) is in the range of 30:1: to 1.5:1, in a more preferred embodiment the ratio is in the range of 15:1 to 2:1. In particular the ratios of CE 8:2:SM27:OSS2 (based on molar concentrations) are about 100:2.7:0.39. In case SM23 is part of the composition, calibration solution, calibration sample, or kit instead of SM27 the same ratios as mentioned before apply

In another particular preferred embodiment, the composition, calibration solution, calibration sample, or kit comprises OSS2, PC4, SM23 and CE18:2. In another particular preferred embodiment, the composition, calibration solution, or calibration sample comprises OSS2, PC4, CE 18:2 and SM27. In a preferred embodiment, in particular for Panel 200, the ratios of PC4:SM27 (based on molar concentrations) is in the range of 50:1: to 2.5:1, in a more preferred embodiment the ratio is in the range of 25:1 to 5:1. In another preferred embodiment, in particular for Panel 200, the ratios of SM27:OSS2 (based on molar concentrations) is in the range of 30:1: to 1.5:1, in a more preferred embodiment the ratio is in the range of 15:1 to 2:1. In another preferred embodiment the ratios of CE18:2:SM27 (based on molar concentrations) is in the range of 500:1: to 2:1, in a more preferred embodiment the ratio is in the range of 100:1 to 10:1. In particular the ratios of CE18:2:PC4:SM27:OSS2 (based on molar concentrations) are about 100:34.2:2.7:0.4. In case SM23 is part of the composition, calibration solution, calibration sample, or kit instead of SM27 the same ratios as mentioned before apply

In addition, the present invention relates to a series of calibration solutions and/or calibration samples and/or compositions of the present invention. Preferably, said series of calibration solutions/calibration samples comprises at least three, more preferably at least four and most preferably at least five different calibration solutions and/or calibration samples and/or compositions. Said calibration solutions and/or calibration solutions and/or compositions shall differ in the predefined amounts of the lipid biomarkers comprised by said solutions/samples/compositions. Thus, the present invention envisages a dilution series of calibration solutions and/or calibration samples of the present invention. In an embodiment, the amounts of the biomarkers comprised by the series of calibration solutions, and/or calibration samples are in a linear relationship so that the highest concentrated one can be used as stock solution.

Preferred amounts of the biomarkers PC4, CE 18:2, SM27 and OSS2 present in the calibration solution(s) are shown in Table 13 of the Examples section. Table 13 lists the stock solutions used for the calibration. Preferred ratios of the biomarkers are shown in Table 13a for panel 200, Table 13b for panel 2, and Table 13c for panel 1 (see in particular column STD1).

Preferably, the calibration solution comprises OSS2, PC4, and/or SM27. Preferably, the concentration is a follows:

PC4: from about 6 to about 193 nmol/ml

OSS2: from about 0.07 to about 2.2 nmol/ml, and/or

SM27: from about 0.5 to about 15.2 nmol/ml

If the calibration solution comprises CE 18:2, the concentration is preferably from about 18 to about 570 nmol/ml.

In an embodiment, a calibration solution or calibration sample comprising PC4, OSS2 and SM27 is used, if the biomarkers of panel 1 are determined. In another embodiment, a calibration solution or calibration sample comprising PC4, OSS2 and SM23 is used, if the biomarkers of panel 1 are determined.

In an embodiment, a calibration solution or calibration sample or kit comprising OSS2, CE 18:2 and SM27 is used, if the biomarkers of panel 2 are determined. In another embodiment, a calibration solution or calibration sample or kit comprising OSS2, CE 18:2 and SM23 is used, if the biomarkers of panel 2 are determined.

In an embodiment, a calibration solution or calibration sample or kit comprising PC4, OSS2, CE 18:2 and SM27 is used, if the biomarkers of panel 200 are determined. In another embodiment, a calibration solution or calibration sample comprising PC4, OSS2, CE 18:2 and SM23 or kit is used, if the biomarkers of panel 200 are determined. However, it can also be envisaged that this calibration solution or calibration sample or kit can be used, if the biomarkers of panel 1 or panel 2 are determined.

Further preferred calibration solutions are described in the examples section, see, e.g., Example 12.

As set forth above, the composition, calibration solution, calibration sample or kit shall comprise the at least three biomarkers. It is to be understood, that the at least three biomarkers do not have to be comprised by the same calibration solution or calibration sample. So a plurality of calibration solutions, or calibration samples can be prepared. Rather, they can be comprised in individual calibration solutions or calibration samples. Thus, if three biomarkers (Marker A, Marker B and Marker C) shall be determined, the individual biomarkers can be comprised in one calibration solution/sample with markers A, B, and C, two calibration solutions/samples (one with A and B, one with C; or one with A and C, and one with B; one with A and C and one with B), or three calibration solutions/samples.

Thus, the present invention relates to a kit comprising i) a single calibration solution, said single calibration solution comprising the at least three biomarkers as set forth in connection with the method of the present invention or ii) a plurality of calibration solutions, said plurality of calibration solutions comprising the at least three biomarkers as set forth in connection with the method of the present invention, wherein preferably in the plurality of calibration solutions each calibration solution comprises at least one of the said at least three biomarkers. More preferably, the calibration solutions in the plurality of calibration solutions do not comprise identical biomarkers. In a preferred embodiment, the at least three biomarkers are the biomarkers of panel 1, 2 or 200, wherein SM23 has been replaced by SM27. E.g, with respect to panel 1, the kit comprises the biomarkers OSS2, PC4, and SM23 or OSS2, PC4, and SM27. Preferred concentrations or ratios for the biomarkers of some panels are described above. In addition, the kit may comprise an the internal standard solution of the present invention. Preferably, the internal standard solution comprises phosphatidylcholine C19:0 C19:0.

In a preferred embodiment of the composition, the calibration solution, the calibration sample, or kit of the present invention, the composition, the calibration solution, the calibration sample, or kit further comprises one or more internal standard compounds. Preferably, the composition, the calibration solution, the calibration sample, or kit further comprises phosphatidylcholine C19:0 C19:0. In an embodiment of the present invention for Panels 1, 2 and/or 200 the calibration sample with the highest concentration of the respective biomarkers, comprises OSS2 and phosphatidylcholine C19:0 C19:0 (based on molar concentrations) in an ratio of 0.05:1 to 2:1, in a preferred embodiment the ratio is in the range of 0.1:1 to 0.5 to 1. In particular for panel 1 the ratios (based on molar concentrations) of PC4:SM27:OSS2:phosphatidylcholine C19:0 C19:0 is about 100:7.9:1.1:5.2. The same ratios apply if instead of SM 27 SM23 is used.

The present invention also relates to an internal standard solution as described herein above. Preferably, the internal standard solution comprises at least one internal standard compound. In an embodiment, the internal standard compound is phosphatidylcholine C19:0 C19:0. Preferably, the at least one internal standard compound is dissolved in a suitable solvent to form together the internal standard solution. Thus, the internal standard solution preferably comprises a suitable solvent and said composition comprising said at least three biomarkers. Preferably, the solvent comprises or is an unpolar liquid solvent, like methylchlorid, dichlormethan, chloroform, 1,2-dichlorethan. Preferably, an extraction solvent as described elsewhere herein is used. Even more, preferably the solvent is a mixture comprising dichloromethane (DCM) and methanol. In an embodiment the extraction solvent comprises methanol and DCM, in particular in a ratio of about 2:1 (preferably volume to volume).

Preferred concentrations of phosphatidylcholine C19:0 C19:0 in the internal standard solution are given in connection with method of the present invention.

Further, the present invention relates to a kit comprising the at least three biomarkers as referred to in the context of the present invention. In a preferred embodiment, the at least three biomarkers are the biomarkers of panel 1, 2 or 200 (in particular panel 1). In another preferred embodiment, the at least three biomarkers are the biomarkers of panel 1, 2 or 200, wherein SM23 has been replaced by SM27. E.g, with respect to panel 1, the kit comprises the biomarkers OSS2, PC4, and SM23 or OSS2, PC4, and SM27.

In addition the present invention relates to a kit comprising the at least three biomarkers as set forth in connection with the method of the present invention and one or more internal standard compounds as referred to herein. In a preferred embodiment, the internal standard compound is phosphatidylcholine C19:0 C19:0 and the at least three biomarkers are the biomarkers of panel 1, 2 or 200 (in particular panel 1). In another preferred embodiment, the internal standard compound is phosphatidylcholine C19:0 C19:0 and the at least three biomarkers are the biomarkers of panel 1, 2 or 200, wherein SM23 has been replaced by SM27. E.g, with respect to panel 1, the kit comprises the biomarkers OSS2, PC4, and SM23 or OSS2, PC4, and SM27.

In addition the present invention relates to a kit comprising i) the calibration solution(s) or the plurality of calibration solutions of the present invention and ii) the internal standard solution of the present invention. Preferably, the internal standard solution comprises phosphatidylcholine C19:0 C19:0.

In addition the present invention relates to a kit comprising i) the calibration sample(s) or the plurality of calibration samples of the present invention and ii) the internal standard solution of the present invention. Preferably, the internal standard solution comprises phosphatidylcholine C19:0 C19:0.

The aforementioned kits can be preferably used for calibration the devices for the determination of the amounts the at least three biomarkers.

The method of the present invention, in a preferred embodiment, furthermore further comprises a step of recommending and/or managing the subject according to the result the diagnosis established in step b). Such a recommendation may, in an aspect, be an adaptation of life style, nutrition and the like aiming to improve the life circumstances, the application of therapeutic measures as set forth elsewhere herein in detail, and/or a regular disease monitoring.

In another preferred embodiment of the aforementioned method, step b) is carried out by an evaluation unit as set forth elsewhere herein.

Further, the present invention also in an aspect pertains to a method of treating heart failure comprising the steps a) and b) of the method for diagnosing heart failure, and the further step c) of treating the subject in case the subject is diagnosed to suffer from heart failure. The method may also comprise the step b1) of selecting a subject who suffers from heart failure. In an embodiment, step b1) is carried out after step b), but before step c). For example, the subject may be treated, if the subject suffers from symptomatic or asymptomatic heart failure.

Further, the present invention also in an aspect pertains to a method of treating heart failure comprising the steps of the method for identifying whether a subject is in need for a therapy of heart failure or a change of therapy comprising the steps of the methods of the present invention, the further step of identifying a subject in need if heart failure is diagnosed and the further step of treating the subject accordingly.

The definitions and explanations of the terms made above apply mutatis mutandis for the following embodiments of the present invention except specified otherwise herein below.

The aforementioned methods for the determination of the at least three biomarkers can be implemented into a device. A device as used herein shall comprise at least the aforementioned means. Moreover, the device, preferably, further comprises means for comparison and evaluation of the detected characteristic feature(s) of the at least three biomarker and, also preferably, the determined signal intensity. The means of the device are, preferably, operatively linked to each other. How to link the means in an operating manner will depend on the type of means included into the device. For example, where means for automatically qualitatively or quantitatively determining the biomarker are applied, the data obtained by said automatically operating means can be processed by, e.g., a computer program in order to facilitate the assessment. Preferably, the means are comprised by a single device in such a case. Said device may accordingly include an analyzing unit for the biomarker and a computer unit for processing the resulting data for the assessment. Preferred devices are those which can be applied without the particular knowledge of a specialized clinician, e.g., electronic devices which merely require loading with a sample.

Alternatively, the methods for the determination of the at least one biomarker can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means. A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS, in particular HPLC-MS, and/or GC-MS are used in the system of the present invention as described in detail elsewhere in the specification. Further comprised shall be means for comparing and/or analyzing the results obtained from the means for determination of the at least three biomarkers (and optionally of NT-proBNP or BNP). The means for comparing and/or analyzing the results may comprise at least one databases and an implemented computer program for comparison of the results. Preferred embodiments of the aforementioned systems and devices are also described in detail below.

Therefore, the present invention relates to a diagnostic device comprising:
a) an analysing unit comprising at least one detector for the at least three biomarkers as referred to herein in connection with the present invention detected by the at least one detector, and, operatively linked thereto;
b) an evaluation unit comprising a computer comprising tangibly embedded a computer program code for carrying out a comparison of the determined amounts of the at least three biomarkers and the reference amounts, and a data base comprising said reference amounts for the said biomarkers, whereby it will be diagnosed whether a subject suffers from heart failure.

Alternatively, the evaluation unit under b) comprises a computer comprising tangibly embedded a computer program code for calculating a score based on the determined amounts of the at least three biomarkers and for carrying out a comparison of the calculated score and the reference score, wherein said evaluation unit further comprises a data base comprising said reference score, whereby it will be diagnosed whether a subject suffers from heart failure.

The terms “score” and “reference score” are determined elsewhere herein.

In an embodiment, the device further comprises at least one further analysing unit comprising at least one detector for NT-proBNP and/or BNP, wherein said further analyzing unit is adapted for determining the amounts BNP and/or NT-proBNP detected by the at least one detector.

Thus, the present invention relates to a diagnostic device comprising:
a) an analysing unit comprising at least one detector for the at least three biomarkers as referred to herein in connection with the present invention, wherein said analyzing unit is adapted for determining the amounts of the said biomarkers detected by the at least one detector, and, optionally, at least one further analysing unit comprising at least one detector for NT-proBNP and/or BNP, wherein said further analyzing unit is adapted for determining the amounts BNP and/or NT-proBNP detected by the at least one detector for NT-proBNP and/or BNP, and operatively linked thereto;
b) an evaluation unit comprising a computer comprising tangibly embedded a computer program code for carrying out a comparison of the determined amounts of the at least three biomarkers and, optionally, of BNP or NT-proBNP, and the reference amounts and a data base comprising said reference amounts for the said biomarkers, whereby it will be diagnosed whether a subject suffers from heart failure.

Alternatively, the evaluation unit under b) comprises a computer comprising tangibly embedded a computer program code for calculating a score based on the determined amounts of the at least three biomarkers and of BNP and/or NT-proBNP, and for carrying out a comparison of the calculated score and the reference score, wherein said evaluation unit further comprises a data base comprising said reference score, whereby it will be diagnosed whether a subject suffers from heart failure.

Preferably, the devices are adapted to carry out the method of the present invention.

Preferably, the computer program code is capable of executing steps of the method of the present invention as specified elsewhere herein in detail. Accordingly, the device can be used for diagnosing heart failure as specified herein based on a sample of a subject.

In a preferred embodiment, the device comprises a further database comprising the kind of regulation and/or fold of regulation values indicated for the respective biomarkers in any one of Tables 1A and/or 1B and a further tangibly embedded computer program code for carrying out a comparison between the determined kind of regulation and/or fold of regulation values and those comprised by the database. In another preferred embodiment, the device comprises a further database comprising the kind of regulation and/or fold of regulation values indicated for the score(s) calculated based on the amounts of the at least three biomarkers, and a further tangibly embedded computer program code for carrying out a comparison between the determined kind of regulation and/or fold of regulation values for the score and those comprised by the database.

Furthermore, the present invention relates to a data collection comprising characteristic values of the at least three biomarkers being indicative for a medical condition or effect as set forth above (i.e. diagnosing heart failure in a subject). Furthermore, the present invention relates to a data collection comprising characteristic values of scores calculated based on the amounts of the at least three biomarkers as set forth herein being indicative for a medical condition or effect as set forth above (i.e. diagnosing heart failure in a subject).

The term “data collection” refers to a collection of data which may be physically and/or logically grouped together. In one embodiment the physical or logical grouping is realized by classification approaches like elastic net, random forest, penalized logistic regression or others known by the person skilled in the art. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a medical condition or effect as set forth above (e.g. a query search). Thus, if an identical or similar data set can be identified in the data collection, the test data set will be associated with the said medical condition or effect. Consequently, the information obtained from the data collection can be used, e.g., as a reference for the methods of the present invention described above. More preferably, the data collection comprises characteristic values of all at least three biomarkers recited above. Also preferably, the data collection comprises scores for the at least three biomarkers as set forth above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, a flash storage medium, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising:
(a) means for comparing characteristic values of the biomarkers of the at least three biomarkers or a score (based on the amounts of the at least three markers) of a sample operatively linked to
(b) a data storage medium as described above.

The system may further comprise means for comparing characteristic values of BNP or NT-proBNP (or a score based thereon).

In another embodiment the present invention also relates to a system comprising:
(a) means for comparing a score based on the amounts of the at least three biomarkers and of BNP or NT-proBNP of a sample operatively linked to
(b) a data storage medium as described above.

The term “system” as used herein relates to different means which are operatively linked to each other. Said means may be implemented in a single device or may be physically separated devices which are operatively linked to each other. The means for comparing characteristic values of biomarkers, preferably, based on an algorithm for comparison as mentioned before. The data storage medium, preferably, comprises the aforementioned data collection or database, wherein each of the stored data sets being indicative for a medical condition or effect referred to above. Thus, the system of the present invention allows identifying whether a test data set is comprised by the data collection stored in the data storage medium. Consequently, the methods of the present invention can be implemented by the system of the present invention.

In a preferred embodiment of the system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

Moreover, the present invention relates to a diagnostic means comprising means for the determination of at the at least three biomarker as referred to in connection with the method of the present invention.

The diagnostic means may further comprise means for the determination of BNP or NT-proBNP, in particular said determination is based on a determination with respective antibodies, and thus said means shall comprise at least one antibody, or fragment thereof, which binds specifically BNP or NT-proBNP. Thus, said means shall allow for the determination of NT-proBNP or BNP. Preferably, said means are adapted to carry out an immunoassay for determining the amount of the marker.

The term “diagnostic means”, preferably, relates to a diagnostic device, system or biological or chemical assay as specified elsewhere in the description in detail.

The expression “means for the determination of at least three biomarker” refers to devices or agents which are capable of specifically recognizing at least three biomarkers. Suitable devices may be spectrometric devices such as mass spectrometry, NMR devices or devices for carrying out chemical or biological assays for the biomarkers. Suitable agents may be compounds which specifically detect the biomarkers. Detection as used herein may be a two-step process, i.e. the compound may first bind specifically to the biomarker to be detected and subsequently generate a detectable signal, e.g., fluorescent signals, chemiluminescent signals, radioactive signals and the like. For the generation of the detectable signal further compounds may be required which are all comprised by the term “means for determination of the at least one biomarker”.

Further, the present invention relates to a diagnostic composition comprising at least three biomarkers referred to above.

The said as least three biomarkers will serve as an indicator for a medical condition or effect in the subject as set forth elsewhere herein. Thus, the biomarker molecules itself may serve as diagnostic compositions, preferably, upon visualization or detection by the means referred to in herein. Thus, a diagnostic composition which indicates the presence of a biomarker according to the present invention may also comprise the said biomarker physically, e.g., a complex of an antibody and the biomarker to be detected may serve as the diagnostic composition. Accordingly, the diagnostic composition may further comprise means for detection of the metabolites as specified elsewhere in this description. Alternatively, if detection means such as MS or NMR based techniques are used, the molecular species which serves as an indicator for the risk condition will be the at least one biomarker comprised by the test sample to be investigated. Thus, the at least three biomarkers referred to in accordance with the present invention shall serve itself as a diagnostic composition due to its identification as an indicator for the disease.

In general, the present invention contemplates the use of at least three biomarkers as referred to herein in connection with the method of diagnosing heart failure, and optionally of BNP or NT-proBNP, in a sample of a subject for diagnosing heart failure or for the preparation of a pharmaceutical and/or diagnostic composition for diagnosing heart failure. Preferred combinations of biomarkers and subforms of heart failure to be diagnosed are disclosed elsewhere herein.

The present invention also relates to a kit for carrying out the method of the present invention, said kit comprising detection agents for each of the biomarkers of the at least three biomarkers as set forth in connection with the method of diagnosing heart failure. In an embodiment, the kit may further comprise a detection agent for NT-proBNP or BNP.

The term “kit” as used herein refers to a collection of the aforementioned components, preferably, provided separately or within a single container. The detection agents may be provided in the kit of the invention in a “ready-to-use” liquid form or in dry form. The kit may further include controls, buffers, and/or reagents. The kit also comprises instructions for carrying out the method of the present invention, as well as information on the reference values. These instructions may be in the form of a manual or may be electronically accessible information. The latter information may be provided on a data storage medium or device such as an optical storage medium (e.g., a Compact Disc) or directly on a computer or data processing device.

Suitable detection agents for the biomarkers have been specified elsewhere herein in detail. For example, the detection agents may be antibodies or aptameres or other molecules which are capable of binding to the biomarkers specifically.

The kit of the invention can be, preferably, used for carrying out the method of the present invention, i.e. for diagnosing heart failure as specified elsewhere herein in detail.

The definitions and explanations given herein above also apply to the following method and use.

Moreover, the present invention relates to a method for diagnosing heart failure in a subject comprising the steps of:
a) determining in a sample of a subject the amount at least one biomarker selected from the group consisting of phosphatidylcholin (C18:1 C18.1), SM(d18:1/18:1), SM(d18:2/17:0), OSS2, PPO1, PPP, SOP2, SPP1, SSP2 and SSS; and
b) comparing the amounts of the said biomarkers as determined in step a) to a reference, whereby heart failure is to be diagnosed.

Moreover, the present invention pertains to the use of at least one biomarker selected from the group consisting of phosphatidylcholin (C18:1 C18.1), SM(d18:1/18:1), SM(d18:2/17:0), OSS2, PPO1, PPP, SOP2, SPP1, SSP2 and SSS in a sample of a subject for the diagnosis of heart failure.

In an embodiment of the method, kits, devices, uses of the present invention, the amounts of biomarkers of panel 300 (see Example 10) are determined. Preferably, said biomarkers are determined as described in Example 10.

Method of Monitoring Heart Failure Therapy

Based on the determination of the amounts of the at least three biomarkers as referred to above in the context of the method for diagnosing heart, it can be even assessed on an individual basis whether a treatment will be effective, or not.

The present invention thus relates to a method for monitoring heart failure therapy in a subject, comprising:
(a) determining the amount of the at least three biomarkers in a first sample of said subject and calculating a first score based on the determined amounts,
(b) determining the amount of the at least three biomarkers in a second sample of said subject and calculating a second score based on the determined amounts; and
(c) comparing the first score to the second score, whereby heart failure therapy in said subject is to be monitored.

In an embodiment of the aforementioned method, the method further comprises the determination of the amount of BNP (brain natriuretic peptide, also known as B-type natriuretic peptide) or, in particular, NT-proBNP (N-terminus of the prohormone brain natriuretic peptide) in the first and second sample.

Thus steps (a) to (c) may be as follows:
(a) determining the amount of the at least three biomarkers and the amount of BNP or NT-proBNP in a first sample of said subject and calculating a first score based on the determined amounts,
(b) determining the amount of the at least three biomarkers and the amount of BNP or NT-proBNP in a second sample of said subject and calculating a second score based on the determined amounts; and
(c) comparing the first score to the second score, whereby heart failure therapy in said subject is to be monitored.

The definitions given herein above in connection of the method for diagnosing heart failure preferably apply to the method of monitoring heart failure therapy as well.

The aforementioned method, preferably, is an in vitro method. Moreover, it may comprise steps in addition to those explicitly mentioned above. For example, further steps may relate to sample pre-treatments or evaluation of the results obtained by the method. Preferably, step (a), (b) and/or (c) may in total or in part be assisted by automation, e.g., by a suitable robotic and sensory equipment for the determination in steps (a) and (b), or a computer-implemented comparison in step (c).

The term “subject” has been defined above in connection with the method of diagnosing heart failure. Preferably, the subject to be tested in accordance with the method for monitoring heart failure therapy shall suffer from heart failure. Moreover, the subject as set forth in connection with the aforementioned method shall be treated for heart failure, and thus shall receive heart failure therapy. Preferably, said subject does not suffer from acute decompensation.

The term “monitoring heart failure therapy” as used herein in the context of the aforementioned method, preferably, relates to assessing whether a subject responds to said therapy, or not. Accordingly, it is assessed whether a subject benefits from said therapy, or not. Preferably, a decrease of the second score as compared to the first score shall be indicative for a subject who responds to heart failure therapy. In contrast, an increase of the second score as compared to the first score shall be indicative for a subject who does not respond to heart failure therapy. Preferably, by carrying out the aforementioned method decisions can be made whether heart failure therapy in said subject shall be continued, stopped or amended.

Preferably, a subject responds to heart failure therapy, if said therapy improves the condition of the subject with respect to heart failure. Preferably, a subject does not respond to said therapy, if said therapy does not the improve the condition of the subject with respect to heart failure. In this case, the therapy may put the subject at risk of adverse side effects without any significant benefit to said subject (thereby generating useless health care costs).

The term “heart failure therapy” as used herein, preferably, includes any therapy for the treatment of heart failure. Preferred therapies are drug-based therapies. Preferably, the therapy of heart failure is a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, and Vitamin-K-Antagonists.

Alternatively, the therapy may be therapy with assist devices such as ventricular assist devices.

The term “score” has been explained in connection with the method of diagnosing heart failure. The explanation applies accordingly.

The term “sample” has been described elsewhere herein. The definition applies accordingly. In the context of the aforementioned method, the amount of the at least three biomarker as referred to herein shall be determined in a first and in a second sample. Preferably, the first sample has been/is obtained before initiation of heart failure therapy, or more preferably, after initiation of heart failure therapy.

If the first sample has been obtained before initiation of heart failure therapy, it is preferred that it has been obtained shortly before said initiation. Preferably, a sample is considered to have been obtained shortly before initiation of heart failure therapy, if it has been obtained within less than one week, or, more preferably, within less than three days, or, most preferably, within less than one day before initiating heart failure therapy.

The “second sample” is particularly understood as a sample which is obtained in order to reflect a change of the second score (i.e. the score in the second sample) to the first score (i.e. the score in the first sample). Thus, the second sample, preferably, shall have been obtained after the first sample. Of course, the second sample shall have been obtained after initiation of heart failure therapy. It is to be understood that the second sample has been obtained not too early after the first sample in order to observe a sufficiently significant change of the score to allow for monitoring heart failure therapy. Therefore, the second sample has been, preferably, obtained at least one week, or, more preferably, at least two weeks, or even more preferably, at least one month or two months, or, most preferably, at least three months after the first sample has been obtained. Also preferably, it is contemplated that the second sample has been obtained within a period of one week to three months after the first sample.

If the first sample has been obtained before initiation of heart failure therapy, the second sample has been, preferably, obtained at least one week, or, more preferably, at least two weeks, or even more preferably, at least one month or two months, or, most preferably, at least three months after initiation of heart failure therapy.

It shall be clear from the above that the determination of the amounts of the at least three biomarkers (and optionally BNP or NT-proBNP) in said first sample referred to in step (a) may take place several days or weeks before the determination of the amounts in said second sample referred to in step (b). Therefore the steps (a), (b) and (c) of the method for monitoring heart failure need not be conduct one after the other in a limited time frame but may well be spread over a longer time period of several days, weeks or even months. Thus, it is to be understood that the aforementioned method allows for short-term, mid-term, and also for long-term monitoring depending on the interval between obtaining the two samples. Thus, the second sample may be obtained within a period of one day to two years or more after the first sample. In one preferred embodiment, the second sample has been obtained one day, or two days, in particular within a period of one to two days, after the first sample (which allows for short-term monitoring). In one another preferred embodiment, the second sample has been obtained one months, or two months, in particular within a period of one to two months, after the first sample (which allows for mid-term monitoring). In a further preferred embodiment, the second sample has been obtained six months, or twelve months, in particular within a period of six to twelve months or more, after the first sample (which allows for long-term monitoring).

It is also envisaged to assess the time course of the scores in samples from the subject to be monitored. Accordingly, the aforementioned method may comprise the additional step of determining the amounts of the at least three biomarkers in at least one further sample from said subject (thus, in a third sample, in a fourth sample, in a fifth sample etc.), calculating at least one further scores, and comparing the, thus, calculated score with the first and/or second score and/or any score that was calculated before said at least one further sample was obtained. For preferred time intervals for obtaining the samples, please see above.

Preferably, the assessment whether the subject responds to heart failure therapy, or not, is based on the comparison of second score from the subject with the first score.

Preferably, a decrease and, more preferably, a significant decrease, and, most preferably, a statistically significant decrease of the second score as compared to the first score is indicative for a subject who responds to heart failure therapy.

A significant decrease, preferably, is a decrease of a size which is considered to be significant for monitoring heart failure. Particularly said decrease is considered statistically significant. The terms “significant” and “statistically significant” are known by the person skilled in the art. Thus, whether a decrease is significant or statistically significant can be determined without further ado by the person skilled in the art using various well known statistic evaluation tools. Preferred significant decreases of the score which are indicative for a subject who responds to heart failure therapy are given herein below

Preferably, a decrease of the second score compared to the first score, preferably, of at least 5%, of at least 10%, more preferably of at least 20%, and, even more preferably, of at least 30%, and most preferably of at least 40% is considered to be significant and, thus, to be indicative for a subject who responds to heart failure therapy. Also preferably, a decrease of the second score compared to the first score, preferably, of at least 0.05, of at least 0.10, more preferably of at least 0.15 and most preferably of at least 0.2 is considered to be significant and, thus, to be indicative for a subject who responds to heart failure therapy.

As set forth above, an increase of the second score compared with the first score (or an, in particular, an essentially unchanged second score as compared with the first score) is indicative for a subject who does not respond to heart failure therapy.

### EMBODIMENTS/ITEMS OF THE PRESENT INVENTION

The definitions and explanations, given in the specification and/or the claims, preferably, apply mutatis mutandis to following preferred embodiments/items of the present invention.
1. A method for diagnosing heart failure comprising the steps of:
   a. determining in a sample of a subject the amounts of at least three biomarkers, wherein said at least three biomarkers are:
      i. at least one triacylglyceride biomarker, at least one cholesterylester biomarker, and at least one phosphatidylcholine biomarker;
      ii. at least one triacylglyceride biomarker, at least one phosphatidylcholine biomarker, and at least one sphingomyelin biomarker;
      iii. at least one triacylglyceride biomarker, at least one cholesterylester biomarker, and at least one sphingomyelin biomarker;
      iv. at least one phosphatidylcholine biomarker, at least one cholesterylester biomarker, and at least one sphingomyelin biomarker;
      v. Cholesterylester C18:2, SSS and Cer(d17:1/24:0);
      vi. at least two sphingomyelin biomarkers selected from the group consisting of SM2, SM3, SM5, SM18, SM23, SM24, and SM28, and at least one triacylglyceride biomarker selected from the group consisting of SOP2, SPP1 and PPO1, (or alternatively at least one triacylglyceride biomarker selected from the group consisting of SOP2, SPP1 and PPP);
      vii. at least two triacylglyceride biomarkers selected from the group consisting of OSS2, SOP2, SPP1 and SSP2, and at least one sphingomyelin biomarker selected from the group consisting of SM23 and SM24;
      viii. SM18, SM24 and SM28; or
      ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 of Table 2,
      x. the biomarkers of panel 200, 201, 202, 203, 204, 205, or 206 of Table 2a and
   b. comparing the amounts as determined in step a. to a reference, whereby heart failure is to be diagnosed,
wherein the at least one triacylglyceride biomarker in i., ii., and iii. is selected from the group consisting of SOP2, OSS2, SPP1, SSP2, PPO1 and PPP, the at least one cholesterylester biomarker in i., iii., and iv. is selected from the group consisting of cholesterylester C18:2 and cholesterylester C18:0, the at least one phosphatidylcholine biomarker in i., ii. and iv. is selected from the group consisting of PC4 and PC8, and the at least one sphingomyelin biomarker in ii., iii., and iv. is selected from the group consisting of SM18, SM24, SM23, SM21, SM28, SM5, SM3, SM29 and SM8.
2. The method of embodiment 1, wherein the heart failure is heart failure with reduced left ventricular ejection fraction (HFrEF).
3. The method of embodiments 1 and 2, wherein at least the amounts of the biomarkers of i., ii., iii., vi, vii. ix or x. (in particular of i., ii., iii., vi., vii. or ix) of embodiment 1 are determined, and wherein the at least one triacylglyceride biomarker in i., ii., and iii. is selected from the group consisting of SOP2, OSS2, SSP2, PPO1 and PPP, and/or (in particular “and”) the least one cholesterylester biomarker in i., and iii. is cholesterylester C18:2, and/or (in particular “and”) the least one phosphatidylcholine biomarker in i. and ii. is PC4, and/or (in particular “and”) the least one sphingomyelin biomarker in ii. and iii. is selected from the group consisting of SM18, SM24, SM23, SM28, SM5, and SM3.
4. The method of any one of embodiments 1 to 3, wherein at least the amounts of the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 in Table 2 are determined.
5. The method of any one of embodiments 1 to 4, wherein at least the amounts of the biomarkers of i., ii., iii, vi, ix or x. are determined, and wherein the at least one triacylglyceride biomarker in i., ii., and iii. is SOP2 and/or OSS2, and/or (in particular “and”) wherein the at least one cholesterylester biomarker in i., and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is selected from the group consisting of SM18, SM24, SM23, and SM3.
6. The method of any one of embodiments 1 to 5, wherein at least the amounts of the biomarkers of panel 1, 2, 3, 4, 5, 6, 7, 8, 9, 10, 11, 12, 13, 14, 15, 16, 17 or 18 in Table 2 are determined.
7. The method of any one of embodiments 1 to 6, wherein at least the amounts of the biomarkers of i., ii., iii, vi, ix or x. (in particular of i., ii, iii, or vi.) are determined, and wherein the at least one triacylglyceride biomarker in i., ii., and iii. is SOP2 and/or OSS2, and/or (in particular “and”) wherein the at least one cholesterylester biomarker in i., and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is SM23.
8. The method of any one of embodiments 1 to 7, wherein at least the amounts of the biomarkers of panel 1, 2, 3 or 4 in Table 2 are determined.
9. The method of any one of embodiments 1 to 7, wherein at least the amounts of OSS2, PC4 and SM23 are determined, in particular wherein at least the amounts of OSS2, PC4 and SM23, and the amount of NT-proBNP or BNP are determined.
10. The method of any one of embodiments 1 to 7, wherein at least the amounts of OSS2, cholesterylester C18:2 and SM23 are determined.
11. The method of any one of embodiments 1 to 7, wherein at least the amounts of the biomarkers of iii. are determined, and wherein the at least one triacylglyceride biomarker is SOP2 and/or OSS2, and/or (in particular “and”) wherein the at least one cholesterylester biomarker is cholesterylester C18:2, and/or (in particular “and”) wherein the at least one sphingomyelin biomarker is SM23.
12. The method of any one of embodiments 1 to 11, wherein at least the amounts of SOP2, OSS2, PC4, Cholesterylester C18:2, SM18, SM28, SM24, SSP2, and SM23 are determined.
13. The method of any one of embodiments 2 to 12, in particular of embodiments 2 to 4, wherein the heart failure with reduced left ventricular ejection fraction is DCMP (dilated cardiomyopathy).
14. The method of embodiment 13, wherein at least the amounts of the biomarkers shown in i., ii., iii. or vii, in particular of in i., ii., or iii. are determined, and wherein the at least one triacylglyceride biomarker in i., ii. and iii. is selected from the group consisting of SOP2, OSS2, and SSP2, and/or (in particular “and”) wherein the at least one cholesterylester biomarker in i. and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is selected from the group consisting of SM24, SM23, SM28, and SM3.
15. The method of embodiments 13 and 14, wherein at least the amounts of the biomarkers of panel 19, 20, 21, 22, 23, 24, 25, 26, 27, 28, 29, 30, 31, 32, 33, 34, 35 or 36 in Table 2 are determined.
16. The method of any one of embodiments 13 to 15, in particular of embodiments 13 and 14, wherein at least the amounts of the biomarkers shown in ii. or iii. are determined, and wherein the at least one triacylglyceride biomarker in ii. and iii. is SOP2 and/or OSS2, and/or (in particular “and”) wherein the at least one cholesterylester biomarker in iii. is cholesterylester C18:2, and/or (in particular “and”) wherein the at least one phosphatidylcholine biomarker in ii. is PC4, and/or (in particular “and”) wherein the at least one sphingomyelin biomarker in ii. and iii. is SM23 and/or SM24.
17. The method of any of embodiments 13 to 16, wherein at least the amounts of the biomarkers of panel 31, 32, 33, 34, 35 and 36 in Table 2 are determined.
18. The method of any one of embodiments 2 to 12, in particular of embodiments 2 to 4, wherein the heart failure with reduced left ventricular ejection fraction is ICMP (ischemic card iomyopathy).
19. The method of embodiment 18, wherein at least the amounts of the biomarkers shown in ii., iii. or vi, in particular in ii., or iii. are determined, and wherein the at least one triacylglyceride biomarker in ii. and iii. is SOP2 and/or OSS2 (in particular “and”), wherein the at least one cholesterylester biomarker in iii. is cholesterylester C18:2, and/or (in particular “and”) wherein the at least one phosphatidylcholine biomarker in ii. is PC4, and/or (in particular “and”) wherein the at least one sphingomyelin biomarker in ii. and iii. is selected from the group consisting of SM18, SM24, and SM23.
20. The method of embodiments 18 and 19, wherein at least the amounts of the biomarkers of panel 37, 38, 39, 40, 41, 42, 43, 44, 45, 46, 47, 48, 49, 50, 51, 52, 53, 54, 55 or 56 in Table 2 are determined.
21. The method of any one of embodiments 18 to 20, wherein at least the amounts of the biomarkers shown in iii. are determined, and wherein the at least one triacylglyceride biomarker is SOP2, and/or (in particular “and”) wherein the at least one cholesterylester biomarker is cholesterylester C18:2, and/or (in particular “and”) wherein the at least one sphingomyelin biomarker is SM18 and/or SM23.
22. The method of any one of embodiments 18 to 21, wherein at least the amounts of the biomarkers of panel 51, 52, 53, 54, 55 or 56 in Table 2 are determined.
23. The method of any one of embodiments 2 to 23, in particular of embodiments 2 to 4, wherein the HFrEF is asymptomatic (or wherein the subject does not show symptoms of heart failure).
24. The method of embodiment 23, and wherein at least the amounts of the biomarkers of, ii., iii. vi, vii., ix or x. (in particular of ii, iii, vi, vii, or ix.) are determined, and wherein the at least one triacylglyceride biomarker in ii., and iii. is selected from the group consisting of SOP2, OSS2, and PPO1, and/or (in particular “and”) wherein the at least one cholesterylester biomarker in iii. is cholesterylester C18:2, and/or (in particular “and”) the least one phosphatidylcholine biomarker in ii. is PC4, and/or (in particular “and”) wherein the at least one sphingomyelin biomarker in ii. and iii. is selected from the group consisting of SM24 and SM23.
25. The method of embodiments 23 and 24, wherein at least the amounts of the biomarkers of panel 7, 8, 9, 10, 11, 12, 19, 20, 21, 22, 23, 24, 37, 38, 39, 40, 41 or 42 in Table 2 are determined.
26. The method of any one of embodiments 23 to 25, in particular of embodiments 23 and 24, wherein at least the amounts of the biomarkers of iii. or vi. (in particular of iii.) of embodiment 1 are determined, and wherein the at least one triacylglyceride biomarker is SOP2, and/or (in particular “and”) wherein the at least one cholesterylester biomarker is cholesterylester C18:2, and/or (in particular “and”) wherein the at least one sphingomyelin biomarker is selected from the group consisting of SM24 and SM23.
27. The method of embodiment 26, wherein at least the amounts of the biomarkers of panel 7, 8, 9, 10, 11, or 12 in Table 2 are determined.
28. The method of any one of embodiments 13 to 17, in particular of any one of embodiments 13, 14 and 16, wherein the DCMP is asymptomatic (or wherein the subject does not show symptoms of heart failure).
29. The method of embodiment 28, wherein at least the amounts of the biomarkers of iii. in embodiment 1 are determined, and wherein the at least one triacylglyceride biomarker is selected from the group consisting of SOP2 and OSS2, and/or (in particular “and”) wherein the at least one cholesterylester biomarker is cholesterylester C18:2, and/or (in particular “and”) wherein the at least one sphingomyelin biomarker is SM23.
30. The method of embodiments 28 or 29, wherein at least the amounts of the biomarkers of panel 19, 20, 21, 22, 23 or 24 in Table 2 are determined.
31. The method of embodiments 18 or 22, wherein the ICMP is asymptomatic (or wherein the subject does not show symptoms of heart failure).
32. The method of embodiment 31, wherein at least the amounts of the biomarkers of iii. or vi (in particular of iii.) are determined, and wherein the at least one triacylglyceride biomarker is SOP2, and/or (in particular “and”) wherein the at least one cholesterylester biomarker is cholesterylester C18:2, and/or (in particular “and”) wherein the at least one sphingomyelin biomarker is selected from the group consisting of SM24 and SM23.
33. The method of embodiments 31 or 32, wherein at least the amounts of the biomarkers of panel 37, 38, 39, 40, 41 or 42 in Table 2 are determined.
34. The method of embodiment 1, wherein the heart failure is heart failure with preserved ejection fraction (HFpEF).
35. The method of embodiment 34, wherein at least the amounts of the biomarkers of ii., vi, or vii. of embodiment 1 are determined, and wherein the at least one triacylglyceride biomarker in ii. is selected from the group consisting of SOP2, SSP2, SPP1 and PPO1, and/or (in particular “and”) the least one phosphatidylcholine biomarker in ii. is selected from the group consisting of PC4 and PC8, and/or (in particular “and”) the least one sphingomyelin biomarker in ii. is selected from the group consisting of SM18, SM24, SM23, SM28, SM5, and SM3.
36. The method of embodiment 35, wherein at least the amounts of the biomarkers of panel 79, 80, 81, 82, 83, 84, 85, 86, 87, 88, 89, 90, 91, 92, 93, 94, 95, 96, 97, 98, 99, 100, 101 or 102, in Table 2 are determined.
37. The method of embodiment 35 or 36, wherein at least the amounts of the biomarkers of ii. of embodiment 1 are determined, and wherein the at least one triacylglyceride biomarker is selected from the group consisting of SSP2, SPP1 and PPO1, and/or (in particular “and”) the least one phosphatidylcholine biomarker is PC4, and/or (in particular “and”) the least one sphingomyelin biomarker is selected from the group consisting of SM24, SM5, and SM3.
38. The method of embodiment 37, wherein at least the amounts of the biomarkers of panel 79, 81, 83, 85, 87, 89, 91, 93, 95, 97, 99 or 101 in Table 2 are determined.
39. The method of any one of embodiment 35 to 38, in particular of any one of embodiments 34 to 37, wherein at least the amounts of the biomarkers of ii. of embodiment 1 are determined, and wherein the at least one triacylglyceride biomarker is SSP2, and/or (in particular “and”) the least one phosphatidylcholine biomarker is PC4, and/or (in particular “and”) the least one sphingomyelin biomarker in ii. is selected from the group consisting of SM24 and SM5.
40. The method of embodiment 39, wherein at least the amounts of the biomarkers of panel 95, 97, 99 or 101 in Table 2 are determined.
41. The method of any one of embodiments 34 to 37, in particular of any one of embodiments 34 to 36, wherein the HFpEF is asymptomatic.
42. The method of embodiment 41, wherein in particular at least the amounts of the biomarkers of ii. or vii. (in particular of ii.) of embodiment 1 are determined and wherein the at least one triacylglyceride biomarker is selected from the group consisting of SPP1 and SSP2, and/or (in particular “and”) the least one phosphatidylcholine biomarker is PC4, and/or (in particular “and”) the least one sphingomyelin biomarker is selected from the group consisting of SM5 and SM3.
43. The method of embodiment 41 or 42, wherein at least the amounts of the biomarkers of panel 79, 80, 81, 82, 83, 84, 85 or 86 in Table 2 are determined.
44. The method of any one embodiments 41 to 43, wherein the at least one triacylglyceride biomarker in ii. is SPP1, and/or (in particular “and”) the least one phosphatidylcholine biomarker in ii. is PC4, and/or (in particular “and”) the least one sphingomyelin biomarker in ii. is SM5.
45. The method of any one of embodiments 41 to 44, wherein at least the amounts of the biomarkers of panel 79, 81, 83, or 85 in Table 2 are determined.
46. The method of any one of embodiments 1 to 45, wherein the subject is a human subject.
47. The method of any one of embodiments to 1 to 46, wherein the sample is blood, serum or plasma.
48. The method of any one of embodiments 1 to 47, wherein the method does not comprise the determination of NT-proBNP or BNP.
49. The method of any one of embodiments 1 to 47, further comprising determining the amount of NT-proBNP or BNP in a sample/the sample from the subject and comparing the amount of NT-proBNP or BNP to a reference.
50. The method of any one of embodiments 1 to 49, further comprising carrying out a correction for confounders.
51. The method of embodiment 50, wherein the confounders are age, BMI and/or gender, in particular age, BMI and gender.
52. The method of any one of embodiments 1 to 51, wherein in step b) a score is calculated based on the determined amounts of the at least three biomarkers, and wherein the reference is a reference score.
53. The method of any one of embodiments 1 to 52, wherein the reference or the reference score is from a subject or group of subjects known not to suffer from heart failure.
54. The method of embodiment 53, wherein a value for each of the at least three biomarkers, or a score in the test sample being essentially identical as compared to the reference or reference score is indicative for the absence of heart failure.
55. The method of any one of embodiments 1 to 52, wherein the reference or the reference score is from a subject or group of subjects known to suffer from heart failure.
56. The method of any one of embodiment 55, wherein a value for each of the at least three biomarkers found, or a score in the test sample being essentially identical as compared to the reference or reference score is indicative for the presence of the heart failure.
57. The method of any one of embodiments 1 to 56, wherein the amounts of the at least three biomarkers are determined by mass spectrometry (MS).
58. The method of embodiment 57, wherein the mass spectrometry is LC-MS, in particular LC-MS/MS, or HPLC-MS, in particular HPLC-MS/MS.
59. The method of embodiments 57 and 58, wherein the mass spectrometry comprises an ionization step in which the at least three biomarkers are ionized
60. The method of embodiment 59, wherein the ionization step is carried out by electrospray ionization, in particular by positive ion mode electrospray ionization.
61. A diagnostic device for carrying out the method according to any one of embodiments 1 to
60, comprising:
   a) an analysing unit comprising at least one detector for the at least three biomarkers as set forth in any one of embodiments 1 to 45, wherein said analyzing unit is adapted for determining the amounts of the said biomarkers detected by the at least one detector, and, operatively linked thereto;
   b) an evaluation unit comprising a computer comprising tangibly embedded a computer program code for carrying out a comparison of the determined amounts of the at least three biomarkers, and reference amounts (or a reference score) and a data base comprising said reference amounts for the said biomarkers, whereby it will be diagnosed whether a subject suffers from heart failure.
62. Use of at least three biomarkers as set forth in any one of embodiments 1 to 45 in a sample of a subject for diagnosing heart failure or for the preparation of a pharmaceutical and/or diagnostic composition for diagnosing heart failure.
63. The method of any one of embodiments 2 to 33, the device of embodiment 61 or the use of embodiment 62, wherein the HFrEF is heart failure with a left ventricular ejection fraction of lower than 50% but larger than 35%.
64. The method of any one of embodiments 1 to 33 or 63, the device of embodiment 61 or 63, or the use of embodiment 62 or 63, wherein the subject is overweight.
65. The method of any one of embodiments 1 to 33 or 63, the device of embodiment 61 or 63, or the use of embodiment 62 or 63, wherein the subject is older than 54 years of age.
66. The method of any one of embodiments 1 to 33 or 63, the device of embodiment 61 or 63, or the use of embodiment 62 or 63, wherein the subject is a female subject, older than 54 years of age, and has a body mass index of less than 25.0 kg/m².
67. The method of any one of embodiments 1 to 33 or 63, the device of embodiment 61 or 63, or the use of embodiment 62 or 63, wherein the subject is younger than 61 years of age, and has a body mass index of more than 26.8 kg/m².

Further Preferred Items of the Present Invention
1. A method for diagnosing heart failure comprising the steps of:
   a. determining in a sample of a subject the amounts of at least three biomarkers wherein said at least three biomarker are
      i. at least one sphingomyelin biomarker (SM) selected from the group consisting of SM18, SM21, SM23, SM24, SM28, SM3, SM5, SM2, SM9 and SM10, in particular at least one sphingomyelin (SM) biomarker selected from the group consisting of SM18, SM21, SM23, SM24, SM28, SM3 and SM5,
      ii. at least one triacylglyceride biomarker selected from the group consisting of OSS2, PPO1, SOP2, SSP2 and SPP1, in particular at least one triacylglyceride biomarker selected from the group consisting of OSS2, PPO1, SOP2, and SSP2, and
      iii. at least one further biomarker selected from the group consisting of Cer(d16:1/24:0), Cer(d18:1/24:1), Cholesterylester C18:2, PC4, PC8, Cer(d18:2/24:0), Cer(d17:1/24:0) and glutamic acid 1, in particular at least one further biomarker selected from the group consisting of Cer(d16:1/24:0), Cer(d18:1/24:1), Cholesterylester C18:2, and PC4,
      and
   b. comparing the amounts as determined in step a. to a reference, whereby heart failure is to be diagnosed.
2. The method of item 1, wherein at least the amounts of
   i. at least one sphingomyelin biomarker selected from the group consisting of SM28, SM23, SM21, and SM5,
   ii. SOP2 and/or OSS2, and
   iii. Cholesterylester C18:2 and/or PC4
are determined.
3. The method of item 1 or 2, wherein at least the amounts of
   i. at least one sphingomyelin biomarker selected from the group consisting of SM28, SM21, and SM5, in particular SM28, or at least one sphingomyelin biomarker selected from the group consisting of SM18, SM21, and SM5, in particular SM18, and
   ii. SOP2, and
   iii. PC4
are determined.
4. The method of any one of items 1 to 3, the heart failure is asymptomatic heart failure, and/or wherein the subject does not show symptoms of heart failure.
5. The method of item 4, wherein at least the amounts of
   i. at least one sphingomyelin biomarker selected from the group consisting of SM18, SM2, SM23, SM24, SM28, SM3, and SM5,
   ii. at least one triacylglyceride biomarker selected from the group consisting of OSS2, PPO1, SOP2, SSP2 and SPP1, and
   iii. at least one further biomarker selected from the group consisting of Cer(d16:1/24:0), Cholesterylester C18:2, PC4 and glutamic acid 1
are determined.
6. The method of items 4 and 5, wherein at least the amounts of
   i. at least one sphingomyelin biomarker selected from the group consisting of SM23, SM2, and SM24,
   ii. at least one triacylglyceride biomarker selected from the group consisting of SOP2, OSS2, and PPO1, and
   iii. Cholesterylester C18:2 and/or PC4, in particular Cholesterylester C18:2,
are determined.
7. The method of any one of items 4 to 6, wherein at least the amounts of SM23 and/or SM2, in particular of SM23, of SOP2, and of PC4 are determined.
8. The method of any one of items 1 to 3, wherein the heart failure is symptomatic heart failure and/or wherein the subject shows symptoms of heart failure.
9. The method of item 8, wherein at least the amounts of
   i. at least one sphingomyelin biomarker selected from the group consisting of SM10, SM18, SM21, SM23, SM24, SM28, SM3 and SM9,
   ii. at least one triacylglyceride biomarker selected from the group consisting of OSS2, PPO1, SOP2, SSP2 and SPP1, and
   iii. at least one further biomarker selected from the group consisting of Cer(d16:1/24:0), Cer(d18:1/24:1), Cer(d17:1/24:0), Cer (d18:2/24:0), Cholesterylester C18:2, PC4 and PC8
are determined.
10. The method of items 8 and 9, wherein at least the amounts of
   i. at least one sphingomyelin biomarker is selected from the group consisting of SM18, SM3, SM24, and SM23, in particular SM18,
   ii. SOP2 and/or OSS2, in particular SOP2, and
   iii. Cholesterylester C18:2 and/or PC4, in particular PC4,
are determined.
11. The method of any one of items 8 to 10, wherein at least the amounts of SM18 and/or SM24, SOP2, and PC4 are determined.
12. The method of any one of items 1 to 11, wherein the heart failure is heart failure with reduced left ventricular ejection fraction (HFrEF).
13. The method of item 12, wherein at least the amounts of
   i. at least one sphingomyelin biomarker selected from the group consisting of SM23, SM18, SM24, SM28, SM2 and SM3, in particular at least one sphingomyelin biomarker selected from the group consisting of SM23, SM18, SM24, and SM28,
   ii. OSS2 and/or SOP2, in particular OSS2,
   iii. Cholesterylester C18:2 and/or PC4
are determined.
14. The method of items 12 and 13, wherein at least the amounts of SM23, OSS2, and Cholesterylester C18:2 are determined.
15. The method of any one of items 12 to 14, wherein the HFrEF is asymptomatic HFrEF, and/or wherein the subject does not show symptoms of heart failure.
16. The method of item 15, wherein at least the amounts of
   i. at least one sphingomyelin biomarker selected from the group consisting of SM23 SM18, SM2, SM24 and SM28,
   ii. SOP2, and
   iii. Cholesterylester C18:2
are determined.
17. The method of items 15 and 16, wherein at least the amounts of SM23, SOP2, and Cholesterylester C18:2 are determined.
18. The method of any one of items 12 to 14, wherein the HFrEF is symptomatic HFrEF, and/or wherein the subject shows symptoms of heart failure.
19. The method of item 18, wherein at least the amounts of
   i. at least one sphingomyelin biomarker selected from the group consisting of SM18, SM23, SM3 or at least one sphingomyelin biomarker selected from the group consisting of SM28, SM23 and SM3,
   ii. OSS2, and
   iii. Cholesterylester C18:2 and/or PC4
are determined.
20. The method of items 18 and 19, wherein at least the amounts of SM18 and/or SM28, of OSS2, and of Cholesterylester C18:2 are determined.
21. The method of any one of items 12 to 14, wherein the heart failure with reduced left ventricular ejection fraction is DCMP (dilated cardiomyopathy).
22. The method of item 21, wherein at least the amounts of
   i. at least one sphingomyelin biomarker selected from the group consisting of SM23, SM24, SM28, and SM3, in particular at least one sphingomyelin biomarker selected from the group consisting of SM23, SM24, and SM28,
   ii. SOP2 and/or OSS2, and
   iii. Cholesterylester C18:2 and/or PC4
are determined.
23. The method of items 21 and 22, wherein at least the amounts of SM23, of SOP2, and of Cholesterylester C18:2 are determined.
24. The method of items 21 to 23, wherein the DCMP is asymptomatic DCMP, and/or wherein the subject does not show symptoms of heart failure.
25. The method of item 21, wherein at least the amounts of
   i. SM23,
   ii. OSS2 and/or SOP2, and
   iii. Cholesterylester C18:2
are determined.
26. The method of items 24 and 25, wherein at least the amounts of SM23, OSS2, and Cholesterylester C18:2 are determined.
27. The method of items 21 to 23, wherein the DCMP is symptomatic DCMP, and/or wherein the subject shows symptoms of heart failure.
28. The method of item 27, wherein at least the amounts of
   i. SM28 and/or SM3,
   ii. SOP2 and/or OSS2, and
   iii. PC4
are determined.
29. The method of items 27 and 28, wherein the at least the amounts of SM28, SOP2, and of PC4 are determined.
30. The method of any one of items 12 to 14, wherein the heart failure with reduced left ventricular ejection fraction is ICMP (ischemic cardiomyopathy).
31. The method of item 30, wherein at least the amounts of
   i. at least one sphingomyelin biomarker selected from the group consisting of SM23, SM24, SM28 and SM18, in particular at least one sphingomyelin biomarker selected from the group consisting of SM23, SM24, and SM28,
   ii. SOP2, and
   iii. Cholesterylester C18:2
are determined.
32. The method of items 30 and 31, wherein at least the amounts of SM23, SOP2, and Cholesterylester C18:2 are determined.
33. The method of items 31 and 32, wherein the ICMP is symptomatic ICMP, and/or wherein the subject shows symptoms of heart failure.
34. The method of item 33, wherein at least the amounts of SM18, SOP2, and Cholesterylester C18:2 are determined.
35. A method for diagnosing asymptomatic heart failure comprising the steps of:
   a. determining in a sample of a subject the amounts of
      i. at least one sphingomyelin biomarker selected from the group consisting of SM23, SM2, and SM24,
      ii. at least one triacylglyceride biomarker selected from the group consisting of SOP2, OSS2, and PPO1, and
      iii. at least one further biomarker selected from Cholesterylester C18:2, PC4, and SM5,
      and
   b. comparing the amounts as determined in step a. to a reference, whereby heart failure is to be diagnosed.
36. A method for diagnosing asymptomatic ICMP comprising the steps of:
   a. determining in a sample of a subject the amounts of at least three markers selected from the group consisting of SM24, SM5, SM23, SOP2 and PPO1, and
   b. comparing the amounts as determined in step a. to a reference, whereby heart failure is to be diagnosed.
37. The method of items 36, wherein at least the amounts of
   i. SM24, SM5 and SOP2, or
   ii. SM23, SM5 and PPO1,
are determined.
38. The method of any one of items 1 to 37, wherein the subject is a human subject.
39. The method of any one of items to 1 to 38, wherein the sample is blood, serum or plasma.
40. The method of any one of items 1 to 39, wherein the method does not comprise the determination of NT-proBNP or BNP.
41. The method of any one of items 1 to 39, further comprising determining the amount of NT-proBNP or BNP in a sample/the sample from the subject and comparing the amount of NT-proBNP or BNP to a reference.
42. The method of any one of items 1 to 41, further comprising carrying out a correction for confounders.
43. The method of item 42, wherein the confounders are age, BMI and/or gender, in particular age, BMI and gender.
44. The method of any one of items 1 to 43, wherein in step b) a score is calculated based on the determined amounts of the at least three biomarkers, and wherein the reference is a reference score.
45. The method of any one of items 1 to 44, wherein the reference or the reference score is from a subject or group of subjects known not to suffer from heart failure.
46. The method of item 45, wherein a value for each of the at least three biomarkers, or a score in the test sample being essentially identical as compared to the reference or reference score is indicative for the absence of heart failure.
47. The method of any one of items 1 to 44, wherein the reference or the reference score is from a subject or group of subjects known to suffer from heart failure.
48. The method of any one of item 47, wherein a value for each of the at least three biomarkers found, or a score in the test sample being essentially identical as compared to the reference or reference score is indicative for the presence of the heart failure.
49. The method of any one of items 1 to 48, wherein the amounts of the at least three biomarkers are determined by mass spectrometry (MS).
50. The method of item 49, wherein the mass spectrometry is LC-MS, in particular LCMS/MS, or HPLC-MS, in particular HPLC-MS/MS.
51. The method of items 49 and 50, wherein the mass spectrometry comprises an ionization step in which the at least three biomarkers are ionized
52. The method of item 51, wherein the ionization step is carried out by electrospray ionization, in particular by positive ion mode electrospray ionization.
53. A diagnostic device for carrying out the method according to any one of items 1 to 52, comprising:
   a) an analysing unit comprising at least one detector for the at least three biomarkers as set forth in any one of items 1 to 37, wherein said analyzing unit is adapted for determining the amounts of the said biomarkers detected by the at least one detector, and, operatively linked thereto;
   b) an evaluation unit comprising a computer comprising tangibly embedded a computer program code for carrying out a comparison of the determined amounts of the at least three biomarkers, and reference amounts (or a reference score) and a data base comprising said reference amounts for the said biomarkers, whereby it will be diagnosed whether a subject suffers from heart failure.
54. Use of at least three biomarkers as set forth in any one of items 1 to 37 in a sample of a subject for diagnosing heart failure or for the preparation of a pharmaceutical and/or diagnostic composition for diagnosing heart failure.
55. The method of any one of items 2 to 52, the device of embodiment 53 or the use of embodiment
54, wherein the HFrEF is heart failure with a left ventricular ejection fraction of lower than 50% but larger than 35%.
56. A composition comprising the at least three biomarkers as set forth in any one of items 1 to 27.
57. A calibration solution comprising the composition of item 56.
58. A calibration sample, comprising the composition of item 56 and delipidized serum or delipidized plasma.

### Further Preferred Embodiments of the Present Invention

1. A method for diagnosing heart failure comprising the steps of:
   a. determining in a sample of a subject the amounts, preferably, the amounts of at least three biomarkers, wherein said at least three biomarkers are
      i. at least one sphingomyelin (SM) biomarker selected from the group consisting of SM18, SM23, SM24, SM3, SM5, SM2, and SM28, and in particular at least one sphingomyelin (SM) biomarker selected from the group consisting of SM18, SM23, SM24, SM3, and SM5,
      ii. at least one triacylglyceride biomarker selected from the group consisting of OSS2, SOP2, SSP2 and PPO1, in particular at least one triacylglyceride biomarker selected from the group consisting of OSS2 and SOP2, and
      iii. at least one further biomarker selected from the group consisting of Cholesterylester C18:2 and PC4, and
   b. comparing the amounts as determined in step a. to a reference, whereby heart failure is to be diagnosed.
2. The method of embodiment 1, wherein at least the amounts of
   i. at least one sphingomyelin biomarker selected from the group consisting of SM18, SM23, SM24, SM3, and SM5,
   ii. at least one triacylglyceride biomarker selected from the group consisting of OSS2 and SOP2, and
   iii. at least one further biomarker selected from the group consisting of Cholesterylester C18:2 and PC4
are determined.
3. The method of embodiment 1 or 2, wherein at least the amounts of
   i. at least one sphingomyelin biomarker selected from the group consisting of SM18, SM3, and SM5, in particular SM18, and
   ii. at least one triacylglyceride biomarker selected from the group consisting of OSS2 and SOP2, in particular SOP2, and
   iii. at least one further biomarker selected from the group consisting of Cholesterylester C18:2 and PC4, in particular PC4
are determined.
4. The method of any one of embodiments 1 to 3, the heart failure is asymptomatic heart failure, and/or wherein the subject does not show symptoms of heart failure.
5. The method of embodiment 4, wherein at least the amounts of
   i. at least one sphingomyelin biomarker selected from the group consisting of SM18, SM2, SM23, SM24, SM28, and SM5,
   ii. at least one triacylglyceride biomarker selected from the group consisting of OSS2, PPO1, SOP2, and SSP2, and
   iii. at least one further biomarker selected from the group consisting of Cholesterylester C18:2 and PC4
are determined.
6. The method of embodiments 4 and 5, wherein at least the amounts of
   i. at least one sphingomyelin biomarker selected from the group consisting of SM23, SM24, SM18, and SM2, in particular SM23,
   ii. at least one triacylglyceride biomarker selected from the group consisting of SOP2, OSS2, and PPO1, in particular SOP2, and
   iii. at least one further biomarker selected from the group consisting of Cholesterylester C18:2 and PC4, in particular Cholesterylester C18:2,
are determined
7. The method of any one of embodiments 4 to 6, wherein at least the amounts of
   i. at least one sphingomyelin biomarker selected from the group consisting of SM23, SM18, and SM2, in particular SM23,
   ii. SOP2, and
   iii. Cholesterylester C18:2 and/or PC4, in particular Cholesterylester C18:2,
are determined.
8. The method of any one of embodiments 1 to 3, wherein the heart failure is symptomatic heart failure, and/or wherein the subject shows symptoms of heart failure.
9. The method of embodiment 8, wherein at least the amounts of
   i. at least one sphingomyelin biomarker selected from the group consisting of SM18, SM23, SM24, SM28, and SM3,
   ii. at least one triacylglyceride biomarker selected from the group consisting of OSS2 and SOP2, and
   iii. at least one further biomarker selected from the group consisting of Cholesterylester C18:2 and PC4
are determined.
10. The method of embodiments 8 and 9, wherein at least the amounts of
   i. SM18,
   ii. SOP2, and
   iii. Cholesterylester C18:2,
are determined.
11. The method of any one of embodiments 8 to 10, wherein at least the amounts of SM18 and/or SM24, in particular SM18, of SOP2, and of PC4 are determined.
12. The method of any one of embodiments 1 to 11, wherein the heart failure is heart failure with reduced left ventricular ejection fraction (HFrEF).
13. The method of embodiment 12, wherein at least the amounts of
   i. at least one sphingomyelin biomarker selected from the group consisting of SM23, SM18, SM3, SM24, SM 28 in particular at least one sphingomyelin selected from the group consisting of SM23 and SM18,
   ii. at least one triacylglyceride biomarker selected from the group consisting of OSS2, SOP2 and PPO1, in particular OSS2, and
   iii. at least one further biomarker selected from the group consisting of Cholesterylester C18:2 and PC4
are determined.
14. The method of embodiments 12 and 13, wherein at least the amounts of SM23, OSS2, and Cholesterylester C18:2 are determined.
15. The method of any one of embodiments 12 to 14, wherein the HFrEF is asymptomatic HFrEF, and/or wherein the subject does not show symptoms of heart failure.
16. The method of embodiment 15, wherein at least the amounts of
   i. at least one sphingomyelin biomarker selected from the group consisting of SM23 and SM24, in particular SM23,
   ii. at least one triacylglyceride biomarker selected from the group consisting of SOP2, and OSS2, in particular SOP2, and
   iii. at least one further biomarker selected from the group consisting of Cholesterylester C18:2 and PC4, in particular Cholesterylester C18:2
are determined.
17. The method of embodiments 15 and 16, wherein at least the amounts of SM23, SOP2, and Cholesterylester C18:2 are determined.
18. The method of any one of embodiments 12 to 14, wherein the HFrEF is symptomatic HFrEF, and/or wherein the subject shows symptoms of heart failure.
19. The method of embodiment 18, wherein at least the amounts of
   i. at least one sphingomyelin biomarker selected from the group consisting of SM18, SM23, SM3, SM28 and SM24, in particular at least one sphingomyelin selected from the group consisting of SM18, SM23 and SM3,
   ii. at least one triacylglyceride biomarker selected from the group consisting of SOP2 and OSS2, and
   iii. at least one further biomarker selected from the group consisting of Cholesterylester C18:2 and PC4
are determined.
20. The method of embodiments 18 and 19, wherein at least the amounts of SM18, of OSS2, and of Cholesterylester C18:2 are determined.
21. The method of any one of embodiments 12 to 14, wherein the heart failure with reduced left ventricular ejection fraction is DCMP (dilated cardiomyopathy).
22. The method of embodiment 21, wherein at least the amounts of
   i. at least one sphingomyelin biomarker selected from the group consisting of SM23, SM24, SM3 and SM28, in particular at least one sphingomyelin biomarker selected from the group consisting of SM23 and SM24,
   ii. at least one triacylglyceride biomarker selected from the group consisting of SOP2, and OSS2, and
   iii. at least one further biomarker selected from the group consisting of Cholesterylester C18:2 and PC4
are determined.
23. The method of embodiments 21 and 22, wherein at least the amounts of SM23, of SOP2, and of Cholesterylester C18:2 are determined.
24. The method of embodiments 21 to 23, wherein the DCMP is asymptomatic DCMP, and/or wherein the subject does not show symptoms of heart failure.
25. The method of embodiment 21, wherein at least the amounts of
   i. at least one sphingomyelin biomarker selected from the group consisting of SM23 and SM24,
   ii. at least one triacylglyceride biomarker selected from the group consisting of SOP2 and OSS2, and
   iii. Cholesterylester C18:2
are determined.
26. The method of embodiments 24 and 25, wherein at least the amounts of SM23, OSS2, and Cholesterylester C18:2 are determined.
27. The method of embodiments 21 to 23, wherein the DCMP is symptomatic DCMP, and/or wherein the subject shows symptoms of heart failure.
28. The method of embodiment 27, wherein at least the amounts of
   i. at least one sphingomyelin biomarker selected from the group consisting of SM3, SM24 and SM28,
   ii. at least one triacylglyceride biomarker selected from the group consisting of SOP2 and OSS2, and
   iii. at least one further biomarker selected from the group consisting of Cholesterylester C18:2 and PC4
are determined.
29. The method of embodiments 27 and 28, wherein the at least the amounts of SM3, OSS2, and of PC4 are determined.
30. The method of any one of embodiments 12 to 14, wherein the heart failure with reduced left ventricular ejection fraction is ICMP (ischemic cardiomyopathy).
31. The method of embodiment 30, wherein at least the amounts of
   i. at least one sphingomyelin biomarker selected from the group consisting of SM18, SM23, and SM24, in particular at least one sphingomyelin biomarker selected from the group consisting of SM18 and SM23,
   ii. at least one triacylglyceride biomarker selected from the group consisting of SOP2 and PPO1, in particular SOP2, and
   iii. at least one further biomarker selected from the group consisting of Cholesterylester C18:2 and PC4, in particular Cholesterylester C18:2
are determined.
32. The method of embodiments 30 and 31, wherein at least the amounts of SM18, SOP2, and Cholesterylester C18:2 are determined.
33. The method of embodiments 31 and 32, wherein the ICMP is asymptomatic ICMP, and/or wherein the subject does not show symptoms of heart failure.
34. The method of embodiment 33, wherein
   i. at least one sphingomyelin biomarker selected from the group consisting of SM24 and SM23,
   ii. at least one triacylglyceride biomarker selected from the group consisting of SOP2 and PPO1, and
   iii. at least one further biomarker selected from the group consisting of Cholesterylester C18:2 and PC4, in particular Cholesterylester C18:2
are determined.
35. The method of embodiment 33, wherein at least the amounts of SM24, SOP2, and Cholesterylester C18:2 are determined.
36. The method of embodiments 31 and 32, wherein the ICMP is symptomatic ICMP, and/or wherein the subject shows symptoms of heart failure.
37. The method of embodiment 33, wherein at least the amounts of SM18, SOP2, and Cholesterylester C18:2 are determined.
38. A method for diagnosing asymptomatic heart failure comprising the steps of:
   a. determining in a sample of a subject the amounts of
      i. at least one sphingomyelin biomarker selected from the group consisting of SM23, SM24, SM18, and SM2, in particular SM23,
      ii. at least one triacylglyceride biomarker selected from the group consisting of SOP2, OSS2, and PPO1, in particular SOP2, and
      iii. at least one further biomarker selected from the group consisting of Cholesterylester C18:2, PC4, SM5 and SSP2, in particular Cholesterylester C18:2, and
   b. comparing the amounts as determined in step a. to a reference, whereby heart failure is to be diagnosed.
39. A method for diagnosing asymptomatic heart failure comprising the steps of:
   a. determining in a sample of a subject the amounts of
      i. at least one sphingomyelin biomarker selected from the group consisting of SM23, SM18, and SM2, in particular SM23,
      ii. SOP2, and
      iii. at least one further biomarker selected from the group consisting of SM5, Cholesterylester C18:2 PC4, in particular SM5, and
   b. comparing the amounts as determined in step a. to a reference, whereby heart failure is to be diagnosed.
40. A method for diagnosing asymptomatic HFrEF comprising the steps of:
   a. determining in a sample of a subject the amounts of
      i. at least one sphingomyelin biomarker selected from the group consisting of SM23 and SM24,
      ii. at least one triacylglyceride biomarker selected from the group consisting of SOP2, and OSS2, in particular SOP2, and
      iii. at least one further biomarker selected from the group consisting of Cholesterylester C18:2 and SM28, in particular Cholesterylester C18:2, and
   b. comparing the amounts as determined in step a. to a reference, whereby heart failure is to be diagnosed.
41. A method for diagnosing asymptomatic DCMP comprising the steps of:
   a. determining in a sample of a subject the amounts of
      i. at least one sphingomyelin biomarker selected from the group consisting of SM23 and SM24, in particular SM23,
      ii. at least one triacylglyceride biomarker selected from the group consisting of SOP2 and OSS2, in particular OSS2 and
      iii. Cholesterylester C18:2 and SSP2, in particular Cholesterylester C18:2, and
   b. comparing the amounts as determined in step a. to a reference, whereby heart failure is to be diagnosed.
42. A method for diagnosing asymptomatic ICMP comprising the steps of:
   a. determining in a sample of a subject the amounts of
      i. at least one sphingomyelin biomarker selected from the group consisting of SM24 and SM23, in particular SM24,
      ii. at least one triacylglyceride biomarker selected from the group consisting of SOP2 and PPO1, in particular SOP2, and
      iii. at least one further biomarker selected from the group consisting of SM5, Cholesterylester C18:2 and PC4, in particular Cholesterylester C18:2, in particular SM5, or in particular PC4, and
   b. comparing the amounts as determined in step a. to a reference, whereby heart failure is to be diagnosed.
43. The method of any one of embodiments 1 to 42, wherein the subject is a human subject.
44. The method of any one of embodiments to 1 to 43, wherein the sample is blood, serum or plasma.
45. The method of any one of embodiments 1 to 44, wherein the method does not comprise the determination of NT-proBNP or BNP.
46. The method of any one of embodiments 1 to 44, further comprising determining the amount of NT-proBNP or BNP in a sample/the sample from the subject and comparing the amount of NT-proBNP or BNP to a reference.
47. The method of any one of embodiments 1 to 46, further comprising carrying out a correction for confounders.
48. The method of embodiment 47, wherein the confounders are age, BMI and gender.
49. The method of any one of embodiments 1 to 48, wherein in step b) a score is calculated based on the determined amounts of the at least three biomarkers, and wherein the reference is a reference score.
50. The method of any one of embodiments 1 to 49, wherein the reference or the reference score is from a subject or group of subjects known not to suffer from heart failure.
51. The method of embodiment 50, wherein a value for each of the at least three biomarkers, or a score in the test sample being essentially identical as compared to the reference or reference score is indicative for the absence of heart failure.
52. The method of any one of embodiments 1 to 49, wherein the reference or the reference score is from a subject or group of subjects known to suffer from heart failure.
53. The method of any one of embodiment 52, wherein a value for each of the at least three biomarkers found, or a score in the test sample being essentially identical as compared to the reference or reference score is indicative for the presence of the heart failure.
54. The method of any one of embodiments 1 to 53, wherein the amounts of the at least three biomarkers are determined by mass spectrometry (MS).
55. The method of embodiment 54, wherein the mass spectrometry is LC-MS, in particular LC-MS/MS, or HPLC-MS, in particular HPLC-MS/MS.
56. The method of embodiments 54 and 55, wherein the mass spectrometry comprises an ionization step in which the at least three biomarkers are ionized
57. The method of embodiment 56, wherein the ionization step is carried out by electrospray ionization, in particular by positive ion mode electrospray ionization.
58. A diagnostic device for carrying out the method according to any one of embodiments 1 to
57, comprising:
   a) an analysing unit comprising at least one detector for the at least three biomarkers as set forth in any one of embodiments 1 to 42, wherein said analyzing unit is adapted for determining the amounts of the said biomarkers detected by the at least one detector, and, operatively linked thereto;
   b) an evaluation unit comprising a computer comprising tangibly embedded a computer program code for carrying out a comparison of the determined amounts of the at least three biomarkers, and reference amounts (or a reference score) and a data base comprising said reference amounts for the said biomarkers, whereby it will be diagnosed whether a subject suffers from heart failure.
59. Use of at least three biomarkers as set forth in any one of embodiments 1 to 42 in a sample of a subject for diagnosing heart failure or for the preparation of a pharmaceutical and/or diagnostic composition for diagnosing heart failure.
60. The method of any one of embodiments 2 to 57, the device of embodiment 58 or the use of embodiment 59, wherein the HFrEF is heart failure with a left ventricular ejection fraction of lower than 50% but larger than 35%.
61. A composition comprising the at least three biomarkers as set forth in any one of embodiments 1 to 42.
62. A calibration solution comprising the composition of embodiment 61.
63. A calibration sample comprising the composition of embodiment 61 and delipidized serum or delipidized plasma.

Items for Panel 1

In a preferred embodiment, the amounts of the biomarkers in panel 1 are determined. The heart failure to be diagnosed may be classified as NYHA class I or II. Preferably, the determination is carried out for the diagnosis of HFrEF, in particular for the diagnosis of HFrEF with a left ventricular ejection fraction of lower than 50% but larger than 35%. Further, it is envisaged that the subject does not show symptoms of heart failure.

In addition, it is envisaged to determine the amount of NT-proBNP in addition of the at least three biomarkers. Preferably, a correction for confounders (in particular age, BMI and gender) is not carried out.

With respect to panel 1, the following items are preferred. The definitions and explanations, given in the specification above, preferably, apply mutatis mutandis to following preferred items of the present invention.
1. A method for diagnosing heart failure comprising the steps of:
   a. determining in a sample of a subject the amounts of the SM23, OSS2 and PC4, and
   b. comparing the amounts as determined in step a. to a reference, whereby heart failure is to be diagnosed
2. The method of item 1, wherein the subject does not show symptoms of heart failure.
3. The method of item 1, wherein the subject shows symptoms of heart failure.
4. The method of any one of item 1 to 3, wherein the heart failure is heart failure with reduced left ventricular ejection fraction (HFrEF), in particular wherein said HFrEF is asymptomatic HFrEF.
5. The method of item 4, wherein said heart failure is heart failure with reduced left ventricular ejection fraction (HFrEF) is dilatative cardiomyopathy (DCMP), in particular wherein said DCMP is asymptomatic DCMP.
6. The method of item 4, wherein said heart failure is heart failure with reduced left ventricular ejection fraction (HFrEF) is ischemic cardiomyopathy (ICMP), in particular wherein said ICMP is asymptomatic ICMP.
7. The method of any one of items 2 to 6, wherein the HFrEF is heart failure with a left ventricular ejection fraction of lower than 50% but larger than 35%.
8. The method of any one of items 1 to 7, wherein the subject is a human subject.
9. The method of any one of items to 1 to 8, wherein the sample is blood, serum or plasma.
10. The method of any one of items 1 to 9, wherein the method does not comprise the determination of NT-proBNP or BNP.
11. The method of any one of items 1 to 9, further comprising determining the amount of NT-proBNP or BNP in a sample/the sample from the subject and comparing the amount of NT-proBNP or BNP to a reference.
12. The method of any one of items 1 to 11, further comprising carrying out a correction for confounders.
13. The method of item 12, wherein the confounders are age, BMI and/or gender, in particular age, BMI and gender.
14. The method of item 1 to 11, wherein no correction for confounders is carried out.
15. The method of item 14, wherein no correction for confounders age, BMI and gender is carried out.
16. The method of any one of items 1 to 15, wherein in step b) a score is calculated based on the determined amounts of the at least three biomarkers, wherein the reference is a reference score, and wherein the score is compared to the reference score.
17. The method of any one of items 1 to 16, wherein the reference or the reference score is from a subject or group of subjects known not to suffer from heart failure.
18. The method of item 17, wherein a value for each of the at least three biomarkers, or a score in the test sample being essentially identical as compared to the reference or reference score is indicative for the absence of heart failure.
19. The method of any one of items 1 to 16, wherein the reference or the reference score is from a subject or group of subjects known to suffer from heart failure.
20. The method of any one of item 19, wherein a value for each of the at least three biomarkers found, or a score in the test sample being essentially identical as compared to the reference or reference score is indicative for the presence of the heart failure.
21. The method of any one of items 1 to 20, wherein the amounts of the at least three biomarkers are determined by mass spectrometry (MS).
22. The method of item 21, wherein the mass spectrometry is LC-MS, in particular LCMS/MS, or HPLC-MS, in particular HPLC-MS/MS.
23. The method of items 22 and 23, wherein the mass spectrometry comprises an ionization step in which the at least three biomarkers are ionized.
24. The method of item 24, wherein the ionization step is carried out by electrospray ionization, in particular by positive ion mode electrospray ionization.
25. The method of any one of items 1 to 24, wherein the subject is overweight.
26. The method of any one of items 2 to 33 or 63, wherein the subject is younger than 61 years of age, and has a body mass index of more than 26.8 kg/m².
27. The method of any one of items 1 to 24, wherein the subject is older than 54 years of age.
28. The method of any one of items 1 to 24, wherein the subject is a female subject, older than 54 years of age, and has a body mass index of less than 25.0 kg/m².
29. The method of items 1 to 28, wherein OSS2 is TAG(C18:1, C18:0, C18:0).
30. The method of items 1 to 29, wherein Phosphatidylcholine (C16:0 C18:2).
31. The method of any one of items 1 to 30, wherein SM23 is Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1).
32. The method of any one of items 31, wherein the amount of SM23 is determined by i) determining the amount of the amount of Sphingomyelin(d18:1/23:1), the amount of Sphingomyelin (d17:1/24:1), or, in particular, the combined amount of Sphingomyelin(d18:1/23:1), Sphingomyelin(d18:2/23:0), and Sphingomyelin(d17:1/24:1).
33. The method of any one of items 1 to 33, wherein the amount of SM23 is determined by determining the combined amount of Sphingomyelin(d18:1/23:1), Sphingomyelin(d18:2/23:0), and Sphingomyelin(d17:1/24:1).
34. A method for diagnosing HFrEF comprising the steps of:
   a. determining in a blood, serum or plasma sample of a subject who does not show symptoms of heart failure the amounts of the biomarkers SM23, OSS2 and PC4 and optionally the amount of NT-proBNP, and
   b. comparing the amounts as determined in step a. to a reference, whereby heart failure is to be diagnosed.
35. A method for diagnosing HFrEF comprising the steps of:
   a) determining in a blood, serum or plasma sample of a subject who does not show symptoms of heart failure the amounts of the biomarkers SM23, OSS2 and PC4, and optionally the amount of BNP or NT-proBNP; and
   b1) calculating a score based on the determined amounts of the biomarkers and, if determined, on the amount of BNP or NT-proBNP, and
   b2) comparing the, thus, calculated score to a reference score, whereby heart failure is to be diagnosed.
36. The method of items 34 or 35, wherein no correction for confounders, in particular no correction for the confounders age, BMI and gender is carried out.
37. The method of any of items 34 to 36, wherein the subject is a subject as defined in items 25 to 28.
38. The method of any one of items 34 to 37, wherein the HFrEF is heart failure with a left ventricular ejection fraction of lower than 50% but larger than 35%.
39. A diagnostic device for carrying out the method according to any one of items 1 to 38, comprising:
   a) an analysing unit comprising at least one detector for the biomarkers SM23, OSS2 and PC4, wherein said analyzing unit is adapted for determining the amounts of the said biomarkers detected by the at least one detector, and, operatively linked thereto;
   b) an evaluation unit comprising a computer comprising tangibly embedded a computer program code for carrying out a comparison of the determined amounts of the biomarkers, and reference amounts and a data base comprising said reference amounts for the said biomarkers, whereby it will be diagnosed whether a subject suffers from heart failure.
40. Use of SM23, OSS2 and PC4 in a sample of a subject for diagnosing heart failure or for the preparation of a pharmaceutical and/or diagnostic composition for diagnosing heart failure.
41. Use according to item 40, wherein the heart failure is a heart failure as set forth in any one of items 4 to 7.
42. Use according to item 40 or 41, wherein the subject is a subject as defined in any one of items 25 to 28.
43. Use according to any one of items 40 to 42, wherein the subject does not show symptoms of heart failure.
44. A composition comprising PC4, OSS2 and SM 27, or comprising PC4, OSS2 and SM23.
45. A calibration solution comprising the composition of embodiment 44.
46. A calibration sample comprising the composition of embodiment 44 and delipidized serum or delipidized plasma.

Further preferred embodiments for panel 1:
1. A diagnostic test for diagnosing heart failure in a subject comprising:
   a. one or more prepared blood, serum or plasma samples obtained from a human subject, wherein the prepared sample was processed for the detection of lipid biomarkers;
   b. one or more analysing units for detecting the biomarkers of at least one biomarker panel,

wherein the biomarker panel comprises detecting the amounts of at least one triacylglyceride, at least one phosphatidylcholine, and at least one sphingomyelin;

wherein the at least one sphingomyelin comprises SM23 and the at least one triacylglycerides comprises OSS2 and the at least one phosphatidylcholine comprises PC4; and

further wherein SM23 comprises the sum amounts of Sphingomyelin(d18:1/23:1), Sphingomyelin(d18:2/23:0), and Sphingomyelin(d17:1/24:1);
c. one or more databases containing patient medical record information, wherein the medical record information comprises the subject's BNP test results, and further wherein the BNP test result comprises the amount of at least one of the following brain natriuretic peptide (BNP), amino-terminal pro-brain natriuretic peptide (NT-proBNP), ANP (atrial natriuretic peptide) or NT-proANP (N-terminus of the prohormone brain natriuretic peptide) in a blood, serum, or plasma sample from a subject;
d. an evaluation unit comprising a computer comprising a program for
   i. calculating a score value for a subject based on the amounts of (b) and (c); and
   ii. comparing that score value of step (i) to a reference value, whereby heart failure in a subject is to be diagnosed.
2. The diagnostic test of embodiment 1, wherein the diagnostic test is used to diagnose heart failure with reduced left ventricular ejection fraction (HFrEF) in a subject.
3. The diagnostic test of embodiment 2, wherein HFrEF is DCMP or ICMP.
4. The diagnostic test of embodiment 2, wherein the HFrEF is heart failure with a left ventricular ejection fraction of lower than 50% but larger than 35%.
5. The diagnostic test of embodiment 1, wherein the amounts of the biomarkers in the at least one biomarker panel are determined by mass spectrometry (MS).
6. The diagnostic test of embodiment 1, wherein the test is used on a subject who does not show symptoms of heart failure.
7. The diagnostic test of embodiment 1, wherein the test is used on a subject who is overweight.
8. The diagnostic test of embodiment 1, wherein the test is used on a subject who is older than 54 years of age.
9. The diagnostic test of embodiment 1, wherein the test is used on a subject who is a female subject, older than 54 years of age, and has a body mass index of less than 25.0 kg/m².
10. The diagnostic test of embodiment 1, wherein the test is used on a subject who is younger than 61 years of age, and has a body mass index of more than 26.8 kg/m².
11. A method for diagnosing heart failure comprising the steps of:
   a. obtaining at least one blood, plasma, or serum sample from a human subject
   b. processing the at least one sample for HPLC-MS/MS analysis, wherein the at least one sample is combined with an extraction solvent to create a combined sample, wherein the extraction solvent separates the lipids in the combined sample from the proteins in the combined sample, and further wherein the combined sample undergoes centrifugation to remove the proteins from the sample and to create the processed sample for HPLC-MS/MS analysis;
   c. using HPLC-MS/MS analysis to determine in the at least one processed sample of the subject the amounts of biomarkers in at least one biomarker panel, wherein the at least one biomarker panel comprises at least three biomarkers and further wherein the at least three biomarkers comprise:
      i. at least one triacylglyceride, at least one phosphatidylcholine, and at least one sphingomyelin, wherein the at least one sphingomyelin comprises SM23 and the at least one triacylglycerides comprises OSS2 and the at least one phosphatidylcholine biomarker comprises PC4 and further wherein SM23 comprises the sum amounts of Sphingomyelin(d18:1/23:1), Sphingomyelin(d18:2/23:0), and Sphingomyelin(d17:1/24:1);
   d. obtaining the subject's BNP test results by using one or more of the following methods:
      i. determining the amount of one or more of the following proteins in the sample: NT-proBNP, BNP, ANP, or NT-proANP wherein the amount of the one or more proteins is determined using an antibody test, and/or
      ii. determining the amount of one or more of the following proteins in the sample: NT-proBNP, BNP, ANP, or NT-proANP wherein the amount of the one or more proteins is derived from the subject's medical record and/or stored in an electronic database;
   e. calculating a score value for a subject based on the amounts determined in steps (c) and (d); and
   f. comparing that score value of step (e) to a reference value, whereby heart failure in a subject is to be diagnosed.
12. The method of embodiment 11, wherein the heart failure is heart failure with reduced left ventricular ejection fraction (HFrEF).
13. The method of any of embodiment 12, wherein HFrEF is DCMP or ICMP.
14. The method of embodiment 12, wherein the HFrEF is heart failure with a left ventricular ejection fraction of lower than 50% but larger than 35%.
15. The method of any one of embodiment 11, wherein the sample is a plasma sample.
16. The method of embodiment 11, wherein the amounts of the at least three biomarkers are determined by mass spectrometry (MS).
17. The method of embodiment 11, wherein the subject does not show symptoms of heart failure.
18. The method of embodiment 11, wherein the subject is overweight.
19. The method of embodiment 11, wherein the subject is older than 54 years of age.
20. The method of embodiment 11, wherein the subject is a female subject, older than 54 years of age, and has a body mass index of less than 25.0 kg/m².
21. The method of embodiment 11, wherein the subject is younger than 61 years of age, and has a body mass index of more than 26.8 kg/m².
22. The method of embodiment 11, wherein the reference is a commercially available lipid standard reference.
23. A method of diagnosing asymptomatic and/or early stage HFrEF in a human subject comprising:
   a. obtaining at least one plasma sample from a patient;
   b. processing the at least one sample for HPLC-MS/MS analysis, wherein the at least one sample is combined with an extraction solvent to create a combined sample, wherein the extraction solvent separates the lipids in the combined sample from the proteins in the combined sample, and further wherein the combined sample undergoes centrifugation to remove the proteins from the sample and to create the processed sample for HPLC-MS/MS analysis;
   c. using a biomarker panel and an LC-MS/MS test to determine the amounts of at least three biomarkers wherein the at least three biomarkers comprise:
      i. at least one triacylglyceride, at least one phosphatidylcholine, and at least one sphingomyelin, wherein the at least one sphingomyelin comprises SM23 and the at least one triacylglycerides comprises OSS2 and the at least one phosphatidylcholine biomarker comprises PC4;

and further wherein SM23 comprises the sum amounts of Sphingomyelin(d18:1/23:1), Sphingomyelin(d18:2/23:0), and Sphingomyelin(d17:1/24:1);
d. obtaining the amount of NT-proBNP protein found in a subject wherein the amount of the protein is obtained by one or more of the following methods:
   i. determining the amount of NT-proBNP in the sample taken from the subject wherein the amount of the one or more proteins is determined using an antibody test, and/or
   ii. determining the amount of NT-proBNP wherein the amount of the protein is derived from the subject's medical record and/or stored in an electronic database;
e. calculating a score value for a subject based on the amounts determined in steps (c) and (d); and
f. comparing that score value of step (e) to a reference value, whereby asymptomatic and/or early stage HFrEF in a subject is to be diagnosed.
24. The method of embodiment 23, wherein the reference value is a cut-off value determined from one or more subjects known to suffer from heart failure and from one or more subjects known not to suffer from heart failure.
25. A method for diagnosing heart failure in a patient currently undergoing therapy for heart failure comprising the steps of:
   a. obtaining at least one blood, plasma, or serum sample from a human subject currently undergoing therapy for heart failure;
   b. processing the at least one sample for HPLC-MS/MS analysis, wherein the at least one sample is combined with an extraction solvent to create a combined sample, wherein the extraction solvent separates the lipids in the combined sample from the proteins in the combined sample, and further wherein the combined sample undergoes centrifugation to remove the proteins from the sample and to create the processed sample for HPLC-MS/MS analysis;
   c. determining in the at least one processed sample of a subject the amounts of biomarkers in at least one biomarker panel, wherein the at least one biomarker panel comprises at least three biomarkers and further wherein the at least three biomarkers comprise:
      i. at least one triacylglyceride, at least one phosphatidylcholine, and at least one sphingomyelin, wherein the at least one sphingomyelin comprises SM23 and the at least one triacylglycerides comprises OSS2 and the at least one phosphatidylcholine biomarker comprises PC4 and further wherein SM23 comprises the sum amounts of Sphingomyelin(d18:1/23:1), Sphingomyelin(d18:2/23:0), and Sphingomyelin(d17:1/24:1);
   d. obtaining the subject's BNP test results by using one or more of the following methods:
      i. determining the amount of one or more of the following proteins in the sample: NT-proBNP, BNP, ANP, or NT-proANP wherein the amount of the one or more proteins is determined using an antibody test, and/or
      ii. determining the amount of one or more of the following proteins in the sample: NT-proBNP, BNP, ANP, or NT-proANP wherein the amount of the one or more proteins is derived from the subject's medical record and/or stored in an electronic database;
   e. calculating a score value for a subject based on the amounts determined in steps (c) and (d); and
   f. comparing that score value of step (e) to a reference value, whereby heart failure in a subject is to be diagnosed; and
   g. determining whether the current heart failure therapy is effective for the subject based on the diagnosis obtained in step (f).

The Figures show:

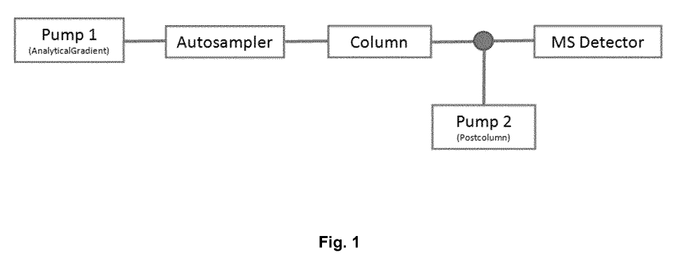

FIG. 1: Flow Diagram of LCMS method applied in the studies underlying the present invention EXAMPLES

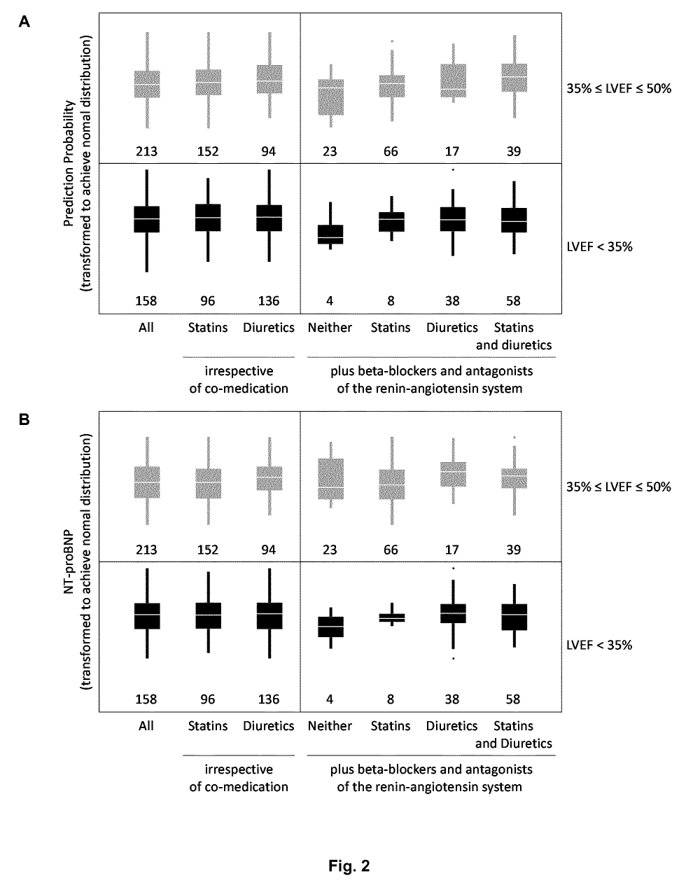

FIG. 2: Effect of medication with diuretics and/or statins on the prediction probabilities of HF patients calculated using Panel 1 including NT-proBNP (A) or on the levels of NT-proBNP (B). Values on the ordinate were transformed using the logit function (A) or were log-transformed (B) to achieve normal distribution. Case numbers are indicated below the boxes.

The following Examples shall illustrate the invention. They shall, however, not be construed as limiting the scope of the invention.

### Example 1: Study Design for the Diagnosis of CHF Subtypes with Reduced Ejection Fraction (HFrEF) Composed of DCMP (Dilated Cardiomyopathy) and ICMP (Ischemic Cardiomyopathy) and Heart Failure with Preserved Ejection Fraction (HFpEF)

A multicentric study with three clinical centers and in total 843 subjects was conducted. The study comprised 194 male and female DCMP, 183 male and female ICMP and 210 male and female HFpEF patients as well as 256 male and female healthy controls in an age range from 35-75 and a BMI range from 20-35 kg/m². NYHA (New York Heart Association) scores of the patients ranged from I to III. Patients and controls were matched for age, gender and BMI. For all patients and controls, a blood sample was collected. Plasma was prepared by centrifugation, and samples were stored at −80° C. until measurements were performed.

Three subgroups of CHF (DCMP, ICMP and HFpEF) were defined on the basis of echocardiography and hemodynamic criteria:
a) Subgroup DCMP: is hemodynamically defined as a systolic pump failure with cardic dilation (echocardiographic enhancement of the left ventricular end diastolic diameter>55 mm and a restricted left ventricular ejection fraction (LVEF) of <50%) without the presence of >50% stenosis
b) Subgroup ICMP: is hemodynamically defined as systolic pump failure due to a coronary insufficiency (>50% coronary stenosis and LVEF of <50%)
c) Subgroup heart failure with preserved ejection fraction (HFpEF): concentric heart hypertrophy (echocardiography: cardiac septum>12 mm and posterior myocardial wall>11 mm) and with a diastolic heart failure (non or mildly impaired pump function with LVEF of ≧50%) without a cardiac septum thickness>18 mm.

NYHA IV patients were excluded as well as patients suffering from apoplex, patients who had myocardial infarction within the last 4 months before testing, patients with altered medications within the last 4 weeks before testing as well as patients who suffered from acute or chronic inflammatory diseases and malignant tumours.

### Example 2: Determination of Metabolites

Significantly altered metabolites in CHF vs healthy respectively HFrEF vs healthy patients have been identified. Metabolites have been selected according to their accessibility with a one-shot LC-MS/MS measurement with a simplified preanalytical process (see analytical method description, below).

In an iterative process in which in-silico performance of metabolite combinations and assay optimization steps have been performed, an LC-MS/MS method for the analysis of CHF was established. This method is capable to analyze all of the biomarkers as listed in Table 1, where the biomarkers are characterized by the combination of retention time and multiple reaction monitoring (MRM) transitions, and further potential lipid metabolites.

Each biomarker may contain more than one analyte, whereby the analytes contained in the same biomarker have the same total number of carbon atoms and the same total number of double bonds.

### Example 3: Analytical Method

Human plasma samples were prepared and subjected to HPLC-MS/MS analysis as described in the following:

10 μl plasma were mixed with 1500 μl extraction solvent containing methanol/dichloromethane (in a ratio of 2:1, v/v) and 10 μl internal standard mixture in a 2 ml safelock microcentrifuge tube (Eppendorf, Germany). The internal standard solution consisted of 25.65 μg/ml lysophosphatidylcholine (LPC) C17:0, 0.386 μg/ml ceramide (Cer) d18:1117:0, 41.28 μg/ml phosphatidylcholine (PC) C19:0 C19:0 (as PC5 listed in Table 8), 22.51 μg/ml cholesteryl heptadecanoate (CE C17:0), and 5.26 μg/ml glyceryl triheptadecanoate (MMM) in extraction solvent. Lipid standards were purchased from Avanti Polar Lipids, CA, U.S.A., Larodan Fine Chemicals, Sweden, Sigma-Aldrich, MO, U.S.A., or Tokyo Chemical Industry, Japan.

Ultrapure water (Milli-Q water system, Millipore) and analytical grade chemicals were used for extraction, dilution or as LC solvents. Quality control and reference sample were prepared from commercially available human plasma (RECIPE Chemicals+Instruments GmbH). Delipidized plasma (Plasma, Human, Defibrinated, Delipidized, 2× Charcoal treated, Highly Purified; USBio) was used for the preparation of calibrators and blanks.

After thoroughly mixing at 20° C. for 5 min, the precipitated proteins were removed by centrifugation for 10 min. An aliquot of the liquid supernatant was transferred to an appropriate glass vial and stored at −20° C. until analysis by LC-MS/MS. This sample preparation method uses protein precipitation as the only purification step to capture all lipids of interest (PC, SM, Cer, CE, TAG), so that a more comprehensive and complex extraction of lipids (as described e.g. by Folch, or Bligh & Dyer) was not necessary. The HPLC-MS/MS systems consisted of an Agilent 1100 LC system (Agilent Technologies, Waldbronn, Germany) coupled to an ABSciex™ API 4000 triple quadrupole mass spectrometer (ABSCIEX, Toronto, Canada). HPLC analysis was performed at 55° C. on commercially available reversed phase separation columns with C18 stationary phases (Ascentis® Express C18 column (5 cm×2.1 mm, 2.7 μm, Phenomenex, Germany).

Up to 5 μL of the crude extract were injected and separated by gradient elution using a mixture of solvents consisting of methanol, water, formic acid, 2-propanol and 2-methoxy-2-methylpropan:
Solvent A: 400 g methanol, 400 g water, 1 g formic acid
Solvent B: 400 g tert-butyl methyl ether (tBME), 200 g 2-propanol, 100 g methanol, 1 g formic acid

Details of the elution gradient are given in Table 7.

Mass spectrometry was carried out by electrospray ionization in positive ion mode using multiple-reaction-monitoring (MRM). The source parameters were: nebulizer gas, 50; heater gas, 60; curtain gas, 25; CAD gas, 4; ion spray voltage, 5500 V; temperature, 400° C.; pause between mass ranges, 5 ms; resolution Q1 and Q3, unit. The MRM settings are given in Table 8. To enhance the ionization efficiency in the electrospray process for some lipids (CE, TAG), ammonium formate buffer dissolved in methanol (Solvent C: 200 g methanol, 30 g 0.1M ammonium formate solution in water) was added post column during the elution time of CE and TAG (see FIG. 1). The method is intended to be compatible with e.g. the ABSciex™ 3200MD benchtop LCMS/MS system.

Furthermore, as some highly abundant lipids are out of the dynamic range of the MS detector, MS parameters—like collision energy—were changed for said highly abundant lipids to get lower signal intensities due to lower fragmentation efficiencies.

Using electrospray isonization (ESI), PC 8 and several SMs with equal numbers of carbons and double bonds were detected together (as to say without separation of said species), since these isobaric species were not separated chromatographically. Chromatography was required to detect the low-concentrated ceramides and to separate phosphatidiyl cholines and sphingomyelins from each other, because the C13-impact of PC to SM or SM to PC disturbs the metabolite performance (in the sense of measurability).

Quantitative results for all compounds were achieved by using the corresponding reference compounds, except for sphingomyelines, for which no commercial reference compounds were available. Sphingomyelines were quantified by adding commercially available sphingomyeline standards with a different chain length than the target metabolites assuming that the detector response is the same. The proof of concept was carried out on SM(d18:1/16:0), SM(d18:1/24:1) and PC C18:0 C18:2. The PC C18:0 C18:2 was included to show that the positive ion formation on the phosphocholine side via electrospray is not only independent on the fatty acid composition, but also independent of the phosphocholine species (sphingosine or glycerol backbone). A relative standard deviation (% RSD) of 20 could be achieved by intercalibrating the standards with each other.

The calibration solutions were prepared in delipidized plasma (commercially available) to simulate a matrix as close as possible to real plasma.

Positive and negative controls were prepared by lyophilization different amounts of commercially available plasma to meet the positive and negative cutoffs for all down regulated metabolites. For the triacylglycerides (TAG) the corresponding TAG was added into the plasma before the lyophilisation process but avoiding the protein to precipitate. The lyophilisated samples were stored in the freezer till sample preparation.

Quality control samples were prepared by extracting commercially available plasma with extraction solvent. Several aliquots of these extracts were stored in the freezer and used for the daily quality control of the instrument performance and sample preparation.

Plasma samples were analyzed in randomized analytical sequence design. Following comprehensive analytical validation steps, the resulting peak areas were divided by the peak areas of an internal standard with similar analytical behavior to reduce analytical variation. The resulting ratios were log 10-transformed to achieve normal distribution.

TABLE 1
Biomarkers used for panel composition and their analytical characteristics.

| | | | | | Transition | Retention |
|---|---|---|---|---|---|---|
| | | | | | (parent/fragment) | Time |
| Biomarker | Analyte 1 | Analyte 2 | Analyte 3 | Group | (Da) | (min) |
| Cer(d16:1/24:0) | Cer(d16:1/24:0) | | | Ceramide | 622.6/236.2 | 2.83 |
| Cer(d17:1/24:0) | Cer(d17:1/24:0) | | | Ceramide | 636.6/250.2 | 2.95 |
| Cer(d18:1/23:0) | Cer(d18:1/23:0) | | | Ceramide | 636.5/264.2 | 2.95 |
| Cer(d18:1/24:1) | Cer(d18:1/24:1) | | | Ceramide | 648.6/264.2 | 2.83 |
| Cer(d18:2/24:0) | Cer(d18:2/24:0) | | | Ceramide | 648.6/262.2 | 2.9 |
| CE C18:0 | CE C18:0 | | | Cholester- | 670.7/369.3 | 4.72 |
| | | | | ylester | | |
| CE C18:2 | CE C18:2 | | | Cholester- | 666.7/369.3 | 4.47 |
| | | | | ylester | | |
| Glutamic acid | Glutamic acid | | | amino acid | 148.0/84.1 | 0.23 |
| PC4 | PC (C16:0 C18:2) | | | Phosphati- | 758.6/184.1 | 1.72 |
| | | | | dylcholine | | |
| PC8 | PC (C18:0 C18:2) | PC (C18:1 C18:1) | | Phosphati- | 786.6/184.1 | 1.88 |
| | | | | dylcholine | | |
| SM10 | SM(d18:1/18:0) | SM(d16:1/20:0) | | Sphingo- | 731.5/184.1 | 1.7 |
| | | | | myeline | | |
| SM18 | SM(d18:1/21:0) | SM(d16:1/23:0) | SM(d17:1/22:0) | Sphingo- | 773.5/184.1 | 1.97 |
| | | | | myeline | | |
| SM2 | SM(d18:1/14:0) | SM(d16:1/16:0) | | Sphingo- | 675.5/184.1 | 1.47 |
| | | | | myeline | | |
| SM21 | SM(d17:1/23:0) | SM(d18:1/22:0) | SM(d16:1/24:0) | Sphingo- | 787.5/184.1 | 2.05 |
| | | | | myeline | | |
| SM23 | SM(d18:1/23:1) | SM(d18:2/23:0) | SM(d17:1/24:1) | Sphingo- | 799.5/184.1 | 2.02 |
| | | | | myeline | | |
| SM24 | SM(d18:1/23:0) | SM(d17:1/24:0) | | Sphingo- | 801.5/184.1 | 2.16 |
| | | | | myeline | | |
| SM28 | SM(d18:1/24:0) | | | Sphingo- | 815.5/184.1 | 2.27 |
| | | | | myeline | | |
| SM29 | SM(d18:2/17:0) | | | Sphingo- | 715.5/184.1 | 1.57 |
| | | | | myeline | | |
| SM3 | SM(d17:1/16:0) | | | Sphingo- | 689.5/184.1 | 1.52 |
| | | | | myeline | | |
| SM5 | SM(d18:1/16:0) | SM(d16:1/18:0) | | Sphingo- | 703.5/184.1 | 1.57 |
| | | | | myeline | | |
| SM8 | SM(d18:2/18:1) | | | Sphingo- | 727.5/184.1 | 1.55 |
| | | | | myeline | | |
| SM9 | SM(d18:1/18:1) | SM(d18:2/18:0) | | Sphingo- | 729.5/184.1 | 1.62 |
| | | | | myeline | | |
| OSS2 | TAG C18:1 C18:0 | | | Triacyl- | 906.9/605.4 | 4.87 |
| | C18:0 | | | glyceride | | |
| PPO1 | TAG C16:0 C16:0 | | | Triacyl- | 850.8/577.5 | 4.64 |
| | C18:1 | | | glyceride | | |
| PPP | TAG C16:0 C16:0 | | | Triacyl- | 824.8/551.5 | 4.61 |
| | C16:0 | | | glyceride | | |
| SOP2 | TAG C18:0 C18:1 | | | Triacyl- | 878.9/577.6 | 4.76 |
| | C16:0 | | | glyceride | | |
| SPP1 | TAG C18:0 C16:0 | | | Triacyl- | 852.9/579.6 | 4.74 |
| | C16:0 | | | glyceride | | |
| SSP2 | TAG C18:0 C18:0 | | | Triacyl- | 880.9/579.6 | 4.85 |
| | C16:0 | | | glyceride | | |
| SSS | TAG C18:0 C18:0 | | | Triacyl- | 908.9/607.6 | 4.96 |
| | C18:0 | | | glyceride | | |

### Example 4: Data Analysis and Statistical Evaluation

For each biomarker listed in Table 1, the direction of change in CHF patients relative to healthy control subjects was calculated by ANOVA (see Tables 1A and 1B). The direction ‘Up’ means that the levels of the biomarker are higher in CHF patients than in healthy control subjects, the direction ‘Down’ means that the levels of the biomarker are lower in CHF patients than in healthy control subjects. The direction of change was found to be the same for all CHF patients compared to healthy control subjects taken together [ANOVA model: CHF+CENTER+(GENDER+AGE+BMI)̂2], for HFpEF patients compared to healthy control subjects, and for HFrEF patients compared to healthy control subjects [CHF_SUBGROUP+CENTER+(GENDER+AGE+BMI)̂2].

TABLE 1A
Results of the ANOVA analysis as described above regarding the different biomarkers from Table 1.

| | CHF_ALL | | | | HFpEF | | | | HFrEF | | | |
|---|---|---|---|---|---|---|---|---|---|---|---|---|
| | | p < | | p- | | p < | | p- | | p < | | p- |
| Biomarker | Direction | 0.05 | Ratio | Value | Direction | 0.05 | Ratio | Value | Direction | 0.05 | Ratio | Value |
| Cer(d16:1/24:0) | Down | Yes | 0.76 | 3.08E−09 | Down | Yes | 0.84 | 1.74E−03 | Down | Yes | 0.72 | 2.72E−11 |
| Cer(d17:1/24:0) | Down | Yes | 0.78 | 1.88E−09 | Down | Yes | 0.82 | 7.38E−05 | Down | Yes | 0.76 | 4.17E−10 |
| Cer(d18:1/23:0) | Down | Yes | 0.86 | 2.22E−06 | Down | Yes | 0.87 | 1.62E−04 | Down | Yes | 0.86 | 6.23E−06 |
| Cer(d18:1/24:1) | Down | No | 0.98 | 5.77E−01 | Down | No | 0.97 | 4.07E−01 | Down | No | 0.99 | 7.62E−01 |
| Cer(d18:2/24:0) | Down | Yes | 0.86 | 4.78E−05 | Down | Yes | 0.90 | 1.24E−02 | Down | Yes | 0.85 | 1.53E−05 |
| CE C18:0 | Down | No | 0.94 | 1.55E−01 | Down | No | 0.99 | 8.34E−01 | Down | No | 0.92 | 5.65E−02 |
| CE C18:2 | Down | Yes | 0.87 | 1.21E−13 | Down | Yes | 0.92 | 1.31E−04 | Down | Yes | 0.85 | 2.72E−17 |
| Glutamic acid | Down | No | 0.93 | 1.31E−01 | Down | Yes | 0.89 | 3.62E−02 | Down | No | 0.96 | 3.62E−01 |
| OSS2 | Up | Yes | 1.75 | 2.67E−11 | Up | Yes | 1.54 | 2.03E−05 | Up | Yes | 1.87 | 2.13E−12 |
| PC4 | Down | Yes | 0.90 | 1.31E−08 | Down | Yes | 0.92 | 1.09E−04 | Down | Yes | 0.89 | 5.37E−09 |
| PC8 | Down | Yes | 0.92 | 6.89E−05 | Down | Yes | 0.93 | 4.44E−03 | Down | Yes | 0.92 | 6.56E−05 |
| PPO1 | Up | Yes | 1.49 | 1.29E−07 | Up | Yes | 1.35 | 1.16E−03 | Up | Yes | 1.57 | 1.81E−08 |
| PPP | Up | Yes | 1.61 | 1.16E−06 | Up | Yes | 1.46 | 1.39E−03 | Up | Yes | 1.70 | 4.38E−07 |
| SM10 | Down | Yes | 0.95 | 2.18E−02 | Down | No | 0.96 | 8.57E−02 | Down | Yes | 0.95 | 2.44E−02 |
| SM18 | Down | Yes | 0.79 | 1.41E−18 | Down | Yes | 0.84 | 8.68E−08 | Down | Yes | 0.76 | 1.55E−21 |
| SM2 | Down | Yes | 0.81 | 3.77E−13 | Down | Yes | 0.85 | 3.14E−06 | Down | Yes | 0.79 | 1.83E−14 |
| SM21 | Down | Yes | 0.84 | 4.51E−15 | Down | Yes | 0.89 | 1.22E−05 | Down | Yes | 0.81 | 2.54E−18 |
| SM23 | Down | Yes | 0.83 | 2.44E−15 | Down | Yes | 0.88 | 1.70E−06 | Down | Yes | 0.81 | 1.07E−17 |
| SM24 | Down | Yes | 0.81 | 2.42E−17 | Down | Yes | 0.86 | 5.47E−07 | Down | Yes | 0.78 | 1.92E−20 |
| SM28 | Down | Yes | 0.83 | 1.22E−13 | Down | Yes | 0.90 | 2.92E−04 | Down | Yes | 0.79 | 8.08E−18 |
| SM29 | Down | Yes | 0.86 | 4.18E−07 | Down | Yes | 0.88 | 3.22E−04 | Down | Yes | 0.85 | 3.49E−07 |
| SM3 | Down | Yes | 0.81 | 4.76E−14 | Down | Yes | 0.83 | 8.12E−08 | Down | Yes | 0.79 | 2.51E−14 |
| SM5 | Down | Yes | 0.89 | 1.94E−10 | Down | Yes | 0.90 | 2.20E−06 | Down | Yes | 0.88 | 3.05E−10 |
| SM8 | Down | Yes | 0.87 | 5.27E−07 | Down | Yes | 0.89 | 2.50E−04 | Down | Yes | 0.87 | 5.99E−07 |
| SM9 | Down | No | 0.97 | 2.21E−01 | Down | No | 0.98 | 4.67E−01 | Down | No | 0.97 | 1.89E−01 |
| SOP2 | Up | Yes | 1.70 | 7.93E−11 | Up | Yes | 1.50 | 3.73E−05 | Up | Yes | 1.81 | 6.21E−12 |
| SPP1 | Up | Yes | 1.75 | 3.54E−09 | Up | Yes | 1.59 | 5.96E−05 | Up | Yes | 1.85 | 1.32E−09 |
| SSP2 | Up | Yes | 1.64 | 2.51E−09 | Up | Yes | 1.50 | 6.41E−05 | Up | Yes | 1.73 | 7.37E−10 |
| SSS | Up | Yes | 1.27 | 1.11E−04 | Up | Yes | 1.22 | 1.06E−02 | Up | Yes | 1.31 | 6.26E−05 |

TABLE 1B
Results of the ANOVA analysis for CHF subgroups ICMP and DCMP
as described above regarding the different biomarkers from Table
1. Only the biomarkers from Panel 1 in Table 2 are listed.

| | ICMP | | | | DCMP | | | |
|---|---|---|---|---|---|---|---|---|
| | | p < | | p- | | p < | | p- |
| Biomarker | Direction | 0.05 | Ratio | Value | Direction | 0.05 | Ratio | Value |
| OSS2 | Up | Yes | 1.77 | 5.01E−08 | Up | Yes | 1.96 | 2.85E−11 |
| PC4 | Down | Yes | 0.86 | 1.52E−10 | Down | Yes | 0.92 | 9.52E−05 |
| SM23 | Down | Yes | 0.78 | 6.64E−19 | Down | Yes | 0.84 | 3.67E−10 |

The data of the study described in Example 1 were utilized for the evaluation of the diagnostic power for the classification of CHF subgroups compared with controls in combination with the peptide NT-proBNP. Metabolite data were corrected for confounding factors or uncorrected metabolite data were used. The ANOVA model for correction for confounders comprised the factors age, BMI, gender and CHF subgroup (ANOVA model: CHF_SUBGROUP+(GENDER+AGE+BMI)̂2; correction factors: GENDER, AGE and BMI). CHF patients were subdivided based on CHF subtype [HFpEF, DCMP, ICMP, or alternatively the joined DCMP+ICMP group named HFrEF (heart failure with reduced ejection fraction) and a measure for the severity of the disease. Two different measures for the severity of the disease were used: NYHA class and LVEF. To this end, CHF patients were subdivided into those having no or only mild symptoms (NYHA class I and early stages of NYHA class II; sometimes referred to as ‘asymptomatic’) and those having more severe symptoms (NYHA classes II and III, sometimes referred to as ‘symptomatic’). Alternatively, CHF patient were subdivided into those with severely reduced LVEF (LVEF<35%) and those with mildly reduced or non-reduced LVEV (LVEF≧35). The latter subgroup comprises HFrEF patient with 35%≦LVEF<50% as well as all HFpEF patients (LVEF≧50%).

A total set of about 850 samples was split into a training or identification set (also referred to as “ID Data” set; about 66% of the samples) and a testing set (also referred to as “VD Data” set; about 33% of the samples). The split was done group-wise, with groups being defined by the combination of gender, type of heart failure and NYHA levels.

The total sample numbers and compositions were as follows:

| ID Data Set: | VD Data Set: |
|---|---|
| CHF: 374 samples | CHF: 205 samples |
| CHF, NYHA I-I/II: 169 samples | CHF, NYHA I-I/II: 93 samples |
| CHF, NYHA II-III: 205 samples | CHF, NYHA II-III: 112 samples |
| CHF, LVEF ≧ 35%: 275 samples* | CHF, LVEF ≧ 35: 145 samples |
| HFpEF: 134 samples | HFpEF: 74 samples |
| ICMP: 118 samples | ICMP: 65 samples |
| ICMP, NYHA I-I/II: 44 samples | ICMP, NYHA I-I/II: 24 samples |
| ICMP, NYHA II-III: 74 samples | ICMP, NYHA II-III: 41 samples |
| DCMP: 122 samples | DCMP: 66 samples |
| DCMP, NYHA I-I/II: 46 samples | DCMP, NYHA I-I/II: 25 samples |
| DCMP, NYHA II-III: 76 samples | DCMP, NYHA II-III: 41 samples |
| HFrEF: 240 samples | HFrEF: 131 samples |
| HFrEF, NYHA I-I/II: 90 samples | HFrEF, NYHA I-I/II: 49 samples |
| HFrEF, NYHA II-III: 150 samples | HFrEF, NYHA II-III: 82 samples |
| HFrEF, 35% ≦ LVEF < 50%: 141 samples* | HFrEF, 35% ≦ LVEF < 50%: 71 samples |
| HFrEF, LVEF < 35%: 98 samples* | HFrEF, LVEF < 35%: 60 samples |
| Controls: 167 samples | Controls: 87 samples** |

*One CHF patient with missing LVEF measurement was not included in these subgroups
**3 controls were subsequently excluded due to HF-related medication; re-analysis showed no significant changes with regard to the diagnostic performance of the biomarker combinations

### Example 5

In Table 2 biomarker panels according to the present invention are listed. In column 1 the respective panel number is given, in column 2 the composition of each panel, in column 3 the number of non-proteinic biomarker is given, in column 4 the CHF subgroup, in column 5 it is indicated if the panel is a so-called “saturated panel” or not, in column 6 it is indicated whether NT-proBNP was determined in addition, in column 7 it is indicated whether an ANOVA correction for confounders age, BMI and gender have been performed, and in column 8 the AUC-value is given.

Prediction probabilities for each patient were calculated with a logistic regression model fitted using the elastic net algorithm as implemented in the R package glmnet (Zou, H. and Hastie, T., 2003: Regression shrinkage and selection via the elastic net, with applications to microarrays. Journal of the Royal Statistical Society: Series B, 67, 301-320; Friedman, J., Hastie, T., and Tibshirani, R, 2010: Regularization Paths for Generalized Linear Models via Coordinate Descent. J. Stat. Softw. 33). The fitting was performed on the data from the ID cohort. The L1 and the L2 penalties were given equal weight. Log-transformed peak area ratios were centered and scaled to unit variance before the analysis. The prediction probability was calculated using the formula

$$p = \frac{1}{1 + {e}^{- ( {w}_{0} + {\sum }_{i = 1}^{n} {w}_{i} {\hat{x}}_{i} )}} ,$$

with the feature \hat{x}ᵢ, being

$${\hat{x}}_{i} = \frac{{x}_{i} - {m}_{i}}{{s}_{i}}$$

wherein xᵢ are the log-transformed peak area ratios (or concentrations, e.g. of NT-proBNP, if taken into account) and mᵢ, sᵢ are feature specific scaling factors and wᵢ are the coefficients of the model [w₀, intercept; w₁, coefficient for NT-proBNP (where applicable); w₂ . . . wₙ, coefficients for the features; n, number of lipid biomarkers in the panel, +BNP or NT-proBNP, if determined or taken into account].

The coefficients for Panel 1+NT-proBNP are: w₀=0.7602899 (Intercept), w₁=1.5745605 (NT-proBNP), w₂=−0.7158906 (SM23), w₃=0.6762354 (OSS2), w₄=−0.4594183 (PC4), n=4. The respective scaling factors are m₁=2.2023800, m₂=−0.5379916, m₃=−2.1316954, m₄=0.8736775 and s₁=0.6364517, s₂=0.1159420, s₃=0.3863649, s₄=0.0834807. NT-proBNP was measured in the unit pg/ml.

A sample with a prediction probability larger than the cutoff of the respective panel is said to be tested positive, and a sample with a prediction probability smaller than or equal to the cutoff is said to be tested negative. The sensitivity is the fraction of HF patients in the respective subgroup that are tested positive, while the specificity is the fraction of healthy control subjects that are tested negative.

The cutoff values were first determined to maximize the Youden index for the detection of HFrEF. The resulting cutoff for Panel 1+NT-proBNP was defined to be 0. 587, for Panel 3 to be 0.522, and for Panel 4 to be 0.574, resulting in the sensitivities and specificities for Panels 1, 3, and 4 as shown in Table 6.

Alternatively, the cutoff values can be determined to achieve the same specificity for the detection of HFrEF in the ID dataset when using the biomarker panel as when using NT-proBNP alone. According to this requirement, the cutoff for Panel 1+NT-proBNP was alternatively defined to be 0.738. Using this alternative cutoff for Panel 1+NT-proBNP, the sensitivity is 61.0% for the detection of CHF, 80.2% for the detection of HFrEF, and 67.6% for the detection of HFrEF with only mildly reduced LVEF (35%≦LVEF<50%). The specificity of Panel 1+NT-proBNP using the alternative cutoff is 97.7%.

Prevalence of HF in the Medicare-eligible population of the US was 9-12% between 1994 and 2003, about half of which is affected by HFrEF [Yancy, C. W., Jessup, M., Bozkurt, B., Butler, J., Casey, D. E., Drazner, M. H., Fonarow, G. C., Geraci, S. A., Horwich, T., Januzzi, J. L., Johnson, M. R., Kasper, E. K., Levy, W. C., Masoudi, F. A., McBride, P. E., McMurray, J. J. V., Mitchell, J. E., Peterson, P. N., Riegel, B., Sam, F., Stevenson, L. W., Tang, W. H. W., Tsai, E. J., Wilkoff, B. L., 2013. 2013 ACCF/AHA Guideline for the Management of Heart Failure. A Report of the American College of Cardiology Foundation/American Heart Association Task Force on Practice Guidelines. Circulation 128, e240-e327; Curtis L H, Whellan D J, Hammill B G, et al, 2008. Incidence and prevalence of heart failure in elderly persons, 1994-2003. Arch Intern Med 168, 418-424. doi:10.1001/archinternmed.2007.80].

Assuming, based on the literature cited above, a prevalence of 10% for CHF and 5% for HFrEF, the resulting positive predictive value (PPV) is 74.7% for CHF and 64.7% for HFrEF. The resulting negative predictive value (NPV) is 95.8% for CHF and 98.9% for HFrEF, as shown in Table 6A. Principally, the diagnostic performance of NT-proBNP could be remarkably increased; e.g for the subgroups CHF and HFrEF, a Net Reclassification Index (NRI=Sensitivity [Test A]−Sensitivity [Test B]+Specificity [Test A]−Specificity [Test B]) of 6.4% and 8.8% was observed, respectively. The NRI for asymptomatic ICMP even reached 10.3%. Thus, the panels of the present invention especially improved diagnostic performance of HFrEF and/or asymptomatic forms of heart failure.

The term “saturated panel” preferably indicates that the addition of a further suitable cardiac marker from Table 1 to the respective panel does not result in a noticeable improvement of the AUC value.

### Example 6: Validation of Performance in Testing Set

The performance of a combination of biomarkers identified in the training data when applied to the validation data was assessed by the Area Under the Curve (AUC) of a receiver operating curve, which was modelled with the binormal model.

The biomarker combinations/panels 103-199 (Table 2) are shown with their calculated performances in different CHF subgroups in the tables below.

### Example 7: Influence of Medication on Performance in Testing Set

The effects of 3-hydroxy-3-methyl-glutaryl-CoA reductase (HMGCR) inhibitors and diuretics on the HF prediction probabilities for HFrEF patients calculated using Panel 1 including NT-proBNP is shown in FIG. 2. Since patients receiving diuretics have on average a lower LVEF than patients not receiving diuretics (not shown), the prediction probabilities were plotted separately for patients with mildly (35% LVEF 50%; top panel) or strongly reduced LVEF (LVEF<35%; bottom panel). HFrEF patients receiving neither diuretics nor statins have a slightly lower prediction probability than patients receiving either or both of these medications (four right-most columns in FIG. 2), resulting in a lower sensitivity in patients with mildly reduced LVEF which receive neither diuretics nor statins (35%≦LVEF≦50%; Table 9). The effect is less obvious when comparing the partially overlapping groups including all patients receiving statins or diuretics (Table 9; three left-most columns in FIG. 2)

### Example 8: Influence of Clinical Parameters on Performance in the Whole Patient Cohort

The influence of clinical parameters on the performance of Panel 1 in comparison with NT-proBNP was assessed by investigating the characteristics of patients which are classified differently using either Panel 1 including NT-proBNP or NT-proBNP alone. In the whole patient cohort, 30 healthy controls were correctly classified as negative using Panel 1 including NT-proBNP, but wrongly classified as positive using NT-proBNP alone. These subjects were significantly older (median 62.5 years versus 53.5 years; p=0.0036 before and p<0.05 after FDR (False Discovery Rate) correction), had a significantly lower BMI (median 23.0 kg/m² versus 24.8 kg/m²; p=0.0197 before and p<0.1 after FDR correction), and comprised a significantly higher proportion of females (24 out of 30 versus 107 out of 216; p=0.0033 before and p<0.05 after FDR correction) than the remaining 216 healthy controls in the cohort.

In the whole patient cohort, 27 HFrEF patients were correctly classified as positive using Panel 1 including NT-proBNP, but wrongly classified as negative using NT-proBNP alone. These subjects were significantly younger (median 55.0 years versus 61.0 years; p=0.0218 before and p<0.1 after FDR correction), had a significantly higher BMI (median 29.9 kg/m² versus 26.8 kg/m²; p=0.0037 before and p<0.05 after FDR correction), had significantly lower high-sensitivity cardiac troponin T (hs-cTnT values; median 4.0 ng/l versus 7.0 ng/l; p=0.0238 before and p<0.1 after FDR correction), had a significantly larger LVEF (median 45.0% versus 35.0%; p=0.0003 before and p<0.01 after FDR correction), had a significantly lower left ventricular end-diastolic dimension (LVEDD; median 50.0 mm versus 55.0 mm; p=0.0020 before and p<0.05 after FDR correction), had a significantly higher septal wall thickness (13.0 mm versus 11.0 mm; p=0.0017 before and p<0.05 after FDR correction), had a significantly higher posterior wall thickness (12.0 mm versus 11.0 mm; p=0.0008 before and p<0.05 after FDR correction), and comprised a significantly higher proportion of patients suffering from Diabetes mellitus (11 out of 27 versus 68 out of 347; p=0.0189 before and p<0.1 after FDR correction) than the remaining 347 HFrEF patients in the cohort.

### Example 9: Further Refinement of the Method of Example 3 for the Determination of Panel 1

An improved quantification of triglycerides, phosphatidylcholines, and sphingomyelins can be achieved using the calibration standards 1,3-distearoyl-2-oleoylglycerol (TAG C18:0 C18:1 C18:0; available from LGC Standards, U.K.), phosphatidylcholine C16:0 C18:2 (available from Avanti Polar Lipids, CA, U.S.A.), and sphingomyeline d18:1/24:1 (available from Avanti Polar Lipids, CA, U.S.A.). The calibration standards are dissolved in the range of 1 to 5 mg/ml solutions using methanol/dichloromethane (2:1, v/v) or dichloromethane as solvent.

The method is intended to be compatible with e.g. the ABSciex™ 3200 MD benchtop LC-MS/MS system.

### Example 10: Further Biomarker Panel, Determined with a Combined/Targeted Lipid Analysis Approach

Using a combination of the method described herein with a method for targeted lipid analysis as e.g. disclosed in Example 1 of WO2013079594, which is suitable for detection of e.g the single metabolite species (also referred to as analytes) represented within the biomarkers from Table 1, or a method for targeted lipid analysis as e.g. disclosed in WO2013079594 alone, further metabolite panels with a good performance for the diagnosis of HFrEF can be identified. One such combination of 6 metabolites is shown in Table 10 (Panel No. 300).

When combined with the value for NT-proBNP, this panel (Panel No. 300) shows estimated AUC-values of up to 0.988 (Subgroup: HFrEF with LVEF<35%), 0.937 ((Subgroup: HFrEF with LVEF between 35% and 50%), 0.942 (Subgroup: HFrEF asymptomatic), 0.969 (Subgroup: HFrEF symptomatic), 0.959 (Subgroup: HFrEF total) 0.979 (ICMP total), or 0.936 (DCMP total).

### Example 11: Analytical Method for the Absolute Quantification of SM23, PC 4, CE C18:2, and OSS2

Human plasma samples were prepared and subjected to HPLC-MS/MS analysis as described in the following:

10 μl plasma were mixed with 1500 μl extraction solvent containing methanol/dichloromethane (in a ratio of 2:1, v/v) and 10 μl internal standard mixture in a 2 ml safelock microcentrifuge tube (Eppendorf, Germany). The internal standard contained 41.28 μg/ml phosphatidylcholine (PC) C19:0 C19:0 (as PC5 listed in Table 8 in extraction solvent. This Lipid standard was purchased from Avanti Polar Lipids, CA, U.S.A.

Ultrapure water (Milli-Q water system, Millipore) and analytical grade chemicals were used for extraction, dilution or as LC solvents. Quality control and reference sample were prepared from commercially available human plasma (RECIPE Chemicals+Instruments GmbH). Delipidized plasma (Plasma, Human, Defibrinated, Delipidized, 2× Charcoal treated, Highly Purified; USBio) was used for the preparation of calibrators and blanks.

After thoroughly mixing at 20° C. for 5 min, the precipitated proteins were removed by centrifugation for 10 min. An aliquot of the liquid supernatant was transferred to an appropriate glass vial and stored at −20° C. until analysis by LC-MS/MS. This sample preparation method uses protein precipitation as the only purification step to capture all lipids of interest (PC4, SM23, OSS2, CE C18:2). The HPLC-MS/MS systems consisted of an Agilent 1100 LC system (Agilent Technologies, Waldbronn, Germany) coupled to an ABSciex™ API 4000 triple quadrupole mass spectrometer (ABSCIEX, Toronto, Canada). HPLC analysis was performed at 55° C. on commercially available reversed phase separation columns with C18 stationary phases (Ascentis® Express C18 column (5 cm×2.1 mm, 2.7 μm, Phenomenex, Germany).

Up to 5 μL of the crude extract were injected and separated by gradient elution using a mixture of solvents consisting of methanol, water, formic acid, 2-propanol and 2-methoxy-2-methylpropan:
Solvent A: 400 g methanol, 400 g water, 1 g formic acid
Solvent B: 400 g tert-butyl methyl ether (tBME), 200 g 2-propanol, 100 g methanol, 1 g formic acid Details of the elution gradient are given in Table 7.

Mass spectrometry was carried out by electrospray ionization in positive ion mode using multiple-reaction-monitoring (MRM). The source parameters were: nebulizer gas, 50; heater gas, 60; curtain gas, 25; CAD gas, 4; ion spray voltage, 5500 V; temperature, 400° C.; pause between mass ranges, 5 ms; resolution Q1 and Q3, unit. The MRM settings are given in Table 12. To enhance the ionization efficiency in the electrospray process for OSS2, ammonium formate buffer dissolved in methanol (Solvent C: 200 g methanol, 100 g 0.1M ammonium formate solution in water) was added post column during the elution time of OSS2 (see FIG. 1). The eluation conditions etc. are given in Table 7 except for the flow rate of Solvent C which was increased in this Example 11 to 300 μl/min during the elution times from 3.30 min to 5.30 min.

The method is intended to be compatible with e.g. the ABSciex™ 3200 MD benchtop LC-MS/MS system.

The above mentioned internal standard PC5 was added to all samples at a known concentration. As a consequence, the determination of the respective peak area in the MS measurement makes it possible to calculate a correction factor in said samples; this correction factor may be used in order to correct the peak area of the biomarkers to be determined for variations of the devices, inherent system errors, or the like.

For each biomarker determined as peak area, the area ratio of the biomarker peak area to the internal standard peak area was determined.

The respective retention times and standards for peak area ratio calculations are shown in Table 12a. Quantitative results for SM23, PC4, CE C18:2, and OSS2 were achieved by using a calibration procedure with a calibration solution as described in the following (Example 12).

### Example 12: Preparation of Calibration Samples

In order to generate calibration curves for the absolute quantification of SM23, PC4, CE C18:2, and OSS2, calibration solutions comprising known amounts of SM27 (Sphingomyelin (d18:1/24:1) for the quantification of SM23, purchased from Avanti Lipids), PC 4, and OSS2 were prepared in MeOH/CH2Cl2 (2/1, v/v).

For the subsequent generation of calibration samples, calibration solutions containing the respective concentrations of SM27, PC4, CE C18:2, and OSS2 in MeOH/CH2Cl2 (2/1) as shown in Table 13 (respective ratios are given in Table 13a) were prepared.

From each of these calibration solutions, 50 μl were added to a mixture of 10 μl delipidized plasma (commercially available) and 750 μl MeOH/CH2Cl2 (2/1). 10 μl internal standard were added, as described above, and further 700 μl MeOH/CH2Cl2 (2/1) were added in order to achieve a final calibration sample volume of 1520 μl.

The resulting 7 calibration samples were then treated the same way as the actual plasma samples for biomarker determination.

### Example 13: Calculation of the Absolute Concentrations of SM23, PC 4, CE C18:2, and OSS2 in the Patient Samples

The absolute concentrations of SM23, PC4, CE C18:2, and OSS2 in any given sample can be determined by comparing the peak area ratios in the sample to the respective peak area ratios in a calibration sample or the calibration curve resulting from the respective peak area ratios of the respective set of calibration samples containing known amounts of SM23, PC4, CE C18:2, and OSS2.

As described above, SM27 was used in the calibration samples for the quantification of SM23.

Using the analytical method described in Example 11 and the calibration samples described in Example 12, above, absolute quantification of SM23, PC4, CE C18:2, and OSS2 was carried out with an additional step involving an ultrapool sample consisting of commercially available human plasma, which was mixed well and split into a sufficient number of aliquots. Aliquots of the ultrapool sample were included with the patient samples measured on each day of the analysis. To calculate absolute concentrations, the peak area ratios of SM23, PC4, CE C18:2, and OSS2 in each patient sample (as described above) where first divided by the median of the respective peak area ratios in the aliquots of the ultrapool sample measured on the same day of the analysis as said patient sample, and then multiplied by the known concentrations of SM23, PC4, CE C18:2, and OSS2, respectively, in the ultrapool sample.

A small number of samples which had been included in the calculation of the results shown in Examples 1 to 10 were now excluded for technical reasons. Concentrations of SM23, PC4, CE C18:2, and OSS2 in the ultrapool sample had previously been determined by comparing their peak area ratios to a calibration curve obtained from calibration samples as described in Example 12, above.

Since 50 μl of each calibration standard, but only 10 μl plasma of each patient sample were used, a multiplication factor of five was applied in order to receive the actual concentration ranges from the patient samples.

The resulting concentrations of SM23, PC4, CE C18:2, and OSS2 as determined for control samples and samples of the selected CHF subgroups HFrEF and HFpEF are shown in Table 14.

### Example 14: Absolute Quantification of SM23, PC 4, and OSS2 (Panel 1)—Absolute Quantification of SM23, CE C18:2, and OSS2 (Panel 2)

The same procedure as described in Examples 11 to 13 was applied for the absolute quantification of SM23, PC 4, and OSS2 (Panel 1), however without determining CE C18:2. Also the same procedure as described in Examples 11 to 13 can be applied for the absolute quantification for absolute quantification of SM23, CE C18:2, and OSS2 (Panel 2), however without determining PC4. The respective information concerning MS-settings, standards for quantification etc. is given in Tables 12, 12a, 13 and 13b (Panel 2) or 13c (Panel 1).

### Example 15: Performance Calculation for Different Subgroups Using Absolute Concentrations for SM23, PC4, and OSS2 (Panel 1+NT-proBNP)

Using absolute concentrations of the metabolite biomarkers, prediction probabilities and performance statistics where calculated for Panel 1+NT-proBNP as described in Example 5 without ANOVA correction. The scaling factors for the metabolite biomarkers measured in the unit μg/dl were m₂=3.106761 (SM23), m₃=2.12519 (OSS2), m₄=4.583751 (PC4) and s₂=0.123062, s₃=0.3926955, s₄=0.0892658. The scaling factors m₁ and sᵢ for NT-proBNP and the weights w₀, w₁, w2, w3, and w4, were the same as in Example 5. The cut-off was determined for a “matched sensitivity” in the subgroup HFrEF as described in Example 5, above, to be 0.743.

To enable a direct comparison between the performance statistics calculated using absolute concentrations versus peak area ratios, the performance calculations based on peak area ratios as previously conducted and described in Examples 1 to 10 were also repeated, excluding the same few samples which were excluded from the calculation of absolute concentrations (see Example 13).

In the testing (“VD”) data set, the AUC for CHF was 0.915 using absolute concentrations compared to 0.917 using peak area ratios. Sensitivity (62.3%) and specificity (97.6%) for CHF at said cutoff value were unchanged. The AUC (0.969) and specificity (97.6%) for HFrEF were unchanged. Sensitivity for HFrEF was 80.9% using absolute concentrations compared to 80.2% using peak area ratios. The AUC for HFrEF asymptomatic was 0.953 using absolute concentrations compared to 0.954 using peak area ratios. Sensitivity (73.5%) and specificity (97.6%) for HFrEF asymptomatic at said cutoff value were unchanged. The AUC for HFrEF LVEF 35% to 50% was 0.946 using absolute concentrations compared to 0.948 using peak area ratios. The sensitivity for HFrEF LVEF 35% to 50% was 69.0% using absolute concentrations compared to 67.6% using peak area ratios. The specificity (97.6%) for HFrEF LVEF 35% to 50% was unchanged. The performance statistics for Panel 1+NT-proBNP calculated using absolute concentrations for the metabolite biomarkers were thus essentially identical to those calculated using peak area ratios.

TABLE 2
Multimarker panel compositions and their respective Area under the curve (AUC) values of receiver operating characteristic (ROC) analysis calculated
for different CHF subgroups including or excluding NT-proBNP, with or without ANOVA correction for confounders age, BMI and gender.

| | | No. of | | | | | |
|---|---|---|---|---|---|---|---|
| Panel | Panel Composition | Biomar | | “Saturated” | With NT- | With | AUC |
| Number | (Biomarkers) | markers | Subgroup | Panel | proBNP | ANOVA | Estimate |
| 1 | SM23; OSS2; PC4 | 3 | HFrEF | yes | yes | no | 0.968 |
| 2 | OSS2; SM23; CE C18:2 | 3 | HFrEF | yes | yes | yes | 0.955 |
| 3 | SOP2; OSS2; PC4; CE C18:2; SM18; | 9 | HFrEF | yes | no | no | 0.89 |
| | SM28; SM24; SSP2; SM23 | | | | | | |
| 4 | OSS2; CE C18:2; SM23 | 3 | HFrEF | no | no | no | 0.876 |
| 5 | SOP2; OSS2; SM18; CE C18:2; SM24; | 8 | HFrEF | yes | no | yes | 0.864 |
| | SM28; Cer(d16:1/24:0); PC4 | | | | | | |
| 6 | SM18; OSS2; CE C18:2 | 3 | HFrEF | no | no | yes | 0.851 |
| 7 | SM23; SOP2; CE C18:2 | 3 | HFrEF asymptomatic | yes | yes | no | 0.952 |
| 8 | SM23; SOP2; CE C18:2 | 3 | HFrEF asymptomatic | yes | yes | yes | 0.934 |
| 9 | SOP2; SM24; SSP2; SM23; CE C18:2; | 8 | HFrEF asymptomatic | yes | no | no | 0.86 |
| | SM28; SM18; PC4 | | | | | | |
| 10 | SM23; SOP2; SM28 | 3 | HFrEF asymptomatic | no | no | no | 0.855 |
| 11 | SM24; SOP2; CE C18:2; SM28; SSP2; | 9 | HFrEF asymptomatic | yes | no | yes | 0.815 |
| | SM18; OSS2; SM2; Cer(d16:1/24:0) | | | | | | |
| 12 | SM24; SOP2; CE C18:2 | 3 | HFrEF asymptomatic | no | no | yes | 0.805 |
| 13 | OSS2; PC4; SM23 | 3 | HFrEF symptomatic | yes | yes | no | 0.978 |
| 14 | OSS2; CE C18:2; SM3 | 3 | HFrEF symptomatic | yes | yes | yes | 0.967 |
| 15 | SM18; SM28; PC4; OSS2; SOP2; | 8 | HFrEF symptomatic | yes | no | no | 0.905 |
| | CE C18:2; PPO1; SM10 | | | | | | |
| 16 | CE C18:2; SOP2; SM18 | 3 | HFrEF symptomatic | no | no | no | 0.89 |
| 17 | SM18; OSS2; SOP2; CE C18:2; PC4; | 10 | HFrEF symptomatic | yes | no | yes | 0.882 |
| | SPP1; SM24; Cer(d16:1/24:0); | | | | | | |
| | SM28; SM21 | | | | | | |
| 18 | SM18; CE C18:2; OSS2 | 3 | HFrEF symptomatic | no | no | yes | 0.867 |
| 19 | SM23; SOP2; CE C18:2 | 3 | DCMP asymptomatic | yes | yes | no | 0.929 |
| 20 | OSS2; SM23; CE C18:2 | 3 | DCMP asymptomatic | yes | yes | yes | 0.91 |
| 21 | CE C18:2; OSS2; SM23; SM24; SSP2 | 5 | DCMP asymptomatic | yes | no | no | 0.803 |
| 22 | OSS2; SM23; CE C18:2 | 3 | DCMP asymptomatic | no | no | no | 0.799 |
| 23 | SM23; CE C18:2; OSS2; SSP2; SM24 | 5 | DCMP asymptomatic | yes | no | yes | 0.738 |
| 24 | SM24; SSP2; OSS2 | 3 | DCMP asymptomatic | no | no | yes | 0.728 |
| 25 | SOP2; PC4; SM3 | 3 | DCMP symptomatic | yes | yes | no | 0.977 |
| 26 | OSS2; PC4; SM3 | 3 | DCMP symptomatic | yes | yes | yes | 0.966 |
| 27 | SSP2; PPO1; SM18; CE C18:2; OSS2; | 8 | DCMP symptomatic | yes | no | no | 0.87 |
| | SOP2; PC4; SM28 | | | | | | |
| 28 | SOP2; SM28; PC4 | 3 | DCMP symptomatic | no | no | no | 0.851 |
| 29 | Cer(d16:1/24:0); SM28; PC4; SM24; | 8 | DCMP symptomatic | yes | no | yes | 0.846 |
| | CE C18:2; SPP1; OSS2; SOP2 | | | | | | |
| 30 | OSS2; CE C18:2; SM24 | 3 | DCMP symptomatic | no | no | yes | 0.824 |
| 31 | SOP2; SM23; PC4 | 3 | DCMP | yes | yes | no | 0.959 |
| 32 | OSS2; CE C18:2; SM23 | 3 | DCMP | yes | yes | yes | 0.946 |
| 33 | SM24; SOP2; OSS2; CE C18:2; SSP2; | 7 | DCMP | yes | no | no | 0.845 |
| | PC4; SM28 | | | | | | |
| 34 | SOP2; SM24; PC4 | 3 | DCMP | no | no | no | 0.833 |
| 35 | OSS2; CE C18:2; SOP2; SM18; SM24; | 8 | DCMP | yes | no | yes | 0.815 |
| | SSP2; SM28; PC4 | | | | | | |
| 36 | OSS2; SM24; CE C18:2 | 3 | DCMP | no | no | yes | 0.803 |
| 37 | SM5; PPO1; SM23 | 3 | ICMP asymptomatic | yes | yes | no | 0.98 |
| 38 | SM5; SOP2; SM24 | 3 | ICMP asymptomatic | yes | yes | yes | 0.966 |
| 39 | SSP2; CE C18:2; SM18; SM23; | 10 | ICMP asymptomatic | yes | no | no | 0.897 |
| | Cer(d16:1/24:0); PC4; SOP2; PPO1; | | | | | | |
| | SM28; SM24 | | | | | | |
| 40 | SM24; SOP2; PC4 | 3 | ICMP asymptomatic | no | no | no | 0.884 |
| 41 | PC4; SM5; CE C18:2; SM2; PPO1; | 9 | ICMP asymptomatic | yes | no | yes | 0.866 |
| | SOP2; SM28; Cer(d16:1/24:0); SM24 | | | | | | |
| 42 | SM24; CE C18:2; SOP2 | 3 | ICMP asymptomatic | no | no | yes | 0.834 |
| 43 | Cer(d18:1/24:1); SOP2; CE C18:2; | 4 | ICMP symptomatic | yes | yes | no | 0.98 |
| | SM18 | | | | | | |
| 44 | SM18; CE C18:2; SOP2 | 3 | ICMP symptomatic | no | yes | no | 0.978 |
| 45 | SOP2; SM24; CE C18:2; SM18 | 4 | ICMP symptomatic | yes | yes | yes | 0.967 |
| 46 | SM18; CE C18:2; SOP2 | 3 | ICMP symptomatic | no | yes | yes | 0.965 |
| 47 | SSP2; PC4; Cer(d18:1/24:1); | 8 | ICMP symptomatic | yes | no | no | 0.937 |
| | SM28; SM24; SOP2; CE C18:2; SM18 | | | | | | |
| 48 | SM18; CE C18:2; SOP2 | 3 | ICMP symptomatic | no | no | no | 0.93 |
| 49 | SM28; CE C18:2; SOP2; SM18 | 4 | ICMP symptomatic | yes | no | yes | 0.913 |
| 50 | SM18; CE C18:2; SOP2 | 3 | ICMP symptomatic | no | no | yes | 0.909 |
| 51 | SM23; CE C18:2; SOP2 | 3 | ICMP | yes | yes | no | 0.977 |
| 52 | SM23; CE C18:2; SOP2 | 3 | ICMP | yes | yes | yes | 0.965 |
| 53 | SOP2; SM24; CE C18:2; SM18; PC4; | 10 | ICMP | yes | no | no | 0.924 |
| | SM28; PPO1; Cer(d16:1/24:0); | | | | | | |
| | OSS2; Cer(d18:1/24:1) | | | | | | |
| 54 | SM18; CE C18:2; SOP2 | 3 | ICMP | no | no | no | 0.915 |
| 55 | SOP2; SM18; SM24; CE C18:2; | 8 | ICMP | yes | no | yes | 0.902 |
| | Cer(d16:1/24:0); SM28; OSS2; PC4 | | | | | | |
| 56 | SM18; CE C18:2; SOP2 | 3 | ICMP | no | no | yes | 0.892 |
| 57 | SOP2; SM23; PC4 | 3 | CHF asymptomatic | yes | yes | no | 0.875 |
| 58 | SOP2; SSP2; SM5; SPP1; SM2; | 9 | CHF asymptomatic | yes | yes | yes | 0.844 |
| | SM3; PC4; CE C18:2; Glutamic acid | | | | | | |
| 59 | SOP2; SM5; SM2 | 3 | CHF asymptomatic | no | yes | yes | 0.84 |
| 60 | SOP2; SM23; SSP2; SM24; CE C18:2; | 7 | CHF asymptomatic | yes | no | no | 0.797 |
| | PC4; OSS2 | | | | | | |
| 61 | SOP2; SM23; CE C18:2 | 3 | CHF asymptomatic | no | no | no | 0.791 |
| 62 | SOP2; SSP2; SM2; CE C18:2; SM24; | 9 | CHF asymptomatic | yes | no | yes | 0.759 |
| | OSS2; SM18; PC4; SM28 | | | | | | |
| 63 | SOP2; SM18; CE C18:2 | 3 | CHF asymptomatic | no | no | yes | 0.742 |
| 64 | SOP2; PC4; OSS2; Cer(d18:1/24:1); | 8 | CHF symptomatic | yes | yes | no | 0.953 |
| | SM24; PC8; SM21; Cer(d17:1/24:0) | | | | | | |
| 65 | SOP2; PC4; SM24 | 3 | CHF symptomatic | no | yes | no | 0.952 |
| 66 | SOP2; SM18; PC4; SM21 | 4 | CHF symptomatic | yes | yes | yes | 0.931 |
| 67 | SOP2; SM18; PC4 | 3 | CHF symptomatic | no | yes | yes | 0.93 |
| 68 | SOP2; PC4; SM28; SM18; | 7 | CHF symptomatic | yes | no | no | 0.874 |
| | Cer(d18:1/24:1); Cer(d18:2/24:0); | | | | | | |
| | SM9 | | | | | | |
| 69 | SOP2; PC4; SM18 | 3 | CHF symptomatic | no | no | no | 0.854 |
| 70 | PC4; SOP2; SM18; SM24; OSS2; | 10 | CHF symptomatic | yes | no | yes | 0.853 |
| | SPP1; SM21; Cer(d16:1/24:0); | | | | | | |
| | SM28; CE C18:2 | | | | | | |
| 71 | SM18; SOP2; PC4 | 3 | CHF symptomatic | no | no | yes | 0.837 |
| 72 | SOP2; PC4; SM5 | 3 | CHF | yes | yes | no | 0.919 |
| 73 | SOP2; SM3; PC4; SM21 | 4 | CHF | yes | yes | yes | 0.893 |
| 74 | SOP2; SM3; PC4 | 3 | CHF | no | yes | yes | 0.889 |
| 75 | SOP2; SM18; PC4; SM28; OSS2; | 7 | CHF | yes | no | no | 0.844 |
| | SSP2; CE C18:2 | | | | | | |
| 76 | SOP2; SM18; PC4 | 3 | CHF | no | no | no | 0.834 |
| 77 | SOP2; OSS2; SM18; SM24; PC4; | 8 | CHF | yes | no | yes | 0.816 |
| | CE C18:2; Cer(d16:1/24:0); SM28 | | | | | | |
| 78 | OSS2; SM18; CE C18:2 | 3 | CHF | no | no | yes | 0.806 |
| 79 | SPP1; SOP2; SM5; PC4; SM3; SSP2 | 6 | HFpEF asymptomatic | yes | yes | no | 0.788 |
| 80 | SPP1; SM5; PC4 | 3 | HFpEF asymptomatic | no | yes | no | 0.782 |
| 81 | SPP1; SM3; SM5; SSP2; PC4; | 9 | HFpEF asymptomatic | yes | yes | yes | 0.739 |
| | Glutamic acid; CE C18:0; | | | | | | |
| | OSS2; SOP2 | | | | | | |
| 82 | SPP1; SM3; SM5 | 3 | HFpEF asymptomatic | no | yes | yes | 0.723 |
| 83 | SPP1; SOP2; SSP2; PC4; SM23; SM5; | 8 | HFpEF asymptomatic | yes | no | no | 0.727 |
| | SM3; CE C18:2 | | | | | | |
| 84 | SPP1; SM23; SOP2 | 3 | HFpEF asymptomatic | no | no | no | 0.717 |
| 85 | SM3; SPP1; SSP2; SM5; SOP2; PC4 | 6 | HFpEF asymptomatic | yes | no | yes | 0.692 |
| 86 | SPP1; SM3; SM5 | 3 | HFpEF asymptomatic | no | no | yes | 0.664 |
| 87 | SOP2; Cer(d18:1/24:1); PC8; | 9 | HFpEF symptomatic | yes | yes | no | 0.882 |
| | PPO1; Cer(d18:1/23:0); PC4; | | | | | | |
| | SM24; SM28; SM9 | | | | | | |
| 88 | SOP2; PC4; SM24 | 3 | HFpEF symptomatic | no | yes | no | 0.87 |
| 89 | Cer(d18:1/23:0); SM24; SOP2; | 9 | HFpEF symptomatic | yes | yes | yes | 0.828 |
| | Cer(d18:1/24:1); PC4; PPO1; | | | | | | |
| | PC8; SSS; SM18 | | | | | | |
| 90 | SM24; SOP2; PC4 | 3 | HFpEF symptomatic | no | yes | yes | 0.81 |
| 91 | PC4; SM28; SM24; Cer(d18:1/24:1); | 10 | HFpEF symptomatic | yes | no | no | 0.808 |
| | PC8; SOP2; SM9; PPO1; SM21; SSP2 | | | | | | |
| 92 | PC4; SM28; SOP2 | 3 | HFpEF symptomatic | no | no | no | 0.748 |
| 93 | SM24; PC4; SM18; SOP2; SM21; SPP1; | 8 | HFpEF symptomatic | yes | no | yes | 0.767 |
| | Cer(d18:1/23:0); SSP2 | | | | | | |
| 94 | SM24; SM18; SOP2 | 3 | HFpEF symptomatic | no | no | yes | 0.746 |
| 95 | SOP2; PC8; SPP1; PC4; SSP2; PPO1; | 9 | HFpEF | yes | yes | no | 0.824 |
| | SM5; SM18; Cer(d17:1/24:0) | | | | | | |
| 96 | SOP2; PC8; SM5 | 3 | HFpEF | no | yes | no | 0.821 |
| 97 | SPP1; PC4; SM3; SOP2; SSP2; | 9 | HFpEF | yes | yes | yes | 0.771 |
| | Cer(d18:1/23:0); SM24; SM5; | | | | | | |
| | Glutamic acid | | | | | | |
| 98 | SPP1; SM3; PC4 | 3 | HFpEF | no | yes | yes | 0.763 |
| 99 | SOP2; PC4; SPP1; PC8; SSP2; SM28; | 10 | HFpEF | yes | no | no | 0.759 |
| | SM24; PPO1; SM18; Cer(d17:1/24:0) | | | | | | |
| 100 | SOP2; PC4; SM24 | 3 | HFpEF | no | no | no | 0.743 |
| 101 | PC4; SPP1; SM24; SSP2; SM3; SOP2; | 10 | HFpEF | yes | no | yes | 0.726 |
| | SM18; Glutamic acid; | | | | | | |
| | Cer(d17:1/24:0); Cer(d18:1/23:0) | | | | | | |
| 102 | SPP1; SM18; PC4 | 3 | HFpEF | no | no | yes | 0.705 |
| 103 | CE C18:2; SSS; Cer(d17:1/24:0) | 3 | (all) | (na) | (na) | (na) | (see |
| | | | | | | | Table 3) |
| 104 | PC4; SOP2; CE C18:2 | 3 | (all) | (na) | (na) | (na) | (see |
| | | | | | | | Tables 4A-D) |
| 105 | CE C18:2; PC4; SM29; SOP2 | 4 | (all) | (na) | (na) | (na) | (see |
| | | | | | | | Tables 4A-D) |
| 106 | CE C18:2; PC8; SM29; SSP2 | 4 | (all) | (na) | (na) | (na) | (see |
| | | | | | | | Tables 4A-D) |
| 107 | CE C18:2; PC4; SM8; SSP2 | 4 | (all) | (na) | (na) | (na) | (see |
| | | | | | | | Tables 4A-D) |
| 108 | CE C18:2; PC8; SM8; SSP2 | 4 | (all) | (na) | (na) | (na) | (see |
| | | | | | | | Tables 4A-D) |
| 109 | CE C18:2; PC4; SM29; PPO1 | 4 | (all) | (na) | (na) | (na) | (see |
| | | | | | | | Tables 4A-D) |
| 110 | CE C18:2; PC8; SM29; PPO1 | 4 | (all) | (na) | (na) | (na) | (see |
| | | | | | | | Tables 4A-D) |
| 111 | CE C18:2; PC4; SM8; PPO1 | 4 | (all) | (na) | (na) | (na) | (see |
| | | | | | | | Tables 4A-D) |
| 112 | CE C18:2; PC8; SM8; PPO1 | 4 | (all) | (na) | (na) | (na) | (see |
| | | | | | | | Tables 4A-D) |
| 113 | CE C18:2; PC4; SM29; PPP | 4 | (all) | (na) | (na) | (na) | (see |
| | | | | | | | Tables 4A-D) |
| 114 | CE C18:2; PC8; SM29; PPP | 4 | (all) | (na) | (na) | (na) | (see |
| | | | | | | | Tables 4A-D) |
| 115 | CE C18:2; PC4; SM8; PPP | 4 | (all) | (na) | (na) | (na) | (see |
| | | | | | | | Tables 4A-D) |
| 116 | CE C18:2; PC8; SM29; SOP2 | 4 | (all) | (na) | (na) | (na) | (see |
| | | | | | | | Tables 4A-D) |
| 117 | CE C18:2; PC8; SM8; PPP | 4 | (all) | (na) | (na) | (na) | (see |
| | | | | | | | Tables 4A-D) |
| 118 | CE C18:2; PC4; SM8; SOP2 | 4 | (all) | (na) | (na) | (na) | (see |
| | | | | | | | Tables 4A-D) |
| 119 | CE C18:2; PC8; SM8; SOP2 | 4 | (all) | (na) | (na) | (na) | (see |
| | | | | | | | Tables 4A-D) |
| 120 | CE C18:2; PC4; SM29; SPP1 | 4 | (all) | (na) | (na) | (na) | (see |
| | | | | | | | Tables 4A-D) |
| 121 | CE C18:2; PC8; SM29; SPP1 | 4 | (all) | (na) | (na) | (na) | (see |
| | | | | | | | Tables 4A-D) |
| 122 | CE C18:2; PC4; SM8; SPP1 | 4 | (all) | (na) | (na) | (na) | (see |
| | | | | | | | Tables 4A-D) |
| 123 | CE C18:2; PC8; SM8; SPP1 | 4 | (all) | (na) | (na) | (na) | (see |
| | | | | | | | Tables 4A-D) |
| 124 | CE C18:2; PC4; SM29; SSP2 | 4 | (all) | (na) | (na) | (na) | (see |
| | | | | | | | Tables 4A-D) |
| 125 | PC4; SOP2; CE C18:2; SM18 | 4 | (all) | (na) | (na) | (na) | (see |
| | | | | | | | Tables 4A-D) |
| 126 | PC4; SOP2; CE C18:0; SM18 | 4 | (all) | (na) | (na) | (na) | (see |
| | | | | | | | Tables 4A-D) |
| 127 | PC4; SOP2; CE C18:2; SM21 | 4 | (all) | (na) | (na) | (na) | (see |
| | | | | | | | Tables 4A-D) |
| 128 | PC4; SOP2; CE C18:0; SM21 | 4 | (all) | (na) | (na) | (na) | (see |
| | | | | | | | Tables 4A-D) |
| 129 | PC4; SOP2; CE C18:2; SM23 | 4 | (all) | (na) | (na) | (na) | (see |
| | | | | | | | Tables 4A-D) |
| 130 | PC4; SOP2; CE C18:0; SM23 | 4 | (all) | (na) | (na) | (na) | (see |
| | | | | | | | Tables 4A-D) |
| 131 | PC4; SOP2; CE C18:2; SM24 | 4 | (all) | (na) | (na) | (na) | (see |
| | | | | | | | Tables 4A-D) |
| 132 | PC4; SOP2; CE C18:0; SM24 | 4 | (all) | (na) | (na) | (na) | (see |
| | | | | | | | Tables 4A-D) |
| 133 | SOP2; CE C18:0; SM18; PC4; SPP1 | 5 | (all) | (na) | (na) | (na) | (see |
| | | | | | | | Tables 4A-D) |
| 134 | SOP2; CE C18:2; SM21; PC4; SPP1 | 5 | (all) | (na) | (na) | (na) | (see |
| | | | | | | | Tables 4A-D) |
| 135 | SOP2; CE C18:0; SM21; PC4; SPP1 | 5 | (all) | (na) | (na) | (na) | (see |
| | | | | | | | Tables 4A-D) |
| 136 | SOP2; CE C18:2; SM23; PC4; SPP1 | 5 | (all) | (na) | (na) | (na) | (see |
| | | | | | | | Tables 4A-D) |
| 137 | SOP2; CE C18:0; SM23; PC4; SPP1 | 5 | (all) | (na) | (na) | (na) | (see |
| | | | | | | | Tables 4A-D) |
| 138 | SOP2; CE C18:2; SM24; PC4; SPP1 | 5 | (all) | (na) | (na) | (na) | (see |
| | | | | | | | Tables 4A-D) |
| 139 | SOP2; CE C18:0; SM24; PC4; SPP1 | 5 | (all) | (na) | (na) | (na) | (see |
| | | | | | | | Tables 4A-D) |
| 140 | SOP2; CE C18:2; SM18; PC4; SSP2 | 5 | (all) | (na) | (na) | (na) | (see |
| | | | | | | | Tables 4A-D) |
| 141 | SOP2; CE C18:0; SM18; PC4; SSP2 | 5 | (all) | (na) | (na) | (na) | (see |
| | | | | | | | Tables 4A-D) |
| 142 | SOP2; CE C18:2; SM21; PC4; SSP2 | 5 | (all) | (na) | (na) | (na) | (see |
| | | | | | | | Tables 4A-D) |
| 143 | SOP2; CE C18:0; SM21; PC4; SSP2 | 5 | (all) | (na) | (na) | (na) | (see |
| | | | | | | | Tables 4A-D) |
| 144 | SOP2; CE C18:2; SM23; PC4; SSP2 | 5 | (all) | (na) | (na) | (na) | (see |
| | | | | | | | Tables 4A-D) |
| 145 | SOP2; CE C18:0; SM23; PC4; SSP2 | 5 | (all) | (na) | (na) | (na) | (see |
| | | | | | | | Tables 4A-D) |
| 146 | SOP2; CE C18:2; SM24; PC4; SSP2 | 5 | (all) | (na) | (na) | (na) | (see |
| | | | | | | | Tables 4A-D) |
| 147 | SOP2; CE C18:0; SM24; PC4; SSP2 | 5 | (all) | (na) | (na) | (na) | (see |
| | | | | | | | Tables 4A-D) |
| 148 | SOP2; CE C18:2; SM18; PC4; PPO1 | 5 | (all) | (na) | (na) | (na) | (see |
| | | | | | | | Tables 4A-D) |
| 149 | SOP2; CE C18:0; SM18; PC4; PPO1 | 5 | (all) | (na) | (na) | (na) | (see |
| | | | | | | | Tables 4A-D) |
| 150 | SOP2; CE C18:2; SM21; PC4; PPO1 | 5 | (all) | (na) | (na) | (na) | (see |
| | | | | | | | Tables 4A-D) |
| 151 | SOP2; CE C18:0; SM21; PC4; PPO1 | 5 | (all) | (na) | (na) | (na) | (see |
| | | | | | | | Tables 4A-D) |
| 152 | SOP2; CE C18:2; SM23; PC4; PPO1 | 5 | (all) | (na) | (na) | (na) | (see |
| | | | | | | | Tables 4A-D) |
| 153 | SOP2; CE C18:0; SM23; PC4; PPO1 | 5 | (all) | (na) | (na) | (na) | (see |
| | | | | | | | Tables 4A-D) |
| 154 | SOP2; CE C18:2; SM24; PC4; PPO1 | 5 | (all) | (na) | (na) | (na) | (see |
| | | | | | | | Tables 4A-D) |
| 155 | SOP2; CE C18:0; SM24; PC4; PPO1 | 5 | (all) | (na) | (na) | (na) | (see |
| | | | | | | | Tables 4A-D) |
| 156 | SOP2; CE C18:2; SM18; PC4; PPP | 5 | (all) | (na) | (na) | (na) | (see |
| | | | | | | | Tables 4A-D) |
| 157 | SOP2; CE C18:0; SM18; PC4; PPP | 5 | (all) | (na) | (na) | (na) | (see |
| | | | | | | | Tables 4A-D) |
| 158 | SOP2; CE C18:2; SM21; PC4; PPP | 5 | (all) | (na) | (na) | (na) | (see |
| | | | | | | | Tables 4A-D) |
| 159 | SOP2; CE C18:0; SM21; PC4; PPP | 5 | (all) | (na) | (na) | (na) | (see |
| | | | | | | | Tables 4A-D) |
| 160 | SOP2; CE C18:2; SM23; PC4; PPP | 5 | (all) | (na) | (na) | (na) | (see |
| | | | | | | | Tables 4A-D) |
| 161 | SOP2; CE C18:0; SM23; PC4; PPP | 5 | (all) | (na) | (na) | (na) | (see |
| | | | | | | | Tables 4A-D) |
| 162 | SOP2; CE C18:2; SM24; PC4; PPP | 5 | (all) | (na) | (na) | (na) | (see |
| | | | | | | | Tables 4A-D) |
| 163 | SOP2; CE C18:0; SM24; PC4; PPP | 5 | (all) | (na) | (na) | (na) | (see |
| | | | | | | | Tables 4A-D) |
| 164 | SOP2; CE C18:2; SM18; PC4; | 5 | (all) | (na) | (na) | (na) | (see |
| | SPP1 | | | | | | Tables 4A-D) |
| 165 | PC4; PPP; CE C18:0; SM18 | 4 | (all) | (na) | (na) | (na) | (see |
| | | | | | | | Tables 4A-D) |
| 166 | PC4; SPP1; CE C18:2; SM21 | 4 | (all) | (na) | (na) | (na) | (see |
| | | | | | | | Tables 4A-D) |
| 167 | PC4; SSP2; CE C18:2; SM21 | 4 | (all) | (na) | (na) | (na) | (see |
| | | | | | | | Tables 4A-D) |
| 168 | PC4; PPO1; CE C18:2; SM21 | 4 | (all) | (na) | (na) | (na) | (see |
| | | | | | | | Tables 4A-D) |
| 169 | PC4; PPP; CE C18:2; SM21 | 4 | (all) | (na) | (na) | (na) | (see |
| | | | | | | | Tables 4A-D) |
| 170 | PC4; SPP1; CE C18:0; SM21 | 4 | (all) | (na) | (na) | (na) | (see |
| | | | | | | | Tables 4A-D) |
| 171 | PC4; SSP2; CE C18:0; SM21 | 4 | (all) | (na) | (na) | (na) | (see |
| | | | | | | | Tables 4A-D) |
| 172 | PC4; PPO1; CE C18:0; SM21 | 4 | (all) | (na) | (na) | (na) | (see |
| | | | | | | | Tables 4A-D) |
| 173 | PC4; SPP1; CE C18:2; SM18 | 4 | (all) | (na) | (na) | (na) | (see |
| | | | | | | | Tables 4A-D) |
| 174 | PC4; PPP; CE C18:0; SM21 | 4 | (all) | (na) | (na) | (na) | (see |
| | | | | | | | Tables 4A-D) |
| 175 | PC4; SPP1; CE C18:2; SM23 | 4 | (all) | (na) | (na) | (na) | (see |
| | | | | | | | Tables 4A-D) |
| 176 | PC4; SSP2; CE C18:2; SM23 | 4 | (all) | (na) | (na) | (na) | (see |
| | | | | | | | Tables 4A-D) |
| 177 | PC4; PPO1; CE C18:2; SM23 | 4 | (all) | (na) | (na) | (na) | (see |
| | | | | | | | Tables 4A-D) |
| 178 | PC4; PPP; CE C18:2; SM23 | 4 | (all) | (na) | (na) | (na) | (see |
| | | | | | | | Tables 4A-D) |
| 179 | PC4; SPP1; CE C18:0; SM23 | 4 | (all) | (na) | (na) | (na) | (see |
| | | | | | | | Tables 4A-D) |
| 180 | PC4; SSP2; CE C18:0; SM23 | 4 | (all) | (na) | (na) | (na) | (see |
| | | | | | | | Tables 4A-D) |
| 181 | PC4; PPO1; CE C18:0; SM23 | 4 | (all) | (na) | (na) | (na) | (see |
| | | | | | | | Tables 4A-D) |
| 182 | PC4; SSP2; CE C18:2; SM18 | 4 | (all) | (na) | (na) | (na) | (see |
| | | | | | | | Tables 4A-D) |
| 183 | PC4; PPP; CE C18:0; SM23 | 4 | (all) | (na) | (na) | (na) | (see |
| | | | | | | | Tables 4A-D) |
| 184 | PC4; SPP1; CE C18:2; SM24 | 4 | (all) | (na) | (na) | (na) | (see |
| | | | | | | | Tables 4A-D) |
| 185 | PC4; SSP2; CE C18:2; SM24 | 4 | (all) | (na) | (na) | (na) | (see |
| | | | | | | | Tables 4A-D) |
| 186 | PC4; PPO1; CE C18:2; SM24 | 4 | (all) | (na) | (na) | (na) | (see |
| | | | | | | | Tables 4A-D) |
| 187 | PC4; PPP; CE C18:2; SM24 | 4 | (all) | (na) | (na) | (na) | (see |
| | | | | | | | Tables 4A-D) |
| 188 | PC4; SPP1; CE C18:0; SM24 | 4 | (all) | (na) | (na) | (na) | (see |
| | | | | | | | Tables 4A-D) |
| 189 | PC4; SSP2; CE C18:0; SM24 | 4 | (all) | (na) | (na) | (na) | (see |
| | | | | | | | Tables 4A-D) |
| 190 | PC4; PPO1; CE C18:0; SM24 | 4 | (all) | (na) | (na) | (na) | (see |
| | | | | | | | Tables 4A-D) |
| 191 | PC4; PPO1; CE C18:2; SM18 | 4 | (all) | (na) | (na) | (na) | (see |
| | | | | | | | Tables 4A-D) |
| 192 | PC4; PPP; CE C18:0; SM24 | 4 | (all) | (na) | (na) | (na) | (see |
| | | | | | | | Tables 4A-D) |
| 193 | PC4; PPP; CE C18:2; SM18 | 4 | (all) | (na) | (na) | (na) | (see |
| | | | | | | | Tables 4A-D) |
| 194 | PC4; SPP1; CE C18:0; SM18 | 4 | (all) | (na) | (na) | (na) | (see |
| | | | | | | | Tables 4A-D) |
| 195 | PC4; SSP2; CE C18:0; SM18 | 4 | (all) | (na) | (na) | (na) | (see |
| | | | | | | | Tables 4A-D) |
| 196 | PC4; PPO1; CE C18:0; SM18 | 4 | (all) | (na) | (na) | (na) | (see |
| | | | | | | | Tables 4A-D) |
| 197 | PC4; SOP2; CE C18:2; SM28 | 4 | (all) | (na) | (na) | (na) | (see |
| | | | | | | | Tables 4A-D) |
| 198 | PC4; SOP2; CE C18:0; SM28 | 4 | (all) | (na) | (na) | (na) | (see |
| | | | | | | | Tables 4A-D) |
| 199 | SM18; SM24; SM28 | 3 | (all) | (na) | (na) | (na) | (see |
| | | | | | | | Tables 4A-D) |

TABLE 2a
Further multimarker panel compositions for
diagnosis of different CHF subgroups.

| | | No. of | | With | |
|---|---|---|---|---|---|
| Panel | Panel Composition | Bio- | Sub- | NT- | AUC |
| Number | (Biomarkers) | markers | group | proBNP | Estimate |
| 200 | OSS2; SM23; CE | 4 | (all) | (na) | (see Table 5a) |
| | C18:2; PC4 | | | | |
| 201 | CE C18:2; SM18; | 3 | (all) | (na) | (see Table 5a) |
| | SSS | | | | |
| 202 | CE C18:2; PC8; | 6 | (all) | YES | (see Table 5a) |
| | SM18; SM2; | | | | |
| | SM24; SSS | | | | |
| 203 | CE C18:2; SM18; | 5 | (all) | NO | (see Table 5a) |
| | SM21; SM24; SSS | | | | |
| 204 | SM24; CE C18:2; | 3 | (all) | (na) | (see Table 5a) |
| | SM2 | | | | |
| 205 | SM2; SSS; CE | 3 | (all) | (na) | (see Table 5a) |
| | C18:2 | | | | |
| 206 | SM24; SSS; CE | 3 | (all) | (na) | (see Table 5a) |
| | C18:2 | | | | |

TABLE 3
Performance of Panel Number 103 in different CHF subgroups,
including or excluding NT-proBNP, with or without ANOVA
correction for confounders age, BMI and gender.

| Panel | With NT- | With | | AUC |
|---|---|---|---|---|
| Number | proBNP | ANOVA | Subgroup | estimate |
| 103 | yes | no | DCMP asymptomatic | 0.903 |
| 103 | yes | yes | DCMP asymptomatic | 0.896 |
| 103 | no | no | DCMP asymptomatic | 0.729 |
| 103 | no | yes | DCMP asymptomatic | 0.667 |
| 103 | yes | no | DCMP symptomatic | 0.961 |
| 103 | yes | yes | DCMP symptomatic | 0.954 |
| 103 | no | no | DCMP symptomatic | 0.805 |
| 103 | no | yes | DCMP symptomatic | 0.791 |
| 103 | yes | no | DCMP | 0.942 |
| 103 | yes | yes | DCMP | 0.937 |
| 103 | no | no | DCMP | 0.795 |
| 103 | no | yes | DCMP | 0.771 |
| 103 | yes | no | HFrEF asymptomatic | 0.93 |
| 103 | yes | yes | HFrEF asymptomatic | 0.925 |
| 103 | no | no | HFrEF asymptomatic | 0.796 |
| 103 | no | yes | HFrEF asymptomatic | 0.768 |
| 103 | yes | no | HFrEF symptomatic | 0.969 |
| 103 | yes | yes | HFrEF symptomatic | 0.96 |
| 103 | no | no | HFrEF symptomatic | 0.855 |
| 103 | no | yes | HFrEF symptomatic | 0.838 |
| 103 | yes | no | HFrEF | 0.956 |
| 103 | yes | yes | HFrEF | 0.949 |
| 103 | no | no | HFrEF | 0.84 |
| 103 | no | yes | HFrEF | 0.818 |
| 103 | yes | no | ICMP asymptomatic | 0.951 |
| 103 | yes | yes | ICMP asymptomatic | 0.947 |
| 103 | no | no | ICMP asymptomatic | 0.838 |
| 103 | no | yes | ICMP asymptomatic | 0.815 |
| 103 | yes | no | ICMP symptomatic | 0.973 |
| 103 | yes | yes | ICMP symptomatic | 0.963 |
| 103 | no | no | ICMP symptomatic | 0.891 |
| 103 | no | yes | ICMP symptomatic | 0.871 |
| 103 | yes | no | ICMP | 0.967 |
| 103 | yes | yes | ICMP | 0.959 |
| 103 | no | no | ICMP | 0.878 |
| 103 | no | yes | ICMP | 0.858 |
| 103 | yes | no | CHF asymptomatic | 0.844 |
| 103 | yes | yes | CHF asymptomatic | 0.825 |
| 103 | no | no | CHF asymptomatic | 0.747 |
| 103 | no | yes | CHF asymptomatic | 0.717 |
| 103 | yes | no | CHF symptomatic | 0.938 |
| 103 | yes | yes | CHF symptomatic | 0.921 |
| 103 | no | no | CHF symptomatic | 0.822 |
| 103 | no | yes | CHF symptomatic | 0.812 |
| 103 | yes | no | CHF | 0.898 |
| 103 | yes | yes | CHF | 0.88 |
| 103 | no | no | CHF | 0.79 |
| 103 | no | yes | CHF | 0.772 |
| 103 | yes | no | HFpEF asymptomatic | 0.718 |
| 103 | yes | yes | HFpEF asymptomatic | 0.668 |
| 103 | no | no | HFpEF asymptomatic | 0.634 |
| 103 | no | yes | HFpEF asymptomatic | 0.592 |
| 103 | yes | no | HFpEF symptomatic | 0.835 |
| 103 | yes | yes | HFpEF symptomatic | 0.794 |
| 103 | no | no | HFpEF symptomatic | 0.658 |
| 103 | no | yes | HFpEF symptomatic | 0.697 |
| 103 | yes | no | HFpEF | 0.78 |
| 103 | yes | yes | HFpEF | 0.743 |
| 103 | no | no | HFpEF | 0.689 |
| 103 | no | yes | HFpEF | 0.679 |

TABLE 4A
AUC values for Panel Number 104 to 199 without NT-proBNP and without ANOVA correction in different CHF subgoups

| | | CHF | CHF | | DCMP | DCMP | | HFpEF | HFpEF | | HFrEF | HFrEF | | ICMP | ICMP |
|---|---|---|---|---|---|---|---|---|---|---|---|---|---|---|---|
| Panel | | asymp- | symp- | | asymp- | symp- | | asymp- | symp- | | asymp- | symp- | | asymp- | symp- |
| Number | CHF | tomatic | tomatic | DCMP | tomatic | tomatic | HFpEF | tomatic | tomatic | HFrEF | tomatic | tomatic | ICMP | tomatic | tomatic |
| 104 | 0.829 | 0.794 | 0.857 | 0.835 | 0.785 | 0.857 | 0.751 | 0.736 | 0.758 | 0.870 | 0.835 | 0.886 | 0.902 | 0.881 | 0.907 |
| 105 | 0.828 | 0.793 | 0.855 | 0.833 | 0.786 | 0.856 | 0.749 | 0.731 | 0.756 | 0.870 | 0.835 | 0.885 | 0.904 | 0.880 | 0.911 |
| 106 | 0.821 | 0.791 | 0.844 | 0.828 | 0.799 | 0.842 | 0.746 | 0.728 | 0.738 | 0.862 | 0.832 | 0.874 | 0.895 | 0.873 | 0.900 |
| 107 | 0.825 | 0.795 | 0.850 | 0.834 | 0.799 | 0.850 | 0.743 | 0.731 | 0.742 | 0.867 | 0.837 | 0.881 | 0.895 | 0.875 | 0.899 |
| 108 | 0.821 | 0.792 | 0.845 | 0.827 | 0.802 | 0.840 | 0.746 | 0.728 | 0.743 | 0.862 | 0.834 | 0.874 | 0.892 | 0.874 | 0.894 |
| 109 | 0.817 | 0.776 | 0.850 | 0.815 | 0.744 | 0.847 | 0.741 | 0.707 | 0.753 | 0.858 | 0.817 | 0.876 | 0.896 | 0.873 | 0.902 |
| 110 | 0.808 | 0.769 | 0.838 | 0.806 | 0.749 | 0.831 | 0.736 | 0.699 | 0.743 | 0.848 | 0.812 | 0.863 | 0.889 | 0.865 | 0.895 |
| 111 | 0.818 | 0.780 | 0.851 | 0.815 | 0.760 | 0.845 | 0.741 | 0.710 | 0.754 | 0.859 | 0.821 | 0.876 | 0.894 | 0.874 | 0.897 |
| 112 | 0.808 | 0.774 | 0.838 | 0.808 | 0.766 | 0.830 | 0.736 | 0.703 | 0.742 | 0.850 | 0.817 | 0.864 | 0.887 | 0.867 | 0.889 |
| 113 | 0.807 | 0.770 | 0.837 | 0.803 | 0.730 | 0.833 | 0.733 | 0.706 | 0.734 | 0.847 | 0.807 | 0.864 | 0.886 | 0.862 | 0.893 |
| 114 | 0.796 | 0.761 | 0.824 | 0.794 | 0.734 | 0.816 | 0.726 | 0.696 | 0.721 | 0.836 | 0.800 | 0.851 | 0.878 | 0.852 | 0.885 |
| 115 | 0.807 | 0.773 | 0.838 | 0.803 | 0.743 | 0.831 | 0.733 | 0.710 | 0.736 | 0.847 | 0.811 | 0.864 | 0.883 | 0.863 | 0.886 |
| 116 | 0.824 | 0.789 | 0.849 | 0.826 | 0.788 | 0.845 | 0.752 | 0.729 | 0.754 | 0.863 | 0.831 | 0.877 | 0.900 | 0.878 | 0.905 |
| 117 | 0.796 | 0.765 | 0.824 | 0.795 | 0.747 | 0.815 | 0.725 | 0.700 | 0.722 | 0.837 | 0.806 | 0.850 | 0.875 | 0.855 | 0.877 |
| 118 | 0.829 | 0.795 | 0.856 | 0.833 | 0.794 | 0.855 | 0.749 | 0.733 | 0.756 | 0.870 | 0.837 | 0.885 | 0.902 | 0.881 | 0.906 |
| 119 | 0.824 | 0.791 | 0.849 | 0.826 | 0.796 | 0.844 | 0.751 | 0.729 | 0.754 | 0.864 | 0.834 | 0.877 | 0.898 | 0.879 | 0.900 |
| 120 | 0.820 | 0.785 | 0.847 | 0.820 | 0.759 | 0.849 | 0.748 | 0.736 | 0.741 | 0.859 | 0.819 | 0.877 | 0.894 | 0.868 | 0.902 |
| 121 | 0.811 | 0.778 | 0.837 | 0.810 | 0.760 | 0.834 | 0.746 | 0.729 | 0.734 | 0.849 | 0.813 | 0.865 | 0.888 | 0.863 | 0.894 |
| 122 | 0.820 | 0.787 | 0.848 | 0.820 | 0.768 | 0.847 | 0.748 | 0.736 | 0.742 | 0.859 | 0.821 | 0.876 | 0.891 | 0.869 | 0.895 |
| 123 | 0.811 | 0.780 | 0.837 | 0.810 | 0.771 | 0.832 | 0.745 | 0.729 | 0.735 | 0.850 | 0.816 | 0.865 | 0.885 | 0.864 | 0.888 |
| 124 | 0.825 | 0.794 | 0.849 | 0.834 | 0.797 | 0.852 | 0.743 | 0.732 | 0.740 | 0.867 | 0.835 | 0.881 | 0.898 | 0.875 | 0.905 |
| 125 | 0.840 | 0.805 | 0.867 | 0.845 | 0.810 | 0.861 | 0.751 | 0.736 | 0.752 | 0.888 | 0.856 | 0.901 | 0.929 | 0.902 | 0.936 |
| 126 | 0.841 | 0.807 | 0.868 | 0.846 | 0.807 | 0.863 | 0.754 | 0.737 | 0.761 | 0.886 | 0.856 | 0.901 | 0.928 | 0.905 | 0.929 |
| 127 | 0.841 | 0.798 | 0.876 | 0.847 | 0.790 | 0.869 | 0.753 | 0.733 | 0.775 | 0.887 | 0.849 | 0.903 | 0.923 | 0.901 | 0.927 |
| 128 | 0.843 | 0.800 | 0.879 | 0.850 | 0.791 | 0.874 | 0.756 | 0.733 | 0.775 | 0.889 | 0.852 | 0.906 | 0.925 | 0.905 | 0.927 |
| 129 | 0.835 | 0.806 | 0.859 | 0.845 | 0.825 | 0.858 | 0.748 | 0.734 | 0.748 | 0.884 | 0.859 | 0.894 | 0.919 | 0.899 | 0.922 |
| 130 | 0.835 | 0.807 | 0.859 | 0.846 | 0.822 | 0.858 | 0.750 | 0.735 | 0.758 | 0.882 | 0.860 | 0.893 | 0.917 | 0.902 | 0.915 |
| 131 | 0.843 | 0.806 | 0.873 | 0.851 | 0.827 | 0.865 | 0.753 | 0.733 | 0.764 | 0.891 | 0.867 | 0.902 | 0.929 | 0.911 | 0.931 |
| 132 | 0.845 | 0.810 | 0.875 | 0.855 | 0.830 | 0.870 | 0.757 | 0.734 | 0.767 | 0.892 | 0.870 | 0.904 | 0.930 | 0.914 | 0.928 |
| 133 | 0.839 | 0.806 | 0.867 | 0.844 | 0.801 | 0.863 | 0.754 | 0.742 | 0.758 | 0.885 | 0.853 | 0.900 | 0.926 | 0.901 | 0.928 |
| 134 | 0.839 | 0.795 | 0.874 | 0.843 | 0.782 | 0.868 | 0.753 | 0.735 | 0.772 | 0.885 | 0.846 | 0.901 | 0.921 | 0.897 | 0.925 |
| 135 | 0.841 | 0.797 | 0.876 | 0.846 | 0.782 | 0.873 | 0.755 | 0.735 | 0.772 | 0.886 | 0.848 | 0.904 | 0.923 | 0.900 | 0.924 |
| 136 | 0.833 | 0.804 | 0.857 | 0.843 | 0.818 | 0.857 | 0.748 | 0.737 | 0.745 | 0.882 | 0.857 | 0.893 | 0.917 | 0.896 | 0.921 |
| 137 | 0.833 | 0.806 | 0.857 | 0.843 | 0.816 | 0.858 | 0.750 | 0.739 | 0.755 | 0.880 | 0.857 | 0.891 | 0.915 | 0.898 | 0.913 |
| 138 | 0.842 | 0.805 | 0.871 | 0.849 | 0.821 | 0.865 | 0.753 | 0.737 | 0.761 | 0.890 | 0.865 | 0.901 | 0.928 | 0.907 | 0.930 |
| 139 | 0.844 | 0.808 | 0.874 | 0.852 | 0.823 | 0.869 | 0.757 | 0.738 | 0.764 | 0.890 | 0.867 | 0.903 | 0.928 | 0.911 | 0.926 |
| 140 | 0.841 | 0.808 | 0.867 | 0.849 | 0.816 | 0.863 | 0.750 | 0.736 | 0.752 | 0.889 | 0.858 | 0.902 | 0.928 | 0.900 | 0.935 |
| 141 | 0.841 | 0.809 | 0.868 | 0.849 | 0.812 | 0.864 | 0.753 | 0.737 | 0.758 | 0.887 | 0.858 | 0.901 | 0.926 | 0.903 | 0.928 |
| 142 | 0.841 | 0.800 | 0.874 | 0.848 | 0.795 | 0.868 | 0.752 | 0.735 | 0.773 | 0.887 | 0.851 | 0.902 | 0.922 | 0.900 | 0.926 |
| 143 | 0.843 | 0.801 | 0.877 | 0.850 | 0.793 | 0.873 | 0.754 | 0.734 | 0.772 | 0.888 | 0.853 | 0.905 | 0.924 | 0.903 | 0.925 |
| 144 | 0.835 | 0.808 | 0.858 | 0.848 | 0.829 | 0.859 | 0.747 | 0.734 | 0.748 | 0.885 | 0.861 | 0.895 | 0.918 | 0.898 | 0.921 |
| 145 | 0.835 | 0.808 | 0.858 | 0.847 | 0.825 | 0.858 | 0.749 | 0.735 | 0.756 | 0.882 | 0.861 | 0.892 | 0.915 | 0.900 | 0.913 |
| 146 | 0.844 | 0.808 | 0.872 | 0.853 | 0.831 | 0.866 | 0.752 | 0.734 | 0.761 | 0.892 | 0.868 | 0.902 | 0.928 | 0.909 | 0.930 |
| 147 | 0.845 | 0.810 | 0.874 | 0.855 | 0.831 | 0.869 | 0.755 | 0.734 | 0.764 | 0.892 | 0.870 | 0.903 | 0.928 | 0.912 | 0.926 |
| 148 | 0.838 | 0.802 | 0.867 | 0.842 | 0.805 | 0.860 | 0.749 | 0.732 | 0.753 | 0.886 | 0.853 | 0.899 | 0.928 | 0.900 | 0.935 |
| 149 | 0.839 | 0.804 | 0.867 | 0.842 | 0.802 | 0.862 | 0.752 | 0.734 | 0.762 | 0.885 | 0.853 | 0.899 | 0.926 | 0.903 | 0.927 |
| 150 | 0.839 | 0.794 | 0.875 | 0.843 | 0.785 | 0.868 | 0.751 | 0.729 | 0.777 | 0.885 | 0.846 | 0.901 | 0.922 | 0.899 | 0.926 |
| 151 | 0.841 | 0.796 | 0.878 | 0.846 | 0.785 | 0.873 | 0.754 | 0.728 | 0.777 | 0.887 | 0.848 | 0.904 | 0.924 | 0.902 | 0.925 |
| 152 | 0.833 | 0.803 | 0.858 | 0.842 | 0.820 | 0.857 | 0.746 | 0.730 | 0.748 | 0.882 | 0.857 | 0.893 | 0.918 | 0.897 | 0.921 |
| 153 | 0.833 | 0.804 | 0.858 | 0.842 | 0.817 | 0.857 | 0.748 | 0.732 | 0.759 | 0.880 | 0.857 | 0.891 | 0.915 | 0.899 | 0.913 |
| 154 | 0.842 | 0.804 | 0.872 | 0.848 | 0.822 | 0.865 | 0.752 | 0.727 | 0.767 | 0.890 | 0.865 | 0.901 | 0.928 | 0.910 | 0.930 |
| 155 | 0.844 | 0.806 | 0.875 | 0.851 | 0.824 | 0.869 | 0.755 | 0.729 | 0.770 | 0.891 | 0.867 | 0.902 | 0.928 | 0.913 | 0.926 |
| 156 | 0.838 | 0.803 | 0.866 | 0.842 | 0.805 | 0.859 | 0.748 | 0.732 | 0.749 | 0.886 | 0.853 | 0.899 | 0.927 | 0.898 | 0.935 |
| 157 | 0.839 | 0.805 | 0.866 | 0.843 | 0.801 | 0.861 | 0.751 | 0.734 | 0.758 | 0.884 | 0.853 | 0.899 | 0.925 | 0.900 | 0.927 |
| 158 | 0.838 | 0.795 | 0.874 | 0.843 | 0.784 | 0.866 | 0.750 | 0.729 | 0.772 | 0.885 | 0.846 | 0.900 | 0.921 | 0.896 | 0.925 |
| 159 | 0.841 | 0.797 | 0.876 | 0.846 | 0.785 | 0.872 | 0.753 | 0.729 | 0.772 | 0.886 | 0.848 | 0.903 | 0.923 | 0.900 | 0.925 |
| 160 | 0.833 | 0.803 | 0.857 | 0.842 | 0.820 | 0.855 | 0.745 | 0.730 | 0.744 | 0.882 | 0.857 | 0.893 | 0.917 | 0.895 | 0.921 |
| 161 | 0.833 | 0.804 | 0.857 | 0.842 | 0.817 | 0.855 | 0.747 | 0.731 | 0.755 | 0.880 | 0.857 | 0.891 | 0.915 | 0.897 | 0.913 |
| 162 | 0.841 | 0.804 | 0.871 | 0.848 | 0.822 | 0.863 | 0.751 | 0.729 | 0.761 | 0.890 | 0.865 | 0.900 | 0.927 | 0.907 | 0.929 |
| 163 | 0.843 | 0.807 | 0.873 | 0.851 | 0.824 | 0.867 | 0.754 | 0.728 | 0.764 | 0.890 | 0.867 | 0.902 | 0.927 | 0.910 | 0.926 |
| 164 | 0.839 | 0.804 | 0.866 | 0.843 | 0.803 | 0.861 | 0.751 | 0.739 | 0.749 | 0.886 | 0.853 | 0.900 | 0.928 | 0.898 | 0.935 |
| 165 | 0.824 | 0.791 | 0.855 | 0.822 | 0.766 | 0.842 | 0.742 | 0.720 | 0.743 | 0.869 | 0.835 | 0.886 | 0.916 | 0.891 | 0.918 |
| 166 | 0.831 | 0.789 | 0.866 | 0.833 | 0.763 | 0.860 | 0.751 | 0.737 | 0.763 | 0.874 | 0.833 | 0.892 | 0.912 | 0.888 | 0.916 |
| 167 | 0.834 | 0.797 | 0.865 | 0.843 | 0.795 | 0.858 | 0.745 | 0.733 | 0.757 | 0.881 | 0.847 | 0.894 | 0.914 | 0.893 | 0.918 |
| 168 | 0.829 | 0.780 | 0.870 | 0.828 | 0.753 | 0.859 | 0.745 | 0.708 | 0.777 | 0.875 | 0.831 | 0.893 | 0.916 | 0.894 | 0.918 |
| 169 | 0.818 | 0.774 | 0.856 | 0.817 | 0.738 | 0.845 | 0.737 | 0.709 | 0.757 | 0.863 | 0.821 | 0.880 | 0.904 | 0.882 | 0.906 |
| 170 | 0.834 | 0.792 | 0.869 | 0.836 | 0.758 | 0.865 | 0.754 | 0.738 | 0.763 | 0.876 | 0.834 | 0.896 | 0.913 | 0.892 | 0.915 |
| 171 | 0.836 | 0.797 | 0.868 | 0.843 | 0.790 | 0.861 | 0.748 | 0.731 | 0.758 | 0.880 | 0.847 | 0.894 | 0.913 | 0.896 | 0.914 |
| 172 | 0.831 | 0.781 | 0.873 | 0.830 | 0.743 | 0.865 | 0.749 | 0.708 | 0.778 | 0.875 | 0.831 | 0.895 | 0.916 | 0.897 | 0.916 |
| 173 | 0.834 | 0.800 | 0.861 | 0.836 | 0.790 | 0.855 | 0.751 | 0.741 | 0.742 | 0.880 | 0.845 | 0.896 | 0.924 | 0.893 | 0.932 |
| 174 | 0.820 | 0.776 | 0.859 | 0.819 | 0.726 | 0.851 | 0.740 | 0.710 | 0.758 | 0.863 | 0.821 | 0.883 | 0.903 | 0.885 | 0.903 |
| 175 | 0.829 | 0.801 | 0.852 | 0.837 | 0.810 | 0.852 | 0.747 | 0.739 | 0.736 | 0.877 | 0.850 | 0.889 | 0.913 | 0.891 | 0.916 |
| 176 | 0.831 | 0.806 | 0.853 | 0.847 | 0.831 | 0.853 | 0.742 | 0.732 | 0.735 | 0.882 | 0.859 | 0.891 | 0.914 | 0.895 | 0.918 |
| 177 | 0.826 | 0.793 | 0.855 | 0.830 | 0.801 | 0.849 | 0.741 | 0.712 | 0.748 | 0.875 | 0.847 | 0.887 | 0.914 | 0.895 | 0.916 |
| 178 | 0.818 | 0.789 | 0.844 | 0.824 | 0.792 | 0.838 | 0.734 | 0.711 | 0.729 | 0.868 | 0.841 | 0.880 | 0.907 | 0.887 | 0.910 |
| 179 | 0.829 | 0.804 | 0.853 | 0.838 | 0.808 | 0.853 | 0.750 | 0.742 | 0.743 | 0.875 | 0.851 | 0.888 | 0.910 | 0.894 | 0.909 |
| 180 | 0.830 | 0.807 | 0.851 | 0.844 | 0.826 | 0.850 | 0.744 | 0.732 | 0.742 | 0.877 | 0.858 | 0.886 | 0.909 | 0.895 | 0.906 |
| 181 | 0.825 | 0.794 | 0.853 | 0.829 | 0.792 | 0.849 | 0.743 | 0.713 | 0.759 | 0.871 | 0.846 | 0.883 | 0.909 | 0.896 | 0.905 |
| 182 | 0.837 | 0.806 | 0.862 | 0.847 | 0.818 | 0.857 | 0.745 | 0.733 | 0.741 | 0.886 | 0.856 | 0.898 | 0.925 | 0.896 | 0.933 |
| 183 | 0.818 | 0.791 | 0.844 | 0.824 | 0.788 | 0.838 | 0.737 | 0.718 | 0.736 | 0.864 | 0.841 | 0.877 | 0.903 | 0.888 | 0.900 |
| 184 | 0.837 | 0.801 | 0.866 | 0.841 | 0.809 | 0.859 | 0.753 | 0.739 | 0.755 | 0.883 | 0.857 | 0.895 | 0.922 | 0.902 | 0.924 |
| 185 | 0.838 | 0.805 | 0.865 | 0.850 | 0.830 | 0.858 | 0.746 | 0.732 | 0.748 | 0.887 | 0.864 | 0.896 | 0.921 | 0.903 | 0.924 |
| 186 | 0.834 | 0.793 | 0.869 | 0.835 | 0.800 | 0.857 | 0.747 | 0.709 | 0.768 | 0.882 | 0.855 | 0.894 | 0.924 | 0.908 | 0.925 |
| 187 | 0.826 | 0.788 | 0.858 | 0.827 | 0.794 | 0.846 | 0.740 | 0.709 | 0.750 | 0.873 | 0.848 | 0.885 | 0.916 | 0.898 | 0.917 |
| 188 | 0.839 | 0.805 | 0.869 | 0.845 | 0.813 | 0.864 | 0.756 | 0.741 | 0.758 | 0.884 | 0.859 | 0.898 | 0.922 | 0.906 | 0.920 |
| 189 | 0.839 | 0.807 | 0.867 | 0.851 | 0.829 | 0.859 | 0.749 | 0.731 | 0.751 | 0.886 | 0.866 | 0.895 | 0.919 | 0.905 | 0.917 |
| 190 | 0.836 | 0.796 | 0.871 | 0.838 | 0.802 | 0.861 | 0.751 | 0.711 | 0.772 | 0.882 | 0.857 | 0.895 | 0.923 | 0.911 | 0.919 |
| 191 | 0.831 | 0.792 | 0.863 | 0.828 | 0.777 | 0.851 | 0.744 | 0.712 | 0.753 | 0.878 | 0.842 | 0.893 | 0.925 | 0.898 | 0.931 |
| 192 | 0.828 | 0.792 | 0.861 | 0.831 | 0.797 | 0.850 | 0.744 | 0.714 | 0.753 | 0.873 | 0.850 | 0.886 | 0.915 | 0.902 | 0.911 |
| 193 | 0.824 | 0.788 | 0.854 | 0.821 | 0.770 | 0.841 | 0.738 | 0.717 | 0.737 | 0.871 | 0.836 | 0.887 | 0.919 | 0.889 | 0.927 |
| 194 | 0.835 | 0.804 | 0.863 | 0.838 | 0.788 | 0.858 | 0.754 | 0.744 | 0.748 | 0.879 | 0.845 | 0.896 | 0.922 | 0.897 | 0.925 |
| 195 | 0.836 | 0.807 | 0.862 | 0.846 | 0.811 | 0.856 | 0.748 | 0.734 | 0.746 | 0.883 | 0.855 | 0.896 | 0.920 | 0.897 | 0.923 |
| 196 | 0.830 | 0.793 | 0.863 | 0.827 | 0.770 | 0.852 | 0.747 | 0.715 | 0.762 | 0.875 | 0.840 | 0.891 | 0.921 | 0.900 | 0.921 |
| 197 | 0.839 | 0.796 | 0.875 | 0.845 | 0.784 | 0.870 | 0.753 | 0.734 | 0.781 | 0.883 | 0.844 | 0.900 | 0.917 | 0.896 | 0.920 |
| 198 | 0.841 | 0.797 | 0.878 | 0.848 | 0.784 | 0.876 | 0.756 | 0.733 | 0.781 | 0.885 | 0.847 | 0.903 | 0.919 | 0.900 | 0.920 |
| 199 | 0.766 | 0.731 | 0.804 | 0.756 | 0.694 | 0.767 | 0.665 | 0.580 | 0.688 | 0.819 | 0.799 | 0.831 | 0.889 | 0.877 | 0.887 |

TABLE 4B
AUC values for Panel Number 104 to 199 with NT-proBNP and without
ANOVA correction in different CHF subgroups

| Panel | | CHF | CHF | | DCMP | DCMP | | HFpEF |
|---|---|---|---|---|---|---|---|---|
| Number | CHF | asymptomatic | symptomatic | DCMP | asymptomatic | symptomatic | HFpEF | asymptomatic |
| 104 | 0.919 | 0.873 | 0.955 | 0.959 | 0.920 | 0.980 | 0.827 | 0.790 |
| 105 | 0.919 | 0.875 | 0.955 | 0.959 | 0.924 | 0.979 | 0.826 | 0.788 |
| 106 | 0.916 | 0.873 | 0.951 | 0.956 | 0.926 | 0.974 | 0.826 | 0.786 |
| 107 | 0.916 | 0.874 | 0.952 | 0.959 | 0.926 | 0.978 | 0.820 | 0.787 |
| 108 | 0.915 | 0.873 | 0.951 | 0.956 | 0.926 | 0.974 | 0.826 | 0.787 |
| 109 | 0.914 | 0.866 | 0.952 | 0.951 | 0.911 | 0.974 | 0.822 | 0.776 |
| 110 | 0.910 | 0.864 | 0.948 | 0.947 | 0.911 | 0.968 | 0.822 | 0.772 |
| 111 | 0.913 | 0.868 | 0.952 | 0.951 | 0.912 | 0.974 | 0.823 | 0.780 |
| 112 | 0.910 | 0.866 | 0.948 | 0.947 | 0.912 | 0.968 | 0.823 | 0.777 |
| 113 | 0.911 | 0.866 | 0.948 | 0.950 | 0.911 | 0.973 | 0.821 | 0.778 |
| 114 | 0.907 | 0.862 | 0.944 | 0.946 | 0.912 | 0.967 | 0.819 | 0.771 |
| 115 | 0.910 | 0.866 | 0.948 | 0.950 | 0.911 | 0.973 | 0.821 | 0.779 |
| 116 | 0.918 | 0.874 | 0.953 | 0.955 | 0.924 | 0.974 | 0.832 | 0.789 |
| 117 | 0.906 | 0.864 | 0.944 | 0.946 | 0.912 | 0.967 | 0.819 | 0.774 |
| 118 | 0.919 | 0.876 | 0.955 | 0.958 | 0.924 | 0.979 | 0.827 | 0.790 |
| 119 | 0.918 | 0.875 | 0.953 | 0.955 | 0.924 | 0.974 | 0.832 | 0.790 |
| 120 | 0.916 | 0.872 | 0.951 | 0.955 | 0.915 | 0.977 | 0.828 | 0.794 |
| 121 | 0.913 | 0.870 | 0.948 | 0.951 | 0.915 | 0.971 | 0.831 | 0.792 |
| 122 | 0.915 | 0.872 | 0.951 | 0.954 | 0.914 | 0.977 | 0.828 | 0.794 |
| 123 | 0.912 | 0.871 | 0.948 | 0.950 | 0.915 | 0.971 | 0.831 | 0.792 |
| 124 | 0.917 | 0.873 | 0.952 | 0.960 | 0.926 | 0.979 | 0.820 | 0.786 |
| 125 | 0.921 | 0.877 | 0.956 | 0.958 | 0.927 | 0.978 | 0.828 | 0.792 |
| 126 | 0.922 | 0.879 | 0.957 | 0.959 | 0.927 | 0.978 | 0.830 | 0.795 |
| 127 | 0.919 | 0.872 | 0.956 | 0.957 | 0.918 | 0.978 | 0.827 | 0.787 |
| 128 | 0.921 | 0.873 | 0.957 | 0.958 | 0.916 | 0.979 | 0.829 | 0.790 |
| 129 | 0.922 | 0.883 | 0.955 | 0.961 | 0.941 | 0.978 | 0.827 | 0.793 |
| 130 | 0.923 | 0.886 | 0.956 | 0.963 | 0.941 | 0.979 | 0.829 | 0.796 |
| 131 | 0.921 | 0.876 | 0.956 | 0.959 | 0.931 | 0.978 | 0.828 | 0.788 |
| 132 | 0.923 | 0.878 | 0.957 | 0.960 | 0.932 | 0.978 | 0.829 | 0.790 |
| 133 | 0.921 | 0.878 | 0.956 | 0.959 | 0.924 | 0.978 | 0.831 | 0.799 |
| 134 | 0.918 | 0.871 | 0.956 | 0.957 | 0.915 | 0.978 | 0.828 | 0.791 |
| 135 | 0.920 | 0.872 | 0.956 | 0.957 | 0.913 | 0.979 | 0.830 | 0.793 |
| 136 | 0.921 | 0.883 | 0.955 | 0.961 | 0.939 | 0.978 | 0.829 | 0.798 |
| 137 | 0.923 | 0.885 | 0.955 | 0.962 | 0.939 | 0.979 | 0.831 | 0.800 |
| 138 | 0.921 | 0.876 | 0.956 | 0.958 | 0.928 | 0.978 | 0.829 | 0.792 |
| 139 | 0.922 | 0.878 | 0.957 | 0.959 | 0.929 | 0.978 | 0.831 | 0.795 |
| 140 | 0.921 | 0.877 | 0.956 | 0.960 | 0.930 | 0.979 | 0.827 | 0.792 |
| 141 | 0.922 | 0.879 | 0.956 | 0.961 | 0.929 | 0.979 | 0.829 | 0.795 |
| 142 | 0.919 | 0.872 | 0.956 | 0.959 | 0.921 | 0.979 | 0.826 | 0.789 |
| 143 | 0.920 | 0.873 | 0.957 | 0.959 | 0.918 | 0.979 | 0.828 | 0.791 |
| 144 | 0.922 | 0.883 | 0.955 | 0.963 | 0.942 | 0.979 | 0.826 | 0.793 |
| 145 | 0.923 | 0.886 | 0.956 | 0.964 | 0.943 | 0.980 | 0.828 | 0.795 |
| 146 | 0.921 | 0.876 | 0.956 | 0.960 | 0.933 | 0.979 | 0.826 | 0.789 |
| 147 | 0.923 | 0.879 | 0.957 | 0.961 | 0.934 | 0.979 | 0.829 | 0.791 |
| 148 | 0.920 | 0.875 | 0.955 | 0.957 | 0.925 | 0.978 | 0.827 | 0.790 |
| 149 | 0.921 | 0.877 | 0.956 | 0.959 | 0.925 | 0.978 | 0.829 | 0.792 |
| 150 | 0.918 | 0.870 | 0.956 | 0.957 | 0.916 | 0.978 | 0.826 | 0.784 |
| 151 | 0.919 | 0.871 | 0.957 | 0.958 | 0.914 | 0.978 | 0.828 | 0.787 |
| 152 | 0.921 | 0.882 | 0.955 | 0.961 | 0.939 | 0.978 | 0.827 | 0.791 |
| 153 | 0.922 | 0.884 | 0.956 | 0.962 | 0.940 | 0.979 | 0.829 | 0.793 |
| 154 | 0.920 | 0.875 | 0.956 | 0.958 | 0.929 | 0.977 | 0.827 | 0.786 |
| 155 | 0.922 | 0.877 | 0.957 | 0.959 | 0.930 | 0.978 | 0.829 | 0.788 |
| 156 | 0.920 | 0.875 | 0.955 | 0.957 | 0.925 | 0.978 | 0.827 | 0.790 |
| 157 | 0.921 | 0.877 | 0.956 | 0.958 | 0.925 | 0.978 | 0.829 | 0.793 |
| 158 | 0.918 | 0.870 | 0.956 | 0.957 | 0.916 | 0.978 | 0.826 | 0.785 |
| 159 | 0.919 | 0.871 | 0.956 | 0.957 | 0.913 | 0.978 | 0.828 | 0.788 |
| 160 | 0.921 | 0.882 | 0.954 | 0.961 | 0.939 | 0.978 | 0.827 | 0.791 |
| 161 | 0.922 | 0.884 | 0.955 | 0.962 | 0.939 | 0.978 | 0.829 | 0.794 |
| 162 | 0.920 | 0.875 | 0.956 | 0.958 | 0.929 | 0.977 | 0.827 | 0.786 |
| 163 | 0.922 | 0.877 | 0.957 | 0.959 | 0.930 | 0.978 | 0.829 | 0.789 |
| 164 | 0.920 | 0.876 | 0.955 | 0.957 | 0.925 | 0.978 | 0.830 | 0.796 |
| 165 | 0.915 | 0.871 | 0.952 | 0.952 | 0.917 | 0.973 | 0.825 | 0.787 |
| 166 | 0.915 | 0.868 | 0.953 | 0.954 | 0.908 | 0.977 | 0.828 | 0.793 |
| 167 | 0.916 | 0.870 | 0.953 | 0.959 | 0.921 | 0.978 | 0.820 | 0.786 |
| 168 | 0.913 | 0.862 | 0.953 | 0.949 | 0.903 | 0.974 | 0.822 | 0.774 |
| 169 | 0.910 | 0.861 | 0.950 | 0.949 | 0.903 | 0.973 | 0.820 | 0.777 |
| 170 | 0.917 | 0.870 | 0.954 | 0.954 | 0.905 | 0.977 | 0.830 | 0.796 |
| 171 | 0.917 | 0.870 | 0.954 | 0.958 | 0.917 | 0.978 | 0.821 | 0.787 |
| 172 | 0.914 | 0.862 | 0.954 | 0.949 | 0.898 | 0.974 | 0.824 | 0.776 |
| 173 | 0.918 | 0.875 | 0.953 | 0.955 | 0.920 | 0.977 | 0.830 | 0.798 |
| 174 | 0.911 | 0.862 | 0.950 | 0.949 | 0.899 | 0.973 | 0.822 | 0.779 |
| 175 | 0.920 | 0.883 | 0.952 | 0.959 | 0.936 | 0.977 | 0.830 | 0.800 |
| 176 | 0.920 | 0.882 | 0.953 | 0.962 | 0.943 | 0.978 | 0.821 | 0.790 |
| 177 | 0.917 | 0.877 | 0.952 | 0.955 | 0.932 | 0.974 | 0.824 | 0.783 |
| 178 | 0.916 | 0.877 | 0.950 | 0.955 | 0.934 | 0.974 | 0.823 | 0.786 |
| 179 | 0.921 | 0.885 | 0.954 | 0.961 | 0.937 | 0.978 | 0.832 | 0.803 |
| 180 | 0.921 | 0.884 | 0.953 | 0.964 | 0.943 | 0.979 | 0.823 | 0.792 |
| 181 | 0.918 | 0.879 | 0.953 | 0.956 | 0.932 | 0.975 | 0.827 | 0.787 |
| 182 | 0.919 | 0.875 | 0.954 | 0.959 | 0.930 | 0.978 | 0.822 | 0.789 |
| 183 | 0.917 | 0.880 | 0.951 | 0.957 | 0.935 | 0.974 | 0.825 | 0.790 |
| 184 | 0.919 | 0.874 | 0.954 | 0.955 | 0.924 | 0.976 | 0.830 | 0.794 |
| 185 | 0.918 | 0.874 | 0.954 | 0.959 | 0.932 | 0.978 | 0.821 | 0.785 |
| 186 | 0.916 | 0.868 | 0.954 | 0.950 | 0.919 | 0.973 | 0.824 | 0.776 |
| 187 | 0.914 | 0.868 | 0.951 | 0.950 | 0.921 | 0.972 | 0.822 | 0.778 |
| 188 | 0.920 | 0.876 | 0.955 | 0.956 | 0.925 | 0.977 | 0.831 | 0.797 |
| 189 | 0.920 | 0.875 | 0.954 | 0.960 | 0.933 | 0.978 | 0.823 | 0.787 |
| 190 | 0.917 | 0.870 | 0.955 | 0.952 | 0.920 | 0.973 | 0.826 | 0.779 |
| 191 | 0.916 | 0.869 | 0.953 | 0.950 | 0.915 | 0.973 | 0.824 | 0.780 |
| 192 | 0.915 | 0.870 | 0.952 | 0.952 | 0.922 | 0.973 | 0.824 | 0.782 |
| 193 | 0.914 | 0.869 | 0.951 | 0.950 | 0.917 | 0.973 | 0.823 | 0.783 |
| 194 | 0.920 | 0.877 | 0.954 | 0.956 | 0.920 | 0.977 | 0.832 | 0.801 |
| 195 | 0.920 | 0.877 | 0.954 | 0.960 | 0.929 | 0.978 | 0.824 | 0.791 |
| 196 | 0.917 | 0.870 | 0.954 | 0.951 | 0.914 | 0.973 | 0.826 | 0.784 |
| 197 | 0.918 | 0.871 | 0.955 | 0.957 | 0.917 | 0.978 | 0.826 | 0.788 |
| 198 | 0.919 | 0.871 | 0.956 | 0.958 | 0.915 | 0.978 | 0.828 | 0.790 |
| 199 | 0.884 | 0.830 | 0.929 | 0.924 | 0.894 | 0.944 | 0.764 | 0.707 |

| Panel | HFpEF | | HFrEF | HFrEF | | ICMP | ICMP |
|---|---|---|---|---|---|---|---|
| Number | symptomatic | HFrEF | asymptomatic | symptomatic | ICMP | asymptomatic | symptomatic |
| 104 | 0.872 | 0.966 | 0.942 | 0.979 | 0.972 | 0.961 | 0.975 |
| 105 | 0.870 | 0.967 | 0.946 | 0.979 | 0.976 | 0.965 | 0.978 |
| 106 | 0.866 | 0.965 | 0.943 | 0.976 | 0.974 | 0.961 | 0.977 |
| 107 | 0.858 | 0.965 | 0.943 | 0.978 | 0.971 | 0.960 | 0.974 |
| 108 | 0.867 | 0.964 | 0.942 | 0.976 | 0.972 | 0.961 | 0.974 |
| 109 | 0.870 | 0.962 | 0.939 | 0.976 | 0.973 | 0.962 | 0.975 |
| 110 | 0.873 | 0.960 | 0.938 | 0.973 | 0.972 | 0.961 | 0.975 |
| 111 | 0.870 | 0.962 | 0.939 | 0.975 | 0.971 | 0.962 | 0.973 |
| 112 | 0.873 | 0.960 | 0.938 | 0.972 | 0.970 | 0.962 | 0.972 |
| 113 | 0.862 | 0.961 | 0.937 | 0.974 | 0.970 | 0.959 | 0.973 |
| 114 | 0.864 | 0.958 | 0.936 | 0.971 | 0.970 | 0.958 | 0.973 |
| 115 | 0.862 | 0.959 | 0.936 | 0.973 | 0.968 | 0.959 | 0.970 |
| 116 | 0.879 | 0.965 | 0.945 | 0.977 | 0.975 | 0.965 | 0.978 |
| 117 | 0.864 | 0.957 | 0.935 | 0.970 | 0.967 | 0.958 | 0.969 |
| 118 | 0.870 | 0.966 | 0.945 | 0.979 | 0.974 | 0.965 | 0.976 |
| 119 | 0.879 | 0.965 | 0.945 | 0.976 | 0.974 | 0.966 | 0.975 |
| 120 | 0.864 | 0.964 | 0.940 | 0.977 | 0.972 | 0.960 | 0.975 |
| 121 | 0.869 | 0.961 | 0.938 | 0.974 | 0.972 | 0.960 | 0.974 |
| 122 | 0.864 | 0.962 | 0.939 | 0.976 | 0.970 | 0.960 | 0.972 |
| 123 | 0.870 | 0.960 | 0.938 | 0.973 | 0.969 | 0.960 | 0.971 |
| 124 | 0.858 | 0.966 | 0.943 | 0.978 | 0.973 | 0.960 | 0.977 |
| 125 | 0.869 | 0.969 | 0.947 | 0.980 | 0.980 | 0.965 | 0.984 |
| 126 | 0.870 | 0.969 | 0.947 | 0.981 | 0.980 | 0.972 | 0.983 |
| 127 | 0.873 | 0.966 | 0.942 | 0.979 | 0.976 | 0.966 | 0.979 |
| 128 | 0.873 | 0.967 | 0.944 | 0.980 | 0.977 | 0.972 | 0.979 |
| 129 | 0.867 | 0.971 | 0.957 | 0.980 | 0.980 | 0.973 | 0.981 |
| 130 | 0.869 | 0.972 | 0.957 | 0.981 | 0.981 | 0.976 | 0.981 |
| 131 | 0.871 | 0.969 | 0.951 | 0.980 | 0.980 | 0.971 | 0.982 |
| 132 | 0.871 | 0.970 | 0.951 | 0.980 | 0.981 | 0.975 | 0.982 |
| 133 | 0.869 | 0.968 | 0.946 | 0.980 | 0.980 | 0.971 | 0.983 |
| 134 | 0.871 | 0.966 | 0.941 | 0.979 | 0.976 | 0.965 | 0.978 |
| 135 | 0.871 | 0.966 | 0.942 | 0.979 | 0.977 | 0.971 | 0.978 |
| 136 | 0.866 | 0.970 | 0.956 | 0.980 | 0.980 | 0.972 | 0.981 |
| 137 | 0.867 | 0.971 | 0.956 | 0.981 | 0.980 | 0.975 | 0.981 |
| 138 | 0.869 | 0.968 | 0.950 | 0.979 | 0.980 | 0.970 | 0.981 |
| 139 | 0.870 | 0.969 | 0.950 | 0.980 | 0.980 | 0.974 | 0.981 |
| 140 | 0.868 | 0.969 | 0.947 | 0.980 | 0.979 | 0.964 | 0.984 |
| 141 | 0.869 | 0.969 | 0.947 | 0.981 | 0.980 | 0.971 | 0.983 |
| 142 | 0.871 | 0.967 | 0.942 | 0.979 | 0.976 | 0.965 | 0.978 |
| 143 | 0.871 | 0.967 | 0.944 | 0.980 | 0.977 | 0.971 | 0.979 |
| 144 | 0.866 | 0.971 | 0.956 | 0.980 | 0.980 | 0.972 | 0.981 |
| 145 | 0.867 | 0.972 | 0.956 | 0.981 | 0.980 | 0.975 | 0.981 |
| 146 | 0.869 | 0.969 | 0.951 | 0.980 | 0.980 | 0.970 | 0.982 |
| 147 | 0.870 | 0.970 | 0.951 | 0.980 | 0.980 | 0.975 | 0.982 |
| 148 | 0.870 | 0.968 | 0.946 | 0.980 | 0.979 | 0.964 | 0.984 |
| 149 | 0.872 | 0.969 | 0.946 | 0.980 | 0.980 | 0.971 | 0.983 |
| 150 | 0.874 | 0.966 | 0.941 | 0.979 | 0.976 | 0.965 | 0.978 |
| 151 | 0.875 | 0.967 | 0.943 | 0.979 | 0.977 | 0.971 | 0.978 |
| 152 | 0.868 | 0.971 | 0.956 | 0.980 | 0.980 | 0.972 | 0.981 |
| 153 | 0.870 | 0.971 | 0.956 | 0.981 | 0.980 | 0.976 | 0.981 |
| 154 | 0.872 | 0.969 | 0.950 | 0.979 | 0.980 | 0.971 | 0.982 |
| 155 | 0.873 | 0.969 | 0.950 | 0.980 | 0.980 | 0.975 | 0.982 |
| 156 | 0.867 | 0.968 | 0.946 | 0.980 | 0.979 | 0.964 | 0.984 |
| 157 | 0.869 | 0.968 | 0.946 | 0.980 | 0.980 | 0.970 | 0.983 |
| 158 | 0.871 | 0.966 | 0.941 | 0.979 | 0.976 | 0.965 | 0.978 |
| 159 | 0.871 | 0.967 | 0.942 | 0.979 | 0.977 | 0.970 | 0.978 |
| 160 | 0.865 | 0.971 | 0.956 | 0.980 | 0.980 | 0.972 | 0.981 |
| 161 | 0.867 | 0.971 | 0.956 | 0.981 | 0.980 | 0.975 | 0.981 |
| 162 | 0.869 | 0.969 | 0.950 | 0.979 | 0.980 | 0.970 | 0.982 |
| 163 | 0.870 | 0.969 | 0.950 | 0.980 | 0.980 | 0.974 | 0.982 |
| 164 | 0.868 | 0.968 | 0.946 | 0.980 | 0.979 | 0.964 | 0.984 |
| 165 | 0.863 | 0.963 | 0.938 | 0.976 | 0.976 | 0.962 | 0.980 |
| 166 | 0.867 | 0.962 | 0.935 | 0.976 | 0.972 | 0.961 | 0.975 |
| 167 | 0.860 | 0.965 | 0.940 | 0.978 | 0.973 | 0.961 | 0.977 |
| 168 | 0.874 | 0.961 | 0.934 | 0.975 | 0.973 | 0.964 | 0.975 |
| 169 | 0.865 | 0.959 | 0.932 | 0.973 | 0.970 | 0.959 | 0.972 |
| 170 | 0.867 | 0.963 | 0.935 | 0.977 | 0.972 | 0.964 | 0.975 |
| 171 | 0.861 | 0.965 | 0.939 | 0.978 | 0.973 | 0.965 | 0.976 |
| 172 | 0.875 | 0.961 | 0.933 | 0.976 | 0.974 | 0.967 | 0.975 |
| 173 | 0.864 | 0.966 | 0.942 | 0.978 | 0.978 | 0.961 | 0.982 |
| 174 | 0.866 | 0.959 | 0.930 | 0.974 | 0.970 | 0.961 | 0.972 |
| 175 | 0.861 | 0.969 | 0.953 | 0.979 | 0.978 | 0.970 | 0.979 |
| 176 | 0.855 | 0.970 | 0.955 | 0.980 | 0.979 | 0.969 | 0.981 |
| 177 | 0.868 | 0.967 | 0.952 | 0.978 | 0.979 | 0.972 | 0.980 |
| 178 | 0.859 | 0.967 | 0.951 | 0.977 | 0.977 | 0.970 | 0.978 |
| 179 | 0.863 | 0.969 | 0.953 | 0.980 | 0.978 | 0.972 | 0.979 |
| 180 | 0.857 | 0.971 | 0.955 | 0.980 | 0.978 | 0.970 | 0.980 |
| 181 | 0.870 | 0.968 | 0.952 | 0.978 | 0.978 | 0.973 | 0.979 |
| 182 | 0.858 | 0.968 | 0.945 | 0.979 | 0.978 | 0.961 | 0.983 |
| 183 | 0.861 | 0.967 | 0.951 | 0.978 | 0.977 | 0.970 | 0.978 |
| 184 | 0.865 | 0.966 | 0.946 | 0.978 | 0.978 | 0.967 | 0.979 |
| 185 | 0.858 | 0.968 | 0.948 | 0.979 | 0.978 | 0.966 | 0.980 |
| 186 | 0.872 | 0.965 | 0.945 | 0.976 | 0.979 | 0.970 | 0.980 |
| 187 | 0.864 | 0.963 | 0.943 | 0.975 | 0.976 | 0.967 | 0.978 |
| 188 | 0.866 | 0.966 | 0.946 | 0.978 | 0.978 | 0.970 | 0.979 |
| 189 | 0.860 | 0.968 | 0.948 | 0.979 | 0.978 | 0.967 | 0.980 |
| 190 | 0.873 | 0.965 | 0.945 | 0.977 | 0.978 | 0.974 | 0.979 |
| 191 | 0.870 | 0.964 | 0.941 | 0.977 | 0.978 | 0.963 | 0.982 |
| 192 | 0.864 | 0.964 | 0.944 | 0.976 | 0.976 | 0.969 | 0.977 |
| 193 | 0.862 | 0.963 | 0.939 | 0.976 | 0.976 | 0.960 | 0.981 |
| 194 | 0.865 | 0.966 | 0.941 | 0.979 | 0.977 | 0.965 | 0.982 |
| 195 | 0.860 | 0.968 | 0.944 | 0.980 | 0.977 | 0.964 | 0.982 |
| 196 | 0.871 | 0.964 | 0.939 | 0.977 | 0.977 | 0.967 | 0.981 |
| 197 | 0.872 | 0.965 | 0.941 | 0.978 | 0.974 | 0.963 | 0.976 |
| 198 | 0.872 | 0.966 | 0.943 | 0.979 | 0.976 | 0.972 | 0.977 |
| 199 | 0.819 | 0.947 | 0.920 | 0.962 | 0.971 | 0.951 | 0.977 |

TABLE 4C
AUC values for Panel Number 104 to 199 without NT-proBNP and with
ANOVA correction for age, BMI and gender in different CHF sub-groups

| Panel | | CHF | CHF | | DCMP | DCMP | | HFpEF |
|---|---|---|---|---|---|---|---|---|
| Number | CHF | asymptomatic | symptomatic | DCMP | asymptomatic | symptomatic | HFpEF | asymptomatic |
| 104 | 0.787 | 0.740 | 0.822 | 0.798 | 0.723 | 0.830 | 0.703 | 0.662 |
| 105 | 0.788 | 0.739 | 0.822 | 0.797 | 0.720 | 0.828 | 0.704 | 0.657 |
| 106 | 0.782 | 0.739 | 0.813 | 0.796 | 0.726 | 0.819 | 0.703 | 0.664 |
| 107 | 0.788 | 0.744 | 0.820 | 0.798 | 0.720 | 0.826 | 0.708 | 0.669 |
| 108 | 0.782 | 0.740 | 0.812 | 0.795 | 0.725 | 0.819 | 0.702 | 0.662 |
| 109 | 0.772 | 0.718 | 0.812 | 0.776 | 0.657 | 0.816 | 0.687 | 0.619 |
| 110 | 0.763 | 0.714 | 0.799 | 0.774 | 0.667 | 0.806 | 0.675 | 0.610 |
| 111 | 0.772 | 0.719 | 0.812 | 0.776 | 0.657 | 0.816 | 0.687 | 0.619 |
| 112 | 0.764 | 0.715 | 0.798 | 0.774 | 0.667 | 0.806 | 0.675 | 0.614 |
| 113 | 0.766 | 0.715 | 0.804 | 0.766 | 0.633 | 0.806 | 0.689 | 0.627 |
| 114 | 0.756 | 0.710 | 0.790 | 0.764 | 0.642 | 0.795 | 0.675 | 0.613 |
| 115 | 0.766 | 0.716 | 0.803 | 0.765 | 0.633 | 0.805 | 0.688 | 0.629 |
| 116 | 0.780 | 0.734 | 0.812 | 0.793 | 0.720 | 0.820 | 0.697 | 0.650 |
| 117 | 0.756 | 0.711 | 0.789 | 0.763 | 0.644 | 0.794 | 0.674 | 0.616 |
| 118 | 0.787 | 0.739 | 0.821 | 0.796 | 0.717 | 0.828 | 0.703 | 0.658 |
| 119 | 0.780 | 0.735 | 0.812 | 0.793 | 0.720 | 0.820 | 0.697 | 0.650 |
| 120 | 0.781 | 0.733 | 0.817 | 0.784 | 0.674 | 0.824 | 0.710 | 0.671 |
| 121 | 0.771 | 0.726 | 0.804 | 0.780 | 0.679 | 0.812 | 0.698 | 0.659 |
| 122 | 0.781 | 0.733 | 0.816 | 0.783 | 0.673 | 0.823 | 0.708 | 0.672 |
| 123 | 0.771 | 0.727 | 0.803 | 0.779 | 0.678 | 0.811 | 0.698 | 0.658 |
| 124 | 0.789 | 0.744 | 0.822 | 0.799 | 0.725 | 0.827 | 0.709 | 0.671 |
| 125 | 0.809 | 0.757 | 0.850 | 0.813 | 0.745 | 0.839 | 0.719 | 0.665 |
| 126 | 0.810 | 0.758 | 0.851 | 0.812 | 0.741 | 0.841 | 0.723 | 0.669 |
| 127 | 0.805 | 0.746 | 0.852 | 0.813 | 0.727 | 0.846 | 0.713 | 0.659 |
| 128 | 0.807 | 0.747 | 0.855 | 0.815 | 0.720 | 0.852 | 0.716 | 0.660 |
| 129 | 0.800 | 0.751 | 0.835 | 0.810 | 0.755 | 0.834 | 0.710 | 0.659 |
| 130 | 0.799 | 0.751 | 0.835 | 0.809 | 0.744 | 0.834 | 0.713 | 0.661 |
| 131 | 0.812 | 0.756 | 0.856 | 0.819 | 0.761 | 0.844 | 0.723 | 0.658 |
| 132 | 0.815 | 0.759 | 0.860 | 0.822 | 0.763 | 0.849 | 0.727 | 0.664 |
| 133 | 0.810 | 0.757 | 0.851 | 0.810 | 0.730 | 0.842 | 0.727 | 0.681 |
| 134 | 0.803 | 0.744 | 0.851 | 0.809 | 0.720 | 0.846 | 0.716 | 0.669 |
| 135 | 0.805 | 0.744 | 0.854 | 0.812 | 0.711 | 0.852 | 0.719 | 0.668 |
| 136 | 0.799 | 0.750 | 0.835 | 0.808 | 0.749 | 0.835 | 0.714 | 0.670 |
| 137 | 0.799 | 0.750 | 0.835 | 0.806 | 0.737 | 0.835 | 0.718 | 0.673 |
| 138 | 0.812 | 0.755 | 0.857 | 0.816 | 0.755 | 0.845 | 0.727 | 0.672 |
| 139 | 0.814 | 0.758 | 0.860 | 0.819 | 0.755 | 0.850 | 0.731 | 0.674 |
| 140 | 0.812 | 0.761 | 0.852 | 0.817 | 0.754 | 0.842 | 0.723 | 0.676 |
| 141 | 0.812 | 0.761 | 0.852 | 0.815 | 0.746 | 0.843 | 0.726 | 0.678 |
| 142 | 0.806 | 0.749 | 0.852 | 0.814 | 0.734 | 0.846 | 0.716 | 0.669 |
| 143 | 0.808 | 0.749 | 0.855 | 0.815 | 0.721 | 0.851 | 0.719 | 0.668 |
| 144 | 0.802 | 0.755 | 0.836 | 0.813 | 0.759 | 0.837 | 0.713 | 0.670 |
| 145 | 0.801 | 0.754 | 0.836 | 0.810 | 0.750 | 0.835 | 0.717 | 0.670 |
| 146 | 0.814 | 0.759 | 0.857 | 0.821 | 0.764 | 0.845 | 0.725 | 0.671 |
| 147 | 0.816 | 0.762 | 0.860 | 0.822 | 0.763 | 0.850 | 0.729 | 0.672 |
| 148 | 0.808 | 0.755 | 0.849 | 0.810 | 0.743 | 0.837 | 0.718 | 0.664 |
| 149 | 0.809 | 0.756 | 0.850 | 0.809 | 0.739 | 0.839 | 0.721 | 0.668 |
| 150 | 0.803 | 0.744 | 0.851 | 0.810 | 0.725 | 0.845 | 0.712 | 0.658 |
| 151 | 0.805 | 0.745 | 0.854 | 0.812 | 0.719 | 0.851 | 0.715 | 0.658 |
| 152 | 0.798 | 0.749 | 0.834 | 0.808 | 0.753 | 0.833 | 0.708 | 0.657 |
| 153 | 0.798 | 0.749 | 0.834 | 0.806 | 0.742 | 0.833 | 0.712 | 0.661 |
| 154 | 0.811 | 0.754 | 0.856 | 0.816 | 0.759 | 0.843 | 0.721 | 0.656 |
| 155 | 0.814 | 0.757 | 0.859 | 0.818 | 0.760 | 0.848 | 0.725 | 0.662 |
| 156 | 0.808 | 0.755 | 0.849 | 0.810 | 0.745 | 0.837 | 0.717 | 0.660 |
| 157 | 0.808 | 0.756 | 0.850 | 0.809 | 0.737 | 0.839 | 0.721 | 0.665 |
| 158 | 0.803 | 0.744 | 0.851 | 0.810 | 0.727 | 0.844 | 0.711 | 0.654 |
| 159 | 0.805 | 0.745 | 0.853 | 0.813 | 0.717 | 0.850 | 0.714 | 0.656 |
| 160 | 0.798 | 0.749 | 0.834 | 0.808 | 0.751 | 0.832 | 0.708 | 0.655 |
| 161 | 0.798 | 0.749 | 0.834 | 0.806 | 0.740 | 0.832 | 0.712 | 0.659 |
| 162 | 0.811 | 0.755 | 0.855 | 0.816 | 0.757 | 0.842 | 0.721 | 0.654 |
| 163 | 0.813 | 0.757 | 0.858 | 0.818 | 0.757 | 0.848 | 0.725 | 0.659 |
| 164 | 0.809 | 0.756 | 0.850 | 0.811 | 0.738 | 0.840 | 0.724 | 0.677 |
| 165 | 0.795 | 0.739 | 0.840 | 0.785 | 0.673 | 0.820 | 0.714 | 0.656 |
| 166 | 0.797 | 0.739 | 0.845 | 0.798 | 0.681 | 0.838 | 0.717 | 0.670 |
| 167 | 0.802 | 0.749 | 0.846 | 0.809 | 0.735 | 0.836 | 0.715 | 0.669 |
| 168 | 0.788 | 0.724 | 0.842 | 0.790 | 0.669 | 0.833 | 0.695 | 0.618 |
| 169 | 0.782 | 0.721 | 0.833 | 0.780 | 0.645 | 0.821 | 0.697 | 0.631 |
| 170 | 0.799 | 0.740 | 0.849 | 0.800 | 0.667 | 0.845 | 0.721 | 0.673 |
| 171 | 0.803 | 0.748 | 0.848 | 0.807 | 0.715 | 0.838 | 0.719 | 0.670 |
| 172 | 0.789 | 0.722 | 0.844 | 0.791 | 0.649 | 0.839 | 0.700 | 0.625 |
| 173 | 0.806 | 0.753 | 0.849 | 0.803 | 0.718 | 0.835 | 0.726 | 0.681 |
| 174 | 0.783 | 0.721 | 0.836 | 0.781 | 0.627 | 0.828 | 0.702 | 0.638 |
| 175 | 0.796 | 0.747 | 0.833 | 0.801 | 0.726 | 0.831 | 0.717 | 0.675 |
| 176 | 0.801 | 0.755 | 0.834 | 0.812 | 0.759 | 0.832 | 0.714 | 0.672 |
| 177 | 0.787 | 0.733 | 0.827 | 0.792 | 0.713 | 0.823 | 0.695 | 0.626 |
| 178 | 0.783 | 0.732 | 0.822 | 0.785 | 0.703 | 0.815 | 0.698 | 0.637 |
| 179 | 0.797 | 0.749 | 0.834 | 0.801 | 0.716 | 0.832 | 0.721 | 0.678 |
| 180 | 0.799 | 0.754 | 0.833 | 0.807 | 0.749 | 0.828 | 0.718 | 0.672 |
| 181 | 0.785 | 0.731 | 0.826 | 0.788 | 0.695 | 0.822 | 0.700 | 0.634 |
| 182 | 0.811 | 0.761 | 0.850 | 0.816 | 0.756 | 0.837 | 0.724 | 0.677 |
| 183 | 0.783 | 0.732 | 0.822 | 0.782 | 0.677 | 0.815 | 0.704 | 0.648 |
| 184 | 0.808 | 0.752 | 0.854 | 0.808 | 0.734 | 0.840 | 0.730 | 0.676 |
| 185 | 0.811 | 0.759 | 0.853 | 0.817 | 0.763 | 0.838 | 0.726 | 0.673 |
| 186 | 0.799 | 0.738 | 0.848 | 0.799 | 0.724 | 0.833 | 0.708 | 0.625 |
| 187 | 0.795 | 0.736 | 0.843 | 0.791 | 0.710 | 0.824 | 0.711 | 0.638 |
| 188 | 0.811 | 0.757 | 0.857 | 0.811 | 0.735 | 0.846 | 0.734 | 0.678 |
| 189 | 0.812 | 0.761 | 0.855 | 0.816 | 0.759 | 0.840 | 0.730 | 0.672 |
| 190 | 0.801 | 0.740 | 0.851 | 0.800 | 0.723 | 0.838 | 0.714 | 0.633 |
| 191 | 0.797 | 0.739 | 0.842 | 0.793 | 0.703 | 0.826 | 0.705 | 0.635 |
| 192 | 0.797 | 0.740 | 0.846 | 0.793 | 0.709 | 0.830 | 0.717 | 0.649 |
| 193 | 0.794 | 0.738 | 0.839 | 0.787 | 0.693 | 0.819 | 0.709 | 0.646 |
| 194 | 0.808 | 0.756 | 0.850 | 0.803 | 0.709 | 0.838 | 0.730 | 0.684 |
| 195 | 0.810 | 0.762 | 0.850 | 0.812 | 0.745 | 0.836 | 0.727 | 0.680 |
| 196 | 0.796 | 0.738 | 0.842 | 0.789 | 0.685 | 0.826 | 0.709 | 0.641 |
| 197 | 0.799 | 0.743 | 0.843 | 0.810 | 0.723 | 0.843 | 0.704 | 0.658 |
| 198 | 0.800 | 0.743 | 0.846 | 0.811 | 0.714 | 0.849 | 0.708 | 0.655 |
| 199 | 0.754 | 0.702 | 0.801 | 0.739 | 0.646 | 0.757 | 0.672 | 0.558 |

| Panel | HFpEF | | HFrEF | HFrEF | | ICMP | ICMP |
|---|---|---|---|---|---|---|---|
| Number | symptomatic | HFrEF | asymptomatic | symptomatic | ICMP | asymptomatic | symptomatic |
| 104 | 0.717 | 0.831 | 0.788 | 0.853 | 0.862 | 0.836 | 0.867 |
| 105 | 0.720 | 0.831 | 0.785 | 0.853 | 0.865 | 0.833 | 0.874 |
| 106 | 0.700 | 0.825 | 0.782 | 0.845 | 0.857 | 0.822 | 0.868 |
| 107 | 0.705 | 0.830 | 0.785 | 0.851 | 0.857 | 0.828 | 0.861 |
| 108 | 0.688 | 0.824 | 0.783 | 0.844 | 0.852 | 0.821 | 0.858 |
| 109 | 0.710 | 0.817 | 0.766 | 0.841 | 0.855 | 0.824 | 0.863 |
| 110 | 0.678 | 0.811 | 0.764 | 0.832 | 0.848 | 0.812 | 0.859 |
| 111 | 0.693 | 0.817 | 0.766 | 0.841 | 0.851 | 0.823 | 0.853 |
| 112 | 0.667 | 0.810 | 0.764 | 0.831 | 0.844 | 0.811 | 0.848 |
| 113 | 0.704 | 0.806 | 0.754 | 0.832 | 0.847 | 0.814 | 0.855 |
| 114 | 0.673 | 0.800 | 0.752 | 0.822 | 0.839 | 0.801 | 0.852 |
| 115 | 0.692 | 0.806 | 0.754 | 0.831 | 0.842 | 0.810 | 0.843 |
| 116 | 0.703 | 0.825 | 0.782 | 0.845 | 0.859 | 0.826 | 0.870 |
| 117 | 0.657 | 0.800 | 0.753 | 0.821 | 0.834 | 0.800 | 0.839 |
| 118 | 0.711 | 0.831 | 0.785 | 0.852 | 0.861 | 0.831 | 0.864 |
| 119 | 0.695 | 0.824 | 0.782 | 0.844 | 0.855 | 0.825 | 0.859 |
| 120 | 0.718 | 0.820 | 0.767 | 0.846 | 0.856 | 0.821 | 0.866 |
| 121 | 0.695 | 0.812 | 0.764 | 0.836 | 0.848 | 0.810 | 0.861 |
| 122 | 0.710 | 0.819 | 0.767 | 0.845 | 0.851 | 0.816 | 0.855 |
| 123 | 0.683 | 0.812 | 0.764 | 0.835 | 0.843 | 0.809 | 0.849 |
| 124 | 0.715 | 0.830 | 0.785 | 0.852 | 0.862 | 0.829 | 0.872 |
| 125 | 0.756 | 0.858 | 0.813 | 0.880 | 0.906 | 0.863 | 0.919 |
| 126 | 0.759 | 0.855 | 0.810 | 0.880 | 0.904 | 0.866 | 0.912 |
| 127 | 0.766 | 0.853 | 0.806 | 0.876 | 0.892 | 0.863 | 0.896 |
| 128 | 0.763 | 0.855 | 0.806 | 0.880 | 0.894 | 0.867 | 0.896 |
| 129 | 0.724 | 0.848 | 0.807 | 0.868 | 0.885 | 0.851 | 0.892 |
| 130 | 0.728 | 0.845 | 0.804 | 0.866 | 0.882 | 0.852 | 0.885 |
| 131 | 0.777 | 0.860 | 0.823 | 0.880 | 0.903 | 0.873 | 0.908 |
| 132 | 0.776 | 0.861 | 0.824 | 0.882 | 0.904 | 0.877 | 0.906 |
| 133 | 0.760 | 0.854 | 0.806 | 0.880 | 0.902 | 0.862 | 0.912 |
| 134 | 0.765 | 0.851 | 0.803 | 0.875 | 0.890 | 0.859 | 0.895 |
| 135 | 0.762 | 0.853 | 0.803 | 0.878 | 0.892 | 0.863 | 0.894 |
| 136 | 0.725 | 0.847 | 0.805 | 0.868 | 0.884 | 0.846 | 0.892 |
| 137 | 0.729 | 0.843 | 0.801 | 0.866 | 0.881 | 0.848 | 0.885 |
| 138 | 0.778 | 0.859 | 0.820 | 0.879 | 0.902 | 0.869 | 0.907 |
| 139 | 0.777 | 0.860 | 0.821 | 0.882 | 0.902 | 0.873 | 0.905 |
| 140 | 0.757 | 0.860 | 0.814 | 0.882 | 0.905 | 0.861 | 0.919 |
| 141 | 0.759 | 0.857 | 0.810 | 0.881 | 0.903 | 0.864 | 0.912 |
| 142 | 0.765 | 0.854 | 0.806 | 0.877 | 0.891 | 0.862 | 0.896 |
| 143 | 0.762 | 0.855 | 0.806 | 0.880 | 0.893 | 0.866 | 0.895 |
| 144 | 0.724 | 0.850 | 0.808 | 0.870 | 0.885 | 0.849 | 0.893 |
| 145 | 0.727 | 0.846 | 0.804 | 0.867 | 0.881 | 0.850 | 0.885 |
| 146 | 0.776 | 0.861 | 0.822 | 0.881 | 0.902 | 0.871 | 0.908 |
| 147 | 0.776 | 0.862 | 0.823 | 0.883 | 0.903 | 0.875 | 0.905 |
| 148 | 0.754 | 0.857 | 0.810 | 0.878 | 0.904 | 0.860 | 0.918 |
| 149 | 0.757 | 0.854 | 0.807 | 0.878 | 0.902 | 0.862 | 0.911 |
| 150 | 0.764 | 0.852 | 0.803 | 0.875 | 0.891 | 0.859 | 0.895 |
| 151 | 0.761 | 0.853 | 0.803 | 0.878 | 0.892 | 0.863 | 0.894 |
| 152 | 0.722 | 0.847 | 0.805 | 0.867 | 0.884 | 0.847 | 0.892 |
| 153 | 0.726 | 0.843 | 0.801 | 0.864 | 0.880 | 0.848 | 0.884 |
| 154 | 0.775 | 0.859 | 0.820 | 0.878 | 0.902 | 0.870 | 0.907 |
| 155 | 0.775 | 0.860 | 0.821 | 0.881 | 0.902 | 0.874 | 0.904 |
| 156 | 0.755 | 0.857 | 0.811 | 0.878 | 0.904 | 0.859 | 0.918 |
| 157 | 0.758 | 0.854 | 0.808 | 0.878 | 0.902 | 0.861 | 0.911 |
| 158 | 0.764 | 0.852 | 0.804 | 0.875 | 0.890 | 0.859 | 0.895 |
| 159 | 0.761 | 0.853 | 0.805 | 0.878 | 0.892 | 0.862 | 0.894 |
| 160 | 0.722 | 0.847 | 0.806 | 0.867 | 0.884 | 0.846 | 0.892 |
| 161 | 0.726 | 0.843 | 0.802 | 0.864 | 0.880 | 0.847 | 0.884 |
| 162 | 0.775 | 0.859 | 0.821 | 0.878 | 0.901 | 0.868 | 0.907 |
| 163 | 0.775 | 0.860 | 0.822 | 0.880 | 0.902 | 0.872 | 0.904 |
| 164 | 0.758 | 0.857 | 0.810 | 0.879 | 0.904 | 0.859 | 0.918 |
| 165 | 0.754 | 0.837 | 0.783 | 0.866 | 0.892 | 0.850 | 0.903 |
| 166 | 0.763 | 0.840 | 0.787 | 0.868 | 0.881 | 0.850 | 0.886 |
| 167 | 0.759 | 0.849 | 0.803 | 0.870 | 0.885 | 0.857 | 0.890 |
| 168 | 0.757 | 0.838 | 0.785 | 0.864 | 0.882 | 0.854 | 0.885 |
| 169 | 0.751 | 0.827 | 0.774 | 0.853 | 0.871 | 0.844 | 0.874 |
| 170 | 0.760 | 0.841 | 0.787 | 0.871 | 0.882 | 0.854 | 0.885 |
| 171 | 0.758 | 0.847 | 0.801 | 0.869 | 0.883 | 0.858 | 0.885 |
| 172 | 0.756 | 0.837 | 0.782 | 0.866 | 0.881 | 0.857 | 0.881 |
| 173 | 0.758 | 0.851 | 0.800 | 0.877 | 0.902 | 0.854 | 0.917 |
| 174 | 0.751 | 0.826 | 0.771 | 0.855 | 0.870 | 0.846 | 0.870 |
| 175 | 0.724 | 0.841 | 0.794 | 0.865 | 0.880 | 0.841 | 0.888 |
| 176 | 0.721 | 0.848 | 0.806 | 0.868 | 0.882 | 0.847 | 0.891 |
| 177 | 0.716 | 0.837 | 0.790 | 0.858 | 0.878 | 0.844 | 0.885 |
| 178 | 0.714 | 0.830 | 0.782 | 0.853 | 0.873 | 0.836 | 0.881 |
| 179 | 0.730 | 0.838 | 0.791 | 0.864 | 0.877 | 0.842 | 0.882 |
| 180 | 0.725 | 0.841 | 0.800 | 0.863 | 0.876 | 0.844 | 0.881 |
| 181 | 0.722 | 0.830 | 0.783 | 0.854 | 0.872 | 0.843 | 0.874 |
| 182 | 0.755 | 0.858 | 0.812 | 0.880 | 0.903 | 0.859 | 0.919 |
| 183 | 0.718 | 0.824 | 0.776 | 0.851 | 0.867 | 0.834 | 0.872 |
| 184 | 0.778 | 0.852 | 0.809 | 0.875 | 0.896 | 0.863 | 0.902 |
| 185 | 0.771 | 0.857 | 0.818 | 0.876 | 0.897 | 0.865 | 0.904 |
| 186 | 0.772 | 0.848 | 0.807 | 0.869 | 0.896 | 0.867 | 0.900 |
| 187 | 0.769 | 0.839 | 0.798 | 0.862 | 0.888 | 0.858 | 0.893 |
| 188 | 0.777 | 0.853 | 0.810 | 0.878 | 0.897 | 0.868 | 0.900 |
| 189 | 0.772 | 0.855 | 0.817 | 0.876 | 0.895 | 0.867 | 0.898 |
| 190 | 0.772 | 0.847 | 0.806 | 0.870 | 0.895 | 0.871 | 0.894 |
| 191 | 0.749 | 0.846 | 0.797 | 0.870 | 0.900 | 0.857 | 0.913 |
| 192 | 0.769 | 0.839 | 0.798 | 0.864 | 0.888 | 0.862 | 0.888 |
| 193 | 0.750 | 0.841 | 0.789 | 0.866 | 0.896 | 0.850 | 0.911 |
| 194 | 0.761 | 0.849 | 0.796 | 0.878 | 0.900 | 0.857 | 0.910 |
| 195 | 0.758 | 0.854 | 0.806 | 0.878 | 0.899 | 0.858 | 0.909 |
| 196 | 0.753 | 0.841 | 0.789 | 0.868 | 0.896 | 0.858 | 0.903 |
| 197 | 0.743 | 0.848 | 0.803 | 0.870 | 0.884 | 0.861 | 0.885 |
| 198 | 0.747 | 0.849 | 0.803 | 0.872 | 0.885 | 0.865 | 0.882 |
| 199 | 0.746 | 0.799 | 0.770 | 0.819 | 0.871 | 0.851 | 0.875 |

TABLE 4D
AUC values for Panel Number 104 to 199 with NT-proBNP and with
ANOVA correction for age, BMI and gender in different CHF sub-groups

| Panel | | CHF | CHF | | DCMP | DCMP | | HFpEF |
|---|---|---|---|---|---|---|---|---|
| Number | CHF | asymptomatic | symptomatic | DCMP | asymptomatic | symptomatic | HFpEF | asymptomatic |
| 104 | 0.892 | 0.842 | 0.930 | 0.949 | 0.908 | 0.969 | 0.772 | 0.723 |
| 105 | 0.892 | 0.842 | 0.931 | 0.948 | 0.907 | 0.969 | 0.772 | 0.719 |
| 106 | 0.889 | 0.842 | 0.927 | 0.948 | 0.912 | 0.964 | 0.770 | 0.719 |
| 107 | 0.891 | 0.842 | 0.929 | 0.950 | 0.912 | 0.968 | 0.770 | 0.721 |
| 108 | 0.889 | 0.842 | 0.926 | 0.948 | 0.912 | 0.964 | 0.770 | 0.719 |
| 109 | 0.886 | 0.833 | 0.927 | 0.940 | 0.895 | 0.963 | 0.763 | 0.700 |
| 110 | 0.882 | 0.832 | 0.922 | 0.939 | 0.896 | 0.959 | 0.759 | 0.698 |
| 111 | 0.885 | 0.833 | 0.926 | 0.940 | 0.895 | 0.963 | 0.764 | 0.701 |
| 112 | 0.882 | 0.833 | 0.921 | 0.939 | 0.895 | 0.958 | 0.760 | 0.699 |
| 113 | 0.885 | 0.834 | 0.925 | 0.941 | 0.896 | 0.963 | 0.767 | 0.708 |
| 114 | 0.881 | 0.833 | 0.920 | 0.939 | 0.897 | 0.958 | 0.761 | 0.703 |
| 115 | 0.885 | 0.834 | 0.924 | 0.941 | 0.896 | 0.963 | 0.766 | 0.708 |
| 116 | 0.890 | 0.841 | 0.928 | 0.946 | 0.908 | 0.964 | 0.771 | 0.716 |
| 117 | 0.881 | 0.833 | 0.919 | 0.939 | 0.896 | 0.958 | 0.761 | 0.703 |
| 118 | 0.892 | 0.842 | 0.930 | 0.948 | 0.907 | 0.968 | 0.772 | 0.719 |
| 119 | 0.889 | 0.841 | 0.927 | 0.946 | 0.907 | 0.964 | 0.771 | 0.717 |
| 120 | 0.890 | 0.841 | 0.929 | 0.945 | 0.900 | 0.967 | 0.778 | 0.729 |
| 121 | 0.886 | 0.839 | 0.924 | 0.943 | 0.900 | 0.962 | 0.774 | 0.723 |
| 122 | 0.889 | 0.840 | 0.928 | 0.945 | 0.899 | 0.967 | 0.777 | 0.729 |
| 123 | 0.886 | 0.839 | 0.923 | 0.943 | 0.900 | 0.962 | 0.773 | 0.723 |
| 124 | 0.892 | 0.843 | 0.930 | 0.950 | 0.912 | 0.968 | 0.771 | 0.722 |
| 125 | 0.895 | 0.844 | 0.935 | 0.948 | 0.910 | 0.968 | 0.776 | 0.723 |
| 126 | 0.896 | 0.844 | 0.936 | 0.948 | 0.910 | 0.968 | 0.779 | 0.725 |
| 127 | 0.893 | 0.841 | 0.934 | 0.947 | 0.906 | 0.968 | 0.772 | 0.721 |
| 128 | 0.894 | 0.841 | 0.935 | 0.947 | 0.903 | 0.968 | 0.775 | 0.721 |
| 129 | 0.895 | 0.847 | 0.933 | 0.950 | 0.920 | 0.968 | 0.774 | 0.720 |
| 130 | 0.896 | 0.849 | 0.934 | 0.951 | 0.920 | 0.968 | 0.776 | 0.722 |
| 131 | 0.896 | 0.843 | 0.936 | 0.948 | 0.913 | 0.967 | 0.776 | 0.719 |
| 132 | 0.897 | 0.845 | 0.937 | 0.948 | 0.913 | 0.967 | 0.778 | 0.721 |
| 133 | 0.895 | 0.844 | 0.936 | 0.948 | 0.908 | 0.968 | 0.783 | 0.733 |
| 134 | 0.892 | 0.840 | 0.933 | 0.947 | 0.903 | 0.968 | 0.775 | 0.727 |
| 135 | 0.893 | 0.840 | 0.934 | 0.947 | 0.901 | 0.968 | 0.778 | 0.729 |
| 136 | 0.895 | 0.847 | 0.932 | 0.949 | 0.918 | 0.968 | 0.778 | 0.727 |
| 137 | 0.896 | 0.849 | 0.933 | 0.951 | 0.918 | 0.968 | 0.780 | 0.731 |
| 138 | 0.895 | 0.843 | 0.935 | 0.947 | 0.911 | 0.967 | 0.780 | 0.726 |
| 139 | 0.896 | 0.845 | 0.936 | 0.948 | 0.911 | 0.967 | 0.782 | 0.729 |
| 140 | 0.896 | 0.844 | 0.935 | 0.950 | 0.914 | 0.969 | 0.777 | 0.725 |
| 141 | 0.896 | 0.845 | 0.936 | 0.950 | 0.913 | 0.969 | 0.779 | 0.728 |
| 142 | 0.893 | 0.842 | 0.934 | 0.949 | 0.909 | 0.969 | 0.772 | 0.723 |
| 143 | 0.894 | 0.841 | 0.935 | 0.949 | 0.906 | 0.968 | 0.775 | 0.724 |
| 144 | 0.895 | 0.847 | 0.932 | 0.952 | 0.922 | 0.969 | 0.774 | 0.722 |
| 145 | 0.897 | 0.849 | 0.933 | 0.953 | 0.922 | 0.969 | 0.777 | 0.725 |
| 146 | 0.896 | 0.844 | 0.936 | 0.949 | 0.916 | 0.968 | 0.776 | 0.722 |
| 147 | 0.897 | 0.845 | 0.937 | 0.950 | 0.916 | 0.968 | 0.778 | 0.723 |
| 148 | 0.894 | 0.842 | 0.935 | 0.947 | 0.909 | 0.967 | 0.775 | 0.720 |
| 149 | 0.895 | 0.843 | 0.935 | 0.948 | 0.909 | 0.967 | 0.778 | 0.723 |
| 150 | 0.892 | 0.839 | 0.933 | 0.947 | 0.904 | 0.967 | 0.771 | 0.719 |
| 151 | 0.893 | 0.839 | 0.934 | 0.947 | 0.902 | 0.967 | 0.773 | 0.719 |
| 152 | 0.894 | 0.846 | 0.932 | 0.950 | 0.919 | 0.967 | 0.773 | 0.718 |
| 153 | 0.896 | 0.847 | 0.933 | 0.951 | 0.919 | 0.968 | 0.775 | 0.720 |
| 154 | 0.895 | 0.842 | 0.935 | 0.947 | 0.912 | 0.967 | 0.774 | 0.716 |
| 155 | 0.896 | 0.843 | 0.936 | 0.948 | 0.912 | 0.967 | 0.777 | 0.718 |
| 156 | 0.894 | 0.842 | 0.935 | 0.947 | 0.909 | 0.967 | 0.776 | 0.720 |
| 157 | 0.895 | 0.843 | 0.935 | 0.947 | 0.908 | 0.968 | 0.778 | 0.723 |
| 158 | 0.891 | 0.839 | 0.933 | 0.947 | 0.904 | 0.967 | 0.771 | 0.718 |
| 159 | 0.892 | 0.839 | 0.934 | 0.947 | 0.901 | 0.967 | 0.773 | 0.716 |
| 160 | 0.894 | 0.846 | 0.932 | 0.949 | 0.918 | 0.967 | 0.773 | 0.717 |
| 161 | 0.895 | 0.847 | 0.933 | 0.951 | 0.918 | 0.968 | 0.776 | 0.720 |
| 162 | 0.894 | 0.842 | 0.935 | 0.947 | 0.911 | 0.967 | 0.775 | 0.716 |
| 163 | 0.896 | 0.843 | 0.936 | 0.948 | 0.912 | 0.967 | 0.778 | 0.719 |
| 164 | 0.895 | 0.843 | 0.935 | 0.947 | 0.908 | 0.968 | 0.781 | 0.730 |
| 165 | 0.890 | 0.838 | 0.931 | 0.941 | 0.900 | 0.962 | 0.776 | 0.719 |
| 166 | 0.890 | 0.839 | 0.931 | 0.944 | 0.898 | 0.967 | 0.777 | 0.731 |
| 167 | 0.892 | 0.841 | 0.931 | 0.949 | 0.911 | 0.968 | 0.770 | 0.723 |
| 168 | 0.886 | 0.831 | 0.929 | 0.939 | 0.893 | 0.963 | 0.763 | 0.702 |
| 169 | 0.885 | 0.833 | 0.927 | 0.940 | 0.894 | 0.962 | 0.766 | 0.710 |
| 170 | 0.891 | 0.840 | 0.932 | 0.944 | 0.895 | 0.966 | 0.779 | 0.732 |
| 171 | 0.892 | 0.841 | 0.932 | 0.948 | 0.907 | 0.967 | 0.773 | 0.723 |
| 172 | 0.886 | 0.830 | 0.930 | 0.938 | 0.889 | 0.962 | 0.766 | 0.702 |
| 173 | 0.894 | 0.843 | 0.934 | 0.945 | 0.904 | 0.967 | 0.783 | 0.733 |
| 174 | 0.886 | 0.833 | 0.928 | 0.939 | 0.890 | 0.962 | 0.768 | 0.710 |
| 175 | 0.894 | 0.847 | 0.931 | 0.948 | 0.915 | 0.967 | 0.780 | 0.731 |
| 176 | 0.895 | 0.848 | 0.931 | 0.952 | 0.924 | 0.968 | 0.773 | 0.723 |
| 177 | 0.890 | 0.839 | 0.929 | 0.943 | 0.909 | 0.963 | 0.766 | 0.701 |
| 178 | 0.890 | 0.841 | 0.928 | 0.944 | 0.911 | 0.962 | 0.770 | 0.711 |
| 179 | 0.895 | 0.849 | 0.932 | 0.949 | 0.915 | 0.967 | 0.783 | 0.733 |
| 180 | 0.896 | 0.849 | 0.932 | 0.953 | 0.924 | 0.968 | 0.776 | 0.725 |
| 181 | 0.891 | 0.840 | 0.930 | 0.944 | 0.909 | 0.963 | 0.769 | 0.708 |
| 182 | 0.895 | 0.844 | 0.934 | 0.950 | 0.915 | 0.968 | 0.776 | 0.725 |
| 183 | 0.891 | 0.843 | 0.929 | 0.945 | 0.912 | 0.963 | 0.773 | 0.716 |
| 184 | 0.894 | 0.843 | 0.934 | 0.944 | 0.907 | 0.966 | 0.782 | 0.729 |
| 185 | 0.895 | 0.844 | 0.934 | 0.949 | 0.917 | 0.967 | 0.774 | 0.721 |
| 186 | 0.889 | 0.835 | 0.932 | 0.939 | 0.901 | 0.962 | 0.768 | 0.700 |
| 187 | 0.889 | 0.837 | 0.930 | 0.940 | 0.903 | 0.961 | 0.771 | 0.709 |
| 188 | 0.895 | 0.845 | 0.935 | 0.945 | 0.908 | 0.966 | 0.784 | 0.732 |
| 189 | 0.896 | 0.844 | 0.935 | 0.949 | 0.917 | 0.966 | 0.777 | 0.723 |
| 190 | 0.890 | 0.836 | 0.933 | 0.940 | 0.901 | 0.962 | 0.771 | 0.703 |
| 191 | 0.889 | 0.835 | 0.931 | 0.940 | 0.898 | 0.963 | 0.768 | 0.706 |
| 192 | 0.890 | 0.838 | 0.931 | 0.941 | 0.904 | 0.962 | 0.774 | 0.712 |
| 193 | 0.889 | 0.837 | 0.931 | 0.940 | 0.900 | 0.962 | 0.773 | 0.714 |
| 194 | 0.895 | 0.845 | 0.935 | 0.945 | 0.904 | 0.967 | 0.785 | 0.736 |
| 195 | 0.896 | 0.845 | 0.935 | 0.950 | 0.914 | 0.967 | 0.779 | 0.727 |
| 196 | 0.889 | 0.835 | 0.932 | 0.940 | 0.897 | 0.962 | 0.771 | 0.709 |
| 197 | 0.891 | 0.840 | 0.931 | 0.947 | 0.906 | 0.967 | 0.770 | 0.722 |
| 198 | 0.892 | 0.840 | 0.932 | 0.947 | 0.903 | 0.967 | 0.772 | 0.723 |
| 199 | 0.870 | 0.814 | 0.916 | 0.923 | 0.890 | 0.941 | 0.737 | 0.656 |

| Panel | HFpEF | | HFrEF | HFrEF | | ICMP | ICMP |
|---|---|---|---|---|---|---|---|
| Number | symptomatic | HFrEF | asymptomatic | symptomatic | ICMP | asymptomatic | symptomatic |
| 104 | 0.808 | 0.954 | 0.933 | 0.967 | 0.960 | 0.951 | 0.961 |
| 105 | 0.810 | 0.954 | 0.932 | 0.967 | 0.962 | 0.951 | 0.962 |
| 106 | 0.803 | 0.953 | 0.931 | 0.965 | 0.961 | 0.948 | 0.961 |
| 107 | 0.798 | 0.954 | 0.931 | 0.967 | 0.959 | 0.947 | 0.959 |
| 108 | 0.797 | 0.953 | 0.931 | 0.965 | 0.958 | 0.948 | 0.959 |
| 109 | 0.806 | 0.950 | 0.926 | 0.963 | 0.960 | 0.949 | 0.960 |
| 110 | 0.800 | 0.949 | 0.926 | 0.962 | 0.959 | 0.949 | 0.960 |
| 111 | 0.800 | 0.949 | 0.927 | 0.963 | 0.958 | 0.949 | 0.957 |
| 112 | 0.795 | 0.948 | 0.927 | 0.961 | 0.957 | 0.949 | 0.957 |
| 113 | 0.804 | 0.948 | 0.925 | 0.962 | 0.958 | 0.947 | 0.958 |
| 114 | 0.797 | 0.947 | 0.925 | 0.960 | 0.958 | 0.947 | 0.958 |
| 115 | 0.797 | 0.948 | 0.925 | 0.962 | 0.956 | 0.947 | 0.955 |
| 116 | 0.809 | 0.953 | 0.932 | 0.965 | 0.962 | 0.951 | 0.962 |
| 117 | 0.791 | 0.947 | 0.925 | 0.960 | 0.955 | 0.947 | 0.955 |
| 118 | 0.804 | 0.954 | 0.932 | 0.967 | 0.960 | 0.950 | 0.960 |
| 119 | 0.805 | 0.953 | 0.932 | 0.965 | 0.960 | 0.951 | 0.960 |
| 120 | 0.809 | 0.951 | 0.927 | 0.965 | 0.959 | 0.947 | 0.960 |
| 121 | 0.806 | 0.950 | 0.927 | 0.963 | 0.959 | 0.947 | 0.959 |
| 122 | 0.802 | 0.950 | 0.927 | 0.965 | 0.957 | 0.947 | 0.957 |
| 123 | 0.799 | 0.949 | 0.927 | 0.963 | 0.957 | 0.947 | 0.957 |
| 124 | 0.804 | 0.954 | 0.931 | 0.967 | 0.961 | 0.948 | 0.961 |
| 125 | 0.818 | 0.956 | 0.934 | 0.969 | 0.969 | 0.952 | 0.974 |
| 126 | 0.820 | 0.956 | 0.934 | 0.969 | 0.969 | 0.959 | 0.973 |
| 127 | 0.817 | 0.955 | 0.932 | 0.968 | 0.966 | 0.957 | 0.965 |
| 128 | 0.816 | 0.955 | 0.933 | 0.968 | 0.966 | 0.962 | 0.965 |
| 129 | 0.804 | 0.958 | 0.941 | 0.969 | 0.968 | 0.957 | 0.968 |
| 130 | 0.805 | 0.959 | 0.941 | 0.970 | 0.968 | 0.961 | 0.968 |
| 131 | 0.823 | 0.957 | 0.938 | 0.968 | 0.970 | 0.958 | 0.970 |
| 132 | 0.823 | 0.957 | 0.938 | 0.969 | 0.970 | 0.963 | 0.970 |
| 133 | 0.820 | 0.956 | 0.933 | 0.969 | 0.968 | 0.958 | 0.973 |
| 134 | 0.816 | 0.954 | 0.931 | 0.967 | 0.965 | 0.955 | 0.965 |
| 135 | 0.816 | 0.955 | 0.932 | 0.968 | 0.966 | 0.961 | 0.965 |
| 136 | 0.804 | 0.958 | 0.940 | 0.969 | 0.968 | 0.956 | 0.968 |
| 137 | 0.805 | 0.958 | 0.940 | 0.969 | 0.967 | 0.960 | 0.967 |
| 138 | 0.823 | 0.956 | 0.937 | 0.968 | 0.970 | 0.957 | 0.970 |
| 139 | 0.823 | 0.957 | 0.937 | 0.969 | 0.969 | 0.961 | 0.970 |
| 140 | 0.818 | 0.957 | 0.934 | 0.969 | 0.969 | 0.951 | 0.974 |
| 141 | 0.819 | 0.957 | 0.933 | 0.970 | 0.968 | 0.959 | 0.973 |
| 142 | 0.816 | 0.955 | 0.932 | 0.968 | 0.965 | 0.956 | 0.965 |
| 143 | 0.815 | 0.955 | 0.932 | 0.968 | 0.966 | 0.961 | 0.965 |
| 144 | 0.803 | 0.958 | 0.940 | 0.969 | 0.968 | 0.957 | 0.968 |
| 145 | 0.805 | 0.959 | 0.940 | 0.970 | 0.967 | 0.961 | 0.968 |
| 146 | 0.822 | 0.957 | 0.937 | 0.969 | 0.970 | 0.957 | 0.970 |
| 147 | 0.822 | 0.957 | 0.937 | 0.969 | 0.969 | 0.962 | 0.970 |
| 148 | 0.817 | 0.956 | 0.933 | 0.969 | 0.968 | 0.950 | 0.974 |
| 149 | 0.819 | 0.956 | 0.933 | 0.969 | 0.968 | 0.958 | 0.973 |
| 150 | 0.816 | 0.954 | 0.931 | 0.967 | 0.965 | 0.955 | 0.965 |
| 151 | 0.815 | 0.955 | 0.932 | 0.968 | 0.965 | 0.960 | 0.965 |
| 152 | 0.803 | 0.958 | 0.940 | 0.969 | 0.967 | 0.956 | 0.968 |
| 153 | 0.805 | 0.958 | 0.940 | 0.969 | 0.967 | 0.960 | 0.967 |
| 154 | 0.822 | 0.957 | 0.937 | 0.968 | 0.969 | 0.957 | 0.970 |
| 155 | 0.822 | 0.957 | 0.937 | 0.969 | 0.969 | 0.961 | 0.970 |
| 156 | 0.817 | 0.956 | 0.933 | 0.969 | 0.969 | 0.950 | 0.974 |
| 157 | 0.819 | 0.956 | 0.933 | 0.969 | 0.968 | 0.958 | 0.973 |
| 158 | 0.815 | 0.954 | 0.931 | 0.967 | 0.965 | 0.955 | 0.965 |
| 159 | 0.815 | 0.955 | 0.932 | 0.968 | 0.966 | 0.960 | 0.965 |
| 160 | 0.803 | 0.958 | 0.940 | 0.969 | 0.968 | 0.956 | 0.968 |
| 161 | 0.804 | 0.958 | 0.940 | 0.970 | 0.967 | 0.960 | 0.968 |
| 162 | 0.821 | 0.957 | 0.937 | 0.968 | 0.970 | 0.957 | 0.970 |
| 163 | 0.822 | 0.957 | 0.937 | 0.969 | 0.969 | 0.961 | 0.970 |
| 164 | 0.818 | 0.956 | 0.933 | 0.969 | 0.969 | 0.950 | 0.974 |
| 165 | 0.816 | 0.950 | 0.925 | 0.965 | 0.964 | 0.950 | 0.970 |
| 166 | 0.814 | 0.951 | 0.927 | 0.965 | 0.962 | 0.952 | 0.961 |
| 167 | 0.809 | 0.954 | 0.931 | 0.967 | 0.963 | 0.953 | 0.963 |
| 168 | 0.812 | 0.950 | 0.926 | 0.964 | 0.963 | 0.954 | 0.962 |
| 169 | 0.808 | 0.948 | 0.924 | 0.962 | 0.960 | 0.952 | 0.959 |
| 170 | 0.814 | 0.951 | 0.926 | 0.965 | 0.961 | 0.955 | 0.961 |
| 171 | 0.810 | 0.954 | 0.930 | 0.966 | 0.963 | 0.955 | 0.962 |
| 172 | 0.813 | 0.949 | 0.924 | 0.963 | 0.962 | 0.958 | 0.961 |
| 173 | 0.819 | 0.954 | 0.929 | 0.967 | 0.967 | 0.948 | 0.972 |
| 174 | 0.809 | 0.947 | 0.922 | 0.962 | 0.959 | 0.954 | 0.958 |
| 175 | 0.803 | 0.956 | 0.937 | 0.967 | 0.966 | 0.954 | 0.966 |
| 176 | 0.798 | 0.958 | 0.940 | 0.969 | 0.967 | 0.954 | 0.967 |
| 177 | 0.800 | 0.954 | 0.936 | 0.966 | 0.966 | 0.956 | 0.966 |
| 178 | 0.799 | 0.954 | 0.935 | 0.965 | 0.965 | 0.954 | 0.965 |
| 179 | 0.805 | 0.956 | 0.937 | 0.968 | 0.965 | 0.956 | 0.966 |
| 180 | 0.800 | 0.958 | 0.939 | 0.969 | 0.966 | 0.954 | 0.966 |
| 181 | 0.802 | 0.954 | 0.935 | 0.966 | 0.965 | 0.958 | 0.965 |
| 182 | 0.814 | 0.957 | 0.933 | 0.969 | 0.968 | 0.948 | 0.973 |
| 183 | 0.801 | 0.954 | 0.934 | 0.966 | 0.964 | 0.954 | 0.964 |
| 184 | 0.822 | 0.954 | 0.933 | 0.966 | 0.967 | 0.955 | 0.967 |
| 185 | 0.816 | 0.956 | 0.936 | 0.968 | 0.968 | 0.954 | 0.969 |
| 186 | 0.820 | 0.952 | 0.932 | 0.965 | 0.968 | 0.957 | 0.968 |
| 187 | 0.818 | 0.951 | 0.931 | 0.963 | 0.966 | 0.955 | 0.966 |
| 188 | 0.823 | 0.954 | 0.933 | 0.967 | 0.967 | 0.957 | 0.967 |
| 189 | 0.817 | 0.956 | 0.935 | 0.968 | 0.967 | 0.955 | 0.968 |
| 190 | 0.820 | 0.952 | 0.931 | 0.965 | 0.967 | 0.960 | 0.967 |
| 191 | 0.814 | 0.952 | 0.928 | 0.965 | 0.967 | 0.950 | 0.972 |
| 192 | 0.818 | 0.951 | 0.931 | 0.964 | 0.965 | 0.956 | 0.965 |
| 193 | 0.815 | 0.951 | 0.927 | 0.965 | 0.966 | 0.948 | 0.971 |
| 194 | 0.820 | 0.953 | 0.928 | 0.968 | 0.966 | 0.952 | 0.971 |
| 195 | 0.816 | 0.956 | 0.931 | 0.969 | 0.966 | 0.951 | 0.972 |
| 196 | 0.816 | 0.951 | 0.926 | 0.966 | 0.965 | 0.955 | 0.970 |
| 197 | 0.808 | 0.954 | 0.932 | 0.967 | 0.963 | 0.955 | 0.962 |
| 198 | 0.810 | 0.954 | 0.933 | 0.967 | 0.964 | 0.962 | 0.962 |
| 199 | 0.796 | 0.941 | 0.918 | 0.955 | 0.963 | 0.948 | 0.968 |

TABLE 5
Performance of three selected panels (panels number 1, 3,
and 4 in Table 2, respectively) with or without NT-proBNP
and with or without ANOVA correction for age, BMI and gender
in different CHF subgroups in the testing (“VD”) data
set, as compared to the known marker NT-proBNP alone.

| | | | | | NT- |
|---|---|---|---|---|---|
| | With | | | Panel | proBNP |
| Panel | NT- | With | | AUC | AUC |
| Number | proBNP | ANOVA | SUBGROUP | estimate | estimate |
| 1 | yes | no | DCMP asymptomatic | 0.887 | 0.845 |
| 1 | yes | no | DCMP symptomatic | 0.965 | 0.944 |
| 1 | yes | no | DCMP | 0.937 | 0.905 |
| 1 | yes | no | HFrEF asymptomatic | 0.950 | 0.911 |
| 1 | yes | no | HFrEF symptomatic | 0.974 | 0.939 |
| 1 | yes | no | HFrEF | 0.966 | 0.926 |
| 1 | yes | no | ICMP asymptomatic | 0.995 | 0.970 |
| 1 | yes | no | ICMP symptomatic | 0.983 | 0.936 |
| 1 | yes | no | ICMP | 0.988 | 0.949 |
| 1 | yes | no | HFrEF LVEF < 35% | 0.995 | 0.987 |
| 1 | yes | no | HFrEF LVEF 35% to | 0.942 | 0.873 |
| | | | 50% | | |
| 1 | yes | no | HFrEF MALE | 0.971 | 0.939 |
| 1 | yes | no | HFrEF FEMALE | 0.935 | 0.928 |
| 3 | no | no | DCMP asymptomatic | 0.800 | 0.845 |
| 3 | no | no | DCMP symptomatic | 0.872 | 0.944 |
| 3 | no | no | DCMP | 0.845 | 0.905 |
| 3 | no | no | HFrEF asymptomatic | 0.854 | 0.911 |
| 3 | no | no | HFrEF symptomatic | 0.887 | 0.939 |
| 3 | no | no | HFrEF | 0.875 | 0.926 |
| 3 | no | no | ICMP asymptomatic | 0.908 | 0.970 |
| 3 | no | no | ICMP symptomatic | 0.900 | 0.936 |
| 3 | no | no | ICMP | 0.904 | 0.949 |
| 3 | no | no | HFrEF LVEF < 35% | 0.914 | 0.987 |
| 3 | no | no | HFrEF LVEF 35% to | 0.841 | 0.873 |
| | | | 50% | | |
| 3 | no | no | HFrEF MALE | 0.862 | 0.939 |
| 3 | no | no | HFrEF FEMALE | 0.845 | 0.928 |
| 4 | no | no | DCMP asymptomatic | 0.774 | 0.845 |
| 4 | no | no | DCMP symptomatic | 0.843 | 0.944 |
| 4 | no | no | DCMP | 0.816 | 0.905 |
| 4 | no | no | HFrEF asymptomatic | 0.833 | 0.911 |
| 4 | no | no | HFrEF symptomatic | 0.862 | 0.939 |
| 4 | no | no | HFrEF | 0.851 | 0.926 |
| 4 | no | no | ICMP asymptomatic | 0.895 | 0.970 |
| 4 | no | no | ICMP symptomatic | 0.880 | 0.936 |
| 4 | no | no | ICMP | 0.886 | 0.949 |
| 4 | no | no | HFrEF LVEF < 35% | 0.887 | 0.987 |
| 4 | no | no | HFrEF LVEF 35% to | 0.821 | 0.873 |
| | | | 50% | | |
| 4 | no | no | HFrEF MALE | 0.839 | 0.939 |
| 4 | no | no | HFrEF FEMALE | 0.822 | 0.928 |

TABLE 5a
Performance of further selected panels (Table 2a) with or
without NT-proBNP and without ANOVA correction for age, BMI
and gender in different CHF subgroups in the testing (“VD”) data
set, as compared to the known marker NT-proBNP alone

| | | | | NT- |
|---|---|---|---|---|
| | With | | Panel | proBNP |
| Panel | NT- | | AUC | AUC |
| No. | proBNP | Subgroup | estimate | Estimate |
| 200 | yes | CHF | 0.908 | 0.848 |
| 200 | yes | ICM | 0.987 | 0.949 |
| 200 | yes | DCM | 0.936 | 0.905 |
| 200 | yes | HFrEF | 0.964 | 0.926 |
| 200 | yes | HFpEF | 0.820 | 0.706 |
| 200 | yes | CHF asymptomatic | 0.875 | 0.801 |
| 200 | yes | ICMP asymptomatic | 0.994 | 0.970 |
| 200 | yes | DCMP asymptomatic | 0.884 | 0.845 |
| 200 | yes | HFrEF asymptomatic | 0.948 | 0.911 |
| 200 | yes | HFpEF asymptomatic | 0.801 | 0.670 |
| 200 | yes | CHF symptomatic | 0.936 | 0.890 |
| 200 | yes | ICMP symptomatic | 0.981 | 0.936 |
| 200 | yes | DCMP symptomatic | 0.964 | 0.944 |
| 200 | yes | HFrEF symptomatic | 0.973 | 0.939 |
| 200 | yes | HFpEF symptomatic | 0.845 | 0.754 |
| 200 | yes | ICMP LVEF 35% to | 0.978 | 0.925 |
| | | 50% | | |
| 200 | yes | DCMP LVEF 35% to | 0.874 | 0.792 |
| | | 50% | | |
| 200 | yes | HFrEF LVEF 35% to | 0.939 | 0.873 |
| | | 50% | | |
| 200 | yes | ICMP LVEF < 35% | 0.999 | 0.989 |
| 200 | yes | DCMP LVEF < 35% | 0.989 | 0.986 |
| 200 | yes | HFrEF LVEF < 35% | 0.995 | 0.987 |
| 200 | no | CHF | 0.813 | 0.848 |
| 200 | no | ICM | 0.902 | 0.949 |
| 200 | no | DCM | 0.824 | 0.905 |
| 200 | no | HFrEF | 0.863 | 0.926 |
| 200 | no | HFpEF | 0.723 | 0.706 |
| 200 | no | CHF asymptomatic | 0.792 | 0.801 |
| 200 | no | ICMP asymptomatic | 0.905 | 0.970 |
| 200 | no | DCMP asymptomatic | 0.784 | 0.845 |
| 200 | no | HFrEF asymptomatic | 0.844 | 0.911 |
| 200 | no | HFpEF asymptomatic | 0.732 | 0.670 |
| 200 | no | CHF symptomatic | 0.830 | 0.890 |
| 200 | no | ICMP symptomatic | 0.899 | 0.936 |
| 200 | no | DCMP symptomatic | 0.849 | 0.944 |
| 200 | no | HFrEF symptomatic | 0.874 | 0.939 |
| 200 | no | HFpEF symptomatic | 0.707 | 0.754 |
| 200 | no | ICMP LVEF 35% to | 0.889 | 0.925 |
| | | 50% | | |
| 200 | no | DCMP LVEF 35% to | 0.747 | 0.792 |
| | | 50% | | |
| 200 | no | HFrEF LVEF 35% to | 0.833 | 0.873 |
| | | 50% | | |
| 200 | no | ICMP LVEF < 35% | 0.930 | 0.989 |
| 200 | no | DCMP LVEF < 35% | 0.880 | 0.986 |
| 200 | no | HFrEF LVEF < 35% | 0.898 | 0.987 |
| 201 | yes | CHF | 0.895 | 0.848 |
| 201 | yes | ICM | 0.981 | 0.949 |
| 201 | yes | DCM | 0.931 | 0.905 |
| 201 | yes | HFrEF | 0.958 | 0.926 |
| 201 | yes | HFpEF | 0.795 | 0.706 |
| 201 | yes | CHF asymptomatic | 0.859 | 0.801 |
| 201 | yes | ICMP asymptomatic | 0.991 | 0.970 |
| 201 | yes | DCMP asymptomatic | 0.873 | 0.845 |
| 201 | yes | HFrEF asymptomatic | 0.940 | 0.911 |
| 201 | yes | HFpEF asymptomatic | 0.778 | 0.670 |
| 201 | yes | CHF symptomatic | 0.927 | 0.890 |
| 201 | yes | ICMP symptomatic | 0.973 | 0.936 |
| 201 | yes | DCMP symptomatic | 0.964 | 0.944 |
| 201 | yes | HFrEF symptomatic | 0.969 | 0.939 |
| 201 | yes | HFpEF symptomatic | 0.819 | 0.754 |
| 201 | yes | ICMP LVEF 35% to | 0.968 | 0.925 |
| | | 50% | | |
| 201 | yes | DCMP LVEF 35% to | 0.859 | 0.792 |
| | | 50% | | |
| 201 | yes | HFrEF LVEF 35% to | 0.926 | 0.873 |
| | | 50% | | |
| 201 | yes | ICMP LVEF < 35% | 0.999 | 0.989 |
| 201 | yes | DCMP LVEF < 35% | 0.989 | 0.986 |
| 201 | yes | HFrEF LVEF < 35% | 0.994 | 0.987 |
| 201 | no | CHF | 0.794 | 0.848 |
| 201 | no | ICM | 0.887 | 0.949 |
| 201 | no | DCM | 0.814 | 0.905 |
| 201 | no | HFrEF | 0.851 | 0.926 |
| 201 | no | HFpEF | 0.690 | 0.706 |
| 201 | no | CHF asymptomatic | 0.764 | 0.801 |
| 201 | no | ICMP asymptomatic | 0.893 | 0.970 |
| 201 | no | DCMP asymptomatic | 0.759 | 0.845 |
| 201 | no | HFrEF asymptomatic | 0.825 | 0.911 |
| 201 | no | HFpEF asymptomatic | 0.694 | 0.670 |
| 201 | no | CHF symptomatic | 0.817 | 0.890 |
| 201 | no | ICMP symptomatic | 0.883 | 0.936 |
| 201 | no | DCMP symptomatic | 0.848 | 0.944 |
| 201 | no | HFrEF symptomatic | 0.866 | 0.939 |
| 201 | no | HFpEF symptomatic | 0.683 | 0.754 |
| 201 | no | ICMP LVEF 35% to | 0.863 | 0.925 |
| | | 50% | | |
| 201 | no | DCMP LVEF 35% to | 0.738 | 0.792 |
| | | 50% | | |
| 201 | no | HFrEF LVEF 35% to | 0.814 | 0.873 |
| | | 50% | | |
| 201 | no | ICMP LVEF < 35% | 0.938 | 0.989 |
| 201 | no | DCMP LVEF < 35% | 0.869 | 0.986 |
| 201 | no | HFrEF LVEF < 35% | 0.895 | 0.987 |
| 202 | yes | CHF | 0.893 | 0.848 |
| 202 | yes | ICM | 0.981 | 0.949 |
| 202 | yes | DCM | 0.929 | 0.905 |
| 202 | yes | HFrEF | 0.957 | 0.926 |
| 202 | yes | HFpEF | 0.790 | 0.706 |
| 202 | yes | CHF asymptomatic | 0.857 | 0.801 |
| 202 | yes | ICMP asymptomatic | 0.992 | 0.970 |
| 202 | yes | DCMP asymptomatic | 0.869 | 0.845 |
| 202 | yes | HFrEF asymptomatic | 0.938 | 0.911 |
| 202 | yes | HFpEF asymptomatic | 0.775 | 0.670 |
| 202 | yes | CHF symptomatic | 0.924 | 0.890 |
| 202 | yes | ICMP symptomatic | 0.972 | 0.936 |
| 202 | yes | DCMP symptomatic | 0.963 | 0.944 |
| 202 | yes | HFrEF symptomatic | 0.968 | 0.939 |
| 202 | yes | HFpEF symptomatic | 0.810 | 0.754 |
| 202 | yes | ICMP LVEF 35% to | 0.969 | 0.925 |
| | | 50% | | |
| 202 | yes | DCMP LVEF 35% to | 0.855 | 0.792 |
| | | 50% | | |
| 202 | yes | HFrEF LVEF 35% to | 0.926 | 0.873 |
| | | 50% | | |
| 202 | yes | ICMP LVEF < 35% | 0.999 | 0.989 |
| 202 | yes | DCMP LVEF < 35% | 0.988 | 0.986 |
| 202 | yes | HFrEF LVEF < 35% | 0.994 | 0.987 |
| 203 | no | CHF | 0.791 | 0.848 |
| 203 | no | ICM | 0.883 | 0.949 |
| 203 | no | DCM | 0.816 | 0.905 |
| 203 | no | HFrEF | 0.850 | 0.926 |
| 203 | no | HFpEF | 0.683 | 0.706 |
| 203 | no | CHF asymptomatic | 0.761 | 0.801 |
| 203 | no | ICMP asymptomatic | 0.887 | 0.970 |
| 203 | no | DCMP asymptomatic | 0.754 | 0.845 |
| 203 | no | HFrEF asymptomatic | 0.819 | 0.911 |
| 203 | no | HFpEF asymptomatic | 0.692 | 0.670 |
| 203 | no | CHF symptomatic | 0.815 | 0.890 |
| 203 | no | ICMP symptomatic | 0.879 | 0.936 |
| 203 | no | DCMP symptomatic | 0.854 | 0.944 |
| 203 | no | HFrEF symptomatic | 0.867 | 0.939 |
| 203 | no | HFpEF symptomatic | 0.669 | 0.754 |
| 203 | no | ICMP LVEF 35% to | 0.855 | 0.925 |
| | | 50% | | |
| 203 | no | DCMP LVEF 35% to | 0.731 | 0.792 |
| | | 50% | | |
| 203 | no | HFrEF LVEF 35% to | 0.806 | 0.873 |
| | | 50% | | |
| 203 | no | ICMP LVEF < 35% | 0.943 | 0.989 |
| 203 | no | DCMP LVEF < 35% | 0.877 | 0.986 |
| 203 | no | HFrEF LVEF < 35% | 0.901 | 0.987 |
| 204 | yes | CHF | 0.872 | 0.848 |
| 204 | yes | ICM | 0.971 | 0.949 |
| 204 | yes | DCM | 0.911 | 0.905 |
| 204 | yes | HFrEF | 0.943 | 0.926 |
| 204 | yes | HFpEF | 0.756 | 0.706 |
| 204 | yes | CHF asymptomatic | 0.832 | 0.801 |
| 204 | yes | ICMP asymptomatic | 0.986 | 0.970 |
| 204 | yes | DCMP asymptomatic | 0.849 | 0.845 |
| 204 | yes | HFrEF asymptomatic | 0.924 | 0.911 |
| 204 | yes | HFpEF asymptomatic | 0.733 | 0.670 |
| 204 | yes | CHF symptomatic | 0.908 | 0.890 |
| 204 | yes | ICMP symptomatic | 0.962 | 0.936 |
| 204 | yes | DCMP symptomatic | 0.949 | 0.944 |
| 204 | yes | HFrEF symptomatic | 0.956 | 0.939 |
| 204 | yes | HFpEF symptomatic | 0.788 | 0.754 |
| 204 | yes | ICMP LVEF 35% to | 0.953 | 0.925 |
| | | 50% | | |
| 204 | yes | DCMP LVEF 35% to | 0.822 | 0.792 |
| | | 50% | | |
| 204 | yes | HFrEF LVEF 35% to | 0.902 | 0.873 |
| | | 50% | | |
| 204 | yes | ICMP LVEF < 35% | 0.999 | 0.989 |
| 204 | yes | DCMP LVEF < 35% | 0.983 | 0.986 |
| 204 | yes | HFrEF LVEF < 35% | 0.991 | 0.987 |
| 204 | no | CHF | 0.749 | 0.848 |
| 204 | no | ICM | 0.852 | 0.949 |
| 204 | no | DCM | 0.779 | 0.905 |
| 204 | no | HFrEF | 0.816 | 0.926 |
| 204 | no | HFpEF | 0.627 | 0.706 |
| 204 | no | CHF asymptomatic | 0.708 | 0.801 |
| 204 | no | ICMP asymptomatic | 0.840 | 0.970 |
| 204 | no | DCMP asymptomatic | 0.705 | 0.845 |
| 204 | no | HFrEF asymptomatic | 0.772 | 0.911 |
| 204 | no | HFpEF asymptomatic | 0.632 | 0.670 |
| 204 | no | CHF symptomatic | 0.783 | 0.890 |
| 204 | no | ICMP symptomatic | 0.858 | 0.936 |
| 204 | no | DCMP symptomatic | 0.824 | 0.944 |
| 204 | no | HFrEF symptomatic | 0.842 | 0.939 |
| 204 | no | HFpEF symptomatic | 0.618 | 0.754 |
| 204 | no | ICMP LVEF 35% to | 0.811 | 0.925 |
| | | 50% | | |
| 204 | no | DCMP LVEF 35% to | 0.681 | 0.792 |
| | | 50% | | |
| 204 | no | HFrEF LVEF 35% to | 0.760 | 0.873 |
| | | 50% | | |
| 204 | no | ICMP LVEF < 35% | 0.934 | 0.989 |
| 204 | no | DCMP LVEF < 35% | 0.850 | 0.986 |
| 204 | no | HFrEF LVEF < 35% | 0.881 | 0.987 |
| 205 | yes | CHF | 0.897 | 0.848 |
| 205 | yes | ICM | 0.983 | 0.949 |
| 205 | yes | DCM | 0.935 | 0.905 |
| 205 | yes | HFrEF | 0.961 | 0.926 |
| 205 | yes | HFpEF | 0.789 | 0.706 |
| 205 | yes | CHF asymptomatic | 0.863 | 0.801 |
| 205 | yes | ICMP asymptomatic | 0.994 | 0.970 |
| 205 | yes | DCMP asymptomatic | 0.883 | 0.845 |
| 205 | yes | HFrEF asymptomatic | 0.947 | 0.911 |
| 205 | yes | HFpEF asymptomatic | 0.776 | 0.670 |
| 205 | yes | CHF symptomatic | 0.926 | 0.890 |
| 205 | yes | ICMP symptomatic | 0.975 | 0.936 |
| 205 | yes | DCMP symptomatic | 0.965 | 0.944 |
| 205 | yes | HFrEF symptomatic | 0.971 | 0.939 |
| 205 | yes | HFpEF symptomatic | 0.806 | 0.754 |
| 205 | yes | ICMP LVEF 35% to | 0.972 | 0.925 |
| | | 50% | | |
| 205 | yes | DCMP LVEF 35% to | 0.870 | 0.792 |
| | | 50% | | |
| 205 | yes | HFrEF LVEF 35% to | 0.931 | 0.873 |
| | | 50% | | |
| 205 | yes | ICMP LVEF < 35% | 0.999 | 0.989 |
| 205 | yes | DCMP LVEF < 35% | 0.990 | 0.986 |
| 205 | yes | HFrEF LVEF < 35% | 0.995 | 0.987 |
| 205 | no | CHF | 0.779 | 0.848 |
| 205 | no | ICM | 0.879 | 0.949 |
| 205 | no | DCM | 0.795 | 0.905 |
| 205 | no | HFrEF | 0.837 | 0.926 |
| 205 | no | HFpEF | 0.676 | 0.706 |
| 205 | no | CHF asymptomatic | 0.756 | 0.801 |
| 205 | no | ICMP asymptomatic | 0.882 | 0.970 |
| 205 | no | DCMP asymptomatic | 0.753 | 0.845 |
| 205 | no | HFrEF asymptomatic | 0.817 | 0.911 |
| 205 | no | HFpEF asymptomatic | 0.687 | 0.670 |
| 205 | no | CHF symptomatic | 0.797 | 0.890 |
| 205 | no | ICMP symptomatic | 0.875 | 0.936 |
| 205 | no | DCMP symptomatic | 0.821 | 0.944 |
| 205 | no | HFrEF symptomatic | 0.848 | 0.939 |
| 205 | no | HFpEF symptomatic | 0.659 | 0.754 |
| 205 | no | ICMP LVEF 35% to | 0.857 | 0.925 |
| | | 50% | | |
| 205 | no | DCMP LVEF 35% to | 0.727 | 0.792 |
| | | 50% | | |
| 205 | no | HFrEF LVEF 35% to | 0.807 | 0.873 |
| | | 50% | | |
| 205 | no | ICMP LVEF < 35% | 0.920 | 0.989 |
| 205 | no | DCMP LVEF < 35% | 0.846 | 0.986 |
| 205 | no | HFrEF LVEF < 35% | 0.872 | 0.987 |
| 206 | yes | CHF | 0.887 | 0.848 |
| 206 | yes | ICM | 0.978 | 0.949 |
| 206 | yes | DCM | 0.924 | 0.905 |
| 206 | yes | HFrEF | 0.953 | 0.926 |
| 206 | yes | HFpEF | 0.780 | 0.706 |
| 206 | yes | CHF asymptomatic | 0.850 | 0.801 |
| 206 | yes | ICMP asymptomatic | 0.992 | 0.970 |
| 206 | yes | DCMP asymptomatic | 0.862 | 0.845 |
| 206 | yes | HFrEF asymptomatic | 0.935 | 0.911 |
| 206 | yes | HFpEF asymptomatic | 0.762 | 0.670 |
| 206 | yes | CHF symptomatic | 0.919 | 0.890 |
| 206 | yes | ICMP symptomatic | 0.968 | 0.936 |
| 206 | yes | DCMP symptomatic | 0.960 | 0.944 |
| 206 | yes | HFrEF symptomatic | 0.964 | 0.939 |
| 206 | yes | HFpEF symptomatic | 0.804 | 0.754 |
| 206 | yes | ICMP LVEF 35% to | 0.964 | 0.925 |
| | | 50% | | |
| 206 | yes | DCMP LVEF 35% to | 0.842 | 0.792 |
| | | 50% | | |
| 206 | yes | HFrEF LVEF 35% to | 0.918 | 0.873 |
| | | 50% | | |
| 206 | yes | ICMP LVEF < 35% | 0.999 | 0.989 |
| 206 | yes | DCMP LVEF < 35% | 0.987 | 0.986 |
| 206 | yes | HFrEF LVEF < 35% | 0.993 | 0.987 |
| 206 | no | CHF | 0.779 | 0.848 |
| 206 | no | ICM | 0.873 | 0.949 |
| 206 | no | DCM | 0.808 | 0.905 |
| 206 | no | HFrEF | 0.841 | 0.926 |
| 206 | no | HFpEF | 0.668 | 0.706 |
| 206 | no | CHF asymptomatic | 0.750 | 0.801 |
| 206 | no | ICMP asymptomatic | 0.883 | 0.970 |
| 206 | no | DCMP asymptomatic | 0.743 | 0.845 |
| 206 | no | HFrEF asymptomatic | 0.811 | 0.911 |
| 206 | no | HFpEF asymptomatic | 0.678 | 0.670 |
| 206 | no | CHF symptomatic | 0.803 | 0.890 |
| 206 | no | ICMP symptomatic | 0.867 | 0.936 |
| 206 | no | DCMP symptomatic | 0.848 | 0.944 |
| 206 | no | HFrEF symptomatic | 0.858 | 0.939 |
| 206 | no | HFpEF symptomatic | 0.652 | 0.754 |
| 206 | no | ICMP LVEF 35% to | 0.846 | 0.925 |
| | | 50% | | |
| 206 | no | DCMP LVEF 35% to | 0.720 | 0.792 |
| | | 50% | | |
| 206 | no | HFrEF LVEF 35% to | 0.795 | 0.873 |
| | | 50% | | |
| 206 | no | ICMP LVEF < 35% | 0.931 | 0.989 |
| 206 | no | DCMP LVEF < 35% | 0.872 | 0.986 |
| 206 | no | HFrEF LVEF < 35% | 0.894 | 0.987 |

TABLE 5b
Performance of four selected panels (panels number 1, 2, 3, and 4 in Table 2,
respectively) with and without NT-proBNP and with or with-out ANOVA correction
for age, BMI and gender in different CHF subgroups in the testing (“VD”)
data set, as compared to the known marker NT-proBNP alone.

| | | AUC: Panel | | AUC: Panel | | |
|---|---|---|---|---|---|---|
| | | (no NT- | AUC: Panel | (with NT- | AUC: Panel | AUC: |
| | | proBNP, | (no NT- | proBNP, | (with NT- | NT-proBNP |
| Panel | | with ANOVA | proBNP, no | with ANOVA | proBNP, no | (no ANOVA |
| No. | Subgroup | correction) | ANOVA correction) | correction) | ANOVA correction) | correction) |
| 1 | CHF | 0.787 | 0.813 | 0.891 | 0.911 | 0.848 |
| 1 | ICMP | 0.876 | 0.899 | 0.984 | 0.988 | 0.949 |
| 1 | DCMP | 0.793 | 0.819 | 0.935 | 0.937 | 0.905 |
| 1 | HFrEF | 0.835 | 0.860 | 0.961 | 0.966 | 0.926 |
| 1 | HFpEF | 0.701 | 0.729 | 0.774 | 0.826 | 0.706 |
| 1 | CHF asymptomatic | 0.767 | 0.797 | 0.857 | 0.879 | 0.801 |
| 1 | ICMP asymptomatic | 0.878 | 0.906 | 0.994 | 0.995 | 0.970 |
| 1 | DCMP asymptomatic | 0.755 | 0.787 | 0.893 | 0.887 | 0.845 |
| 1 | HFrEF asymptomatic | 0.815 | 0.845 | 0.952 | 0.950 | 0.911 |
| 1 | HFpEF asymptomatic | 0.711 | 0.742 | 0.756 | 0.810 | 0.670 |
| 1 | CHF symptomatic | 0.804 | 0.825 | 0.920 | 0.938 | 0.890 |
| 1 | ICMP symptomatic | 0.874 | 0.894 | 0.976 | 0.983 | 0.936 |
| 1 | DCMP symptomatic | 0.818 | 0.840 | 0.959 | 0.965 | 0.944 |
| 1 | HFrEF symptomatic | 0.846 | 0.868 | 0.967 | 0.974 | 0.939 |
| 1 | HFpEF symptomatic | 0.685 | 0.708 | 0.796 | 0.846 | 0.754 |
| 1 | ICMP LVEF 35% to 50% | 0.861 | 0.888 | 0.973 | 0.980 | 0.925 |
| 1 | DCMP LVEF 35% to | 0.718 | 0.749 | 0.886 | 0.880 | 0.792 |
| | 50% | | | | | |
| 1 | HFrEF LVEF 35% to 50% | 0.805 | 0.833 | 0.939 | 0.942 | 0.873 |
| 1 | ICMP LVEF <35% | 0.909 | 0.924 | 0.999 | 0.999 | 0.989 |
| 1 | DCMP LVEF <35% | 0.847 | 0.870 | 0.989 | 0.989 | 0.986 |
| 1 | HFrEF LVEF <35% | 0.870 | 0.890 | 0.995 | 0.995 | 0.987 |
| 2/4 | CHF | 0.772 | 0.798 | 0.885 | 0.903 | 0.848 |
| 2/4 | ICMP | 0.863 | 0.886 | 0.981 | 0.985 | 0.949 |
| 2/4 | DCMP | 0.788 | 0.816 | 0.932 | 0.934 | 0.905 |
| 2/4 | HFrEF | 0.826 | 0.851 | 0.957 | 0.962 | 0.926 |
| 2/4 | HFpEF | 0.676 | 0.703 | 0.761 | 0.810 | 0.706 |
| 2/4 | CHF asymptomatic | 0.744 | 0.777 | 0.847 | 0.867 | 0.801 |
| 2/4 | ICMP asymptomatic | 0.869 | 0.895 | 0.993 | 0.993 | 0.970 |
| 2/4 | DCMP asymptomatic | 0.736 | 0.774 | 0.884 | 0.878 | 0.845 |
| 2/4 | HFrEF asymptomatic | 0.800 | 0.833 | 0.946 | 0.944 | 0.911 |
| 2/4 | HFpEF asymptomatic | 0.680 | 0.712 | 0.736 | 0.788 | 0.670 |
| 2/4 | CHF symptomatic | 0.794 | 0.815 | 0.917 | 0.933 | 0.890 |
| 2/4 | ICMP symptomatic | 0.859 | 0.880 | 0.971 | 0.978 | 0.936 |
| 2/4 | DCMP symptomatic | 0.823 | 0.843 | 0.959 | 0.964 | 0.944 |
| 2/4 | HFrEF symptomatic | 0.840 | 0.862 | 0.965 | 0.972 | 0.939 |
| 2/4 | HFpEF symptomatic | 0.667 | 0.688 | 0.794 | 0.840 | 0.754 |
| 2/4 | ICMP LVEF 35% to 50% | 0.846 | 0.876 | 0.968 | 0.976 | 0.925 |
| 2/4 | DCMP LVEF 35% to | 0.701 | 0.738 | 0.873 | 0.866 | 0.792 |
| | 50% | | | | | |
| 2/4 | HFrEF LVEF 35% to 50% | 0.788 | 0.821 | 0.931 | 0.935 | 0.873 |
| 2/4 | ICMP LVEF <35% | 0.902 | 0.915 | 0.999 | 0.999 | 0.989 |
| 2/4 | DCMP LVEF <35% | 0.853 | 0.874 | 0.989 | 0.989 | 0.986 |
| 2/4 | HFrEF LVEF <35% | 0.869 | 0.887 | 0.994 | 0.994 | 0.987 |
| 3 | CHF | 0.801 | 0.826 | (nd) | (nd) | 0.848 |
| 3 | ICMP | 0.876 | 0.904 | (nd) | (nd) | 0.949 |
| 3 | DCMP | 0.824 | 0.845 | (nd) | (nd) | 0.905 |
| 3 | HFrEF | 0.850 | 0.875 | (nd) | (nd) | 0.926 |
| 3 | HFpEF | 0.711 | 0.739 | (nd) | (nd) | 0.706 |
| 3 | CHF asymptomatic | 0.775 | 0.805 | (nd) | (nd) | 0.801 |
| 3 | ICMP asymptomatic | 0.881 | 0.908 | (nd) | (nd) | 0.970 |
| 3 | DCMP asymptomatic | 0.771 | 0.800 | (nd) | (nd) | 0.845 |
| 3 | HFrEF asymptomatic | 0.825 | 0.854 | (nd) | (nd) | 0.911 |
| 3 | HFpEF asymptomatic | 0.717 | 0.748 | (nd) | (nd) | 0.670 |
| 3 | CHF symptomatic | 0.822 | 0.844 | (nd) | (nd) | 0.890 |
| 3 | ICMP symptomatic | 0.872 | 0.900 | (nd) | (nd) | 0.936 |
| 3 | DCMP symptomatic | 0.857 | 0.872 | (nd) | (nd) | 0.944 |
| 3 | HFrEF symptomatic | 0.865 | 0.887 | (nd) | (nd) | 0.939 |
| 3 | HFpEF symptomatic | 0.701 | 0.724 | (nd) | (nd) | 0.754 |
| 3 | ICMP LVEF 35% to 50% | 0.851 | 0.887 | (nd) | (nd) | 0.925 |
| 3 | DCMP LVEF 35% to | 0.742 | 0.771 | (nd) | (nd) | 0.792 |
| | 50% | | | | | |
| 3 | HFrEF LVEF 35% to 50% | 0.808 | 0.841 | (nd) | (nd) | 0.873 |
| 3 | ICMP LVEF <35% | 0.929 | 0.942 | (nd) | (nd) | 0.989 |
| 3 | DCMP LVEF <35% | 0.883 | 0.897 | (nd) | (nd) | 0.986 |
| 3 | HFrEF LVEF <35% | 0.900 | 0.914 | (nd) | (nd) | 0.987 |

TABLE 6
Clinical performance of three selected panels combined with NT-
proBNP using a predefined cut off determined to maximize the
Youden index based on the ID data set in selected subgroups.

| Control | Control | | NT proBNP | | |
|---|---|---|---|---|---|
| | VD Dataset | | Negative | Positive | Specificity |
| | Panel | Negative | 57 | 11 | 87% |
| | number 3 | Positive | 19 | 0 | |
| | | Specificity | 78% | | |
| | Control | | NT proBNP | | |
| | VD Dataset | | Negative | Positive | Specificity |
| | Panel | Negative | 63 | 11 | 87% |
| | number 4 | Positive | 13 | 0 | |
| | | Specificity | 85% | | |
| | Control | | NT proBNP | | |
| | VD Dataset | | Negative | Positive | Specificity |
| | Panel | Negative | 73 | 10 | 87% |
| | number 1 | Positive | 3 | 1 | |
| | | Specificity | 95% | | |
| ALL_CHF | ALL_CHF | | NT proBNP | | |
| | VD Dataset | | Negative | Positive | Sensitivity |
| | Panel | Negative | 24 | 34 | 65% |
| | number 3 | Positive | 48 | 99 | |
| | | Sensitivity | 72% | | |
| | ALL_CHF | | NT proBNP | | |
| | VD Dataset | | Negative | Positive | Sensitivity |
| | Panel | Negative | 31 | 43 | 65% |
| | number 4 | Positive | 41 | 90 | |
| | | Sensitivity | 64% | | |
| | ALL_CHF | | NT proBNP | | |
| | VD Dataset | | Negative | Positive | Sensitivity |
| | Panel | Negative | 45 | 18 | 65% |
| | number 1 | Positive | 27 | 115 | |
| | | Sensitivity | 69% | | |
| ALL_CHF, | ALL_CHF, NYHA I - I/II | | NT proBNP | | |
| NYHA I - I/II | VD Dataset | | Negative | Positive | Sensitivity |
| | Panel | Negative | 17 | 14 | 55% |
| | number 3 | Positive | 25 | 37 | |
| | | Sensitivity | 67% | | |
| | ALL_CHF, NYHA I - I/II | | NT proBNP | | |
| | VD Dataset | | Negative | Positive | Sensitivity |
| | Panel | Negative | 21 | 16 | 55% |
| | number 4 | Positive | 21 | 35 | |
| | | Sensitivity | 60% | | |
| | ALL_CHF, NYHA I - I/II | | NT proBNP | | |
| | VD Dataset | | Negative | Positive | Sensitivity |
| | Panel | Negative | 30 | 11 | 55% |
| | number 1 | Positive | 12 | 40 | |
| | | Sensitivity | 56% | | |
| ALL_CHF, | ALL_CHF, LVEF > 35% | | NT proBNP | | |
| LVEF > 35% | VD Dataset | | Negative | Positive | Sensitivity |
| | Panel | Negative | 24 | 27 | 51% |
| | number 3 | Positive | 47 | 47 | |
| | | Sensitivity | 65% | | |
| | ALL_CHF, LVEF > 35% | | NT proBNP | | |
| | VD Dataset | | Negative | Positive | Sensitivity |
| | Panel | Negative | 31 | 33 | 51% |
| | number 4 | Positive | 40 | 41 | |
| | | Sensitivity | 56% | | |
| | ALL_CHF, LVEF > 35% | | NT proBNP | | |
| | VD Dataset | | Negative | Positive | Sensitivity |
| | Panel | Negative | 45 | 15 | 51% |
| | number 1 | Positive | 26 | 59 | |
| | | Sensitivity | 59% | | |
| HFpEF | HFpEF | | NT proBNP | | |
| | VD Dataset | | Negative | Positive | Sensitivity |
| | Panel | Negative | 18 | 15 | 35% |
| | number 3 | Positive | 30 | 11 | |
| | | Sensitivity | 55% | | |
| | HFpEF | | NT proBNP | | |
| | VD Dataset | | Negative | Positive | Sensitivity |
| | Panel | Negative | 23 | 17 | 35% |
| | number 4 | Positive | 25 | 9 | |
| | | Sensitivity | 46% | | |
| | HFpEF | | NT proBNP | | |
| | VD Dataset | | Negative | Positive | Sensitivity |
| | Panel | Negative | 32 | 11 | 35% |
| | number 1 | Positive | 16 | 15 | |
| | | Sensitivity | 42% | | |
| HFrEF | HFrEF | | NT proBNP | | |
| | VD Dataset | | Negative | Positive | Sensitivity |
| | Panel | Negative | 6 | 19 | 82% |
| | number 3 | Positive | 18 | 88 | |
| | | Sensitivity | 81% | | |
| | HFrEF | | NT proBNP | | |
| | VD Dataset | | Negative | Positive | Sensitivity |
| | Panel | Negative | 8 | 26 | 82% |
| | number 4 | Positive | 16 | 81 | |
| | | Sensitivity | 74% | | |
| | HFrEF | | NT proBNP | | |
| | VD Dataset | | Negative | Positive | Sensitivity |
| | Panel | Negative | 13 | 7 | 82% |
| | number 1 | Positive | 11 | 100 | |
| | | Sensitivity | 85% | | |
| HFrEF, 35% < | HFrEF, 35% < LVEF < 50% | | NT proBNP | | |
| LVEF < 50% | VD Dataset | | Negative | Positive | Sensitivity |
| | Panel | Negative | 6 | 12 | 68% |
| | number 3 | Positive | 17 | 36 | |
| | | Sensitivity | 75% | | |
| | HFrEF, 35% < LVEF < 50% | | NT proBNP | | |
| | VD Dataset | | Negative | Positive | Sensitivity |
| | Panel | Negative | 8 | 16 | 68% |
| | number 4 | Positive | 15 | 32 | |
| | | Sensitivity | 66% | | |
| | HFrEF, 35% < LVEF < 50% | | NT proBNP | | |
| | VD Dataset | | Negative | Positive | Sensitivity |
| | Panel | Negative | 13 | 4 | 68% |
| | number 1 | Positive | 10 | 44 | |

| | |
|---|---|
| Sensitivity | 76% |

TABLE 6A
Clinical performance of Panel 1 combined with NT-proBNP compared to NT-proBNP alone

| | | | Panel 1 combined with NT-proBNP | | | | | | |
|---|---|---|---|---|---|---|---|---|---|
| Subgroup | Cases | Controls | AUC | Sens. | Spec. | LR+ | LR− | PPV | NPV |
| CHF | 205 | 87 | 0.911 | 61.0% | 97.7% | 26.5 | 0.399 | 74.7% | 95.8% |
| ICMP | 65 | 87 | 0.988 | 89.2% | 97.7% | 38.8 | 0.110 | | |
| DCMP | 66 | 87 | 0.937 | 71.2% | 97.7% | 31.0 | 0.295 | | |
| HFrEF | 131 | 87 | 0.966 | 80.2% | 97.7% | 34.9 | 0.203 | 64.7% | 98.9% |
| HFpEF | 74 | 87 | 0.826 | 27.0% | 97.7% | 11.8 | 0.747 | | |
| CHF asymptomatic | 93 | 87 | 0.879 | 51.6% | 97.7% | 22.5 | 0.495 | | |
| ICMP asymptomatic | 24 | 87 | 0.995 | 95.8% | 97.7% | 41.7 | 0.043 | | |
| DCMP asymptomatic | 25 | 87 | 0.887 | 52.0% | 97.7% | 22.6 | 0.491 | | |
| HFrEF asymptomatic | 49 | 87 | 0.950 | 73.5% | 97.7% | 32.0 | 0.272 | | |
| HFpEF asymptomatic | 44 | 87 | 0.810 | 27.3% | 97.7% | 11.9 | 0.744 | | |
| CHF symptomatic | 112 | 87 | 0.938 | 68.8% | 97.7% | 29.9 | 0.320 | | |
| ICMP symptomatic | 41 | 87 | 0.983 | 85.4% | 97.7% | 37.1 | 0.150 | | |
| DCMP symptomatic | 41 | 87 | 0.965 | 82.9% | 97.7% | 36.1 | 0.175 | | |
| HFrEF symptomatic | 82 | 87 | 0.974 | 84.1% | 97.7% | 36.6 | 0.162 | | |
| HFpEF symptomatic | 30 | 87 | 0.846 | 26.7% | 97.7% | 11.6 | 0.751 | | |
| ICMP LVEF 35% | 43 | 87 | 0.980 | 83.7% | 97.7% | 36.4 | 0.167 | | |
| to 50% | | | | | | | | | |
| DCMP LVEF 35% | 28 | 87 | 0.880 | 42.9% | 97.7% | 18.6 | 0.585 | | |
| to 50% | | | | | | | | | |
| HFrEF LVEF 35% | 71 | 87 | 0.942 | 67.6% | 97.7% | 29.4 | 0.332 | | |
| to 50% | | | | | | | | | |
| ICMP LVEF <35% | 22 | 87 | 0.999 | 100.0% | 97.7% | 43.5 | 0.000 | | |
| DCMP LVEF <35% | 38 | 87 | 0.989 | 92.1% | 97.7% | 40.1 | 0.081 | | |
| HFrEF LVEF <35% | 60 | 87 | 0.995 | 95.0% | 97.7% | 41.3 | 0.051 | | |

| | NT-proBNP alone | | | | | | | Assumed |
|---|---|---|---|---|---|---|---|---|
| Subgroup | AUC | Sens | Spec. | LR+ | LR− | PPV | NPV | Prevalence |
| CHF | 0.848 | 64.9% | 87.4% | 5.13 | 0.402 | 36.3% | 95.7% | 10% |
| ICMP | 0.949 | 87.7% | 87.4% | 6.94 | 0.141 | | | |
| DCMP | 0.905 | 75.8% | 87.4% | 5.99 | 0.278 | | | |
| HFrEF | 0.926 | 81.7% | 87.4% | 6.46 | 0.210 | 25.4% | 98.9% | 5% |
| HFpEF | 0.706 | 35.1% | 87.4% | 2.78 | 0.743 | | | |
| CHF asymptomatic | 0.801 | 54.8% | 87.4% | 4.34 | 0.517 | | | |
| ICMP asymptomatic | 0.970 | 95.8% | 87.4% | 7.58 | 0.048 | | | |
| DCMP asymptomatic | 0.845 | 64.0% | 87.4% | 5.06 | 0.412 | | | |
| HFrEF asymptomatic | 0.911 | 79.6% | 87.4% | 6.29 | 0.234 | | | |
| HFpEF asymptomatic | 0.670 | 27.3% | 87.4% | 2.16 | 0.833 | | | |
| CHF symptomatic | 0.890 | 73.2% | 87.4% | 5.79 | 0.307 | | | |
| ICMP symptomatic | 0.936 | 82.9% | 87.4% | 6.56 | 0.195 | | | |
| DCMP symptomatic | 0.944 | 82.9% | 87.4% | 6.56 | 0.195 | | | |
| HFrEF symptomatic | 0.939 | 82.9% | 87.4% | 6.56 | 0.195 | | | |
| HFpEF symptomatic | 0.754 | 46.7% | 87.4% | 3.69 | 0.611 | | | |
| ICMP LVEF 35% | 0.925 | 81.4% | 87.4% | 6.44 | 0.213 | | | |
| to 50% | | | | | | | | |
| DCMP LVEF 35% | 0.792 | 46.4% | 87.4% | 3.67 | 0.613 | | | |
| to 50% | | | | | | | | |
| HFrEF LVEF 35% | 0.873 | 67.6% | 87.4% | 5.35 | 0.371 | | | |
| to 50% | | | | | | | | |
| ICMP LVEF <35% | 0.989 | 100.0% | 87.4% | 7.91 | 0.000 | | | |
| DCMP LVEF <35% | 0.986 | 97.4% | 87.4% | 7.70 | 0.030 | | | |
| HFrEF LVEF <35% | 0.987 | 98.3% | 87.4% | 7.78 | 0.019 | | | |

TABLE 7
Elution gradient and post-column addition for LC-MS/MS

| Elution Gradient | | | | Post-Column Addition | | |
|---|---|---|---|---|---|---|
| Time | Flow Rate | Solvent | Solvent | Time | Flow | Solvent |
| [min] | [μl/min] | A [%] | B [%] | [min] | [μl/min] | C [%] |
| 0.00 | 400 | 100 | 0 | 0.00 | 0 | 100 |
| 0.10 | 400 | 100 | 0 | | | |
| 0.20 | 400 | 100 | 0 | gradient continued | | |
| 0.40 | 500 | 58 | 42 | | | |
| gradient continued | | | | 3.20 | 0 | 100 |
| 3.30 | 500 | 32 | 68 | 3.30 | 200 | 100 |
| 5.00 | 600 | 15 | 85 | gradient continued | | |
| gradient continued | | | | 5.30 | 200 | 100 |
| 5.50 | 600 | 15 | 85 | 5.40 | 0 | 100 |
| 5.70 | 400 | 100 | 0 | gradient continued | | |
| 7.50 | 400 | 100 | 0 | 7.50 | 0 | 100 |

TABLE 8
MRM settings (Q1, first mass transition; Q3, second mass transition; DP, declustering
potential; EP, entrance potential; CE, collision energy; CXP, collision cell exit
potential) and retention time (RT) for each metabolite biomarker. PC5 was scanned
three times with different collision energies, as indicated by the names PC5-40,
PC5-60, and PC5-70.

| | Internal Standard | | | | | | | | |
|---|---|---|---|---|---|---|---|---|---|
| | For Calculation of | Dwell Time | | | | | | | |
| Metabolite Biomarker | Peak Area Ratios | [msec] | Q1 [Da] | Q3 [Da] | DP [V] | EP [V] | CE [V] | CXP [V] | RT [min] |
| Glutamic acid 1 | LPC C17:0 | 10 | 148.0 | 84.1 | 36 | 10 | 21 | 6 | 0.23 |
| Glutamic acid 2 | LPC C17:0 | 10 | 148.0 | 102.1 | 36 | 10 | 15 | 8 | 0.23 |
| LPC C17:0* | n/a | 10 | 510.3 | 184.1 | 85 | 10 | 40 | 15 | 1.22 |
| Cer(d18:1/17:0)* | n/a | 15 | 552.4 | 264.2 | 65 | 10 | 31 | 10 | 2.16 |
| Cer(d18:1/23:0) | Cer(d18:1/17:0) | 15 | 636.5 | 264.2 | 65 | 10 | 31 | 10 | 2.95 |
| Cer(d18:1/24:1) | Cer(d18:1/17:0) | 15 | 648.6 | 264.2 | 65 | 10 | 31 | 10 | 2.83 |
| Cer(d18:2/24:0) | Cer(d18:1/17:0) | 15 | 648.6 | 262.2 | 65 | 10 | 31 | 10 | 2.9 |
| Cer(d16:1/24:0) | Cer(d18:1/17:0) | 15 | 622.6 | 236.2 | 65 | 10 | 31 | 10 | 2.83 |
| Cer(d17:1/24:0) | Cer(d18:1/17:0) | 15 | 636.6 | 250.2 | 65 | 10 | 31 | 10 | 2.95 |
| SM2 | PC 5-40 | 10 | 675.5 | 184.1 | 85 | 10 | 40 | 15 | 1.47 |
| SM3 | PC 5-40 | 10 | 689.5 | 184.1 | 85 | 10 | 40 | 15 | 1.52 |
| SM29 | PC 5-40 | 10 | 715.5 | 184.1 | 85 | 10 | 40 | 15 | 1.57 |
| SM8 | PC 5-40 | 10 | 727.5 | 184.1 | 85 | 10 | 40 | 15 | 1.55 |
| SM9 | PC 5-40 | 10 | 729.5 | 184.1 | 85 | 10 | 40 | 15 | 1.62 |
| SM10 | PC 5-40 | 10 | 731.5 | 184.1 | 85 | 10 | 40 | 15 | 1.7 |
| SM18 | PC 5-40 | 10 | 773.5 | 184.1 | 85 | 10 | 40 | 15 | 1.97 |
| SM21 | PC 5-40 | 10 | 787.5 | 184.1 | 85 | 10 | 40 | 15 | 2.05 |
| SM23 | PC 5-40 | 10 | 799.5 | 184.1 | 85 | 10 | 40 | 15 | 2.02 |
| SM24 | PC 5-40 | 10 | 801.5 | 184.1 | 85 | 10 | 40 | 15 | 2.16 |
| SM5 | PC 5-60 | 10 | 703.5 | 184.1 | 85 | 10 | 60 | 15 | 1.57 |
| SM28 | PC 5-40 | 10 | 815.5 | 184.1 | 85 | 10 | 40 | 15 | 2.27 |
| PC 4 | PC 5-70 | 10 | 758.6 | 184.1 | 85 | 10 | 70 | 15 | 1.72 |
| PC 8 | PC 5-60 | 10 | 786.6 | 184.1 | 85 | 10 | 60 | 15 | 1.88 |
| PC 5-40* | n/a | 10 | 818.60 | 184.1 | 85 | 10 | 40 | 15 | 2.38 |
| PC 5-60* | n/a | 10 | 818.60 | 184.1 | 85 | 10 | 60 | 15 | 2.38 |
| PC 5-70* | n/a | 10 | 818.60 | 184.1 | 85 | 10 | 70 | 15 | 2.38 |
| CE C17:0* | n/a | 10 | 656.7 | 369.3 | 56 | 10 | 22 | 20 | 4.65 |
| CE C18:0 | PC 5-40 | 10 | 670.7 | 369.3 | 56 | 10 | 22 | 20 | 4.72 |
| CE C18:2 | PC 5-40 | 10 | 666.7 | 369.3 | 56 | 10 | 40 | 20 | 4.47 |
| PPP | PC 5-40 | 10 | 824.8 | 551.5 | 75 | 10 | 35 | 10 | 4.61 |
| PPO1 | PC 5-40 | 10 | 850.8 | 577.5 | 75 | 10 | 35 | 10 | 4.64 |
| SPP1 | PC 5-40 | 10 | 852.9 | 579.6 | 75 | 10 | 35 | 10 | 4.74 |
| MMM* | n/a | 10 | 866.8 | 579.6 | 75 | 10 | 35 | 10 | 4.8 |
| SOP2 | PC 5-40 | 10 | 878.9 | 577.6 | 75 | 10 | 35 | 10 | 4.76 |
| SSP2 | PC 5-40 | 10 | 880.9 | 579.6 | 75 | 10 | 35 | 10 | 4.85 |
| OSS2 | PC 5-40 | 10 | 906.9 | 605.4 | 75 | 10 | 35 | 10 | 4.87 |
| SSS | PC 5-40 | 10 | 908.9 | 607.6 | 75 | 10 | 35 | 10 | 4.96 |

*internal standard

TABLE 9
Effects of medication on the sensitivity of Panel 1 and NT-proBNP alone in HFrEF patients.

| | HFrEF | | | HFrEF (35% ≦ EF ≦ 50%) | | | HFrEF (EF <35%) | | |
|---|---|---|---|---|---|---|---|---|---|
| | | NT- | | | NT- | | | NT- | |
| | | proBNP | Lipid Panel | | proBNP | Lipid Panel | | proBNP | Lipid Panel |
| Medication | # Subjects | Sens. [%] | Sens. [%] | # Subjects | Sens. [%] | Sens. [%] | # Subjects | Sens. [%] | Sens. [%] |
| HFrEF* | 371 | 80.3 | 79.5 | 213 | 70.9 | 71.4 | 158 | 93.0 | 90.5 |
| HFrEF receiving | 248 | 80.2 | 81.9 | 152 | 71.1 | 75.0 | 96 | 94.8 | 92.7 |
| statins** | | | | | | | | | |
| HFrEF receiving | 230 | 88.3 | 86.1 | 94 | 80.9 | 78.7 | 136 | 93.4 | 91.2 |
| diuretics** | | | | | | | | | |
| Neither*** | 27 | 59.3 | 55.6 | 23 | 56.5 | 52.2 | not shown; number of subjects <10 | | |
| Statins*** | 74 | 71.6 | 75.7 | 66 | 68.2 | 72.7 | not shown; number of subjects <10 | | |
| Diuretics*** | 55 | 92.7 | 81.8 | 17 | 88.2 | 70.6 | 38 | 94.7 | 86.8 |
| Diuretics and | 97 | 90.7 | 87.6 | 39 | 82.1 | 79.5 | 58 | 96.6 | 93.1 |
| statins*** | | | | | | | | | |

*All HFrEF patients
**HFrEF patients receiving the indicated medication regardless of any additional medication
***HFrEF patients receiving exactly the indicated medication plus beta-blockers and medication affecting the renin-angiotensin-system, but no antidiabetic treatment (untreated diabetic patients not excluded)

TABLE 10
Metabolites constituting a further lipid panel (Panel
No. 300) and their changes in patients with systolic
HF compared to healthy controls (ID cohort).

| Metabolite | Lipid Class | Direction |
|---|---|---|
| Cer d17:1/24:0 | Ceramides | down |
| CE 18:2 | Cholesterylesters | down |
| Linoleic acid (18:2^{Δ9cis, Δ12cis}) | Phosphatidyl- | down |
| from PC | cholines | |
| SM d17:1/24:1 | Sphingomyelins | down |
| Stearic acid (18:0) from TAG | Triglycerides | up |
| Oleic acid (18:1^{Δ9cis}) from | Triglycerides | up |
| TAG | | |

TABLE 12
Mass-spectrometer settings optimized for the absolute
quantification of SM23, PC 4, CE C18:2, and OSS2 (Q1,
first mass transition; Q3, second mass transition; DP,
declustering potential; EP, entrance potential; CE,
collision energy; CXP, collision cell exit potential)
for each metabolite biomarker or standard. PC5 as internal
standard was scanned two times with different collision
energies.

| Biomarker or | Dwell | | | | | | |
|---|---|---|---|---|---|---|---|
| Standard for | Time | Q1 | Q3 | DP | EP | CE | CXP |
| Quantification | [msec] | [Da] | [Da] | [V] | [V] | [V] | [V] |
| SM23 | 10 | 799.5 | 184.1 | 60 | 10 | 40 | 15 |
| SM27** | 10 | 813.5 | 184.1 | 60 | 10 | 40 | 15 |
| PC 4 | 10 | 758.6 | 184.1 | 60 | 10 | 85 | 15 |
| PC 5-40* | 10 | 818.60 | 184.1 | 60 | 10 | 40 | 15 |
| PC 5-70* | 10 | 818.62 | 184.1 | 60 | 10 | 85 | 15 |
| CE C18:2 | 10 | 666.7 | 369.3 | 30 | 10 | 45 | 20 |
| OSS2 | 10 | 906.9 | 605.4 | 30 | 10 | 35 | 10 |

*internal standard,
**external standard for the calibration of SM23

TABLE 12a
Biomarkers and Standards with respective
retention times used for quantification

| Biomarker or | | Internal Standard | Calibration |
|---|---|---|---|
| Standard for | RT | For Calculation of | Curve Used for |
| Quantification | [min] | Peak Area Ratios | Quantification |
| SM23 | 2.02 | PC 5-40 | SM27 |
| PC 4 | 1.72 | PC 5-70 | PC4 |
| CE C18:2 | 4.47 | PC 5-40 | CE 18:2 |
| OSS2 | 4.87 | PC 5-40 | OSS2 |
| SM27 | 2.06 | PC5-40 | SM27 |

TABLE 13
Calibration solutions in MeOH/CH2Cl2 (2/1) for calibration samples preparation

| Concentration | | STD1 | STD2 | STD3 | STD4 | STD5 | STD6 | STD7 |
|---|---|---|---|---|---|---|---|---|
| μg/ml | PC4 | 146.423 | 117.138 | 87.854 | 58.569 | 29.285 | 14.642 | 4.881 |
| | CE 18:2 | 365.760 | 292.608 | 219.456 | 146.304 | 73.152 | 36.576 | 12.192 |
| | SM27 | 12.358 | 9.886 | 7.415 | 4.943 | 2.472 | 1.236 | 0.4119 |
| | OSS2 | 1.974 | 1.5792 | 1.1844 | 0.7896 | 0.3948 | 0.1974 | 0.0658 |
| nmol/ml | PC4 | 193.1542 | 154.5234 | 115.8925 | 77.26169 | 38.63085 | 19.31542 | 6.438475 |
| | CE 18:2 | 563.5053 | 450.8042 | 338.1032 | 225.4021 | 112.7011 | 56.35053 | 18.78351 |
| | SM27 | 15.18735 | 12.14988 | 9.112412 | 6.074941 | 3.037471 | 1.518735 | 0.506245 |
| | OSS2 | 2.219314 | 1.775451 | 1.331588 | 0.887726 | 0.443863 | 0.221931 | 0.073977 |

TABLE 13a
Concentration ratios of the compounds within the calibration
samples/calibration solutions for calibration sample preparation,
e.g. for the determination of PC4, CE18:2, SM23, and OSS2 (Panel 200)

| Ratio (from | | | | | | | |
|---|---|---|---|---|---|---|---|
| molar | | | | | | | |
| concentrations; | | | | | | | |
| CE | | | | | | | |
| C18:2 set to | | | | | | | |
| “1”) | STD1 | STD2 | STD3 | STD4 | STD5 | STD6 | STD7 |
| PC 4 | 0.342773 | 0.274218 | 0.205664 | 0.137109 | 0.068555 | 0.034277 | 0.011426 |
| CE 18:2 | 1 | 0.8 | 0.6 | 0.4 | 0.2 | 0.1 | 0.033333 |
| SM27 | 0.026952 | 0.021561 | 0.016171 | 0.010781 | 0.00539 | 0.002695 | 0.000898 |
| OSS2 | 0.003938 | 0.003151 | 0.002363 | 0.001575 | 0.000788 | 0.000394 | 0.000131 |

TABLE 13b
Concentration ratios of the compounds within the calibration
samples/calibration solutions for calibration sample preparation,
e.g. for the determination of CE18:2, SM23, and OSS2 (Panel 2)

| Ratio (from | | | | | | | |
|---|---|---|---|---|---|---|---|
| molar | | | | | | | |
| concentrations; | | | | | | | |
| CE | | | | | | | |
| C18:2 set to “1”) | STD1 | STD2 | STD3 | STD4 | STD5 | STD6 | STD7 |
| CE 18:2 | 1 | 0.8 | 0.6 | 0.4 | 0.2 | 0.1 | 0.033333 |
| SM27 | 0.026952 | 0.021561 | 0.016171 | 0.010781 | 0.00539 | 0.002695 | 0.000898 |
| OSS2 | 0.003938 | 0.003151 | 0.002363 | 0.001575 | 0.000788 | 0.000394 | 0.000131 |

TABLE 13C
Concentration ratios of the compounds within the calibration
samples/calibration solutions for calibration sample preparation,
e.g. for the determination of PC4, SM23, and OSS2 (Panel 1)

| Ratio (from | | | | | | | |
|---|---|---|---|---|---|---|---|
| molar | | | | | | | |
| concentrations; | | | | | | | |
| PC4 set | | | | | | | |
| to “1”) | STD1 | STD2 | STD3 | STD4 | STD5 | STD6 | STD7 |
| PC 4 | 1 | 0.8 | 0.6 | 0.4 | 0.2 | 0.1 | 0.033333 |
| SM27 | 0.078628 | 0.062902 | 0.047177 | 0.031451 | 0.015726 | 0.007863 | 0.002621 |
| OSS2 | 0.01149 | 0.009192 | 0.006894 | 0.004596 | 0.002298 | 0.001149 | 0.000383 |

TABLE 14
Absolute concentrations of the metabolite biomarkers SM 23, PC4, CE C18:2, and
OSS2 in the CHF subgroups HFrEF and HFpEF as well as in the control subjects

| | Mean* | | | | | | RSD** | | |
|---|---|---|---|---|---|---|---|---|---|
| | Control | | HFrEF | | HFpEF | | Control | HFrEF | HFpEF |
| SM 23 | 1493 | μg/dl | 1138 | μg/dl | 1320 | μg/dl | 19% | 22% | 24% |
| PC4 | 41996 | μg/dl | 35854 | μg/dl | 38006 | μg/dl | 13% | 18% | 18% |
| CE C18:2 | 124300 | μg/dl | 105799 | μg/dl | 115489 | μg/dl | 10% | 16% | 15% |
| OSS2 | 83 | μg/dl | 190 | μg/dl | 154 | μg/dl | 53% | 57% | 60% |

*The arithmetic mean was calculated on log₁₀-transformed data and then back-transformed to the original scale
**The relative standard deviation (RSD) was calculated from the standard deviation (SD) of the log₁₀-transformed data as RSD = 1-10^{-SD}

1. A method for diagnosing heart failure comprising the steps of:
a. determining in a sample of a subject the amounts of at least three biomarkers, wherein said at least three biomarkers are:
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin;
iv. at least one phosphatidylcholine, at least one cholesterylester, and at least one sphingomyelin;
v. Cholesterylester C18:2, SSS and Cer(d17:1/24:0);
vi. at least two sphingomyelins selected from the group consisting of SM2, SM3, SM5, SM18, SM23, SM24, and SM28, and at least one triacylglyceride selected from the group consisting of SOP2, SPP1 or PPO1;
vii. at least two triacylglycerides selected from the group consisting of OSS2, SOP2, SPP1 and SSP2, and at least one sphingomyelin selected from the group consisting of SM23 and SM24;
viii. SM18, SM24 and SM28;
ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199 of Table 2, or
x. the biomarkers of panel 200, 201, 202, 203, 204, 205, or 206 of Table 2a, and
b. comparing the amounts as determined in step a. to a reference, whereby heart failure is to be diagnosed,
wherein the at least one triacylglyceride biomarker in i., ii., and iii. is selected from the group consisting of SOP2, OSS2, SPP1, SSP2, PPO1 and PPP, the at least one cholesterylester biomarker in i., iii., and iv. is selected from the group consisting of cholesterylester C18:2 and cholesterylester C18:0, the at least one phosphatidylcholine biomarker in i., ii. and iv. is selected from the group consisting of PC4 and PC8, and the at least one sphingomyelin biomarker in ii., iii., and iv. is selected from the group consisting of SM18, SM24, SM23, SM21, SM28, SM5, SM3, SM29 and SM8.

2. The method of claim 1, wherein the heart failure is heart failure with reduced left ventricular ejection fraction (HFrEF).

3. The method of claims 1 and 2, wherein at least the amounts of the biomarkers of i., ii., and iii. of claim 1 are determined, and wherein the at least one triacylglyceride biomarker in i., ii., and iii. is selected from the group consisting of SOP2, OSS2, SSP2, PPO1 and PPP, in particular selected from the group consisting of SOP2, OSS2, SSP2, and PPO1, and the least one cholesterylester biomarker in i., and iii. is cholesterylester C18:2, and the least one phosphatidylcholine biomarker in i. and ii. is PC4, and the least one sphingomyelin biomarker in ii. and iii. is selected from the group consisting of SM18, SM24, SM23, SM28, SM5, and SM3.

4. The method of any one of claims 1 to 3, wherein at least the amounts of the biomarkers of i., ii., and iii. are determined, and wherein the at least one triacylglyceride biomarker in i., ii., and iii. is SOP2 and/or OSS2, and wherein the at least one cholesterylester biomarker in i., and iii. is cholesterylester C18:2, and wherein the at least one phosphatidylcholine biomarker in i. and ii. is PC4, and wherein the at least one sphingomyelin biomarker in ii. and iii. is selected from the group consisting of SM18, SM24, SM23, and SM3, in particular
wherein at least the amounts of the biomarkers of i., ii., or iii are determined, and wherein the at least one triacylglyceride in i., ii., and iii. is SOP2 and/or OSS2, and wherein the at least one cholesterylester in i., and iii. is cholesterylester C18:2, and wherein the at least one phosphatidylcholine in i. and ii. is PC4, and wherein the at least one sphingomyelin in ii. and iii. is SM23.

5. The method of any one of claims 1 to 4, wherein at least the amounts of OSS2, PC4 and SM23 are determined (panel 1) or wherein at least the amounts of OSS2, cholesterylester C18:2 and SM23 are determined (panel 2, or panel 4).

6. The method of any one of claims 1 to 5, wherein at least the amounts of the biomarkers of iii. are determined, and wherein the at least one triacylglyceride biomarker is SOP2 and/or OSS2, and wherein the at least one cholesterylester biomarker is cholesterylester C18:2, and wherein the at least one sphingomyelin biomarker is SM23.

7. The method of any one of claims 1 to 6, wherein at least the amounts of SOP2, OSS2, PC4, Cholesterylester C18:2, SM18, SM28, SM24, SSP2, and SM23 are determined (panel 3).

8. The method of any one of claims 2 to 7, wherein the subject does not show symptoms of heart failure.

9. The method of claim 8, wherein at least the amounts of the biomarkers of panel 7, 8, 9, 11, or 12 in Table 2 are determined.

10. The method of any of claims 2 to 9, wherein HFrEF is DCMP or ICMP.

11. The method of any one of claims 1 to 10, wherein the subject is a human subject, and/or wherein the sample is blood, serum or plasma.

12. The method of any one of claims 1 to 11, wherein the amounts of the at least three biomarkers are determined by mass spectrometry (MS).

13. The method according to any one of claims 1 to 12, wherein the HFrEF is heart failure with a left ventricular ejection fraction of lower than 50% but larger than 35%.

14. The method according to any one of claims 1 to 13, wherein the subject is overweight.

15. The method according to any one of claims 1 to 13, wherein the subject is older than 54 years of age.

16. The method according to any one of claims 1 to 13, wherein the subject is a female subject, older than 54 years of age, and has a body mass index of less than 25.0 kg/m².

17. The method according to any one of claims 1 to 13, wherein the subject is younger than 61 years of age, and has a body mass index of more than 26.8 kg/m².

18. The method according to any one of claims 1 to 17, wherein the amounts of OSS2, PC4 and SM23 (panel 1) are determined.

19. The method of any one of claims 1 to 18, further comprising the determination of the amount of NT-proBNP, BNP, ANP, or NT-proANP in step a).

20. A diagnostic device for carrying out the method according to any one of claims 1 to 19, comprising:
a) an analysing unit comprising at least one detector for at least three biomarkers as set forth in any one of claims 1 to 9 and 18, wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the at least three biomarkers, and reference amounts and a data base comprising said reference amounts for the said biomarkers, whereby it will be diagnosed whether a subject suffers from heart failure.

21. Use of at least three biomarkers as set forth in any one of claims 1 to 9 and 18 in a sample of a subject for diagnosing heart failure or for the preparation of a pharmaceutical and/or diagnostic composition for diagnosing heart failure.
